(19) United States
(12) Patent Application Publication
(10) Pub. No.: US 2014/0294817 A1
(43) Pub. Date: Oct. 2, 2014

(54) POLYPEPTIDES COMPRISING Fc FRAGMENTS OF IMMUNOGLOBULIN G (IgG) AND METHODS OF USING THE SAME

(71) Applicants: LeukoSight, Inc., College Park, MD (US); University of Maryland, College Park, MD (US)

(72) Inventors: David M. Mosser, Hyattsville, MD (US); Shanjin Cao, Bronx, NY (US)

(21) Appl. No.: 14/308,112

(22) Filed: Jun. 18, 2014

Related U.S. Application Data
(63) Continuation of application No. 12/629,136, filed on Dec. 2, 2009.
(60) Provisional application No. 61/119,858, filed on Dec. 4, 2008.

(51) Int. Cl. C07K 16/24

(52) U.S. Cl. CPC C07K 16/244
USPC 424/133.1

(57) ABSTRACT

Polypeptides comprising at least a first and second Fc fragment of IgG that can be used to induce a stimulated cell to produce the anti-inflammatory cytokine Interleukin-10 and methods of using the same are disclosed herein.

### CROSS REFERENCE TO RELATED APPLICATIONS

The application claims the benefit of priority to U.S. Provisional Patent Application 61/119,858, filed Dec. 4, 2008, the disclosure of the entirety of which is incorporated herein by reference.

### REFERENCE TO SEQUENCE LISTING

This application contains a Sequence Listing in accordance with 37 C.F.R. §§1.821-1.825. The material in the Sequence Listing text file is herein incorporated by reference in its entirety in accordance with 37 C.F.R. §1.52(e)(5). The electronically submitted Sequence Listing, entitled “080619 Sequence Listing_ST25.txt” contains one 337 Kb text file and was created on Oct. 7, 2009 using an IBM-PC machine format.

### TECHNICAL FIELD

The present invention relates to polypeptides comprising Fc fragments of immunoglobulin G (IgG) and methods of using the same, for example, as an anti-inflammatory agent for treating inflammatory conditions or as a laboratory reagent.

### BACKGROUND

Leukocytes are cells in the immune system that defend the body against both infectious disease and foreign material. In response to infection or inflammatory stimuli, leukocytes produce proinflammatory cytokines, such as interleukin (IL)-12, Tumor Necrosis Factor-alpha (TNF-α), IL-1, IL-6, and IL-8.

Interleukin-10 (IL-10), an anti-inflammatory cytokine also produced by leukocytes, is used to regulate an inflammatory response. For example, IL-10 has been shown to inhibit proinflammatory cytokine production by leukocytes, particularly IL-12 production in macrophages (Sutterwala et al., J. Experimental Medicine 185:1977-1985, 1997). Furthermore, IL-10 has also been tested as a treatment for various autoimmune diseases including arthritis (Hart et al. Immunology 84: 536-542, 1995) and colitis (Davidson et al., J. Experimental Medicine 184: 241-251, 1996).

The Fc-gamma receptor (FcγR) is a receptor located on the surface of leukocytes, which specifically binds the Fc region of IgG.

An immune complex is an antigen with multiple IgG's attached, which allow for the immune complex to bind to the FcγR via the Fc region of the various IgG molecules. Previous research has demonstrated that immune complexes could induce stimulated leukocytes to produce high levels of IL-10 (Sutterwala et al., J. Experimental Medicine 185:1977-1985, 1997).

Despite the potential for using immune complexes for therapeutic treatment, these immune complexes are large and heterogeneous consisting of several IgG molecules, thus, it is difficult to control size and valency of the immune complexes. Therefore, these large immune complexes would not be appropriate for therapeutic use in humans because they become lodged in tissue and cause tissue pathology/toxicity.

Thus, there is a need for small recombinant polypeptides that can ligate and cross-link the FcγR on stimulated leukocytes to produce IL-10 without causing toxicity.

### BRIEF SUMMARY

Disclosed herein are various non-limiting embodiments generally related to polypeptides comprising at least a first and second Fc fragment of IgG. The first and second Fc fragments are cloned so that they may be attached to one another in a tandem series.

In one embodiment, the present disclosure provides a polypeptide comprising at least a first and second Fc fragment of IgG. The at least one first Fc fragment of IgG may comprise at least one C_{H}2 domain and at least one hinge region and the first and second Fc fragments of IgG may be bound through at least one hinge region.

In another embodiment, the present disclosure provides a polypeptide as set forth herein, wherein the first and second Fc fragments of IgG form a chain and the polypeptide further comprises multiple substantially similar chains bound to at least one other of said multiple chains in a substantially parallel relationship. The chains may form a dimer or a multimer.

In another embodiment, the present disclosure provides a polypeptide as set forth herein, wherein the polypeptide is configured to bind and cross-link at least two FcγRs on a stimulated cell thereby inducing the stimulated cell to produce an anti-inflammatory cytokine interleukin-10 upon binding and cross-linking the at least two FcγRs.

The polypeptides comprising at least a first and second Fc fragment of IgG have several uses, including, but not limited to, use as an anti-inflammatory agent for treating conditions that have inflammation as one of the symptoms or as a laboratory reagent.

It should be understood that this invention is not limited to the embodiments disclosed in the summary, and it is intended to cover modifications that are within the spirit and scope of the invention, as defined by the claims.

### BRIEF DESCRIPTION OF FIGURES

The foregoing summary, as well as the following detailed description, will be better understood when read in conjunction with the appended figures. In the figures:

FIG. 1A shows a diagram of the various gene sequences of the first Fc fragment of IgG.

FIG. 1B shows a diagram of the various gene sequences of the first and second Fc fragment of IgG.

FIGS. 1C-D show a schematic diagram of the construction of a polypeptide comprising a first and second Fc fragment of IgG in monomeric (FIG. 1C) and dimeric form (FIG. 1D). Hinge regions are indicated by open circles. C_{H}2 and C_{H}3 domains are indicated by squares.

FIGS. 2A-B show the cDNA sequence for a polypeptide comprising a first and second Fc fragment of rabbit IgG. The first and second Fc fragments of rabbit IgG comprise one hinge region, one C_{H}2 domain, and one C_{H}3 domain (SEQ ID NO: 1).

FIG. 2C shows the protein sequence for a polypeptide comprising a first and second Fc fragment of rabbit IgG. The first and second Fc fragments of rabbit IgG comprise one hinge region, one C_{H}2 domain, and one C_{H}3 domain (SEQ ID NO: 2).

FIGS. 2D-E shows the cDNA sequence for a polypeptide comprising a first and second Fc fragment of rabbit IgG further comprising extra nucleotides that encode five tyrosine for nanoparticle binding (SEQ ID NO: 3).

FIG. 2F shows a protein sequence for a polypeptide comprising a first and second Fc fragment of rabbit IgG further comprising five tyrosine for nanoparticle binding (SEQ ID NO: 4).

FIG. 3 shows a diagram of sixteen different murine BALB/c polypeptides comprising first and second Fc fragments of murine BALB/c IgG in dimeric form.

FIG. 4A shows the cDNA sequence for a polypeptide comprising a first Fc fragment of murine BALB/c IgG1 and second Fc fragment of murine BALB/c IgG1 (SEQ ID NO: 5).

FIG. 4B shows the protein sequence for a polypeptide comprising a first Fc fragment of murine BALB/c IgG1 and second Fc fragment of murine BALB/c IgG1 (SEQ ID NO: 6).

FIG. 5A shows the cDNA sequence for a polypeptide comprising a first Fc fragment of murine BALB/c IgG2a and second Fc fragment of murine BALB/c IgG1 (SEQ ID NO: 7).

FIG. 5B shows the protein sequence for a polypeptide comprising a first Fc fragment of murine BALB/c IgG2a and second Fc fragment of murine BALB/c IgG1 (SEQ ID NO: 8).

FIG. 6A shows the cDNA sequence for a polypeptide comprising a first Fc fragment of murine BALB/c IgG2b and second Fc fragment of murine BALB/c IgG1 (SEQ ID NO: 9).

FIG. 6B shows the protein sequence for a polypeptide comprising a first Fc fragment of murine BALB/c IgG2b and second Fc fragment of murine BALB/c IgG1 (SEQ ID NO: 10).

FIG. 7A shows the cDNA sequence for a polypeptide comprising a first Fc fragment of murine BALB/c IgG3 and second Fc fragment of murine BALB/c IgG1 (SEQ ID NO: 11).

FIG. 7B shows the protein sequence for a polypeptide comprising a first Fc fragment of murine BALB/c IgG3 and second Fc fragment of murine BALB/c IgG1 (SEQ ID NO: 12).

FIG. 8A shows the cDNA sequence for a polypeptide comprising a first Fc fragment of murine BALB/c IgG1 and second Fc fragment of murine BALB/c IgG2a (SEQ ID NO: 13).

FIG. 8B shows the protein sequence for a polypeptide comprising a first Fc fragment of murine BALB/c IgG1 and second Fc fragment of murine BALB/c IgG2a (SEQ ID NO: 14).

FIGS. 9A-B shows the cDNA sequence for a polypeptide comprising a first Fc fragment of murine BALB/c IgG2a and second Fc fragment of murine BALB/c IgG2a (SEQ ID NO: 15).

FIG. 9C shows the protein sequence for a polypeptide comprising a first Fc fragment of murine BALB/c IgG2a and second Fc fragment of murine BALB/c IgG2a (SEQ ID NO: 16).

FIGS. 10A-B shows the cDNA sequence for a polypeptide comprising a first Fc fragment of murine BALB/c IgG2b and second Fc fragment of murine BALB/c IgG2a (SEQ ID NO: 17).

FIG. 10C shows the protein sequence for a polypeptide comprising a first Fc fragment of murine BALB/c IgG2b and second Fc fragment of murine BALB/c IgG2a (SEQ ID NO: 18).

FIGS. 11A-B shows the cDNA sequence for a polypeptide comprising a first Fc fragment of murine BALB/c IgG3 and second Fc fragment of murine BALB/c IgG2a (SEQ ID NO: 19).

FIG. 11C shows the protein sequence for a polypeptide comprising a first Fc fragment of murine BALB/c IgG3 and second Fc fragment of murine BALB/c IgG2a (SEQ ID NO: 20).

FIGS. 12A-B shows the cDNA sequence for a polypeptide comprising a first Fc fragment of murine BALB/c IgG1 and second Fc fragment of murine BALB/c IgG2b (SEQ ID NO: 21).

FIG. 12C shows the protein sequence for a polypeptide comprising a first Fc fragment of murine BALB/c IgG1 and second Fc fragment of murine BALB/c IgG2b (SEQ ID NO: 22).

FIGS. 13A-B shows the cDNA sequence for a polypeptide comprising a first Fc fragment of murine BALB/c IgG2a and second Fc fragment of murine BALB/c IgG2b (SEQ ID NO: 23).

FIG. 13C shows the protein sequence for a polypeptide comprising a first Fc fragment of murine BALB/c IgG2a and second Fc fragment of murine BALB/c IgG2b (SEQ ID NO: 24).

FIGS. 14A-B shows the cDNA sequence for a polypeptide comprising a first Fc fragment of murine BALB/c IgG2b and second Fc fragment of murine BALB/c IgG2b (SEQ ID NO: 25).

FIG. 14C shows the protein sequence for a polypeptide comprising a first Fc fragment of murine BALB/c IgG2b and second Fc fragment of murine BALB/c IgG2b (SEQ ID NO: 26).

FIGS. 15A-B shows the cDNA sequence for a polypeptide comprising a first Fc fragment of murine BALB/c IgG3 and second Fc fragment of murine BALB/c IgG2b (SEQ ID NO: 27).

FIG. 15C shows the protein sequence for a polypeptide comprising a first Fc fragment of murine BALB/c IgG3 and second Fc fragment of murine BALB/c IgG2b (SEQ ID NO: 28).

FIGS. 16A-B shows the cDNA sequence for a polypeptide comprising a first Fc fragment of murine BALB/c IgG1 and second Fc fragment of murine BALB/c IgG3 (SEQ ID NO: 29).

FIG. 16C shows the protein sequence for a polypeptide comprising a first Fc fragment of murine BALB/c IgG1 and second Fc fragment of murine BALB/c IgG3 (SEQ ID NO: 30).

FIGS. 17A-B shows the cDNA sequence for a polypeptide comprising a first Fc fragment of murine BALB/c IgG2a and second Fc fragment of murine BALB/c IgG3 (SEQ ID NO: 31).

FIG. 17C shows the protein sequence for a polypeptide comprising a first Fc fragment of murine BALB/c IgG2a and second Fc fragment of murine BALB/c IgG3 (SEQ ID NO: 32).

FIGS. 18A-B shows the cDNA sequence for a polypeptide comprising a first Fc fragment of murine BALB/c IgG2b and second Fc fragment of murine BALB/c IgG3 (SEQ ID NO: 33).

FIG. 18C shows the protein sequence for a polypeptide comprising a first Fc fragment of murine BALB/c IgG2b and second Fc fragment of murine BALB/c IgG3 (SEQ ID NO: 34).

FIGS. 19A-B shows the cDNA sequence for a polypeptide comprising a first Fc fragment of murine BALB/c IgG3 and second Fc fragment of murine BALB/c IgG3 (SEQ ID NO: 35).

FIG. 19C shows the protein sequence for a polypeptide comprising a first Fc fragment of murine BALB/c IgG3 and second Fc fragment of murine BALB/c IgG3 (SEQ ID NO: 36).

FIG. 20 shows a diagram of sixteen different murine C57BL/6 polypeptides comprising first and second Fc fragments of murine C57BL/6 IgG in dimeric form.

FIGS. 21A-B shows the cDNA sequence for a polypeptide comprising a first Fc fragment of murine C57BL/6 IgG1 and second Fc fragment of murine C57BL/6 IgG1 (SEQ ID NO: 37).

FIG. 21C shows the protein sequence for a polypeptide comprising a first Fc fragment of murine C57BL/6 IgG1 and second Fc fragment of murine C57BL/6 IgG1 (SEQ ID NO: 38).

FIGS. 22A-B shows the cDNA sequence for a polypeptide comprising a first Fc fragment of murine C57BL/6 IgG2b and second Fc fragment of murine C57BL/6 IgG1 (SEQ ID NO: 39).

FIG. 22C shows the protein sequence for a polypeptide comprising a first Fc fragment of murine C57BL/6 IgG2b and second Fc fragment of murine C57BL/6 IgG1 (SEQ ID NO: 40).

FIGS. 23A-B shows the cDNA sequence for a polypeptide comprising a first Fc fragment of murine C57BL/6 IgG2c and second Fc fragment of murine C57BL/6 IgG1 (SEQ ID NO: 41).

FIG. 23C shows the protein sequence for a polypeptide comprising a first Fc fragment of murine C57BL/6 IgG2c and second Fc fragment of murine C57BL/6 IgG1 (SEQ ID NO: 42).

FIGS. 24A-B shows the cDNA sequence for a polypeptide comprising a first Fc fragment of murine C57BL/6 IgG3 and second Fc fragment of murine C57BL/6 IgG1 (SEQ ID NO: 43).

FIG. 24C shows the protein sequence for a polypeptide comprising a first Fc fragment of murine C57BL/6 IgG3 and second Fc fragment of murine C57BL/6 IgG1 (SEQ ID NO: 44).

FIGS. 25A-B shows the cDNA sequence for a polypeptide comprising a first Fc fragment of murine C57BL/6 IgG1 and second Fc fragment of murine C57BL/6 IgG2b (SEQ ID NO: 45).

FIG. 25C shows the protein sequence for a polypeptide comprising a first Fc fragment of murine C57BL/6 IgG1 and second Fc fragment of murine C57BL/6 IgG2b (SEQ ID NO: 46).

FIGS. 26A-B shows the cDNA sequence for a polypeptide comprising a first Fc fragment of murine C57BL/6 IgG2b and second Fc fragment of murine C57BL/6 IgG2b (SEQ ID NO: 47).

FIG. 26C shows the protein sequence for a polypeptide comprising a first Fc fragment of murine C57BL/6 IgG2b and second Fc fragment of murine C57BL/6 IgG2b (SEQ ID NO: 48).

FIGS. 27A-B shows the cDNA sequence for a polypeptide comprising a first Fc fragment of murine C57BL/6 IgG2c and second Fc fragment of murine C57BL/6 IgG2b (SEQ ID NO: 49).

FIG. 27C shows the protein sequence for a polypeptide comprising a first Fc fragment of murine C57BL/6 IgG2c and second Fc fragment of murine C57BL/6 IgG2b (SEQ ID NO: 50).

FIGS. 28A-B shows the cDNA sequence for a polypeptide comprising a first Fc fragment of murine C57BL/6 IgG3 and second Fc fragment of murine C57BL/6 IgG2b (SEQ ID NO: 51).

FIG. 28C shows the protein sequence for a polypeptide comprising a first Fc fragment of murine C57BL/6 IgG3 and second Fc fragment of murine C57BL/6 IgG2b (SEQ ID NO: 52).

FIGS. 29A-B shows the cDNA sequence for a polypeptide comprising a first Fc fragment of murine C57BL/6 IgG1 and second Fc fragment of murine C57BL/6 IgG2c (SEQ ID NO: 53).

FIG. 29C shows the protein sequence for a polypeptide comprising a first Fc fragment of murine C57BL/6 IgG1 and second Fc fragment of murine C57BL/6 IgG2c (SEQ ID NO: 54).

FIGS. 30A-B shows the cDNA sequence for a polypeptide comprising a first Fc fragment of murine C57BL/6 IgG2b and second Fc fragment of murine C57BL/6 IgG2c (SEQ ID NO: 55).

FIG. 30C shows the protein sequence for a polypeptide comprising a first Fc fragment of murine C57BL/6 IgG2b and second Fc fragment of murine C57BL/6 IgG2c (SEQ ID NO: 56).

FIG. 31A shows the cDNA sequence for a polypeptide comprising a first Fc fragment of murine C57BL/6 IgG2c and second Fc fragment of murine C57BL/6 IgG2c (SEQ ID NO: 57).

FIG. 31B shows the protein sequence for a polypeptide comprising a first Fc fragment of murine C57BL/6 IgG2c and second Fc fragment of murine C57BL/6 IgG2c (SEQ ID NO: 58).

FIGS. 32A-B shows the cDNA sequence for a polypeptide comprising a first Fc fragment of murine C57BL/6 IgG3 and second Fc fragment of murine C57BL/6 IgG2c (SEQ ID NO: 59).

FIG. 32C shows the protein sequence for a polypeptide comprising a first Fc fragment of murine C57BL/6 IgG3 and second Fc fragment of murine C57BL/6 IgG2c (SEQ ID NO: 60).

FIGS. 33A-B shows the cDNA sequence for a polypeptide comprising a first Fc fragment of murine C57BL/6 IgG1 and second Fc fragment of murine C57BL/6 IgG3 (SEQ ID NO: 61).

FIG. 33C shows the protein sequence for a polypeptide comprising a first Fc fragment of murine C57BL/6 IgG1 and second Fc fragment of murine C57BL/6 IgG3 (SEQ ID NO: 62).

FIGS. 34A-B shows the cDNA sequence for a polypeptide comprising a first Fc fragment of murine C57BL/6 IgG2b and second Fc fragment of murine C57BL/6 IgG3 (SEQ ID NO: 63).

FIG. 34C shows the protein sequence for a polypeptide comprising a first Fc fragment of murine C57BL/6 IgG2b and second Fc fragment of murine C57BL/6 IgG3 (SEQ ID NO: 64).

FIGS. 35A-B shows the cDNA sequence for a polypeptide comprising a first Fc fragment of murine C57BL/6 IgG2c and second Fc fragment of murine C57BL/6 IgG3 (SEQ ID NO: 65).

FIG. 35C shows the protein sequence for a polypeptide comprising a first Fc fragment of murine C57BL/6 IgG2c and second Fc fragment of murine C57BL/6 IgG3 (SEQ ID NO: 66).

FIGS. 36A-B shows the cDNA sequence for a polypeptide comprising a first Fc fragment of murine C57BL/6 IgG3 and second Fc fragment of murine C57BL/6 IgG3 (SEQ ID NO: 67).

FIG. 36C shows the protein sequence for a polypeptide comprising a first Fc fragment of murine C57BL/6 IgG3 and second Fc fragment of murine C57BL/6 IgG3 (SEQ ID NO: 68).

FIG. 37 shows a diagram of ten different human polypeptides comprising first and second Fc fragments of human IgG in dimeric form.

FIG. 38A shows the cDNA sequence for a polypeptide comprising a first Fc fragment of human IgG1 and second Fc fragment of human IgG1 (SEQ ID NO: 69).

FIG. 38B shows the protein sequence for a polypeptide comprising a first Fc fragment of Human IgG1 and second Fc fragment of Human IgG1 (SEQ ID NO: 70).

FIG. 39A shows the cDNA sequence for a polypeptide comprising a first Fc fragment of Human IgG2 and second Fc fragment of Human IgG1 (SEQ ID NO: 71).

FIG. 39B shows the protein sequence for a polypeptide comprising a first Fc fragment of Human IgG2 and second Fc fragment of Human IgG1 (SEQ ID NO: 72).

FIG. 40A shows the cDNA sequence for a polypeptide comprising a first Fc fragment of Human IgG3 and second Fc fragment of Human IgG1 (SEQ ID NO: 73).

FIG. 40B shows the protein sequence for a polypeptide comprising a first Fc fragment of Human IgG3 and second Fc fragment of Human IgG1 (SEQ ID NO: 74).

FIG. 41A shows the cDNA sequence for a polypeptide comprising a first Fc fragment of Human IgG4 and second Fc fragment of Human IgG1 (SEQ ID NO: 75).

FIG. 41B shows the protein sequence for a polypeptide comprising a first Fc fragment of Human IgG4 and second Fc fragment of Human IgG1 (SEQ ID NO: 76).

FIG. 42A shows the cDNA sequence for a polypeptide comprising a first Fc fragment of Human IgG1 and second Fc fragment of Human IgG2 (SEQ ID NO: 77).

FIG. 42B shows the protein sequence for a polypeptide comprising a first Fc fragment of Human IgG1 and second Fc fragment of Human IgG2 (SEQ ID NO: 78).

FIG. 43A shows the cDNA sequence for a polypeptide comprising a first Fc fragment of Human IgG2 and second Fc fragment of Human IgG2 (SEQ ID NO: 79).

FIG. 43B shows the protein sequence for a polypeptide comprising a first Fc fragment of Human IgG2 and second Fc fragment of Human IgG2 (SEQ ID NO: 80).

FIG. 44A shows the cDNA sequence for a polypeptide comprising a first Fc fragment of Human IgG3 and second Fc fragment of Human IgG2 (SEQ ID NO: 81).

FIG. 44B shows the protein sequence for a polypeptide comprising a first Fc fragment of Human IgG3 and second Fc fragment of Human IgG2 (SEQ ID NO: 82).

FIG. 45A shows the cDNA sequence for a polypeptide comprising a first Fc fragment of Human IgG4 and second Fc fragment of Human IgG2 (SEQ ID NO: 83).

FIG. 45B shows the protein sequence for a polypeptide comprising a first Fc fragment of Human IgG4 and second Fc fragment of Human IgG2 (SEQ ID NO: 84).

FIG. 46A shows the cDNA sequence for a polypeptide comprising a first Fc fragment of Human IgG1 and second Fc fragment of Human IgG3 (SEQ ID NO: 85).

FIG. 46B shows the protein sequence for a polypeptide comprising a first Fc fragment of Human IgG1 and second Fc fragment of Human IgG3 (SEQ ID NO: 86).

FIG. 47A shows the cDNA sequence for a polypeptide comprising a first Fc fragment of Human IgG2 and second Fc fragment of Human IgG3 (SEQ ID NO: 87).

FIG. 47B shows the protein sequence for a polypeptide comprising a first Fc fragment of Human IgG2 and second Fc fragment of Human IgG3 (SEQ ID NO: 88).

FIG. 48A shows the cDNA sequence for a polypeptide comprising a first Fc fragment of Human IgG3 and second Fc fragment of Human IgG3 (SEQ ID NO: 89).

FIG. 48B shows the protein sequence for a polypeptide comprising a first Fc fragment of Human IgG3 and second Fc fragment of Human IgG3 (SEQ ID NO: 90).

FIG. 49A shows the cDNA sequence for a polypeptide comprising a first Fc fragment of Human IgG4 and second Fc fragment of Human IgG3 (SEQ ID NO: 91).

FIG. 49B shows the protein sequence for a polypeptide comprising a first Fc fragment of Human IgG4 and second Fc fragment of Human IgG3 (SEQ ID NO: 92).

FIG. 50A shows the cDNA sequence for a polypeptide comprising a first Fc fragment of Human IgG1 and second Fc fragment of Human IgG4 (SEQ ID NO:93).

FIG. 50B shows the protein sequence for a polypeptide comprising a first Fc fragment of Human IgG1 and second Fc fragment of Human IgG4 (SEQ ID NO: 94).

FIG. 51A shows the cDNA sequence for a polypeptide comprising a first Fc fragment of Human IgG2 and second Fc fragment of Human IgG4 (SEQ ID NO: 95).

FIG. 51B shows the protein sequence for a polypeptide comprising a first Fc fragment of Human IgG2 and second Fc fragment of Human IgG4 (SEQ ID NO: 96).

FIG. 52A shows the cDNA sequence for a polypeptide comprising a first Fc fragment of Human IgG3 and second Fc fragment of Human IgG4 (SEQ ID NO: 97).

FIG. 52B shows the protein sequence for a polypeptide comprising a first Fc fragment of Human IgG3 and second Fc fragment of Human IgG4 (SEQ ID NO: 98).

FIG. 53A shows the cDNA sequence for a polypeptide comprising a first Fc fragment of Human IgG4 and second Fc fragment of Human IgG4 (SEQ ID NO: 99).

FIG. 53B shows the protein sequence for a polypeptide comprising a first Fc fragment of Human IgG4 and second Fc fragment of Human IgG4 (SEQ ID NO: 100).

FIG. 54A shows the secretion of polypeptides comprising a first and second Fc fragment of rabbit IgG from transfected HeLa cells.

FIG. 54B shows a western blot of polypeptides comprising a first and second Fc fragment of rabbit IgG in monomeric and dimeric form. The polypeptides were present in supernatants from HeLa cells transfected with a pFuse vector comprising a first and second Fc fragment of rabbit IgG cDNA.

FIG. 55A shows the binding of polypeptides comprising a first and second Fc fragment of rabbit IgG in dimeric form to macrophages.

FIG. 55B shows flow cytometry analysis of polypeptides comprising a first and second fragment of rabbit IgG in dimeric form bound to F4/80+macrophages.

FIG. 56A depicts a HeLa cell transfected with a plasmid that includes an FcγR gene (i.e., FcγRI, FcγRIIb, FcγRIII, or FcγRIV). A red fluorescent protein tag (RFP) is attached to the intracellular portion of the FcγR to identify the FcγR transfected cells via fluorescence detection.

FIG. 56B shows the binding of polypeptides comprising a first and second Fc fragment of rabbit IgG in dimeric form to FcγRI.

FIG. 56C shows the binding of polypeptides comprising a first and second Fc fragment of rabbit IgG in dimeric form to FcγRIIb.

FIG. 56D shows the binding of polypeptides comprising a first and second Fc fragment of rabbit IgG in dimeric form to FcγRIII.

FIG. 56E shows the binding of polypeptides comprising a first and second Fc fragment of rabbit IgG in dimeric form to FcγRIV.

FIG. 57A shows the induction of IL-10 (left panel) and inhibition of IL-12p40 (right panel) by polypeptides comprising a first and second Fc fragment of rabbit IgG in dimeric form.

FIG. 57B shows the decrease in TNFα production by cells exposed to supernatants of macrophages treated with Lipopolysaccharide (LPS) and polypeptides comprising a first and second Fc fragment of rabbit IgG in dimeric form.

FIGS. 58A-B show the induction of IL-10 (FIG. 58A) and inhibition of IL-12p40 (FIG. 58B) by sixteen different murine BALB/c polypeptides comprising first and second Fc fragments of murine BALB/c IgG in dimeric form. The first and second Fc fragments of murine BALB/c IgG may comprise murine BALB/c IgG1, IgG2a, IgG2b, IgG3, and any combinations thereof.

FIG. 59A shows a decrease in IL-10 production in cells from FcγR 7-chain knockout (KO) mice that are treated with LPS and polypeptides comprising a first and second Fc fragment of rabbit IgG in dimeric form compared to the IL-10 production in cells from wild type mice that are treated with LPS and polypeptides comprising a first and second Fc fragment of rabbit IgG in dimeric form.

FIG. 59B shows a similar level of IL-12 production in cells from FcγR γ-chain knockout (KO) mice that are treated with LPS and polypeptides comprising a first and second Fc fragment of rabbit IgG in dimeric form compared to IL-12 production in cells from wild type mice that are treated with LPS and polypeptides comprising a first and second Fc fragment of rabbit IgG in dimeric form.

FIG. 60 shows the protection of mice against experimentally induced Immune Thrombocytopenic Purpural (ITP) by using polypeptides comprising a first and second Fc fragment of rabbit IgG in dimeric form.

FIG. 61A shows Saturation Binding Curves, which demonstrate an enhanced binding of polypeptides comprising a first and second Fc fragment of rabbit IgG in dimeric form to FcγRI on cells compared to the binding of rabbit IgG to FcγRI on cells.

FIG. 61B shows Saturation Binding Curves, which demonstrate an enhanced binding of polypeptides comprising a first and second Fc fragment of rabbit IgG in dimeric form to FcγRIIb on cells compared to the binding of rabbit IgG to FcγRIIb on cells.

FIG. 61C shows Saturation Binding Curves, which demonstrate an enhanced binding of polypeptides comprising a first and second Fc fragment of rabbit IgG in dimeric form to FcγRIII on cells compared to the binding of rabbit IgG to FcγRIII on cells.

FIG. 61D shows Saturation Binding Curves, which demonstrate an enhanced binding of polypeptides comprising a first and second Fc fragment of rabbit IgG in dimeric form to FcγRIV on cells compared to the binding of rabbit IgG to FcγRIV on cells.

### DETAILED DESCRIPTION

As disclosed herein, polypeptides comprising Fc fragments of IgG are provided. Such polypeptides are small in size and thus, after dimerizing are able to bind and cross-link at least two FcγRs on stimulated leukocytes thereby inducing IL-10 production without causing tissue pathology or toxicity. The IL-10 produced from these cells can have important and potent biological consequences, such as reversing the lethal effects of severe inflammatory conditions, as set forth herein. The polypeptides comprising at least a first and second Fc fragment of IgG have several uses, including, but not limited to use as an anti-inflammatory agent for treating conditions that have inflammation as one of the symptoms or as a laboratory reagent.

It is to be understood that certain descriptions of the present disclosure have been simplified to illustrate only those elements and limitations that are relevant to a clear understanding of the present disclosure, while eliminating, for purposes of clarity, other elements. Those of ordinary skill in the art, upon considering the present description, will recognize that other elements and/or limitations may be desirable in order to implement embodiments of the present disclosure. However, because such other elements and/or limitations may be readily ascertained by one of ordinary skill upon considering the present description, and are not necessary for a complete understanding of the present invention, a discussion of such elements and limitations is not provided herein. As such, it is to be understood that the description set forth herein is merely exemplary to embodiments of the present description and is not intended to limit the scope of the claims.

Other than in the examples herein, or unless otherwise expressly specified, all of the numerical ranges, amounts, values and percentages, such as those for amounts of materials, elemental contents, times and temperatures of reaction, ratios of amounts, and others, in the following portion of the specification and attached claims may be read as if prefaced by the word “about”, even though the term “about” may not expressly appear with the value, amount, or range. Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains error necessarily resulting from the standard deviation found in its underlying respective testing measurements. Furthermore, when numerical ranges are set forth herein, these ranges are inclusive of the recited range end points (end points may be used).

Also, it should be understood that any numerical range recited herein is intended to include all sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10. The terms “one,” “a,” or “an” as used herein are intended to include “at least one” or “one or more,” unless otherwise indicated.

All referenced patents, patent applications, publications, sequence listings, electronic copies of sequence listings, or other disclosure material identified herein are incorporated by reference in whole but only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

As set forth herein, various non-limiting embodiments of the present disclosure are directed to polypeptides comprising at least a first and second Fc fragment of IgG. In other embodiments, the polypeptides may comprise multiple Fc fragments of IgG.

As set forth herein, the terms “polypeptide,” “peptide,” and “protein” are used interchangeably to refer to a polymer of amino acid residues. The polypeptide may be obtained from various means known in the art, including, but not limited to, cellular extraction, cellular supernatant, protein extraction procedure, or artificial/chemical synthesis, and any combinations thereof. The polypeptide may be a recombinant polypeptide. The term “recombinant polypeptide”, as used herein, is intended to include polypeptides comprising at least a first and second Fc fragment of IgG that may be prepared, expressed, created or isolated by recombinant means, such as a polypeptide comprising at least a first and second Fc fragment of IgG isolated from an animal (e.g., a mouse) that is transgenic for a polynucleotide that encodes a polypeptide comprising at least a first and second Fc fragment of IgG, polypeptides comprising at least a first and second Fc fragment of IgG expressed using a recombinant expression vector transfected into a host cell, polypeptides comprising at least a first and second Fc fragment of IgG isolated from a recombinant, combinatorial polypeptide library, or polypeptides comprising at least a first and second Fc fragment of IgG prepared, expressed, created or isolated by any other means that involves splicing of IgG gene sequences to other DNA sequences.

As used herein, the term “gene” refers to a segment of nucleic acid, DNA or RNA, which encodes and is capable of expressing a specific gene product. A gene often produces a protein or polypeptide as its gene product, but in its broader sense, a gene can produce any desired product, whether the product is a polypeptide or nucleic acid.

As used herein, the term “nucleic acid” and “polynucleotide” refers to a polymer of ribonucleic acids or deoxyribonucleic acids, including RNA, mRNA, rRNA, tRNA, small nuclear RNAs, cDNA, DNA, PNA, RNA/DNA copolymers, or analogues thereof. A nucleic acid may be obtained from a cellular extract, genomic or extragenomic DNA, viral RNA or DNA, or artificially/chemically synthesized molecules.

As used herein, the term “cDNA” refers to complementary or “copy” DNA. Generally cDNA is synthesized by a DNA polymerase using any type of RNA molecule (e.g., typically mRNA) as a template. Alternatively, the cDNA may be obtained by directed chemical syntheses.

As used herein, the term “complementary” refers to nucleic acid sequences capable of base-pairing according to the standard Watson-Crick complementary rules, or being capable of hybridizing to a particular nucleic acid segment under relatively stringent conditions. Nucleic acid polymers are optionally complementary across only portions of their entire sequences.

As used herein, the term “RNA” refers to a polymer of ribonucleic acids, including RNA, mRNA, rRNA, tRNA, and small nuclear RNAs, as well as to RNAs that comprise ribonucleotide analogues to natural ribonucleic acid residues, such as 2-O-methylated residues.

As used herein, the term “primer” refers to any nucleic acid that is capable of hybridizing at its 3′-end to a complementary nucleic acid molecule and that provides a free 3′-hydroxyl terminus which can be extended by a nucleic acid polymerase.

As used herein, the term “upstream” refers to the relative position in DNA or RNA toward the 5′-end of the DNA or RNA molecule.

As used herein, the term “downstream” refers to the relative position in DNA or RNA toward the 3′-end of the DNA or RNA molecule.

As used herein, the term “vector” refers to a means for introducing a foreign nucleotide sequence into a cell, including without limitation, a plasmid or virus. Such vectors may operate under the control of a host cell's gene expression machinery. A vector may contain sequences that facilitate replication and/or maintenance of a segment of foreign nucleic acid in the host cell. In use, the vector is introduced into a host cell for replication and/or expression of the segment of foreign DNA or for delivery of the foreign DNA into the host genome. A typical plasmid vector contains: (i) an origin of replication, so that the vector can be maintained and/or replicated in a host cell; (ii) a selectable marker, such as an antibiotic resistance gene to facilitate propagation of the plasmid; and (iii) a polylinker site containing several different restriction endonuclease recognition and cut sites to facilitate cloning of a foreign DNA sequence. pCRII T/A TOPO and pFuse-Fc2 discussed below in the Examples, are two such plasmid vectors.

As used herein, a “transfected cell” or “transformed cell” refers to a cell into which (or into an ancestor of which) a nucleic acid of the invention has been introduced.

As used herein, a nanoparticle refers to a small cluster of atoms ranging from 1 to 100 nanometers in size.

As used herein, the term “host cell” refers to any prokaryotic or eukaryotic cell where a desired nucleic acid sequence has been introduced into the cell. The metabolic processes and pathways of such a host cell are capable of maintaining, replicating, and/or expressing a vector containing a foreign gene or nucleic acid. There are a variety of suitable host cells, including but not limited to, bacterial, fungal, insect, yeast, mammalian, and plant cells, that may be utilized in various ways (for example, as a carrier to maintain a plasmid comprising a desired sequence). Representative mammalian host cells include, but are not limited to, HeLa cells, Chinese Hamster Ovary (CHO) cells and NS1 cell lines.

As used herein, a “knockout mouse” refers to a mouse that contains within its genome a specific gene that has been inactivated by the method of gene targeting. A knockout mouse includes both the heterozygote mouse (i.e., one defective allele and one wild-type allele) and the homozygous mutant (i.e., two defective alleles).

Nucleic acids may be introduced into cells according to standard methodologies including electroporation, or any other transformation or nucleic acid transfer method known in the art.

As used herein the term “Fc fragment of IgG” refers to a portion of the nucleotide sequence of the Fc region of IgG or a portion of an amino acid sequence of the Fc region of IgG. An Fc fragment of IgG may include at least one C_{H}2 domain and at least one hinge region. An Fc fragment of IgG may further include a C_{H}3 domain. Fragments of a nucleotide sequence of IgG may encode Fc fragments of IgG that retain the biological activity of the corresponding Fc portion of IgG.

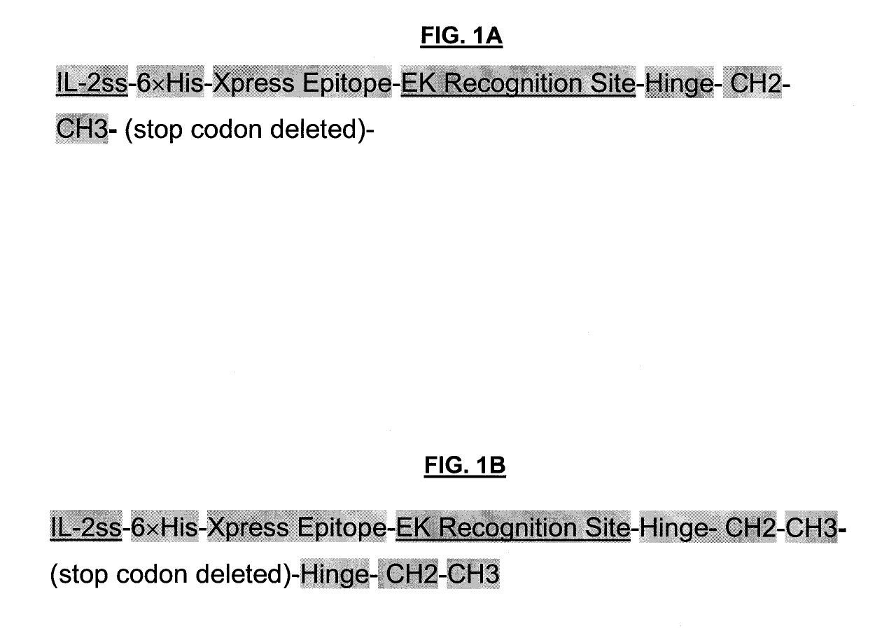

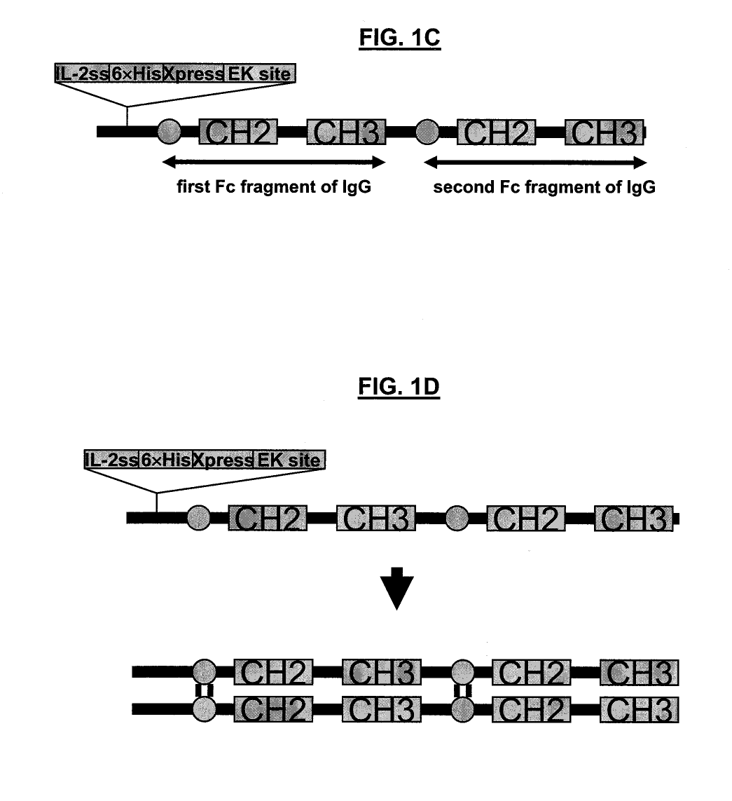

In certain embodiments of the present disclosure, at least one first Fc fragment of IgG may comprise at least one C_{H}2 domain and at least one hinge region. As used herein, the term “constant or C_{H} domain” includes a nucleotide or amino acid sequence that is constant between different IgG molecules. As used herein, the term “hinge region” includes a portion of the IgG heavy chain that may be used to join a first Fc fragment of IgG to a second Fc fragment of IgG to form a chain wherein the first and second Fc fragments of IgG are bound through the hinge region (See FIG. 1C). The hinge region of the Fc fragment of IgG may permit the attachment of multiple Fc fragments of IgG to one another in a series to form a chain. Each Fc fragment of IgG, including a first Fc fragment of IgG, a second Fc fragment of IgG or any additional Fc fragments of IgG that may be attached to the first Fc fragment of IgG or the second Fc fragment of IgG, has two ends. Therefore, the term “in a series” and “end-to-end” are used interchangeably to refer to an Fc fragment of IgG attached to another Fc fragment of IgG to form a chain. As used herein, the term “chain” and “polypeptide in monomeric form” are used interchangeably to include a first Fc fragment of IgG attached to one or more additional Fc fragments of IgG in a tandem series. In addition, the hinge may also permit the attachment of multiple chains to one another. Thus, the claimed polypeptide may include one chain, two chains, or multiple chains. For example, in the polypeptide comprising two chains, the hinge regions of a preexisting chain may bind to hinge regions of a second chain to form a dimer or “polypeptide in dimeric form” (See FIG. 1D). Furthermore, in the polypeptide comprising multiple chains, the hinge regions of the multiple chains may bind to the hinge regions of the first chain and second chain to form a multimer.

In certain other embodiments, the at least one first Fc fragment of IgG may comprise at least one C_{H}2 domain, at least one C_{H}3 domain, and at least one hinge region. In other embodiments, the first Fc fragment of IgG and second Fc fragment of IgG may comprise at least one C_{H}2 domain, at least one C_{H}3 domain, and at least one hinge region. In other embodiments, the additional Fc fragments of IgG that are attached to the first Fc fragment of IgG and second Fc fragment of IgG in a series may comprise at least one C_{H}2 domain, at least one C_{H}3 domain, and a least one hinge region.

In certain specific embodiments, the at least one first Fc fragment of IgG may comprise one C_{H}2 domain and one hinge region. In other embodiments, the first and second Fc fragments of IgG may comprise one C_{H}2 domain and one hinge region. In other embodiments, additional Fc fragments of IgG that are attached to the first and second Fc fragments of IgG may comprise one C_{H}2 domain and one hinge region. In certain specific embodiments, the at least one first Fc fragment of IgG may comprise one C_{H}2 domain, one C_{H}3 domain, and one hinge region. In other embodiments, the first and second Fc fragments of IgG may comprise one C_{H}2 domain, one C_{H}3 domain, and one hinge region. In other embodiments, additional Fc fragments of IgG that are attached to the first and second Fc fragments of IgG in a series may comprise one C_{H}2 domain, one C_{H}3 domain, and one hinge region.

In certain other embodiments, the at least one first Fc fragment of IgG may include an orientation in the following manner: the hinge region followed by the C_{H}2 domain. In other embodiments, the at least one first Fc fragment of IgG may include an orientation in the following manner: the hinge region followed by the C_{H}2 domain followed by the C_{H}3 domain. In other embodiments, the at least one first Fc fragment of IgG may include an orientation in the following manner: the hinge region followed by the C_{H}3 domain. In other embodiments, the at least one first Fc fragment of IgG may include an orientation in the following manner: the hinge region followed by the C_{H}3 domain followed by the C_{H}2 domain. In certain other embodiments, the at least one first Fc fragment of IgG may include an orientation in the following manner: the C_{H}2 domain followed by hinge region. In other embodiments, the at least one first Fc fragment of IgG may include an orientation in the following manner: the C_{H}2 domain followed by the hinge region followed by the C_{H}3 domain. In other embodiments, the at least one first Fc fragment of IgG may include an orientation in the following manner: the C_{H}2 domain followed by the C_{H}3 domain followed by the hinge region. In other embodiments, the at least one first Fc fragment of IgG may include an orientation in the following manner: the C_{H}3 domain followed by the hinge region. In other embodiments, the at least one first Fc fragment of IgG may include an orientation in the following manner: the C_{H}3 domain followed by the hinge region followed by the C_{H}2 domain. In other embodiments, the at least one first Fc fragment of IgG may include an orientation in the following manner: the C_{H}3 domain followed by the C_{H}2 domain followed by the hinge region.

In certain other embodiments, the second Fc fragment of IgG may include an orientation in the following manner: the hinge region followed by the C_{H}2 domain. In other embodiments, the second Fc fragment of IgG may include an orientation in the following manner: the hinge region followed by the C_{H}2 domain followed by the C_{H}3 domain. In other embodiments, the second Fc fragment of IgG may include an orientation in the following manner: the hinge region followed by the C_{H}3 domain. In other embodiments, the second Fc fragment of IgG may include an orientation in the following manner: the hinge region followed by the C_{H}3 domain followed by the C_{H}2 domain. In certain other embodiments, the second Fc fragment of IgG may include an orientation in the following manner: the C_{H}2 domain followed by hinge region. In other embodiments, the second Fc fragment of IgG may include an orientation in the following manner: the C_{H}2 domain followed by the hinge region followed by the C_{H}3 domain. In other embodiments, the second Fc fragment of IgG may include an orientation in the following manner: the C_{H}2 domain followed by the C_{H}3 domain followed by the hinge region. In other embodiments, the second Fc fragment of IgG may include an orientation in the following manner: the C_{H}3 domain followed by the hinge region. In other embodiments, the second Fc fragment of IgG may include an orientation in the following manner: the C_{H}3 domain followed by the hinge region followed by the C_{H}2 domain. In other embodiments, the second Fc fragment of IgG may include an orientation in the following manner: the C_{H}3 domain followed by the C_{H}2 domain followed by the hinge region.

In certain embodiments, a polypeptide comprising at least a first and second Fc fragment of IgG may include a first and second Fc fragment of IgG comprising any combination of the orientations set forth herein.

In certain other embodiments, a polypeptide comprising a first and second Fc fragment of IgG and additional Fc fragments of IgG attached to the first and second Fc fragments of IgG in a series may include an orientation in the following manner: the hinge region followed by the C_{H}2 domain. In other embodiments, the additional Fc fragments of IgG that are attached to the first and second Fc fragments of IgG in a series may include an orientation in the following manner: the hinge region followed by the C_{H}2 domain followed by the C_{H}3 domain. In other embodiments, the additional Fc fragments of IgG that are attached to the first and second Fc fragments of IgG in a series may include an orientation in the following manner: the hinge region followed by the C_{H}3 domain. In other embodiments, the additional Fc fragments of IgG that are attached to the first and second Fc fragments of IgG in a series may include an orientation in the following manner: the hinge region followed by the C_{H}3 domain followed by the C_{H}2 domain. In certain other embodiments, the additional Fc fragments of IgG that are attached to the first and second Fc fragments of IgG in a series may include an orientation in the following manner: the C_{H}2 domain followed by hinge region. In other embodiments, the additional Fc fragments of IgG that are attached to the first and second Fc fragments of IgG in a series may include an orientation in the following manner: the C_{H}2 domain followed by the hinge region followed by the C_{H}3 domain. In other embodiments, the additional Fc fragments of IgG that are attached to the first and second Fc fragments of IgG in a series may include an orientation in the following manner: the C_{H}2 domain followed by the C_{H}3 domain followed by the hinge region. In other embodiments, the additional Fc fragments of IgG that are attached to the first and second Fc fragments of IgG in a series may include an orientation in the following manner: the C_{H}3 domain followed by the hinge region. In other embodiments, the additional Fc fragments of IgG that are attached to the first and second Fc fragments of IgG in a series may include an orientation in the following manner: the C_{H}3 domain followed by the hinge region followed by the C_{H}2 domain. In other embodiments, the additional Fc fragments IgG that are attached to the first and second Fc fragments of IgG in a series may include an orientation in the following manner: the C_{H}3 domain followed by the C_{H}2 domain followed by the hinge region.

In certain embodiments, a polypeptide comprising additional Fc fragments of IgG that are attached to the first and second Fc fragments of IgG in a series may include additional Fc fragments of IgG comprising any combination of the orientations set forth herein.

In certain embodiments, the at least first and second Fc fragment of IgG may be bound through the at least one hinge region. As used herein, “bound through” refers to the first Fc fragment of IgG being attached to the second Fc fragment of IgG. “Bound through” may also refer to additional Fc fragments of IgG that are attached to the first and second Fc fragments of IgG. These Fc fragments of IgG may be attached or bound to one another in a series or end to end.

In certain embodiments, at least one first and second Fc fragment of IgG may form a chain. In other embodiments, multiple substantially similar chains may bind to at least one other of said multiple chains in a substantially parallel relationship. As used herein, the term “substantially similar” means at least two chains that each comprise at least one hinge region as a common entity. As used herein, the term “substantially parallel” means at least two chains comprising at least one hinge region that may bind to one another at the hinge region(s), causing the chains to be arranged in a near or essentially, horizontal orientation. For example, a first chain may bind to a second chain in a substantially parallel manner to form a dimer. Furthermore, additional chains may bind to the first and second chains in a substantially parallel manner to form a multimer.

In certain embodiments of the present disclosure, the Fc fragments of IgG may include Fc fragments of mammalian IgG. In other embodiments, the Fc fragments of IgG may include Fc fragments of murine IgG, Fc fragments of rabbit IgG, Fc fragments of human IgG, and any combinations thereof.

IgG from several different murine strains may be used including, but not limited, to murine BALB/c and murine C57BL/6 strains. Murine BALB/c have different IgG subtypes, including IgG1, IgG2a, IgG2b and IgG3. Murine C57BL/6 have different IgG subtypes including IgG1, IgG2b, IgG2c and IgG3.

In certain embodiments, the Fc fragments of murine IgG may include, for example, Fc fragments of murine BALB/c IgG1, Fc fragments of murine BALB/c IgG2a, Fc fragments of murine BALB/c IgG2b, Fc fragments of murine BALB/c IgG3, Fc fragments of murine C57BL/6 IgG1, Fc fragments of murine C57BL/6 IgG2b, Fc fragments of murine C57BL/6 IgG2c, Fc fragments of murine C57BL/6 IgG3, and any combinations thereof.

In certain embodiments, the Fc fragments of human IgG may include, for example, Fc fragments of human IgG1, Fc fragments of human IgG2, Fc fragments of human IgG3, Fc fragments of human IgG4, and any combinations thereof.

In certain embodiments, the polypeptide comprising at least a first and second Fc fragment of IgG may further comprise a bound polytyrosine tag. One of ordinary skill in the art would recognize that the polypeptides comprising at least a first and second Fc fragment of IgG may be attached to chitosan-containing nanoparticles via the bound polytyrosine tag. Thus, in other embodiments, the polypeptide comprising at least a first and second Fc fragment of IgG may further comprise bound nanoparticles. In other embodiments, the polypeptide comprising at least a first and second Fc fragment of IgG may further comprise a bound histidine tag. As used herein, the term “tag” refers to any detectable moiety. A tag may be used to distinguish a particular polypeptide comprising at least a first and second Fc fragment of IgG from others that are untagged or tagged differently, or the tag may be used to enhance detection or purification.

In certain embodiments, the polypeptide may be synthetic or recombinant.

Without wishing to be bound by theory, a polypeptide comprising at least a first and second Fc fragment of IgG may form a chain. Two parallel chains form a dimer and multiple parallel chains form a multimer. The polypeptide comprising at least a first and second Fc fragment of IgG in dimeric form may be configured to bind and cross-link at least two FcγRs by the protein sequence of the first Fc fragment of IgG binding and cross-linking one FcγR and the protein sequence of the second Fc fragment of IgG binding and cross-linking a second FcγR. As used herein “configured to bind” refers to the nucleotide or polypeptide sequence arrangement that permits binding of the polypeptide comprising at least a first and second Fc fragment of IgG in dimeric form to at least two FcγRs on a stimulated cell. As used herein “configured to bind and cross-link” refers to the nucleotide or polypeptide sequence arrangement that permits binding and cross-linking of the polypeptide comprising at least a first and second Fc fragment of IgG in dimeric form or multimeric form to at least two FcγRs on a stimulated cell, thereby causing cellular induction of IL-10.

Without wishing to be bound by theory, both binding and cross-linking of at least two FcγRs may be necessary to thereby induce IL-10 production. For example, polypeptides comprising at least a first and second Fc fragment of IgG in dimeric form may bind and cross-link at least two FcγRs, thereby inducing IL-10. Polypeptides comprising at least a first and second Fc fragment of IgG in multimeric form may bind and cross-link at least two FcγRs; thereby inducing IL-10. In contrast, polypeptides comprising at least a first and second Fc fragment of IgG in monomeric form are not configured to bind at least two FcγRs on a stimulated cell. Thus, the polypeptides comprising at least a first and second Fc fragment of IgG in monomeric form may be unable to bind and cross-link at least two FcγRs on a stimulated cell and thereby do not induce IL-10 production. In addition, polypeptides containing only a first Fc fragment of IgG in dimeric form may bind at least two FcγRs, but will not cross-link the receptors; thus, IL-10 will not be induced.

In certain embodiments, the polypeptide comprising at least a first and second Fc fragment of IgG may be configured to bind and cross-link at least two FcγRs on a stimulated cell.

In certain other embodiments, the polypeptide comprising at least a first and second Fc fragment of IgG may be configured to bind or bind and cross-link at least two FcγRs on a stimulated cell, such as mammalian FcγRs. In other embodiments, the polypeptide comprising at least a first and second Fc fragment of IgG may be configured to bind or bind and cross-link at least two murine FcγRs, at least two human FcγRs, at least two rabbit FcγRs, and any combinations thereof.

In certain other embodiments, the polypeptide comprising at least a first and second Fc fragment of IgG may be configured to bind or bind and cross-link at least two FcγRs, such as FcγR type I, FcγR type Ill, FcγR IV, and any combinations thereof.

In certain embodiments, the polypeptide comprising at least a first and second Fc fragment of IgG in dimeric form may be configured to thereby induce the anti-inflammatory cytokine, IL-10, upon binding and cross-linking at least two FcγRs on a stimulated cell. The polypeptide comprising at least a first and second Fc fragment of IgG in dimeric form may be configured to downregulate production of proinflammatory cytokines upon binding and cross-linking at least two FcγRs on a stimulated cell. As used herein, “downregulate” refers to a decrease in production of proinflammatory cytokines compared to the level of production of proinflammatory cytokines produced by a stimulated cell that is not treated with a polypeptide comprising at least a first and second Fc fragment of IgG in dimeric form. Proinflammatory cytokines that may be downregulated include, but are not limited to, IL-12 and IL-23.

In certain embodiments, the stimulated cell may include a leukocyte. In other specific embodiments, the stimulated cell may include macrophages, dendritic cells and B-cells.

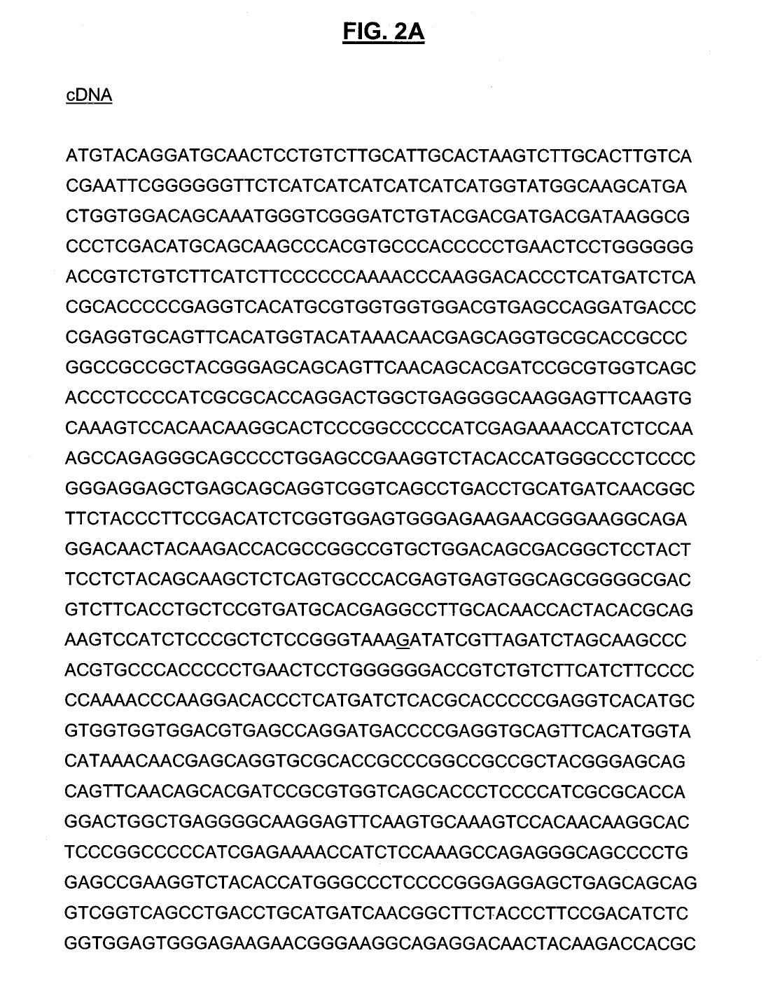

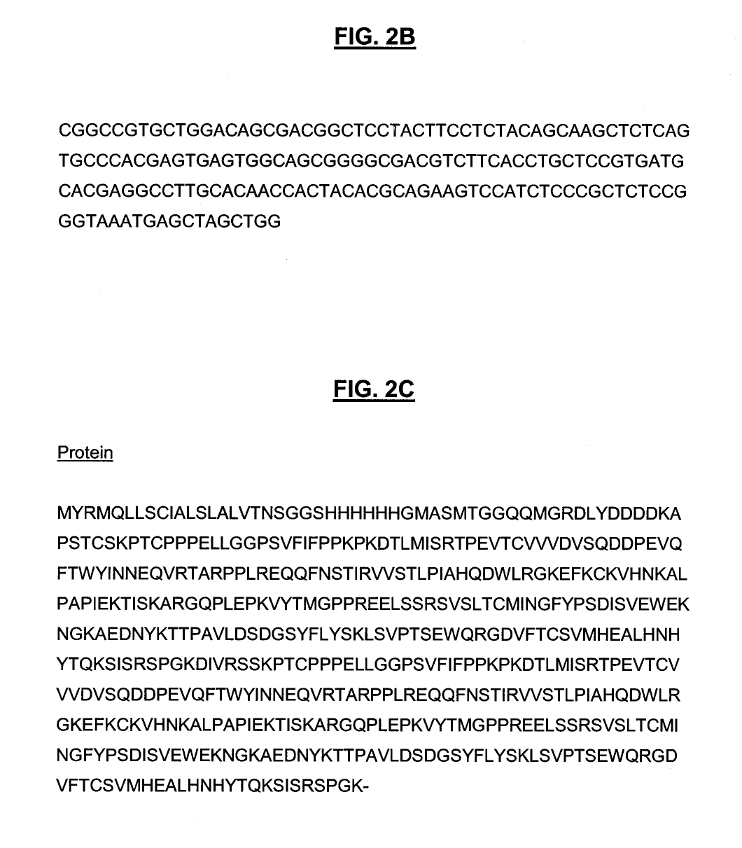

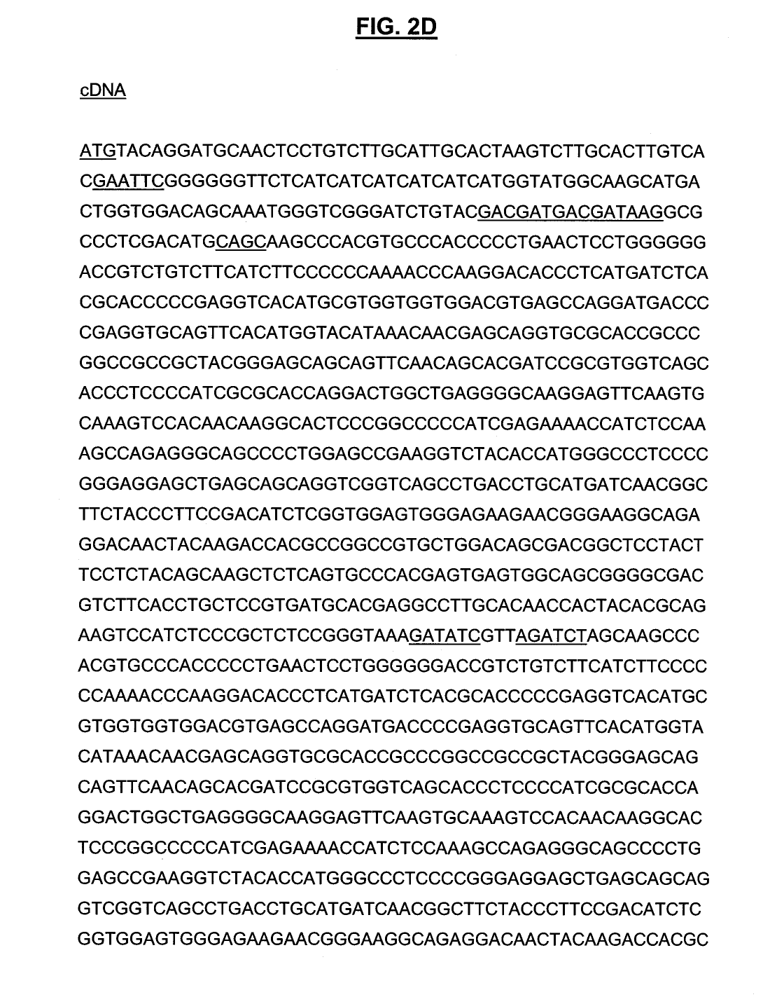

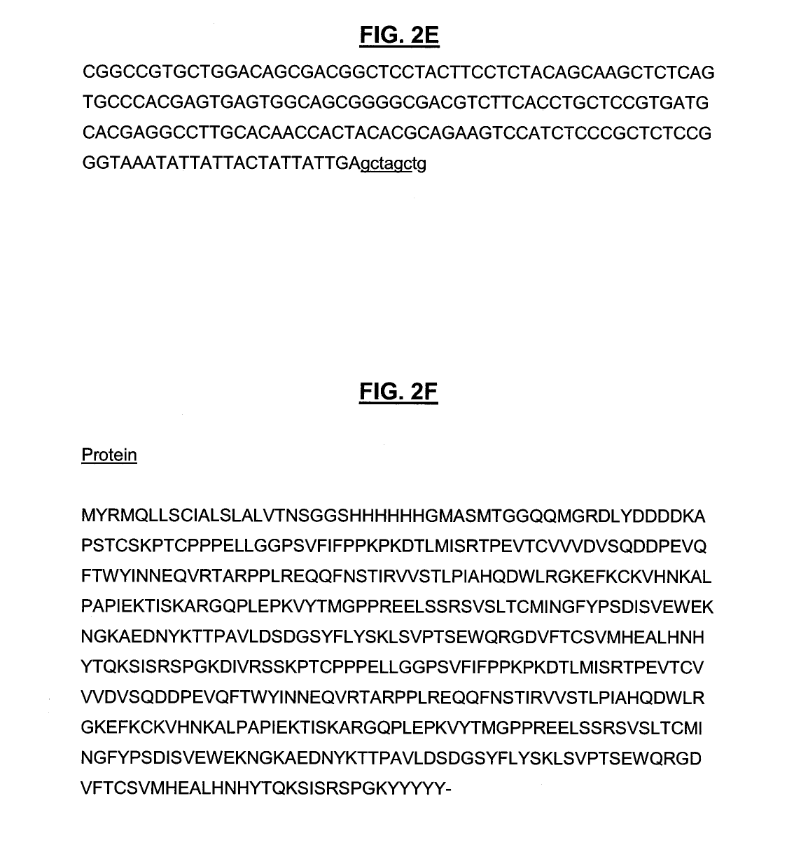

In certain embodiments, a polynucleotide comprising a nucleotide sequence, such as SEQ ID NO: 1, is disclosed wherein the polynucleotide sequence encodes a polypeptide comprising at least a first and second Fc fragment of rabbit IgG (FIGS. 2A-B). In other embodiments, a variant of the polynucleotide SEQ ID NO: 1 is disclosed. As used herein, “polynucleotide variant” refers to polynucleotide sequence that is similar to another polynucleotide sequence.

In certain embodiments, a polypeptide comprising at least a first and second Fc fragment of rabbit IgG is disclosed comprising a rabbit amino acid sequence, such as SEQ ID NO: 2 (FIG. 2C). In other embodiments, a variant of the polypeptide SEQ ID NO: 2 is disclosed. As used herein, “polypeptide variant” refers to polypeptide sequence that is similar to another polypeptide sequence.

In certain embodiments, a polypeptide comprising at least a first and second Fc fragment of murine IgG is disclosed wherein the polypeptide is encoded by a polynucleotide comprising a murine nucleotide sequence selected from a group consisting of SEQ ID NOS: 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, and 67.

In certain embodiments, a polypeptide comprising at least a first and second Fc fragment of murine IgG is disclosed comprising a murine amino acid sequence selected from a group consisting of SEQ ID NOS: 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, and 68.

In certain embodiments, a polypeptide comprising at least a first and second Fc fragment of human IgG is disclosed wherein the polypeptide is encoded by a polynucleotide comprising a human nucleotide sequence selected from a group consisting of SEQ ID NOS: 69, 71, 73, 75, 77, 79, 81, 83, 85, 87, 89, 91, 93, 95, 97, and 99.

In certain embodiments, a polypeptide comprising at least a first and second Fc fragment of human IgG is disclosed comprising a human amino acid sequence selected from a group consisting of SEQ ID NOS: 70, 72, 74, 76, 78, 80, 82, 84, 86, 88, 90, 92, 94, 96, 98, and 100.

The following terms are used to describe the sequence relationships between two or more polynucleotides or polypeptides: (a) “sequence identity” or “sequence identical,” (b) “substantial identity.”

Computer implementations of mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the GCG Wisconsin Genetics Software Package, Version 10 (available from Accelrys Inc., 9685 Scranton Road, San Diego, Calif., USA). Alignments using these programs can be performed using the default parameters. The CLUSTAL program is well described by Higgins et al. (1988) Gene 73:237-244 (1988); Higgins et al. (1989) CABIOS 5:151-153; Corpet et al. (1988) Nucleic Acids Res. 16:10881-90; Huang et al. (1992) CABIOS 8:155-65; and Pearson et al. (1994) Meth. Mol. Biol. 24:307-331. The ALIGN program is based on the algorithm of Myers and Miller (1988) supra. A PAM120 weight residue table, a gap length penalty of 12, and a gap penalty of 4 can be used with the ALIGN program when comparing amino acid sequences. The BLAST programs of Altschul et at (1990) J. Mol. Biol. 215:403 are based on the algorithm of Karlin and Altschul (1990) supra. BLAST nucleotide searches can be performed with the BLASTN program, score=100, word length=12, to obtain nucleotide sequences homologous to a nucleotide sequence encoding a protein of the invention. BLAST protein searches can be performed with the BLASTX program, score=50, word length=3, to obtain amino acid sequences homologous to a protein or polypeptide of the invention. To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al. (1997) supra. When utilizing BLAST, Gapped BLAST, and PSI-BLAST, the default parameters of the respective programs (e.g., BLASTN for nucleotide sequences, BLASTX for proteins) can be used. (See the National Center for Biotechnology Information website on the world-wide web at ncbi.nlm.nih.gov.). Alignment may also be performed manually by inspection.

As used herein, “sequence identity” or “sequence identical” in the context of two nucleic acid or polypeptide sequences makes reference to the nucleotides or amino acids in the two sequences that are the same when aligned.

The term “substantial identity” of polynucleotide or polypeptide sequences means that a polynucleotide or polypeptide sequence comprises a sequence that has at least 70% sequence identity, in certain embodiments at least 80%, in certain other embodiments at least 90%, and in other embodiments at least 95% sequence identity, compared to a reference sequence using one of the alignment programs described using standard parameters.

In certain embodiments, sequences are disclosed having at least 70%, preferably at least 85%, more preferably at least 90% and most preferably at least 95% sequence identity with the sequences presented in SEQ ID NO. 1 and/or SEQ ID NO. 2.

The polypeptides comprising at least a first and second Fc fragment of IgG have several uses, including, but not limited to, use as an anti-inflammatory agent for treating conditions that have inflammation as one of the symptoms or as a laboratory reagent.

Specifically, the polypeptides comprising at least a first and second Fc fragment of IgG may be used as a treatment to reduce a proinflammatory immune response in a patient. In certain embodiments, the polypeptides comprising at least a first and second Fc fragment of IgG may be used as a treatment to reduce inflammation in a patient, wherein the patient has a condition, which includes inflammation as one symptom.

Current treatments, such as IVIG, are used to reduce inflammation in a number of inflammatory conditions as described in Tables 1 and 2 of Constantine M M et al., 2007. IVIG utilization in the Canadian Atlantic provinces: a report of the Atlantic Collaborative IVIG utilization working group. Transfusion 47:2072-80, which is incorporated by referenced herein in its entirety. The use of the polypeptides comprising at least a first and second Fc fragment of IgG would be used to reduce inflammation for the same set of conditions. In certain specific embodiments, conditions that may be treated by the disclosed polypeptides may include sepsis, endotoxemia, rheumatoid arthritis, inflammatory bowel disease, Idiopathic Thrombocytopenic Purpura (ITP), multiple sclerosis, myasthenia gravis, polymyositis, Kawasaki disease, dermatomyositis, chronic inflammatory demyelinating polyneuropathy (CIDP), Guillain-Barre syndrome, Experimental Autoimmune Encephalomyelitis (EAE), diabetes mellitus, Systemic Lupus Erythematosus (SLE), colitis, amyotrophic lateral sclerosis (ALS), cardiovascular disease, autism, and obesity.

More specifically, the polypeptides comprising at least a first and second Fc fragment of IgG may be used as a replacement for intravenous immunoglobulin (IVIG).

The polypeptides set forth herein have several advantages over IVIG treatment. First, IVIG is obtained from human donors. Therefore, it is difficult and extremely expensive to process. For example, the amount or dose of IVIG administered to patients with inflammatory diseases is 2-3 mg/kg (high dose IVIG). Presently, the cost of IVIG ranges from $50 to $75 per gram. Therefore, a single treatment of high dose IVIG to a 75 kg patient can cost in excess of $10,000. Second, there are safety concerns associated with the use of any human blood products. Third, a large amount of IVIG must be administered and this often can be associated with infusion reactions. Finally, there is a serious shortage of IVIG. A recent report from the Office of the Inspector General indicated that 57% of the responding physicians reported that they were unable to provide patients with adequate amounts of IVIG during the first quarter of 2006 and none of the distributors were able to fulfill all customer requests for IVIG as set forth in Levinson, D. R. Intravenous Immune Globulin: Medicare payment and availability. Report to DHHS, OEI-03-05-00404, April 27, which is incorporated by reference herein in its entirety. The polypeptides comprising at least a first and second Fc fragment of IgG are inexpensive and easy to produce and thus, are available as an unlimited supply.

As disclosed herein, treatments may include administering to a patient a therapeutically effective amount of polypeptides comprising at least a first and second Fc fragment of IgG. As used herein, the term “therapeutically effective amount” refers to an amount of a polypeptide comprising at least a first and second Fc fragment of IgG effective to reduce or prevent inflammation in an inflammatory condition or disease in a human or non-human mammal. A therapeutically effective amount may be determined in several different ways depending on the disease that is treated. For example, ITP is a disease that results in platelet cell destruction. Therefore, a simple assay measuring platelet cell numbers in patient blood by flow cytometry may be performed to determine the therapeutically effective amount to use of polypeptides comprising at least a first and second Fc fragment of IgG. The therapeutically effective amount will reduce platelet cell destruction thereby reducing inflammation and allow the number of platelets to increase in the blood of a patient receiving the therapeutically effective amount of polypeptides comprising at least a first and second Fc fragment of IgG as set forth in Tremblay T. et al., Picogram doses of LPS exacerbate antibody-mediated thrombocytopenia and reduce the therapeutic efficacy of intravenous immunoglobulin in mice, British Journal of Hematology, 139: 297-302, which is incorporated by reference herein in its entirety. For other diseases, IL-10 and IL-12 can be measured in patient serum. The therapeutically effective amount will increase IL-10 levels in the patient serum and decrease IL-12 levels. The therapeutically effective amount for other conditions can be determined in the same manner or by other techniques well known in the art.

As used herein, the term “administering” and grammatical variations thereof are used herein interchangeably to refer to the delivery of a polypeptide comprising at least a first and second Fc fragment of IgG either systemically or to a local site within the subject. The polypeptides may be administered intravenously, orally, or by tissue injection. As used herein, the term “subject” refers to any human or non-human mammal. In the case of human subjects, the terms “subject” and “patient” may be used interchangeably.

In certain embodiments, a method may be employed wherein the polypeptide comprising the first and second Fc fragment of IgG set forth herein is used as a laboratory reagent. For example, in certain embodiments, the polypeptides set forth herein may be used to block Fc-gamma receptors on a population of cells by adding an effective amount of the polypeptides to the cells. Polypeptides comprising the first and second Fc fragment of IgG set forth herein may be used to block FcγRs on all cells that express FcγRs. One of ordinary skill in the art would know all cells that express FcγRs. Specifically, polypeptides comprising the first and second Fc fragment of IgG may be used to block FcγRs on polymorphonuclear leukocytes (PMNs), macrophages, dendritic cells, and B-cells.

Without wishing to be bound by theory, the polypeptide comprising a least a first and second Fc fragment of IgG in dimeric form is configured to bind and block FcγRs by the protein sequence of the first Fc fragment of IgG binding to one FcγR and the protein sequence of the second Fc fragment of IgG binding to a second FcγR.

As used herein, the term “block” refers to binding to a receptor so that the receptor is inhibited or unable to bind a molecule that it normally is able to bind. For example, by blocking an Fc-gamma receptor, the receptor is unable to bind any IgG-based antibodies.

As used herein, the term “effective amount” refers to an amount of a polypeptide comprising at least a first and second Fc fragment of IgG in dimeric form that is effective to block Fc-gamma receptors.

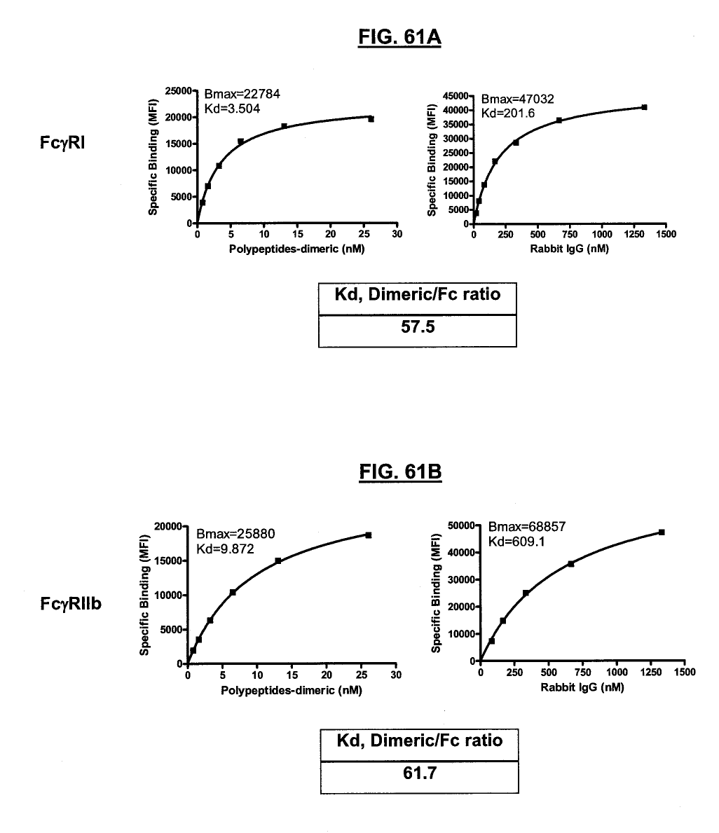

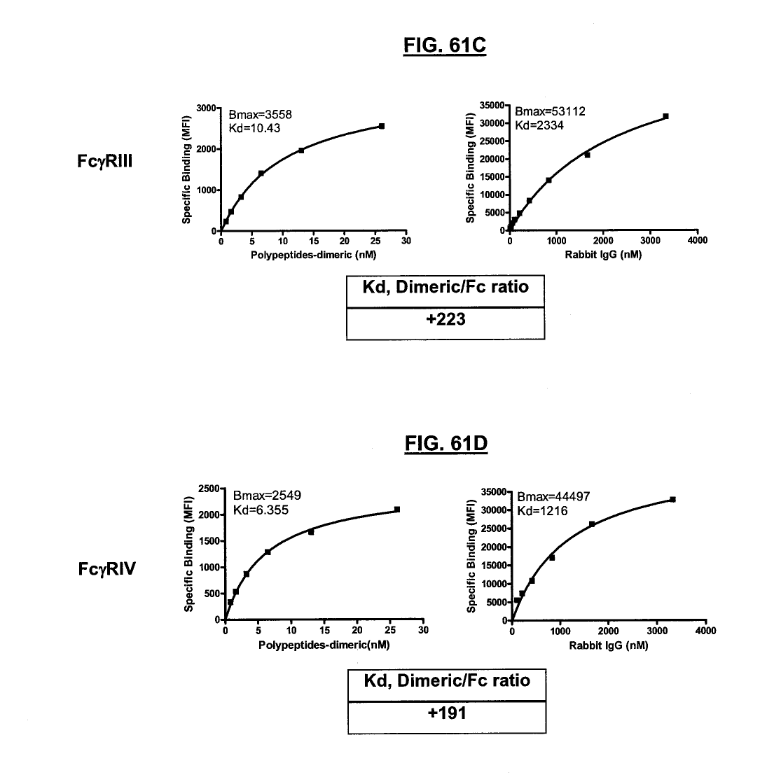

Prior laboratory agents used to block FcγRs could only be used to block murine FcγRs and not human FcγRs. In contrast, polypeptides comprising at least the first and second Fc fragment of IgG in dimeric form, as set forth herein, are able to block both murine and human FcγRs. In addition, prior laboratory agents used to block FcγRs could be used to block, for example, murine FcγRs, however, not all types of FcγRs are blocked. In contrast, polypeptides comprising at least the first and second Fc fragment of IgG in dimeric form are able to block Fc-gamma receptors selected from a group consisting of FcγRI, FcγRIIb, FcγRIII and FcγRIV (FIGS. 61A-D).

In certain embodiments, polypeptides comprising at least a first and second Fc fragment of IgG in dimeric form are able to bind to FcγRs with a higher affinity compared to IgG, i.e., Fc portion of IgG. For example, polypeptides comprising at least the first and second Fc fragment of IgG in dimeric form are able to bind FcγRI with an affinity of at least 3.5 nM (FIG. 61A, left panel) compared to rabbit IgG (“Fc”) which binds to the FcγRI with an affinity of 201 nM (FIG. 61A, right panel). This results in polypeptides comprising at least the first and second Fc fragment of IgG in dimeric form having at least a 57.5 fold enhancement of binding for FcγRI. Polypeptides comprising at least the first and second Fc fragment of IgG in dimeric form are able to bind FcγRIIb with an affinity of at least 9.8 nM (FIG. 61B, left panel) compared to rabbit IgG (“Fc”) which binds to the FcγRIIb with an affinity of 609 nM (FIG. 61B, right panel). This results in polypeptides comprising at least the first and second Fc fragment of IgG in dimeric form having at least a 61.7 fold enhancement of binding for FcγRIIb. Polypeptides comprising at least the first and second Fc fragment of IgG in dimeric form are able to bind FcγRIII with an affinity of at least 10.4 nM (FIG. 61C, left panel) compared to rabbit IgG (“Fc”) which binds to the FcγRIII with an affinity of 2334 nM (FIG. 61C, right panel). This results in polypeptides comprising at least the first and second Fc fragment of IgG in dimeric form having at least a 223 fold enhancement of binding for FcγRIII. Polypeptides comprising at least the first and second Fc fragment of IgG in dimeric form are able to bind FcγRIV with an affinity of at least 6.3 nM (FIG. 61D, left panel) compared to rabbit IgG (“Fc”) which binds to the FcγRIV with an affinity of 1216 nM (FIG. 61D, right panel). This results in polypeptides comprising at least the first and second Fc fragment of IgG in dimeric form having at least a 191 fold enhancement of binding for FcγRIV.

The various embodiments of the present disclosure may be better understood when read in conjunction with the following Examples.

### EXAMPLES

The following examples illustrate various non-limiting embodiments of the polypeptides of the present disclosure and are not restrictive of the invention as otherwise described herein.

### Example 1

### Cloning of pFuse Vector Comprising a Second Fc Fragment of Rabbit IgG cDNA

A rabbit spleen was purchased from Rockland Immunochemicals (Philadelphia, Pa.). Total RNA was isolated from the spleen using RNAzol™ and cDNA was transcribed from the total RNA using reverse transcription.

The second Fc fragment of rabbit IgG cDNA was amplified by polymerase chain reaction (PCR) using the following primers:

(SEQ ID NO: 101)

| | |
|---|---|
| sense: | 5′ - TAGATCTAGCAAGCCCACGTGCC-3′ |

(SEQ ID NO: 102)

| | |
|---|---|
| antisense: | 5′-CCAGCTAGCTCATTTACCCGGAGAGCG-3′ |

The amplified second Fc fragment of rabbit IgG cDNA comprised cDNA of the rabbit IgG hinge-C_{H}2-C_{H}3 domain. The second Fc fragment of rabbit IgG cDNA was then cloned into pCRII T/A TOPO (Invitrogen™) and sequenced. The pCR II T/A TOPO vector comprising the second Fc fragment of rabbit IgG cDNA was then digested to remove the second Fc fragment of rabbit IgG cDNA. The second Fc fragment of rabbit IgG cDNA was then subcloned into a pFuse-Fc2 vector, which contains an IL-2 signal sequence located upstream from the multiple cloning site. The IL-2 signal sequence is required for protein expression. Thus, using this approach a pFuse vector comprising the second fragment of rabbit IgG cDNA was constructed.

### Example 2

### Cloning of pFuse Vector Comprising a First and Second Fc Fragment of Rabbit IgG cDNA

The first Fc fragment of rabbit IgG cDNA was amplified by PCR using the following primers:

sense:
(SEQ ID NO: 103)
5′-ACGAATTCGGGGGGTTCTC-3′
antisense:
(SEQ ID NO: 104)
5′-CTAGATCTAACGATATCTTTACCCGGAGAGCGGGAGA-3′

The amplified first Fc fragment of rabbit IgG cDNA comprised a 6-histidine tag (6×His) followed by an Xpress epitope and EK recognition site on the N-terminal portion of the cDNA located upstream of the rabbit IgG hinge-C_{H}2-C_{H}3 domain (See FIG. 1A). In addition, a stop codon in the C-terminal portion of the C_{H}3 domain was deleted. The 6×His is a polyhistidine metal-binding tag that may be used for purification purposes. The Xpress epitope tag may be used for detection purposes. The EK recognition site is also called the enterokinase recognition site and is also used for purification purposes.

The first Fc fragment of rabbit IgG cDNA as set forth above was cloned into pCRII T/A TOPO (Invitrogen™) and sequenced. The pCR II T/A TOPO vector comprising the first Fc fragment of rabbit IgG cDNA was then digested to remove the first Fc fragment of rabbit IgG cDNA.

The first Fc fragment of rabbit IgG cDNA was then subcloned into the pFuse vector comprising the second Fc fragment of rabbit IgG cDNA as described in Example 1. The first Fc fragment of rabbit IgG cDNA was subcloned upstream of the second Fc fragment of rabbit IgG cDNA to construct a pFuse vector comprising the first and second Fc fragments of rabbit IgG cDNA (See FIGS. 1B-C; FIGS. 2A-B).

### Example 3

### Cloning of pFuse Vector Comprising a First and Second Fc Fragment of Rabbit IgG cDNA with Extra Nucleotides that Encode Five Tyrosine

To facilitate the binding of nanoparticles to polypeptides comprising a first and second Fc fragment of rabbit IgG, nucleotides were added to the C-terminal portion of the second Fc fragment of rabbit IgG cDNA in the pFuse vector comprising the first and second Fc fragment of rabbit IgG cDNA. These nucleotides were added by reamplifying the first and second Fc fragment of rabbit IgG cDNA from the pFuse vector comprising the first and second Fc fragment of rabbit IgG cDNA using the following primers:

sense:
(SEQ ID NO: 105)
5′-TTAGATCTAGCAAGCCCACGTGCCCA-3′
antisense:
(SEQ ID NO: 106)
5′-CAGCTAGCTCAATAATAGTAATAATATTTACCCGGAGAGCGGGA-3′

The first and second Fc fragment of rabbit IgG cDNA further comprising extra nucleotides for nanoparticle binding was then cloned into pCRII T/A TOPO (Invitrogen™) and sequenced. The pCR II T/A TOPO vector comprising the first and second Fc fragment of rabbit IgG cDNA further comprising extra nucleotides for nanoparticle binding was then digested to remove the first and second Fc fragment of rabbit IgG cDNA further comprising extra nucleotides. In addition, the pFuse vector comprising the first and second Fc fragments of rabbit IgG cDNA described in Example 2 was digested to remove the first and second Fc fragments of rabbit IgG cDNA. The first and second Fc fragment of rabbit IgG cDNA further comprising extra nucleotides was then subcloned into the digested pFuse vector that no longer comprised the first and second Fc fragments of rabbit IgG cDNA to construct a pFuse vector comprising the first and second Fc fragments of rabbit IgG cDNA further comprising extra nucleotides for nanoparticle binding (See FIGS. 2D-E).

### Example 4

### Cloning of pFuse Vector Comprising a Second Fc Fragment of Murine BALB/c IgG cDNA

Spleens were isolated from BALB/c mice (National Institute of Health) and total RNA was isolated from the spleens. The murine BALB/c IgG cDNA was reverse transcribed from the RNA. Mice contain different isotypes of IgG. For example, isotypes of IgG for BALB/c mice include IgG1, IgG2a, IgG2b and IgG3.

The second Fc fragment of murine BALB/c IgG1 cDNA, IgG2a cDNA, IgG2b cDNA, or IgG3 cDNA was amplified by PCR using the following primers:

mIgG1 sense:
(SEQ ID NO: 107)
5′-TTAGATCTGTGCCCAGGGATTGTGGT-3′
mIgG1 antisense:
(SEQ ID NO: 108)
5′-CAGCTAGCTCATTTACCAGGAGAGTGGGAG-3′
mIgG2a sense:
(SEQ ID NO: 109)
5′-TTAGATCTGAGCCCAGAGGGCCCACA-3′
mIgG2a antisense:
(SEQ ID NO: 110)
5′-CAGCTAGCTCATTTACCCGGAGTCCG-3′
mIgG2b sense:
(SEQ ID NO: 111)
5′-TTAGATCTGAGCCCAGCGGGCCCATT-3′
mIgG2b antisense:
(SEQ ID NO: 112)
5′-CAGCTAGCTCATTTACCCGGAGACCG-3′
mIgG3 sense:
(SEQ ID NO: 113)
5′-TTAGATCTGAGCCTAGAATACCCAAGCCCA-3′
mIgG3 antisense:
(SEQ ID NO: 114)
5′-CAGCTAGCTCATTTACCAGGGGAGCGA-3′

Using the same approach as set forth in Example 1, a pFuse vector comprising a second Fc fragment of murine BALB/c IgG1 cDNA, IgG2a cDNA, IgG2b cDNA, or IgG3 cDNA was constructed. The second Fc fragment of murine BALB/c IgG1 cDNA, IgG2a cDNA, IgG2b cDNA, or IgG3 cDNA comprised a hinge region, C_{H}2 domain and C_{H}3 domain.

### Example 5

### Cloning of pFuse Vector Comprising a First and Second Fc Fragment of Murine BALB/c IgG cDNA

The first Fc fragment of murine BALB/c IgG1 cDNA, IgG2a cDNA, IgG2b cDNA, or IgG3 cDNA was amplified by PCR using the following primers:

mIgG fragment 1 sense:
(SEQ ID NO: 115)
5′-ACGAATTCGGGGGGTTCTC-3′
mIgG1 fragment 1 antisense:
(SEQ ID NO: 116)
5′-CTAGATCTAACGATATCTTTACCAGGAGAGTGGGAGAGG-3′
mIgG2a fragment 1 antisense:
(SEQ ID NO: 117)
5′-CTAGATCTAACGATATCTTTACCCGGAGTCCGGG-3′
mIgG2b fragment 1 antisense:
(SEQ ID NO: 118)
5′-CTAGATCTAACGATATCTTTACCCGGAGACCGG-3′
mIgG3 fragment 1 anti-sense:
(SEQ ID NO: 119)
5′-CTAGATCTAACGATATCTTTACCAGGGGAGCGAGAC-3′

Using the same approach as set forth in Example 2, a pFuse vector comprising a first Fc fragment of murine BALB/c IgG1 cDNA, IgG2a cDNA, IgG2b cDNA, or IgG3 cDNA was constructed.

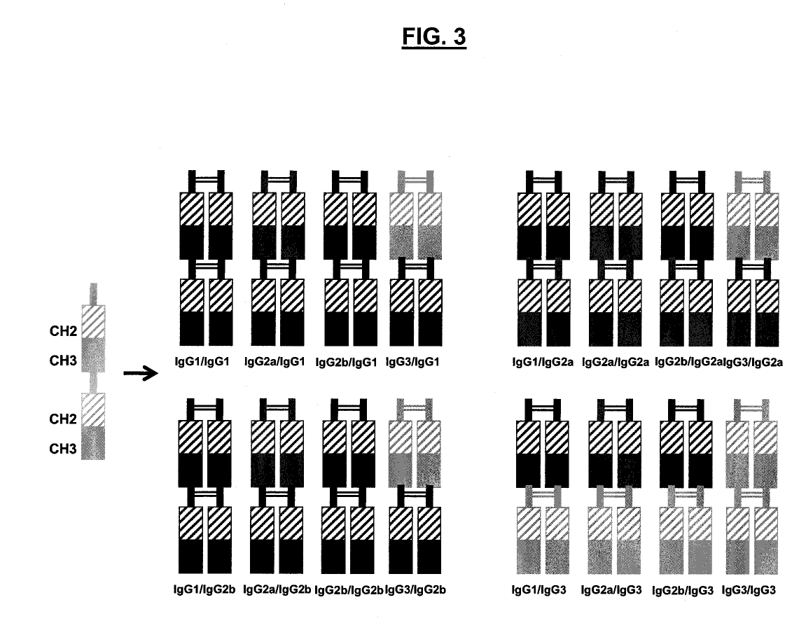

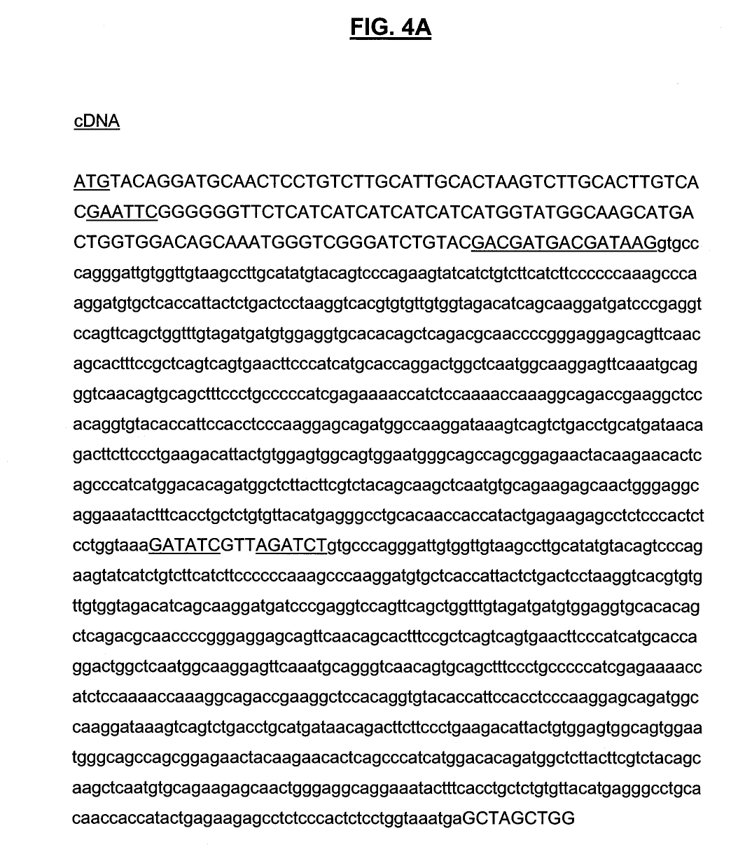

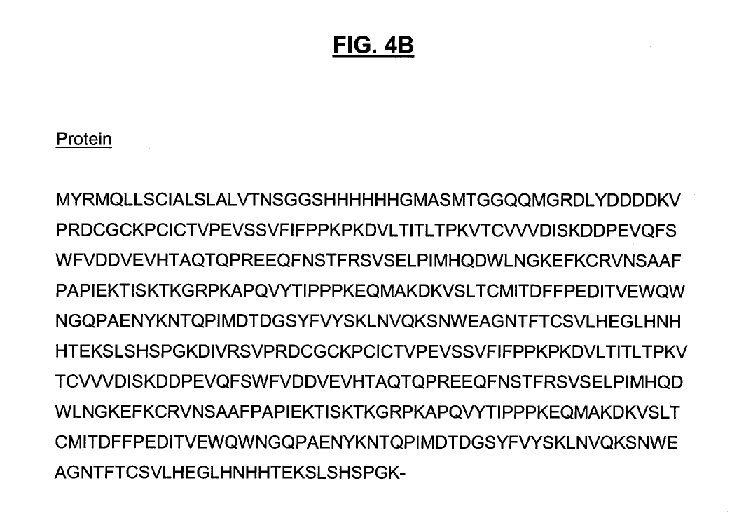

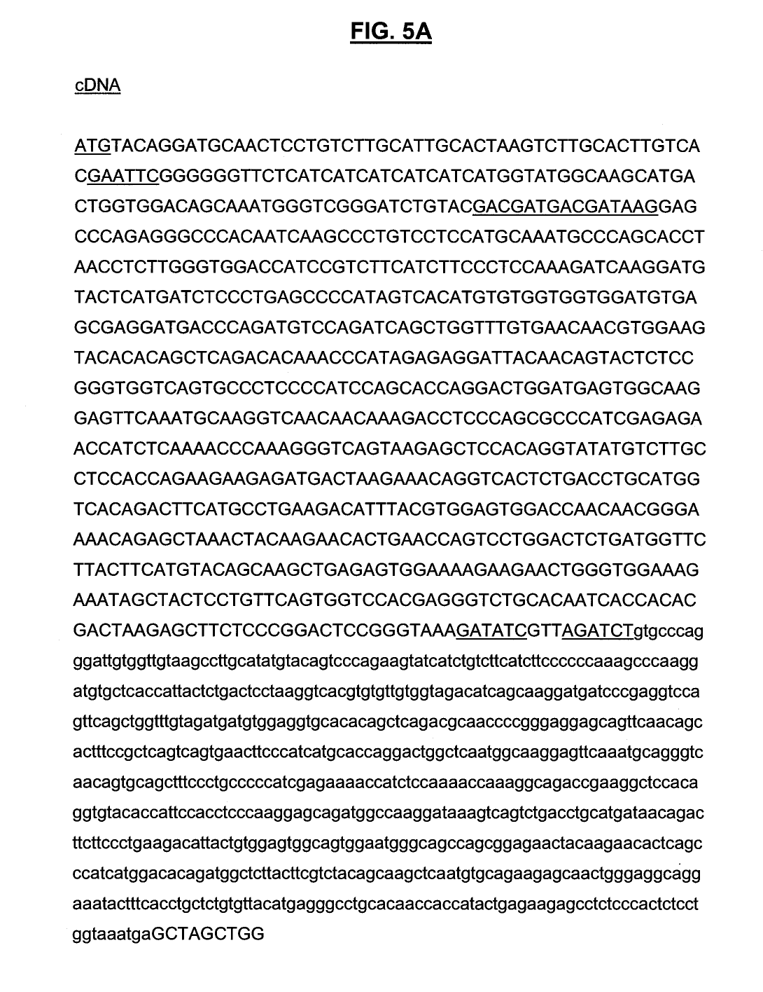

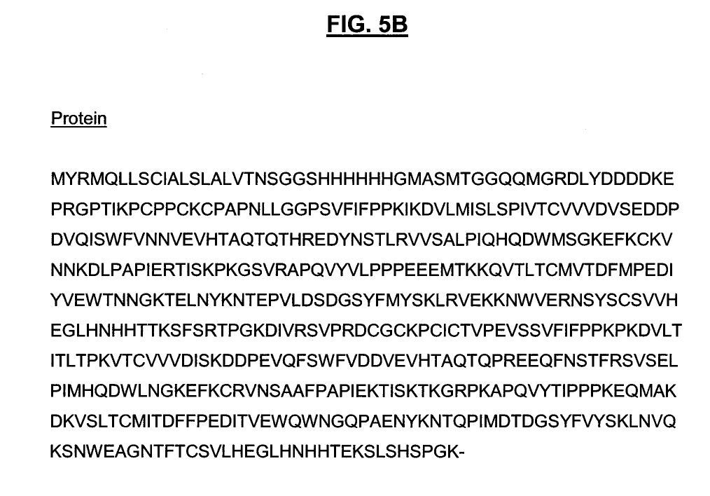

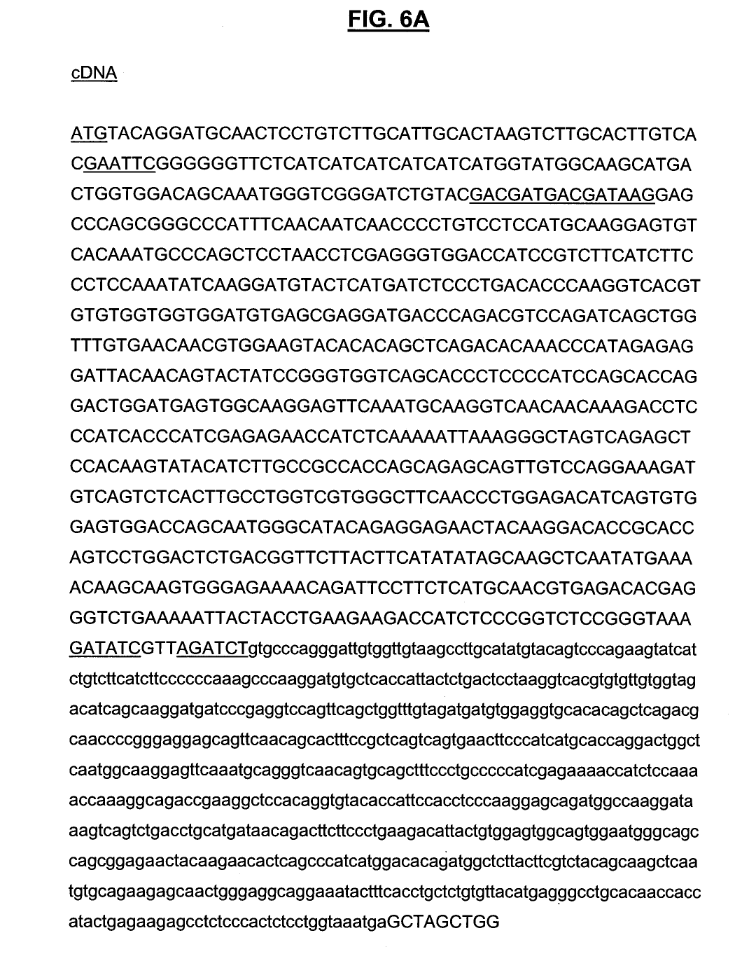

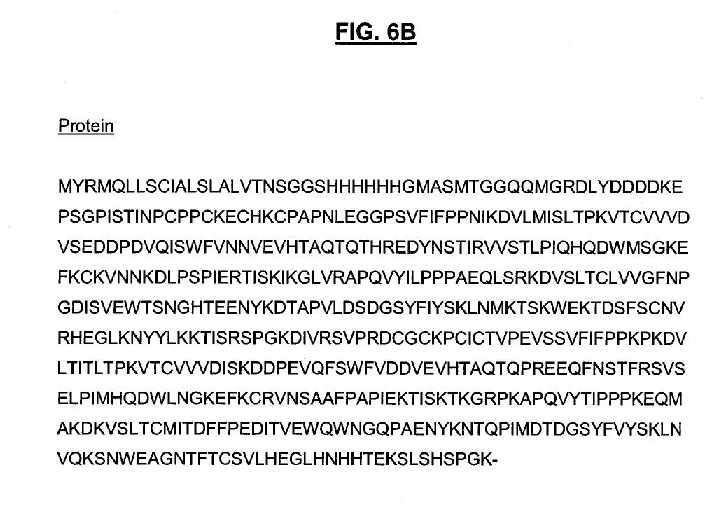

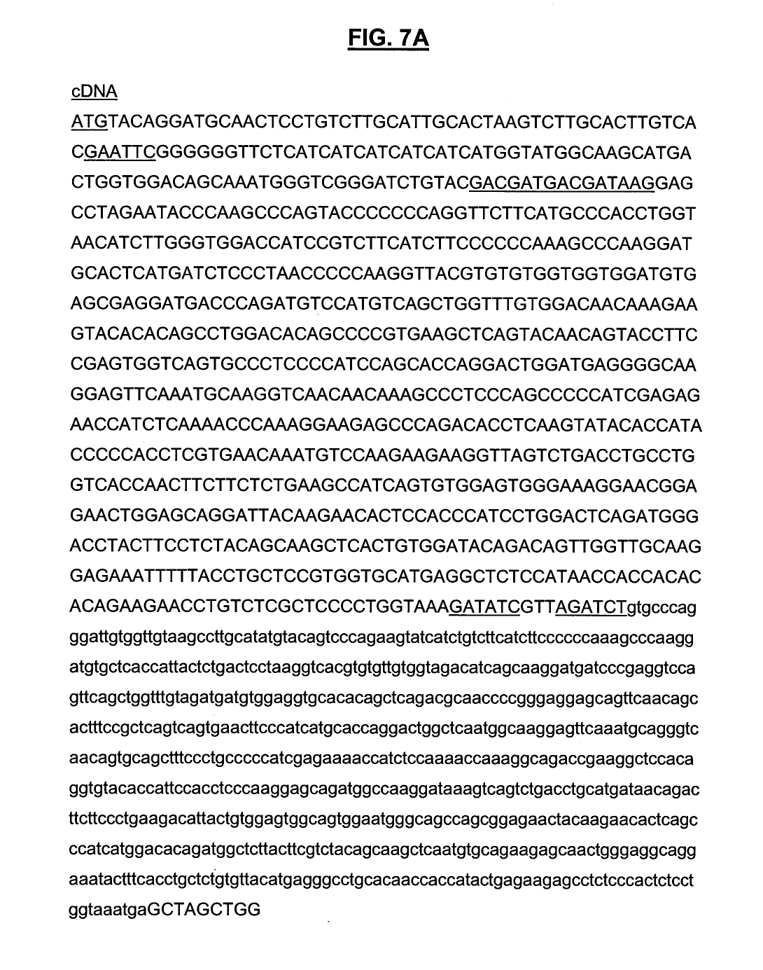

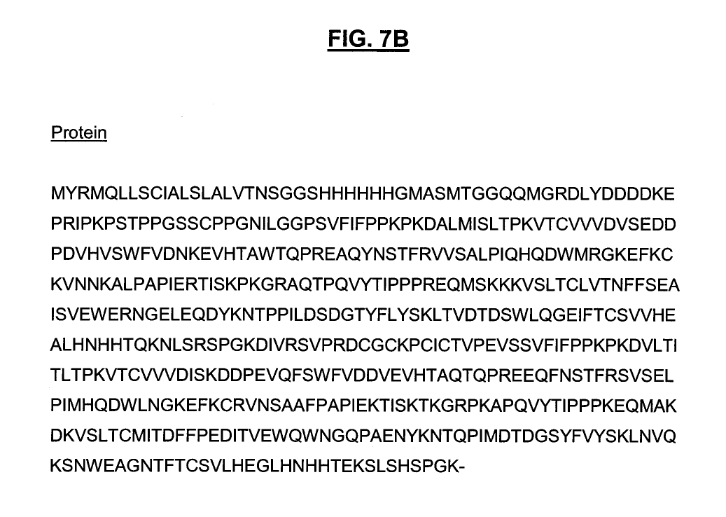

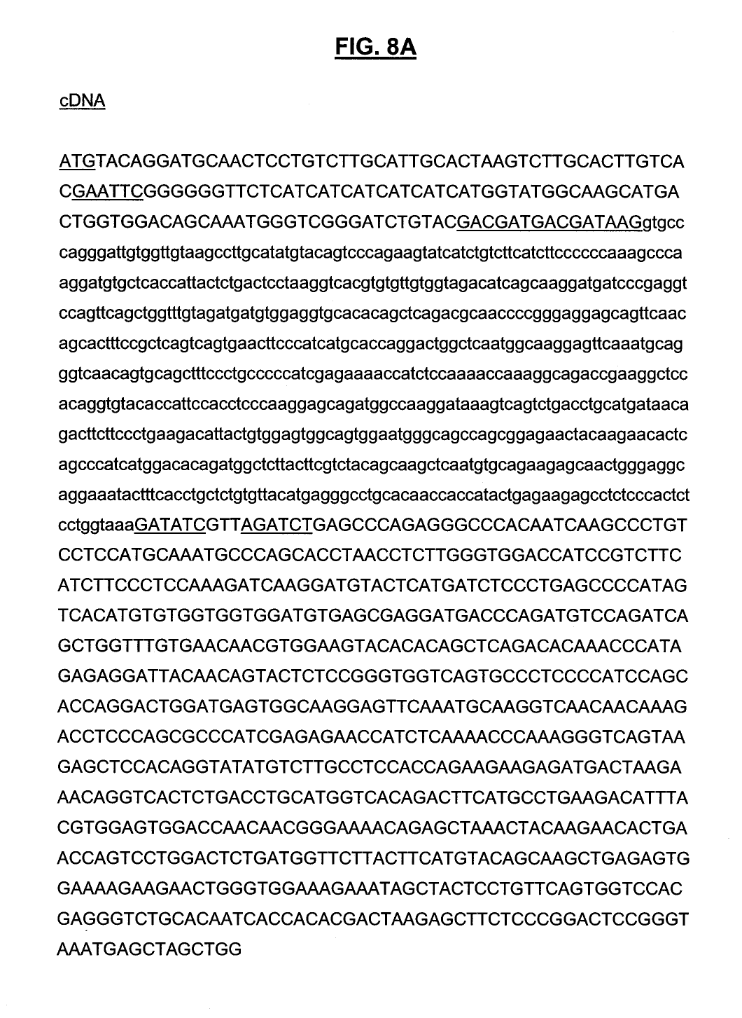

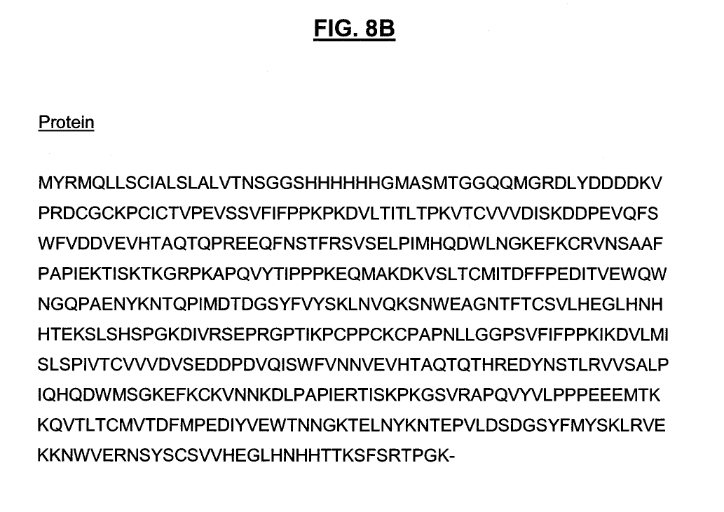

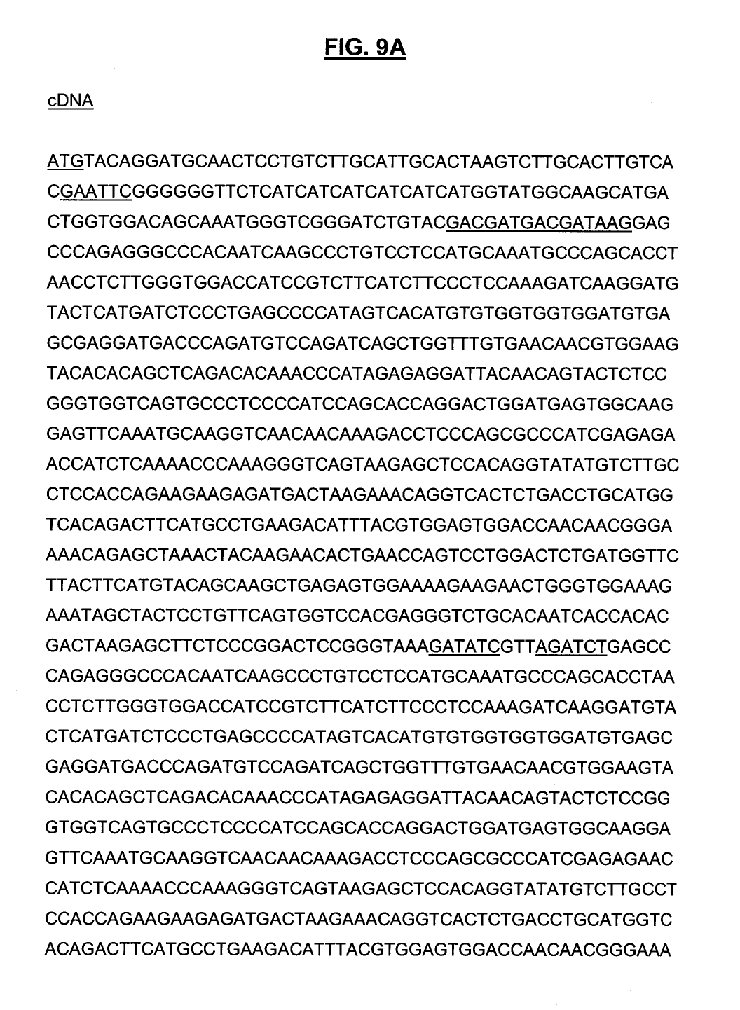

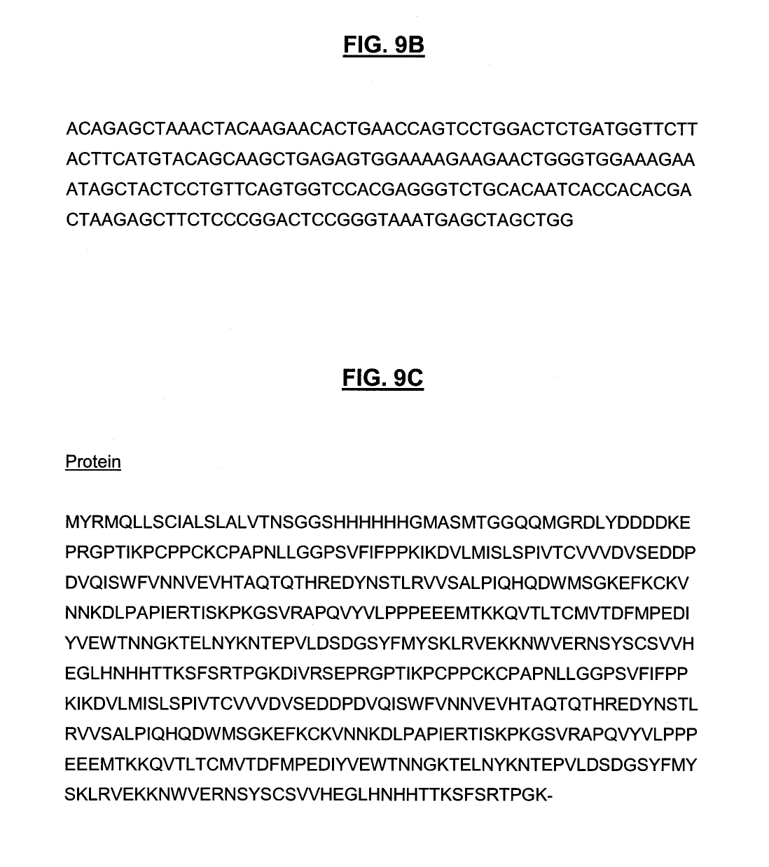

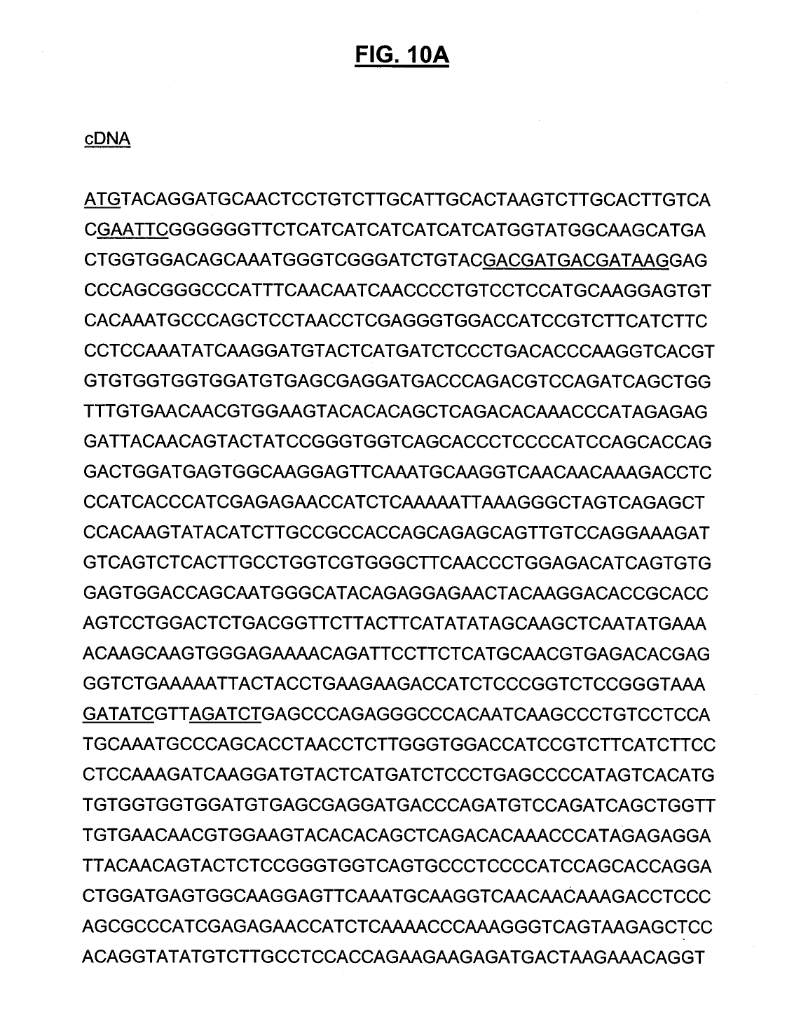

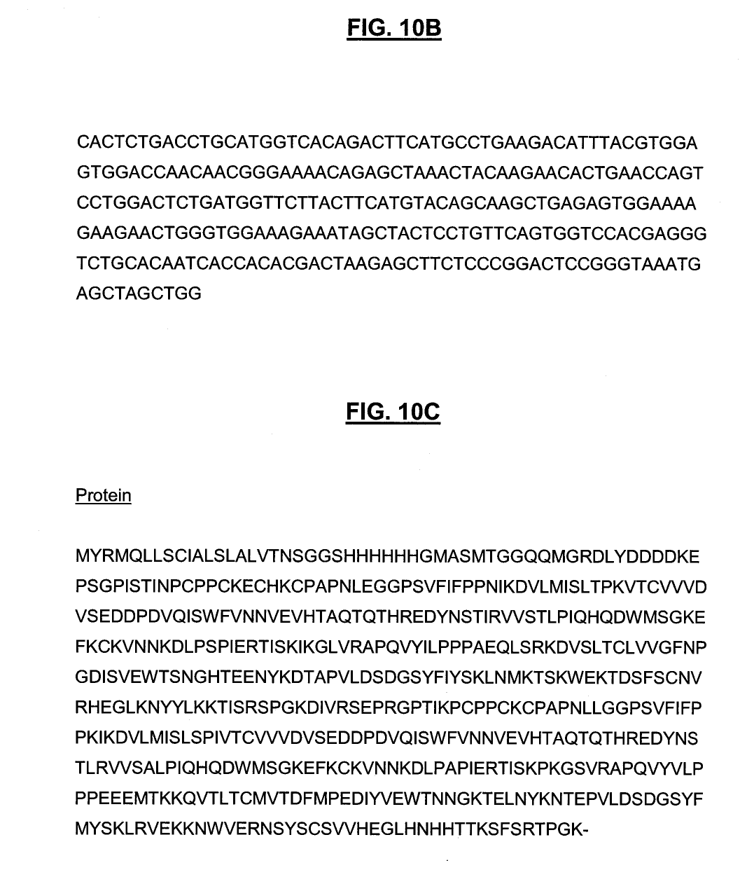

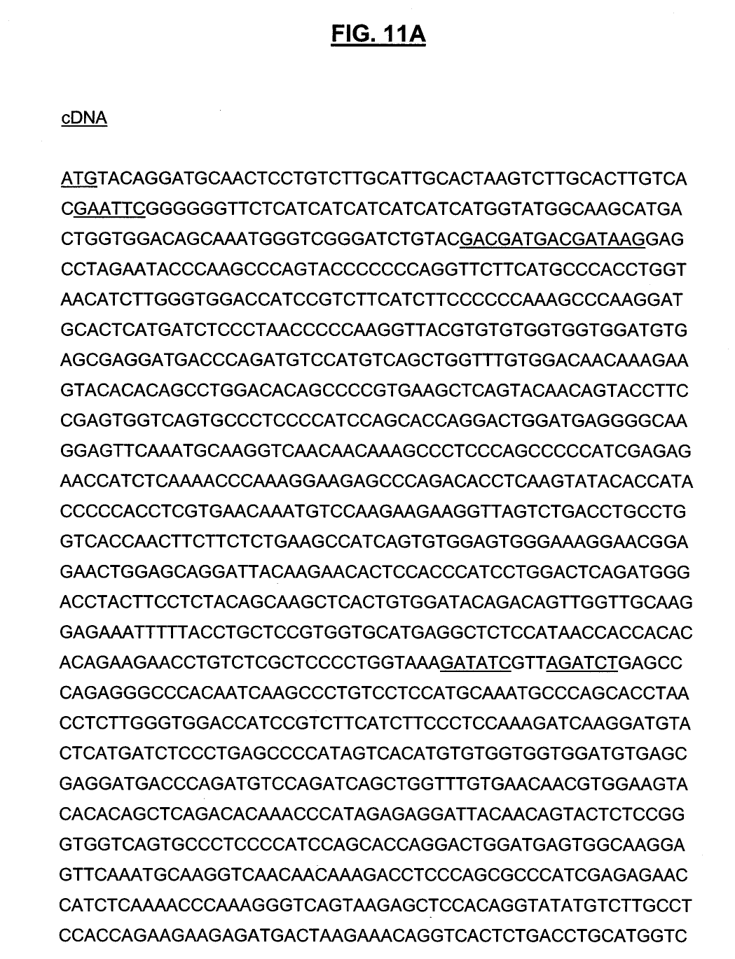

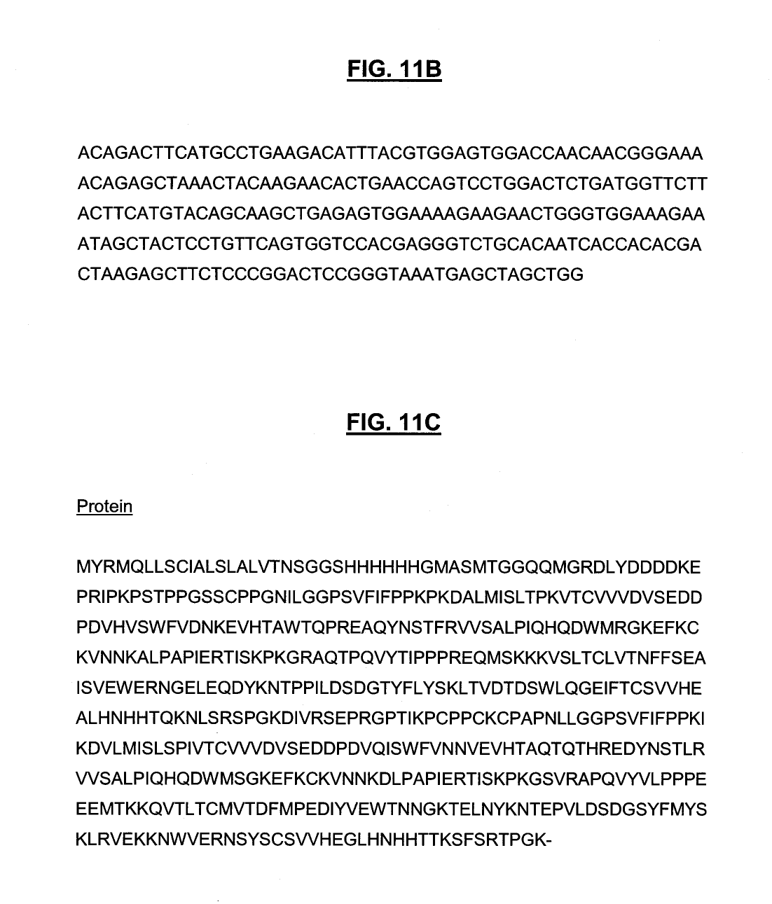

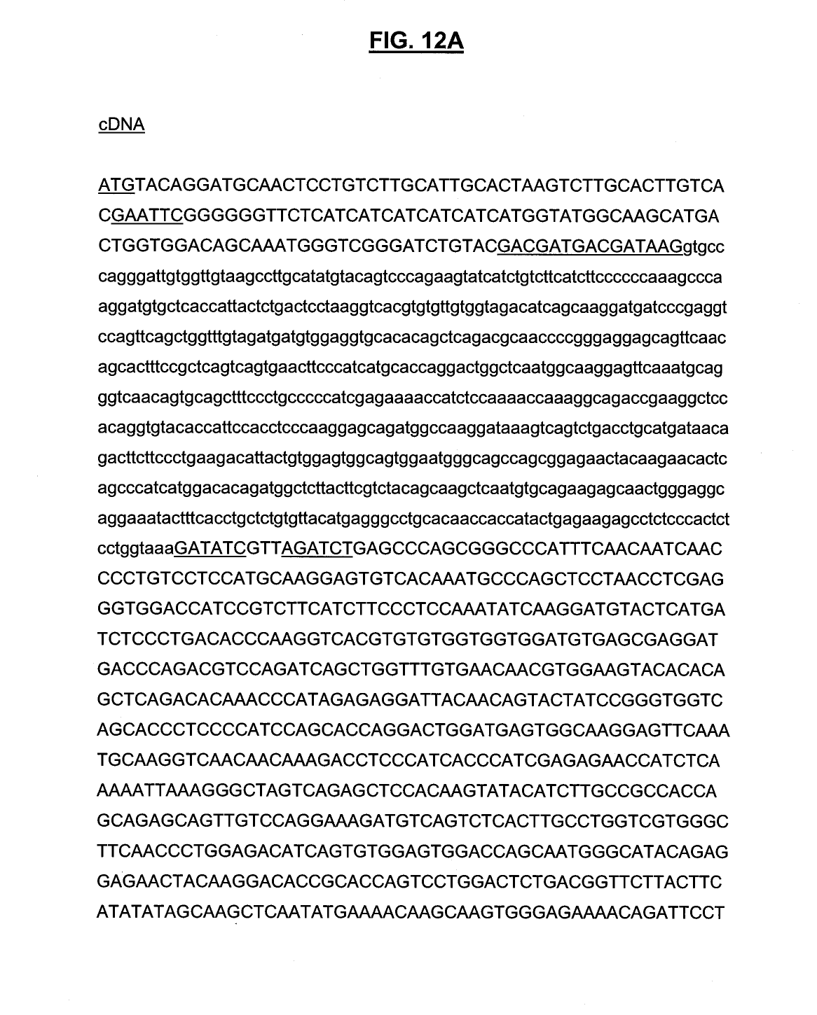

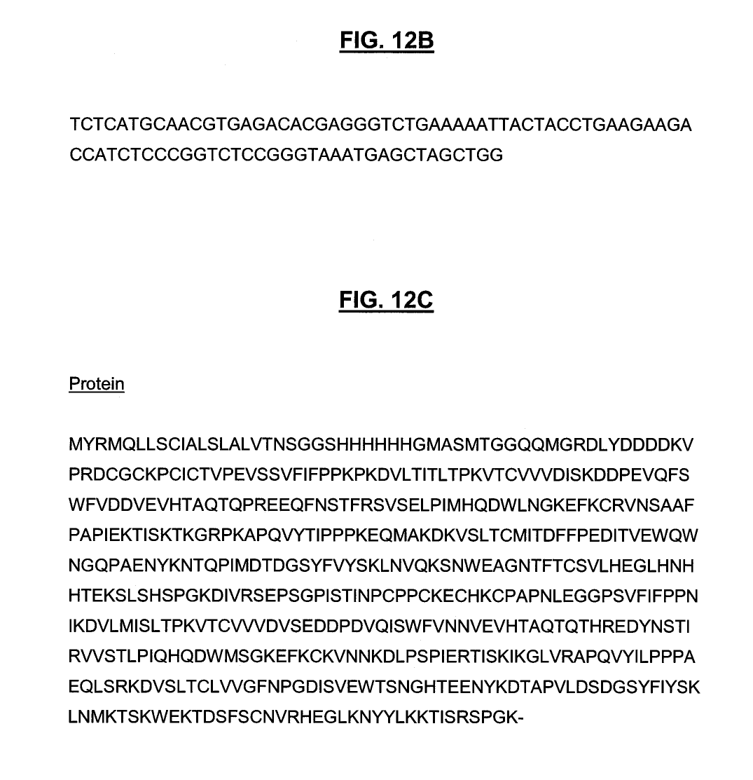

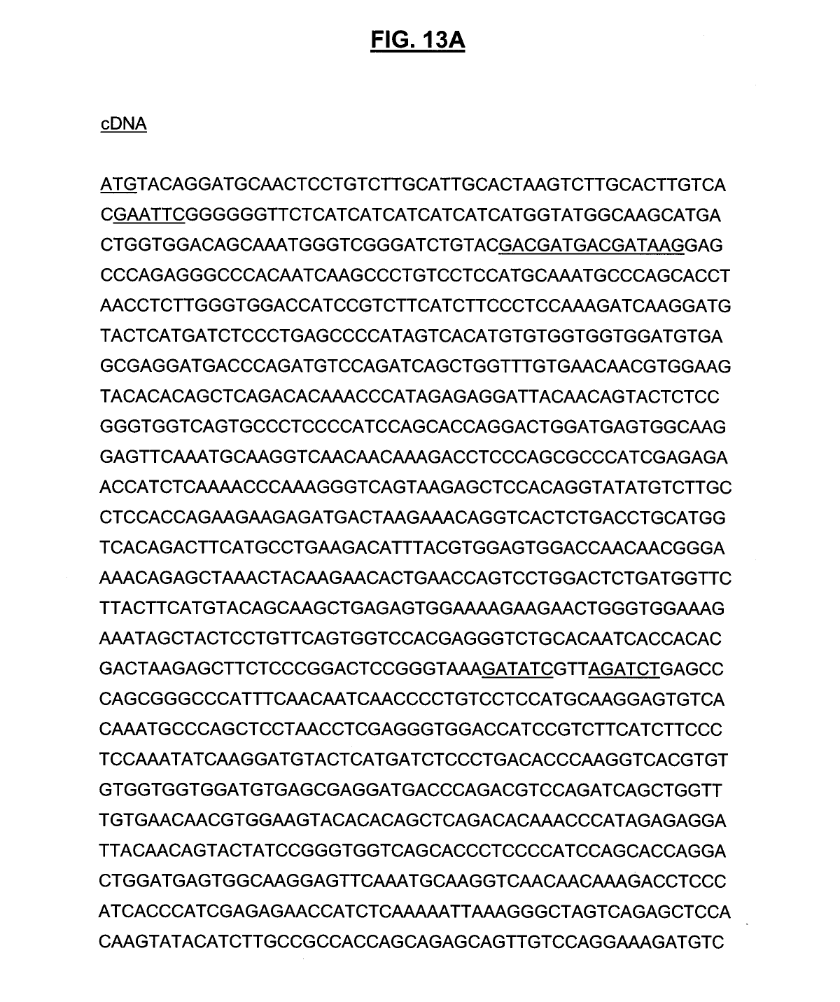

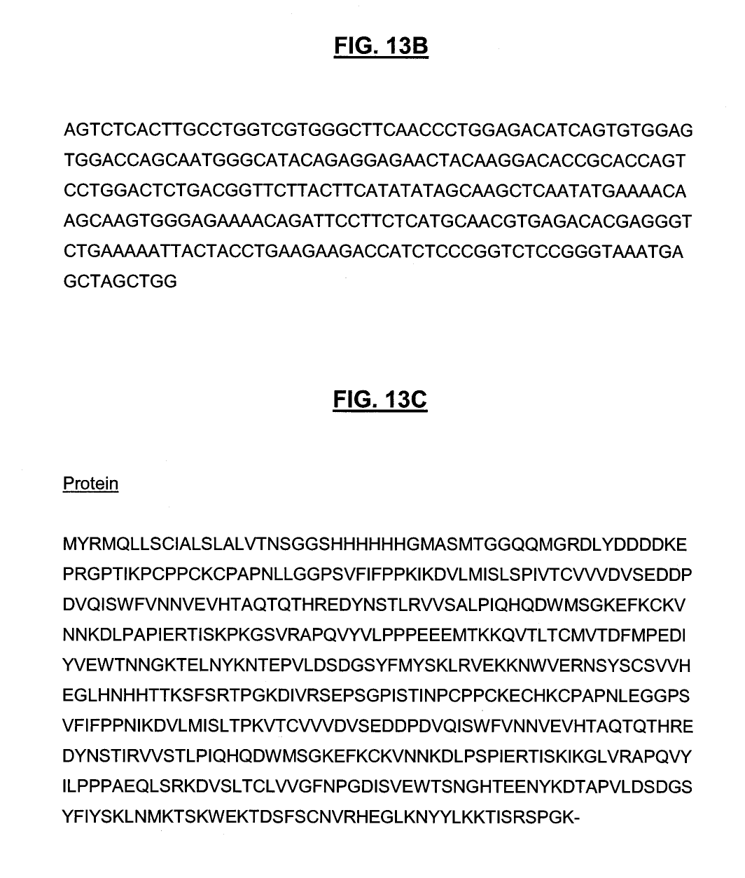

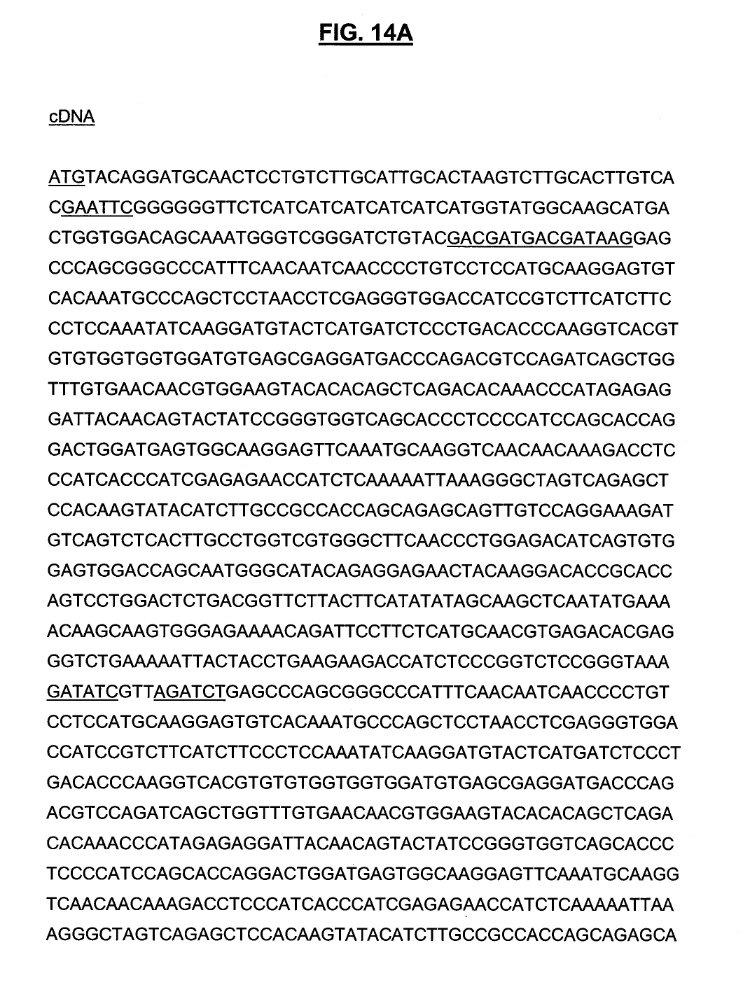

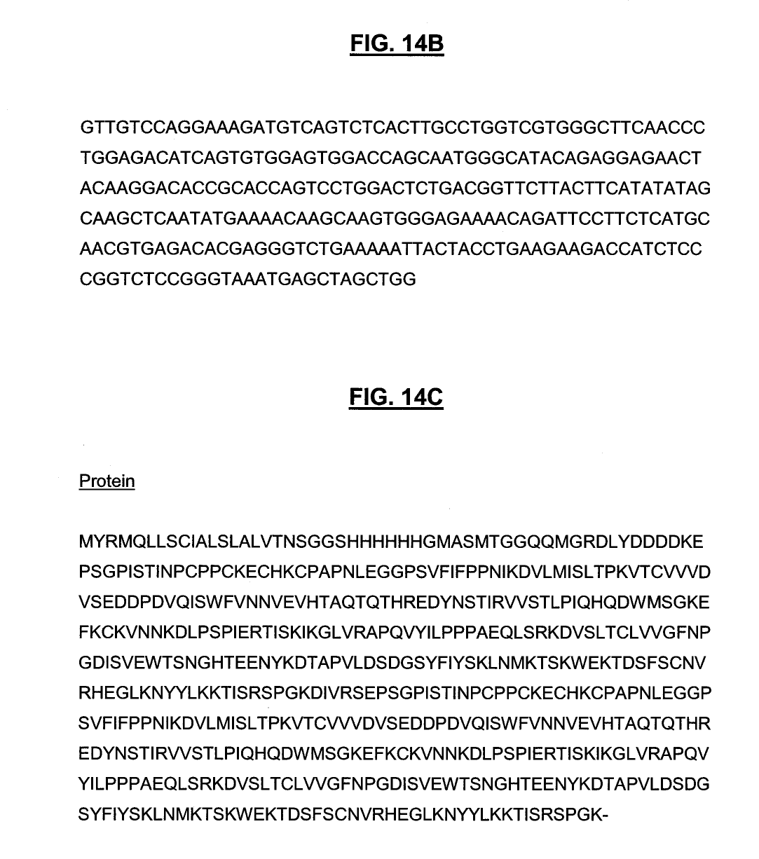

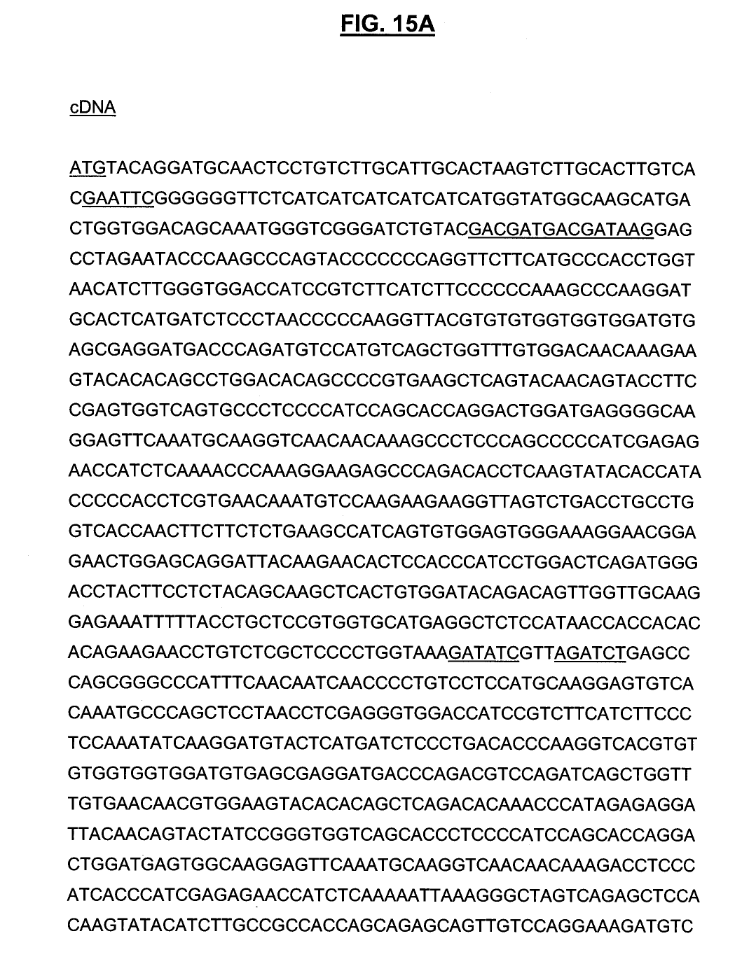

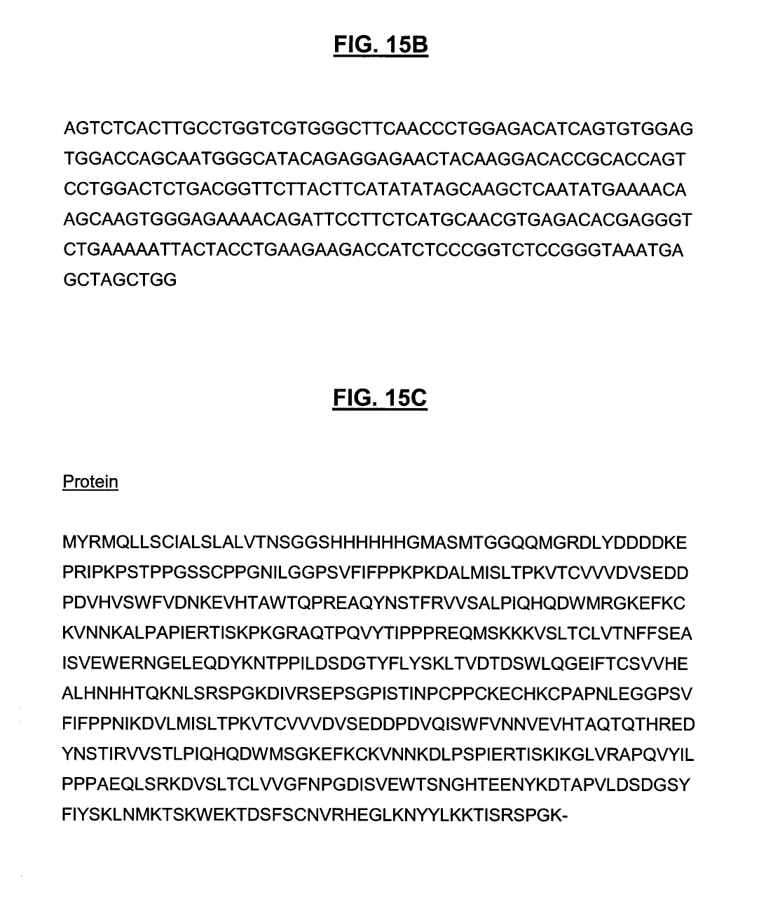

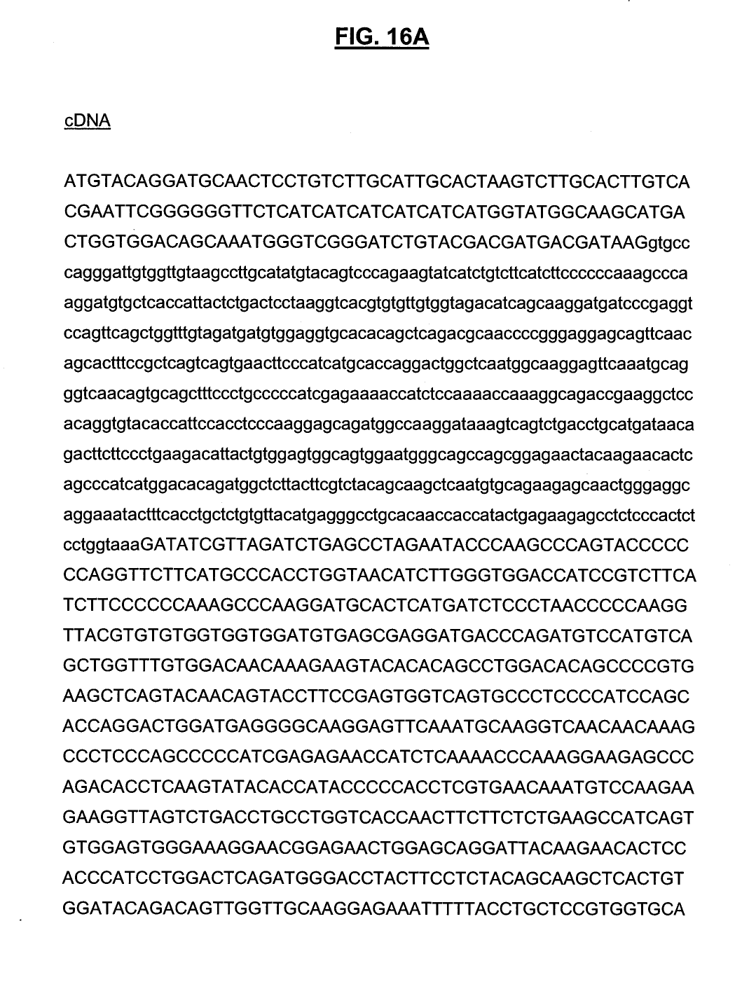

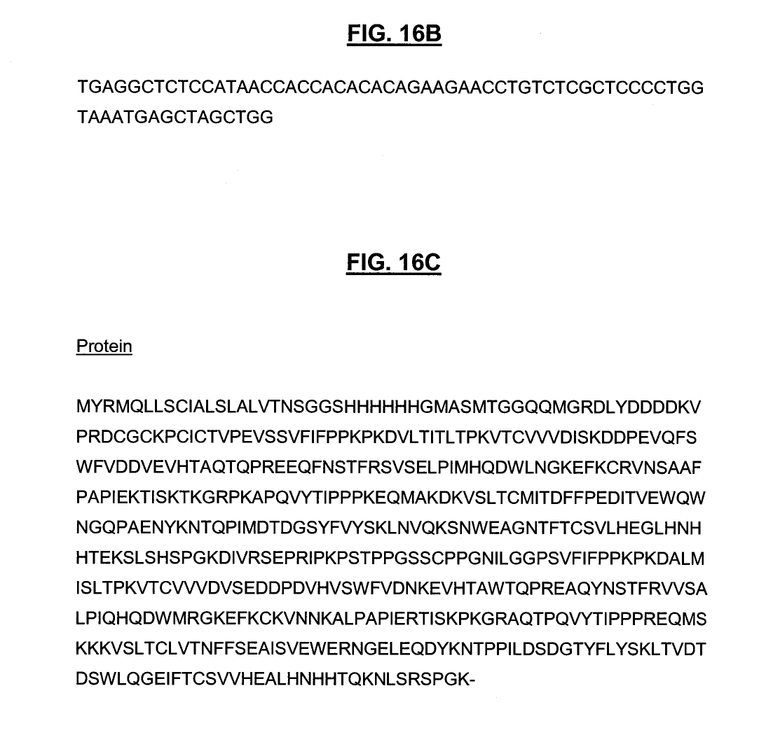

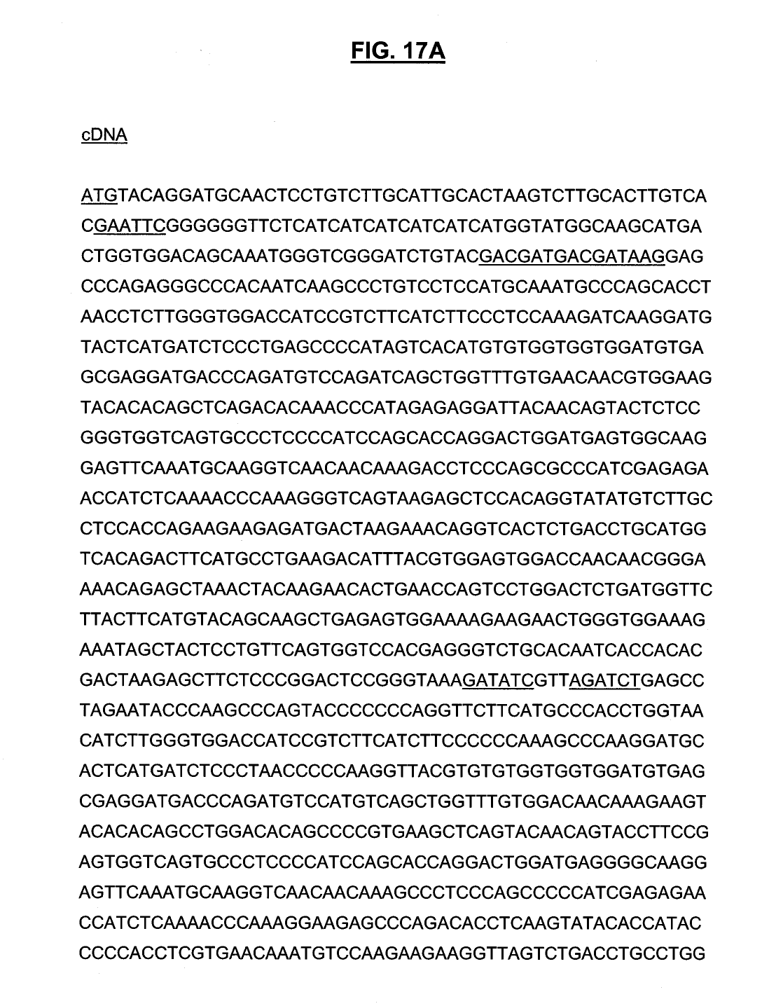

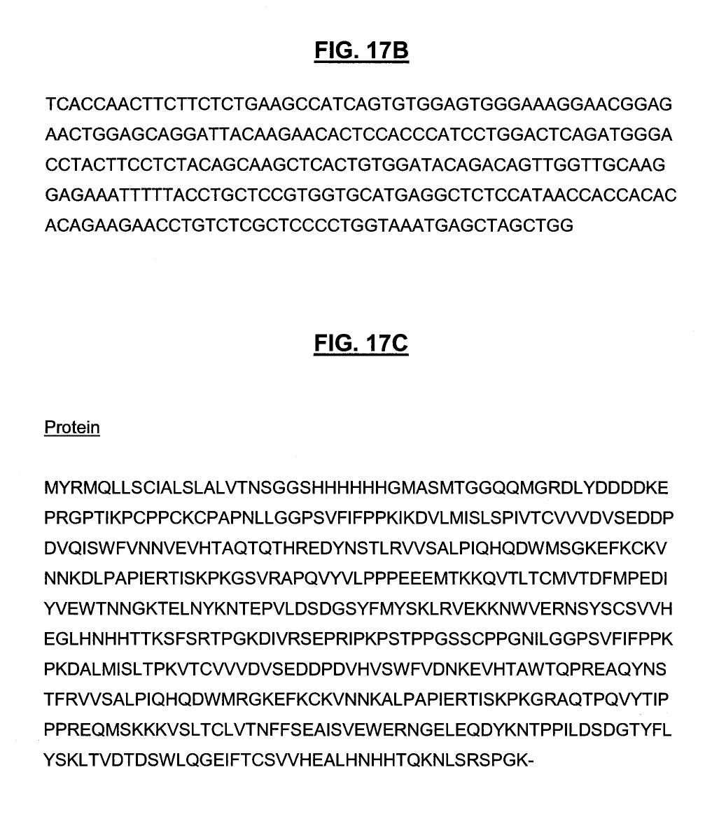

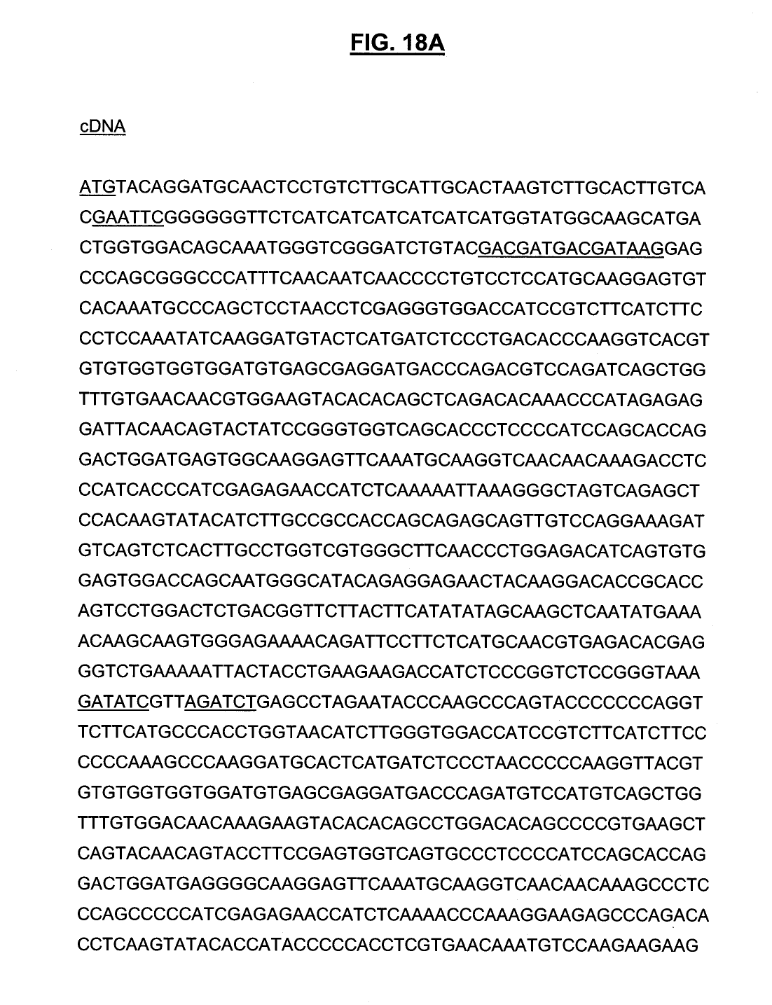

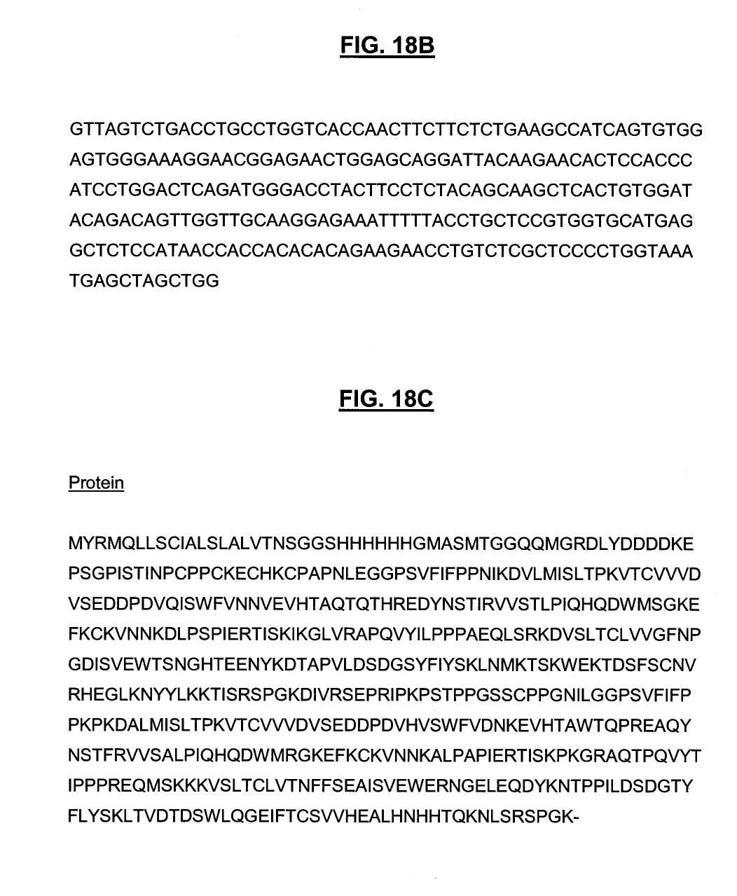

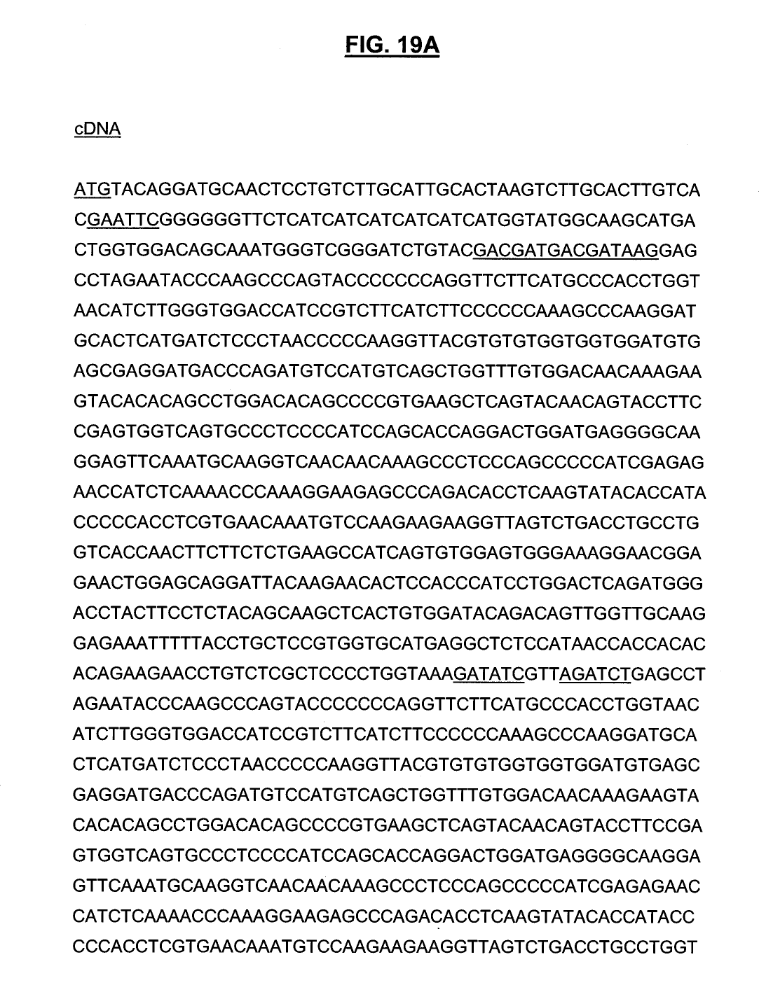

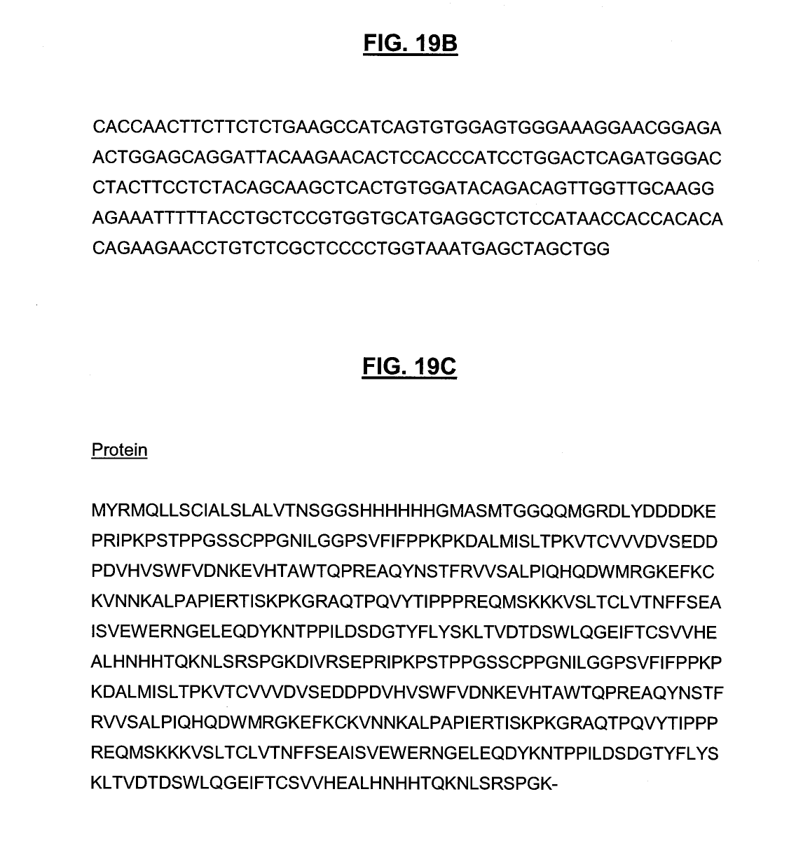

The murine BALB/c IgG1 cDNA, IgG2a cDNA, IgG2b cDNA, or IgG3 cDNA was then subcloned into the pFuse vector comprising the second Fc fragment of murine BALB/c IgG1 cDNA, IgG2a cDNA, IgG2b cDNA, or IgG3 cDNA described in Example 4. The first Fc fragment was subcloned upstream of the second Fc fragment to construct a pFuse vector comprising the first and second Fc fragment of murine BALB/c IgG1 cDNA, IgG2a cDNA, IgG2b cDNA, or IgG3 cDNA and any combinations of fragments thereof (See FIGS. 3-19).

### Example 6

### Cloning of pFuse Vector Comprising a Second Fc Fragment of Murine C57BL/6 IgG cDNA

Spleens were isolated from C57BL/6 mice (Taconic) and total RNA was isolated from the spleens. The murine C57BL/6 IgG cDNA was reverse transcribed from the RNA. Mice contain different isotypes of IgG. For example, isotypes of IgG for C57BL/6 mice include IgG1, IgG2b, IgG2c and IgG3.

The second Fc fragment of murine C57BL/6 IgG1 cDNA, IgG2b cDNA, IgG2c cDNA, or IgG3 cDNA was amplified by PCR using the following primers:

mIgG1 sense:
(SEQ ID NO: 120)
5′-TTAGATCTGTGCCCAGGGATTGTGGT-3′
mIgG1 antisense:
(SEQ ID NO: 121)
5′-CAGCTAGCTCATTTACCAGGAGAGTGGGAG-3′
mIgG2b sense:
(SEQ ID NO: 122)
5′-TTAGATCTGAGCCCAGCGGGCCCATT-3′
mIgG2b antisense:
(SEQ ID NO: 123)
5′-CAGCTAGCTCATTTACCCGGAGACCG-3′
mIgG2c sense:
(SEQ ID NO: 124)
5′-TTAGATCTGAGCCCAGAGTGCCCATA-3′
mIgG2c antisense:
(SEQ ID NO: 125)
5′-CAGCTAGCTCATTTACCCAGAGACCGG-3′
mIgG3 sense:
(SEQ ID NO: 126)
5′-TTAGATCTGAGCCTAGAATACCCAAGCCCA-3′
mIgG3 antisense:
(SEQ ID NO: 127)
5′-CAGCTAGCTCATTTACCAGGGGAGCGA-3′

Using the same approach as set forth in Example 1, a pFuse vector comprising a second Fc fragment of murine C57BL/6 IgG1 cDNA, IgG2b cDNA, IgG2c cDNA, or IgG3 cDNA was constructed. The second Fc fragment of murine C57BL/6 IgG1 cDNA, IgG2b cDNA, IgG2c cDNA, or IgG3 cDNA comprised a hinge region, C_{H}2 domain and C_{H}3 domain.

### Example 7

### Cloning of pFuse Vector Comprising a First and Second Fc Fragment of Murine C57BL/6 IgG cDNA

The first Fc fragment of murine C57BL/6 IgG1 cDNA, IgG2b cDNA, IgG2c cDNA, or IgG3 cDNA was amplified by PCR using the following primers:

mIgG fragment 1 sense:
(SEQ ID NO: 128)
5′-ACGAATTCGGGGGGTTCTC-3′
mIgG1 fragment 1 antisense:
(SEQ ID NO: 129)
5′-CTAGATCTAACGATATCTTTACCAGGAGAGTGGGAGAGG-3′
mIgG2b fragment 1 antisense:
(SEQ ID NO: 130)
5′-CTAGATCTAACGATATCTTTACCCGGAGACCGG-3′
mIgG2c fragment 1 antisense:
(SEQ ID NO: 131)
5′-CTAGATCTAACGATATCTTTACCCAGAGACCGGGAG-3′
mIgG3 fragment 1 antisense:
(SEQ ID NO: 132)
5′-CTAGATCTAACGATATCTTTACCAGGGGAGCGAGAC-3′

Using the same approach as set forth in Example 2, a pFuse vector comprising a first Fc fragment of murine C57BL/6 IgG1 cDNA, IgG2b cDNA, IgG2c cDNA, or IgG3 cDNA was constructed.

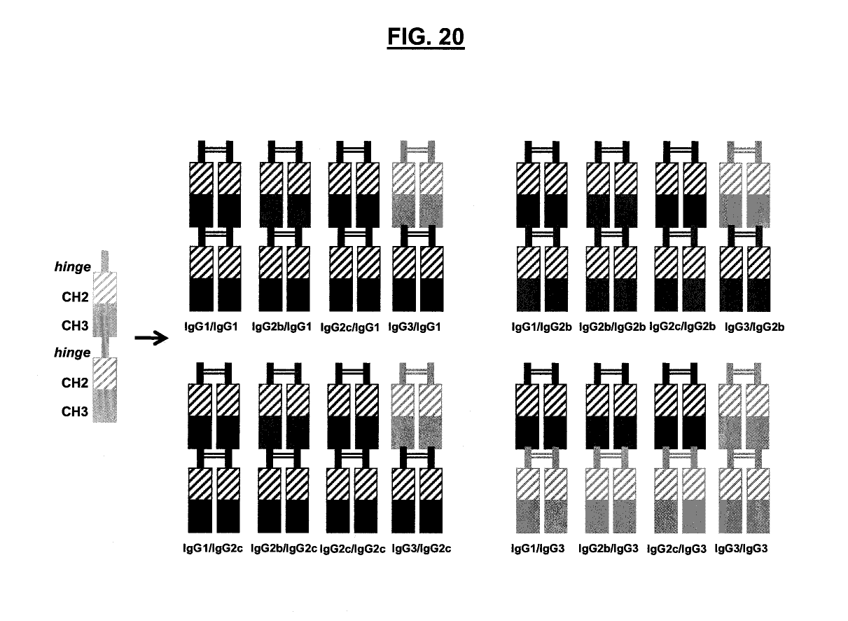

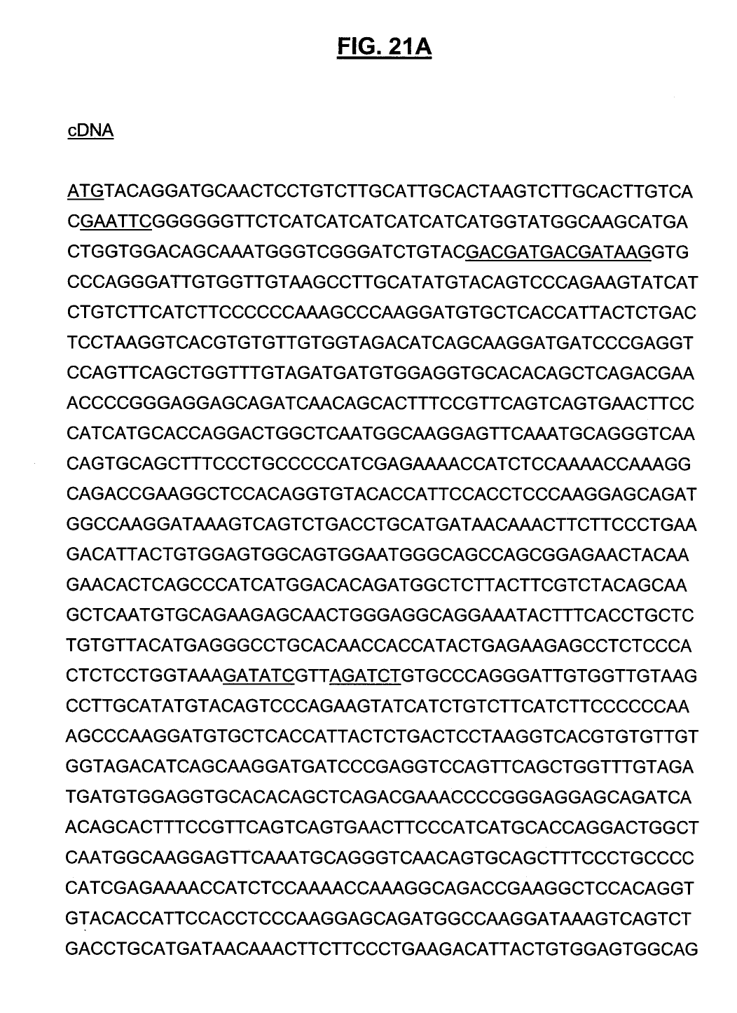

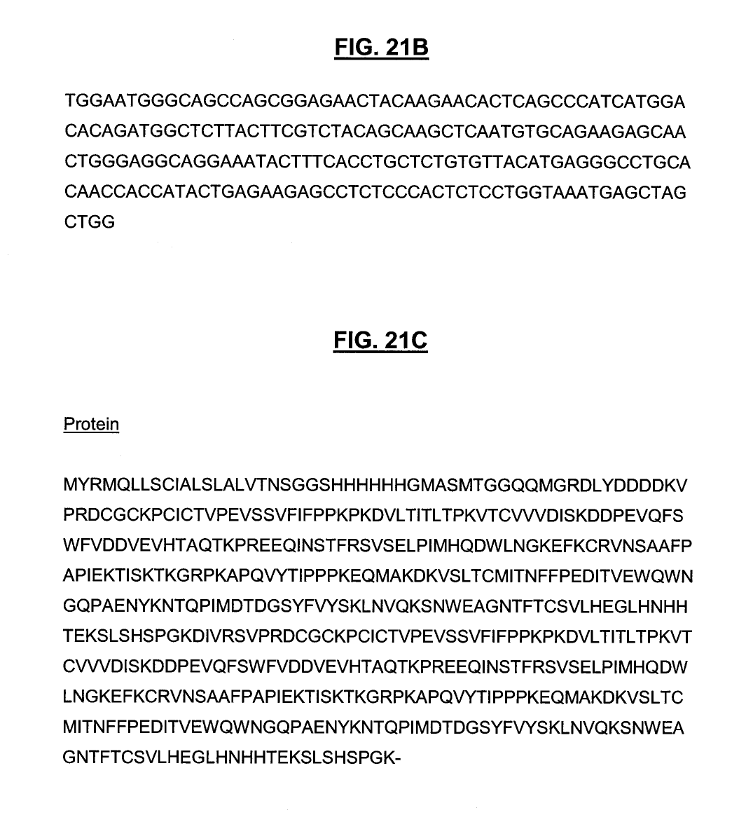

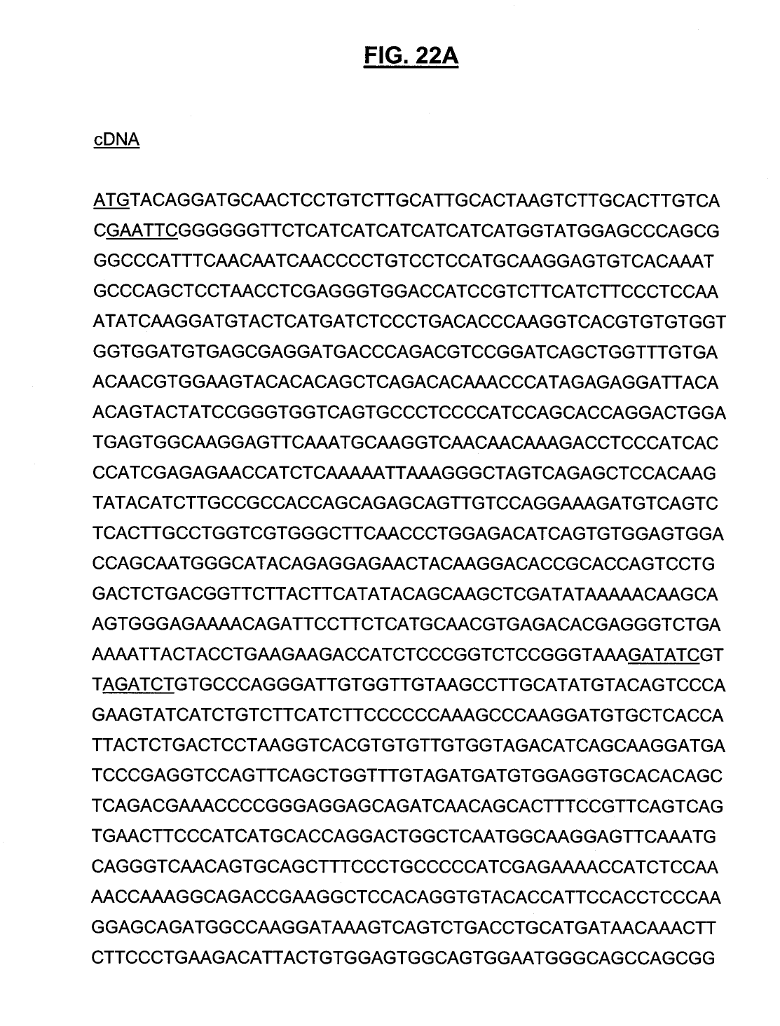

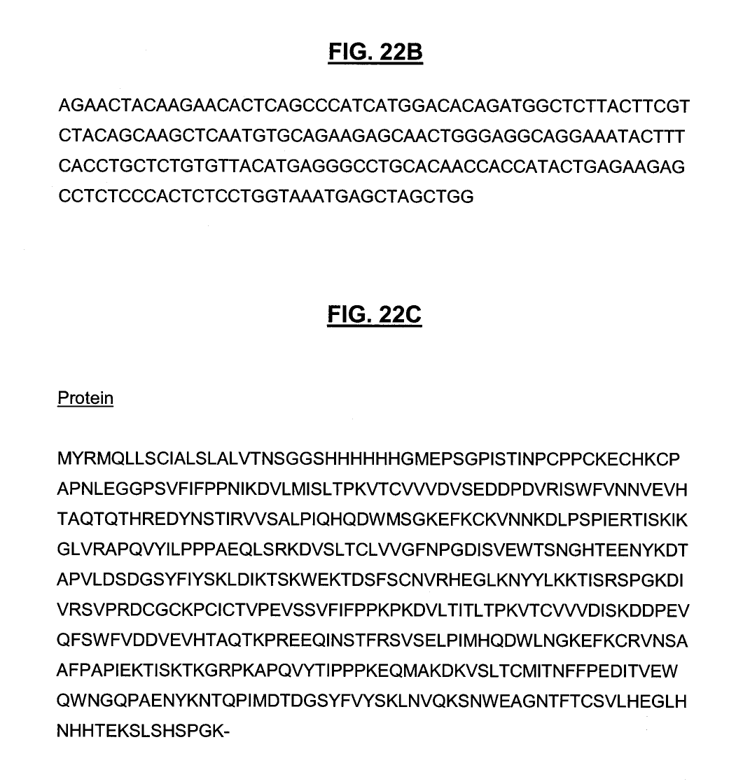

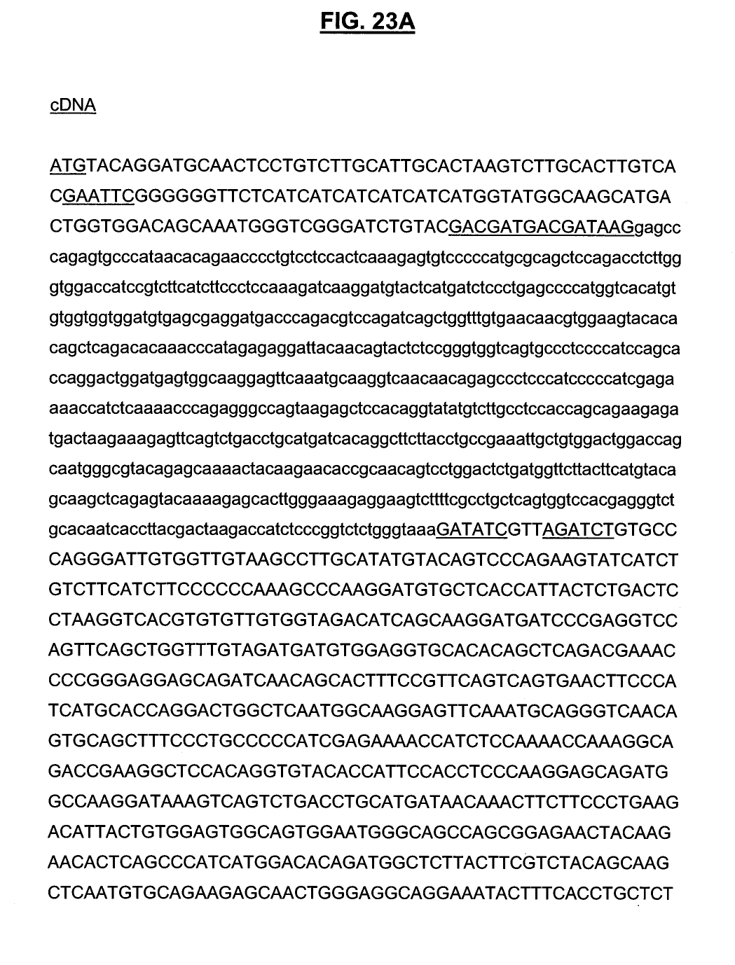

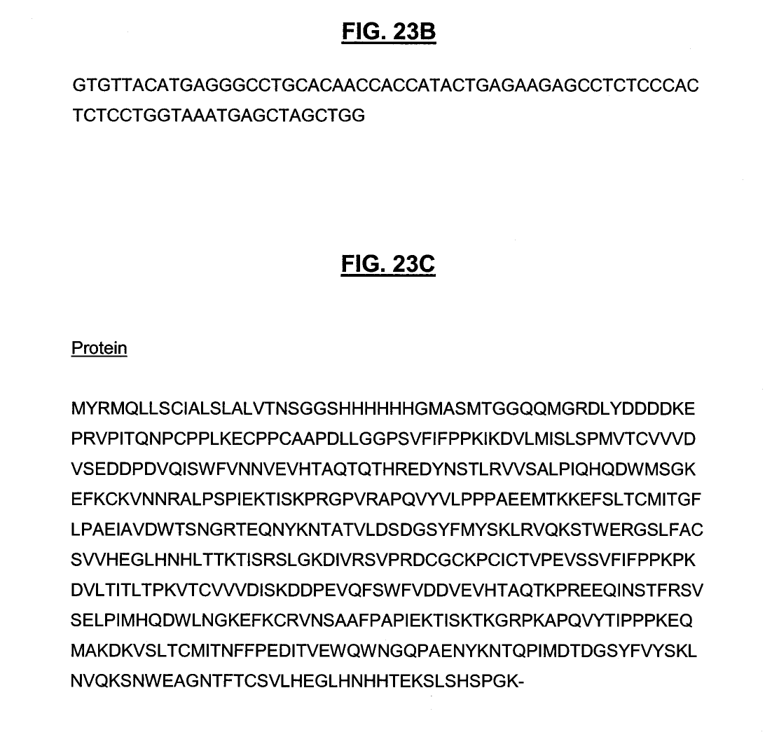

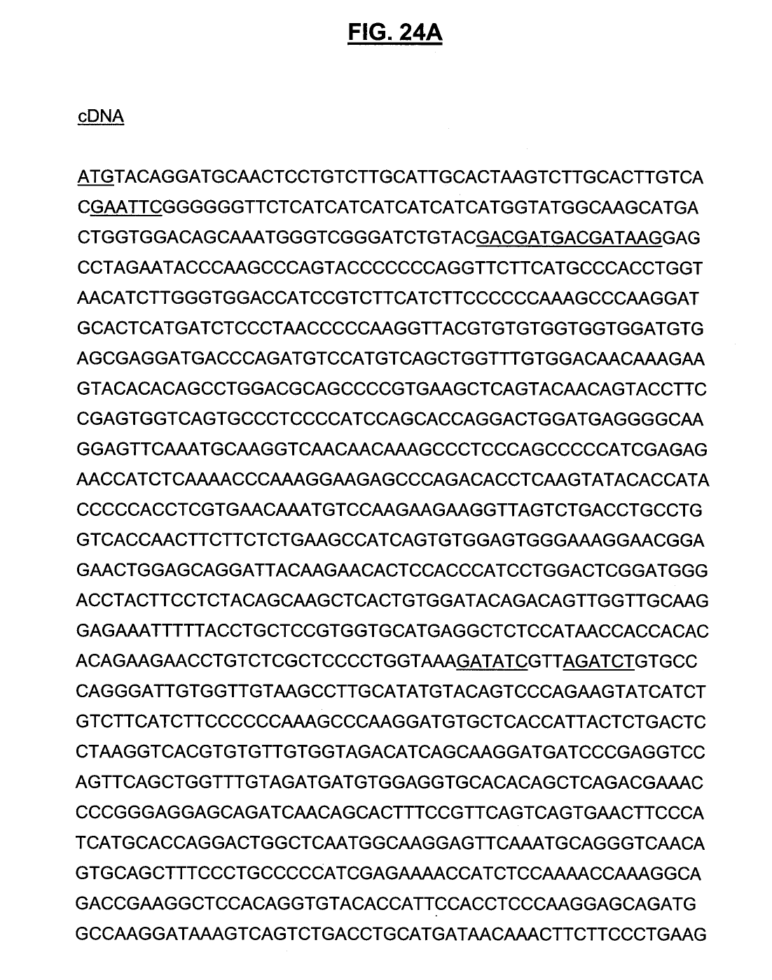

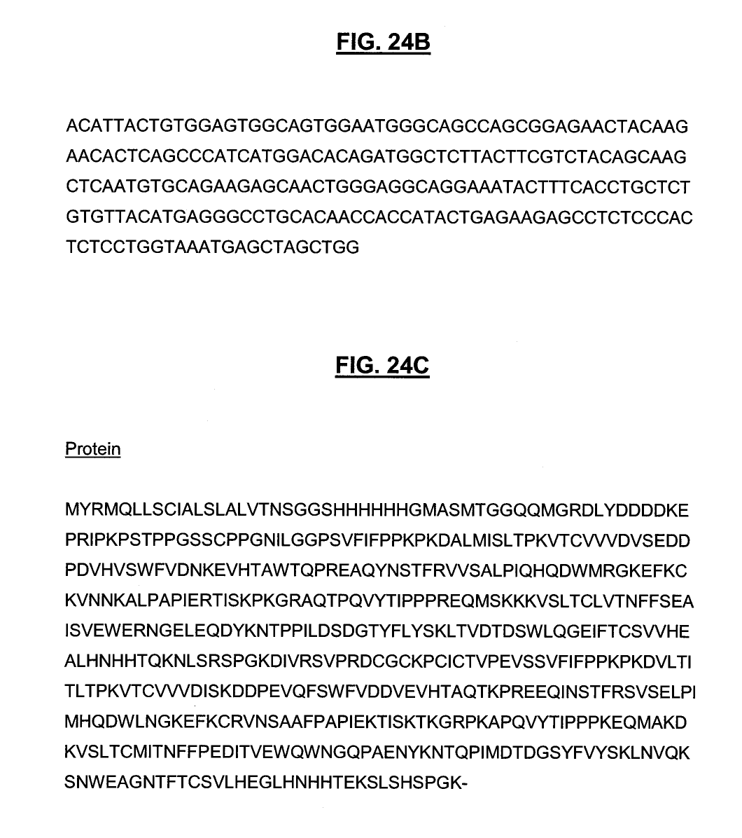

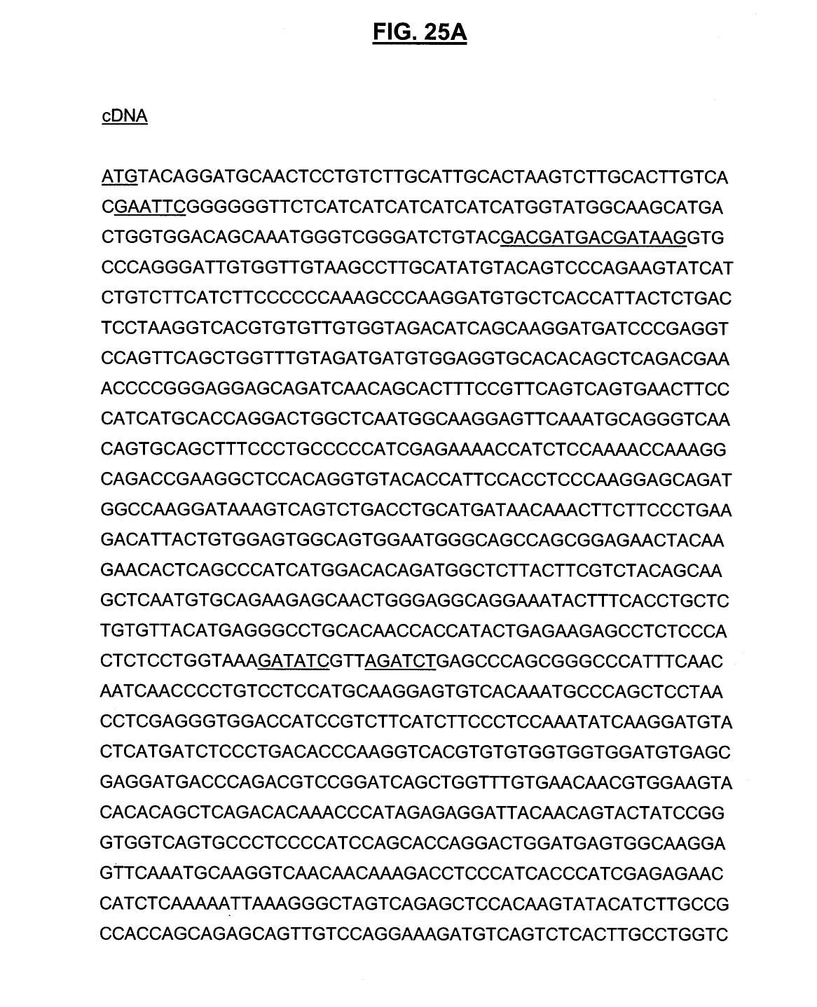

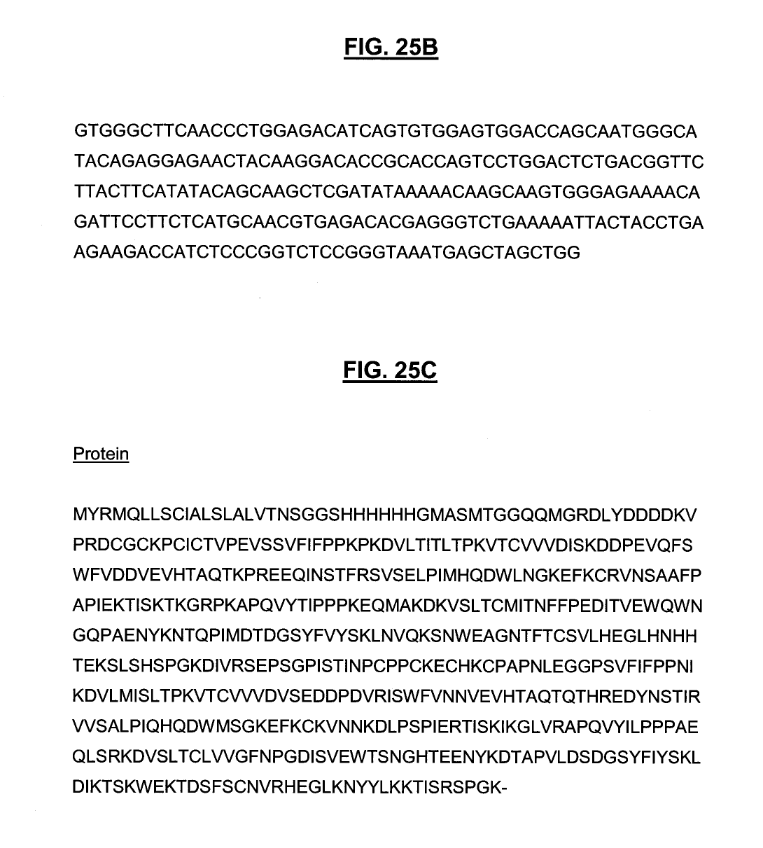

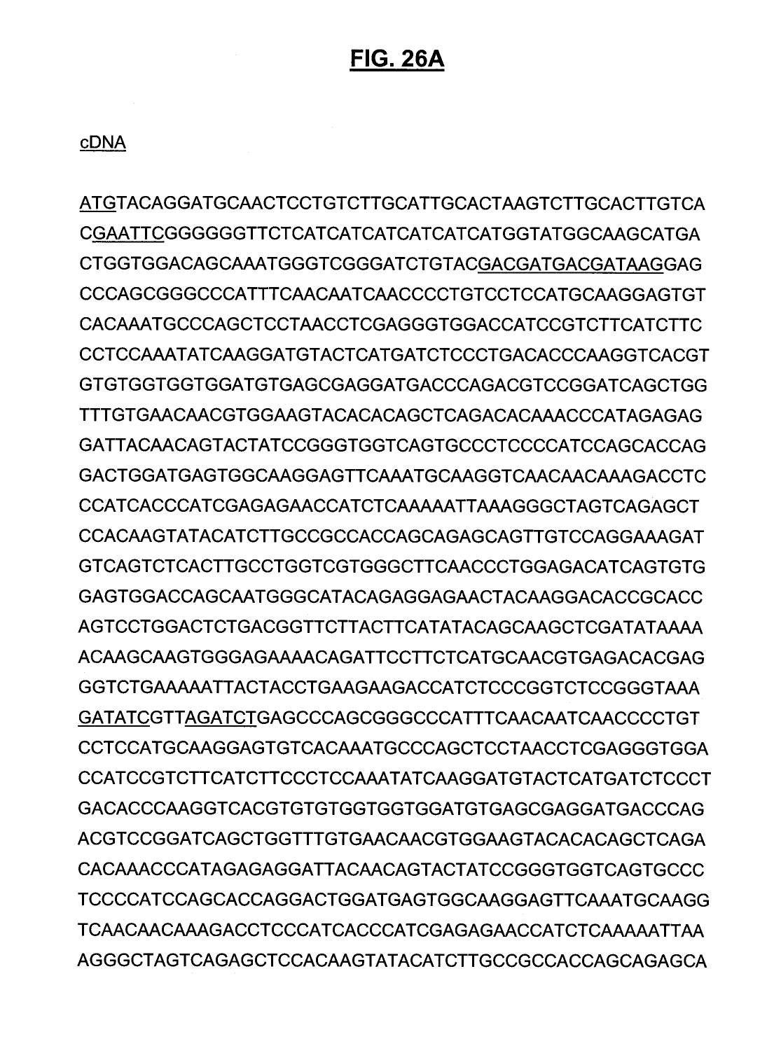

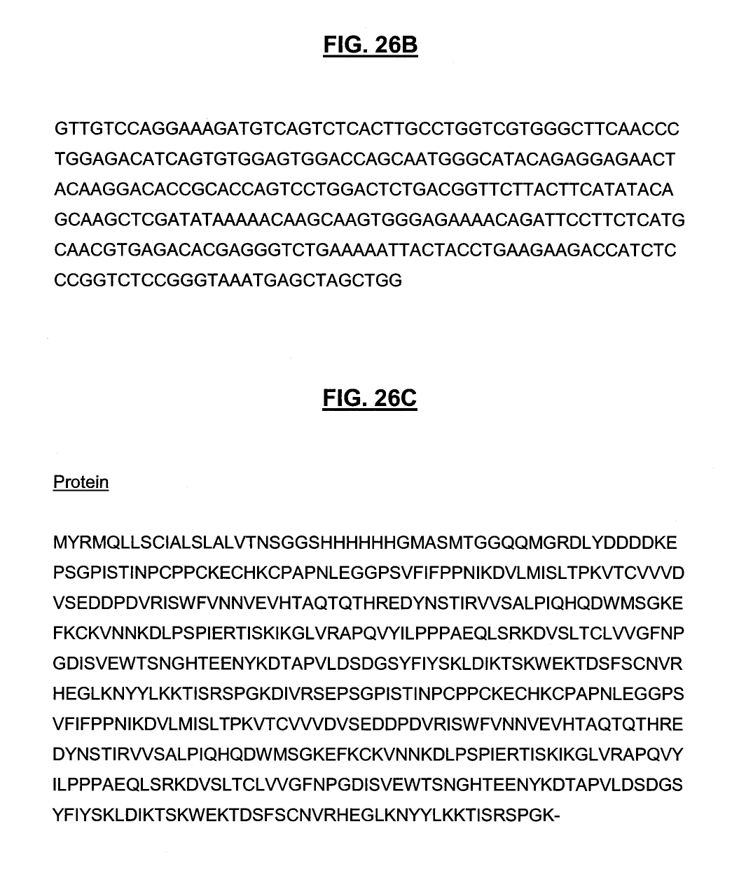

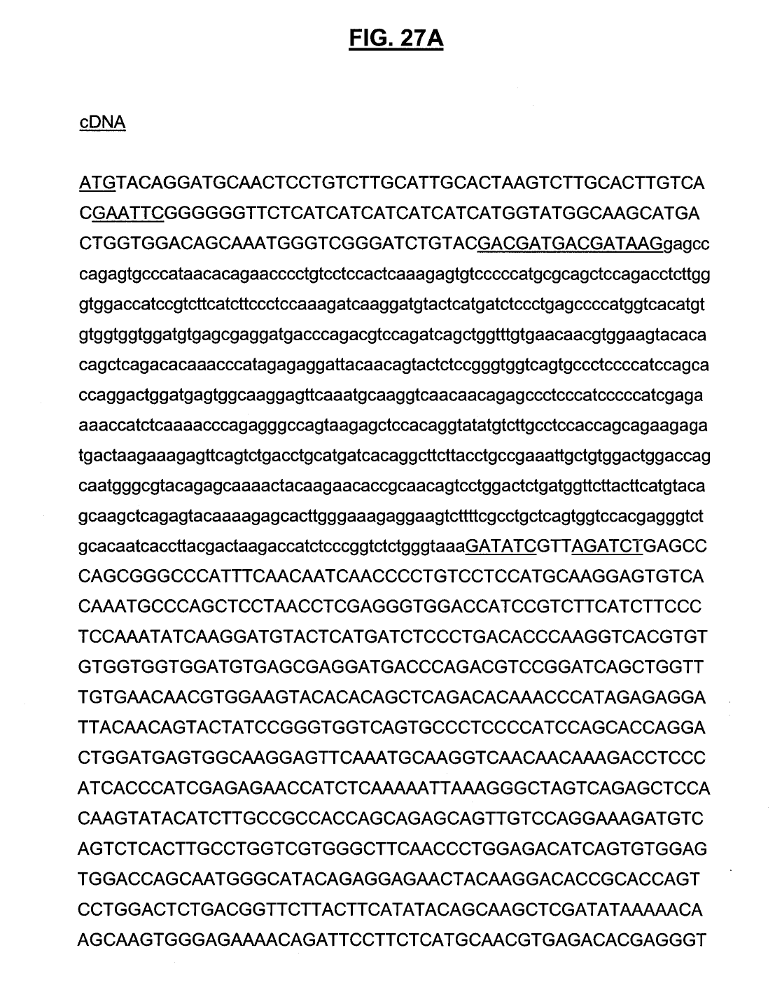

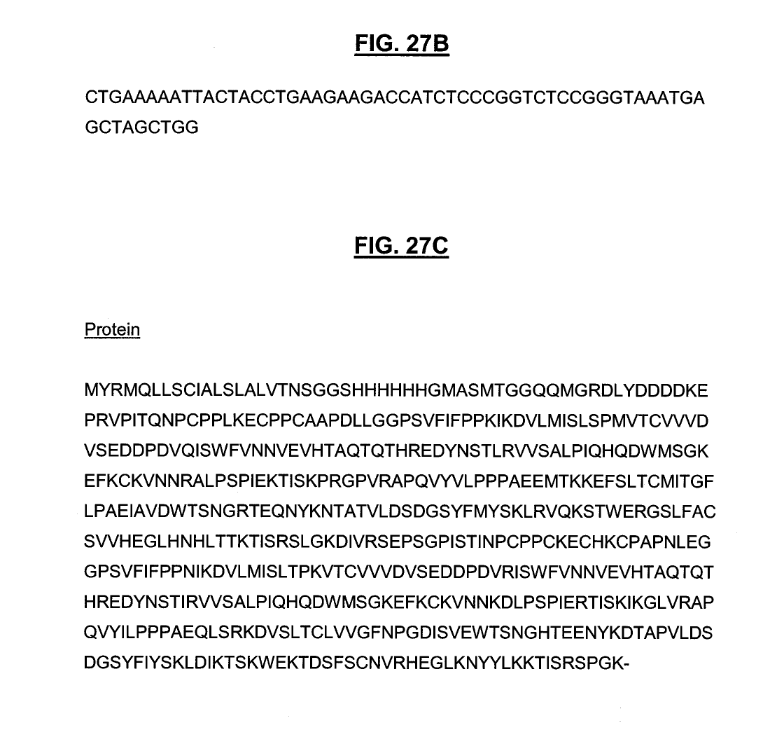

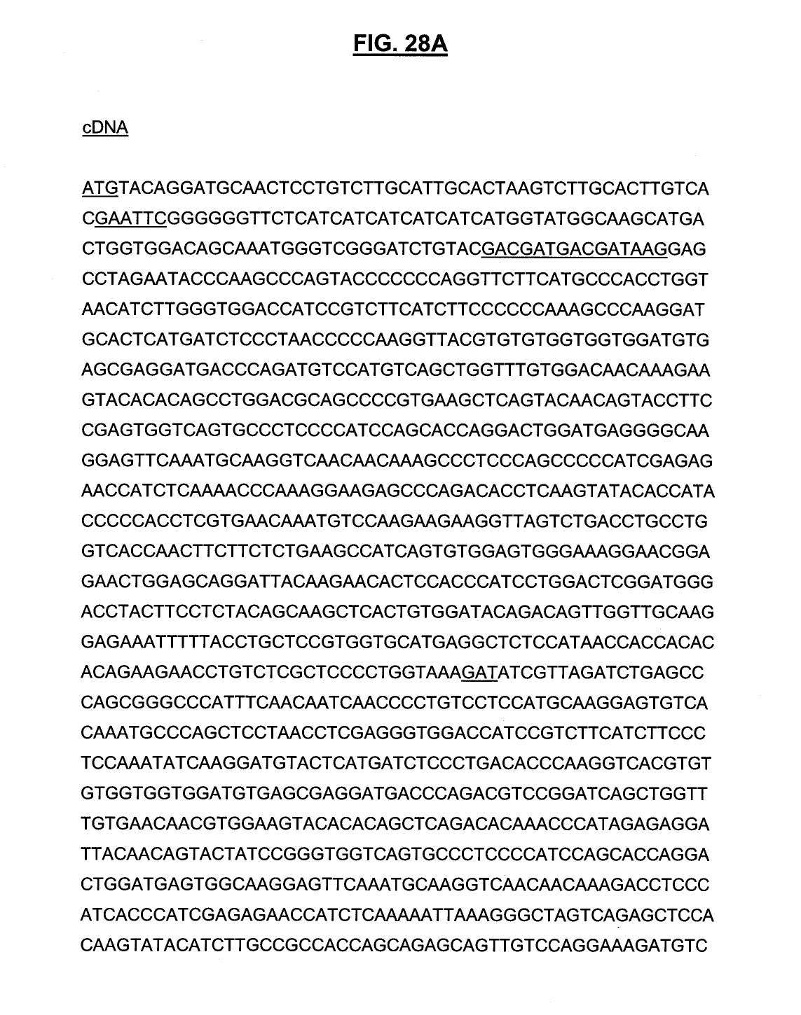

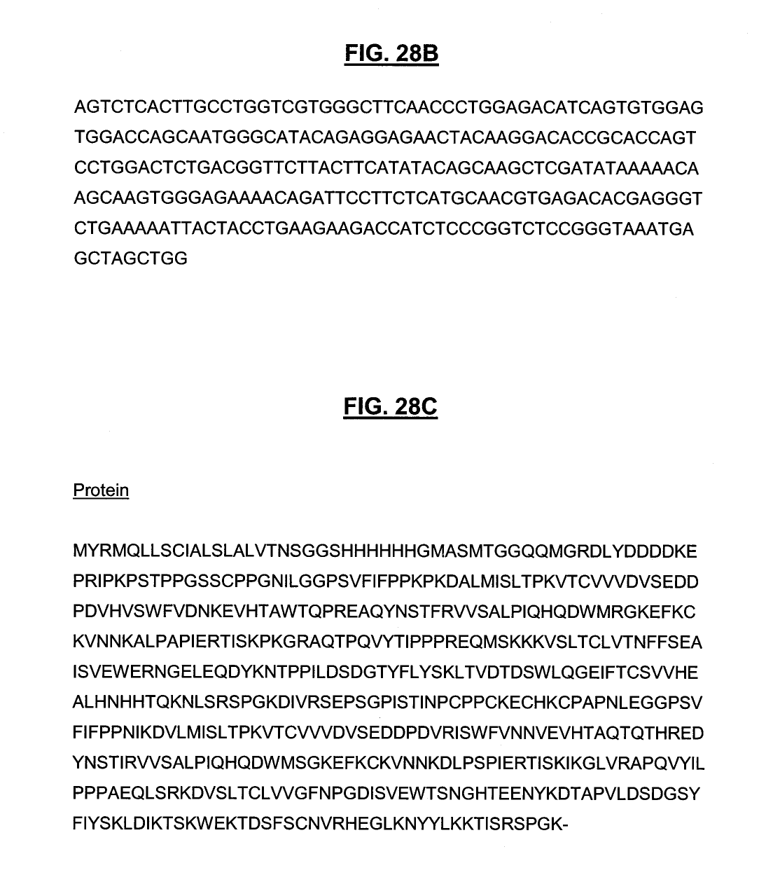

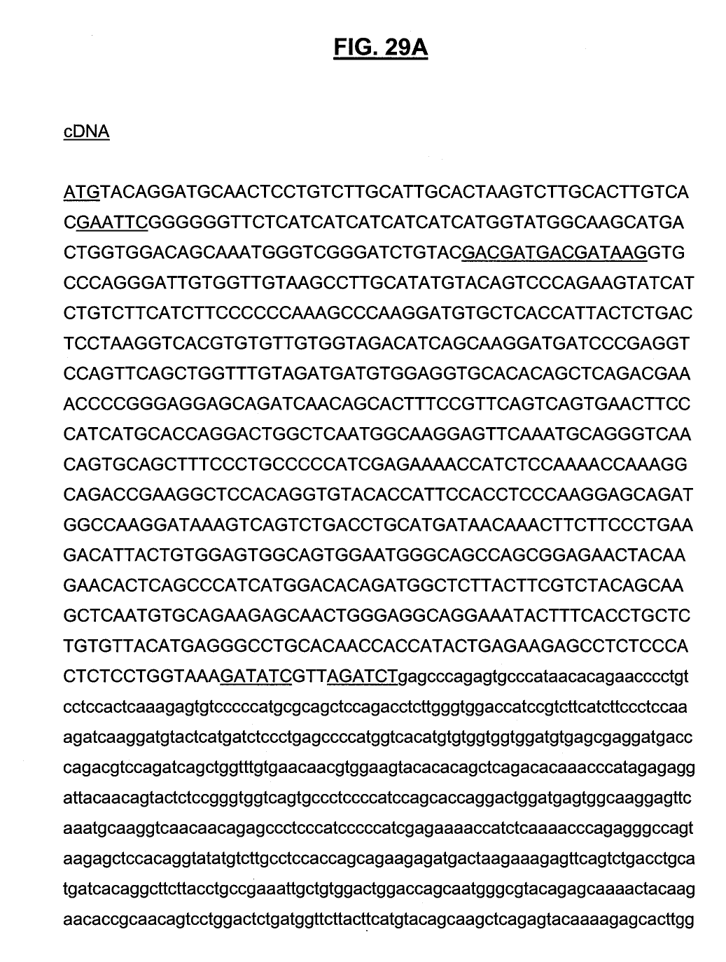

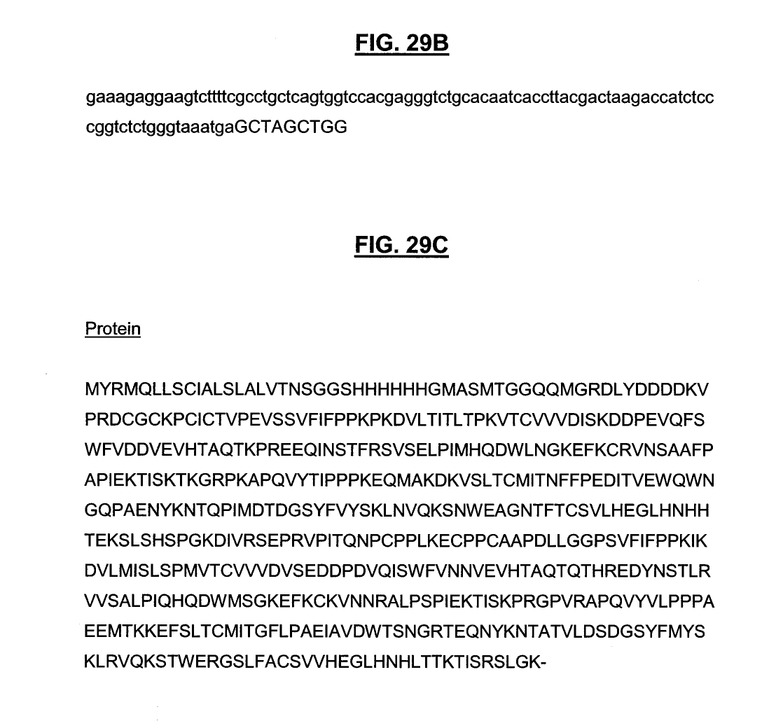

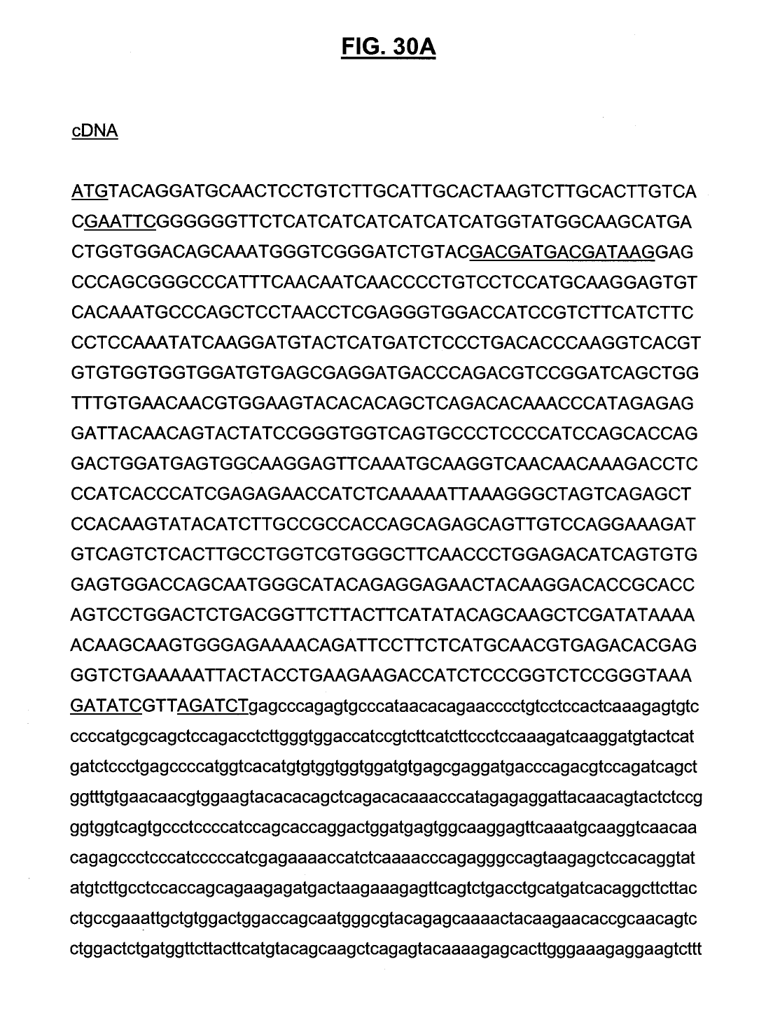

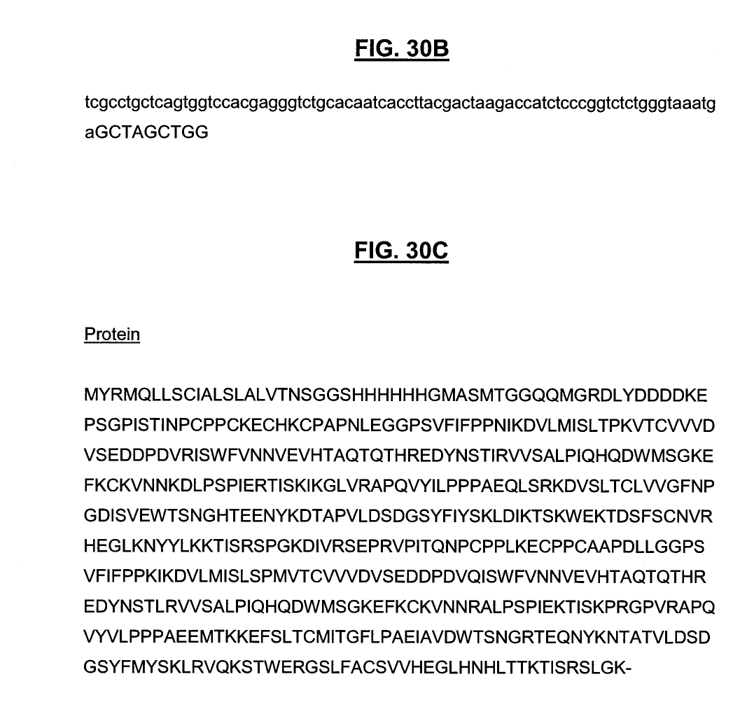

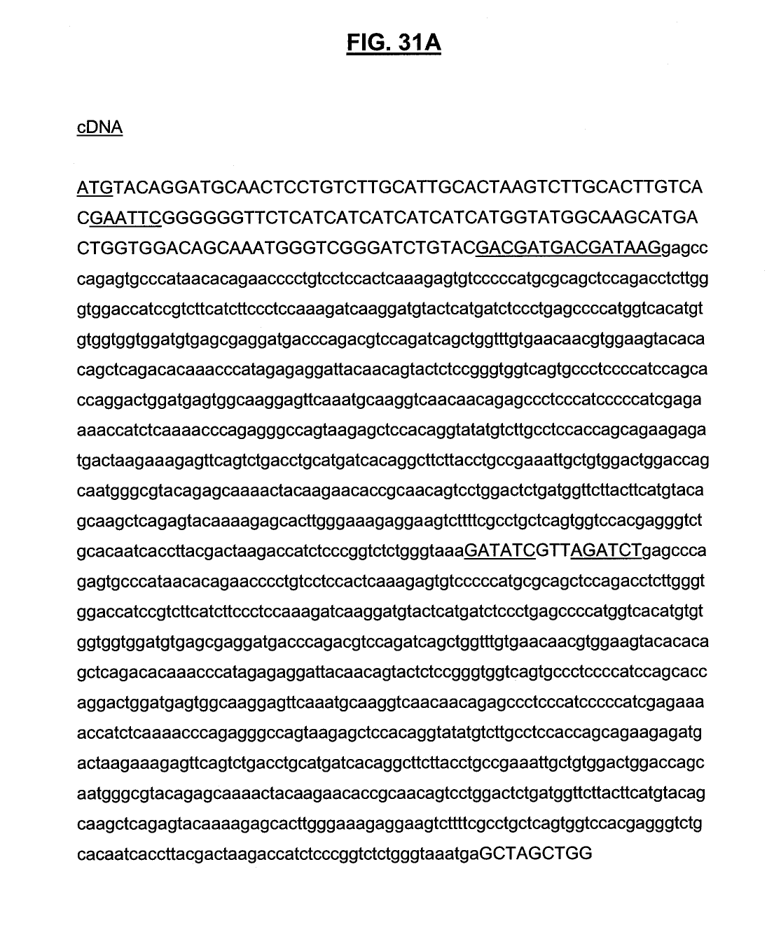

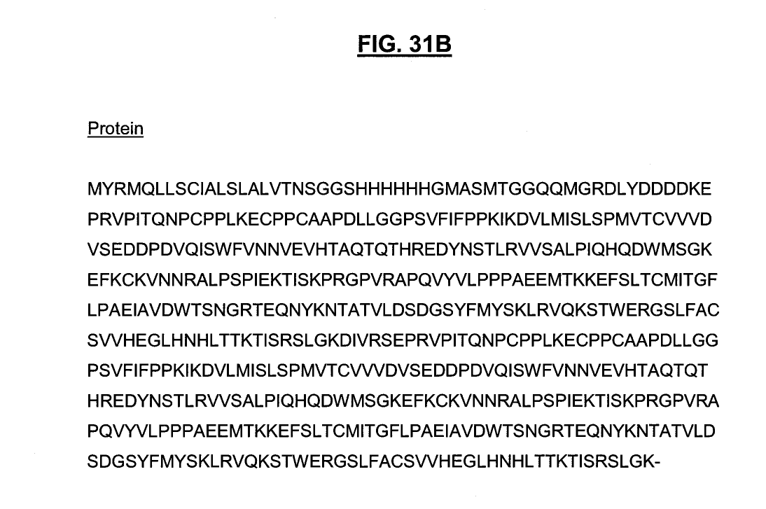

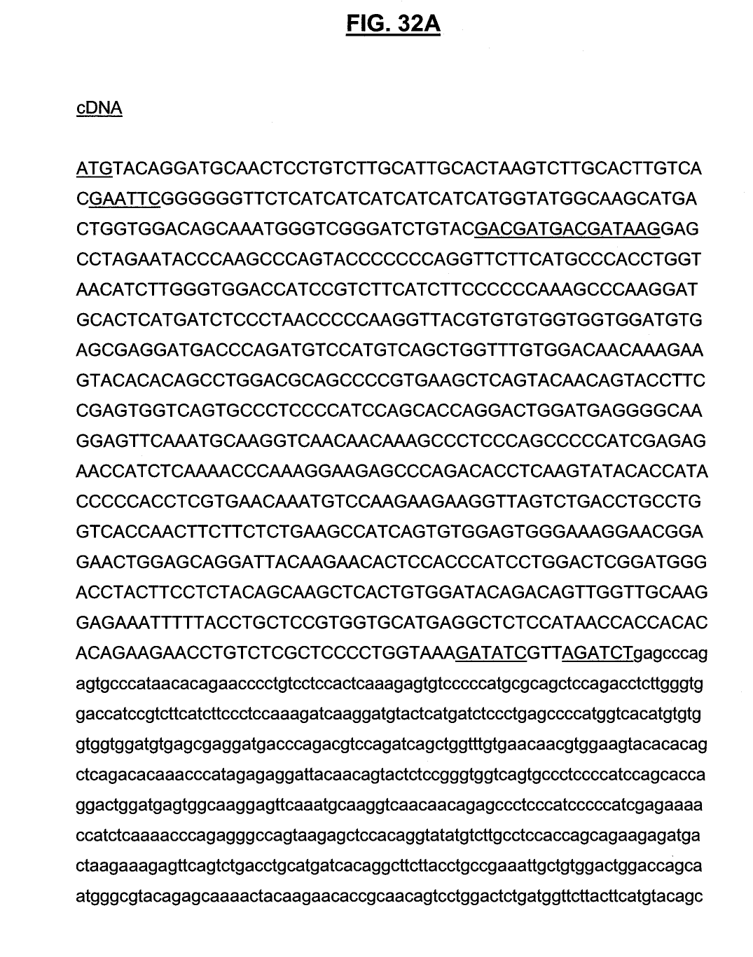

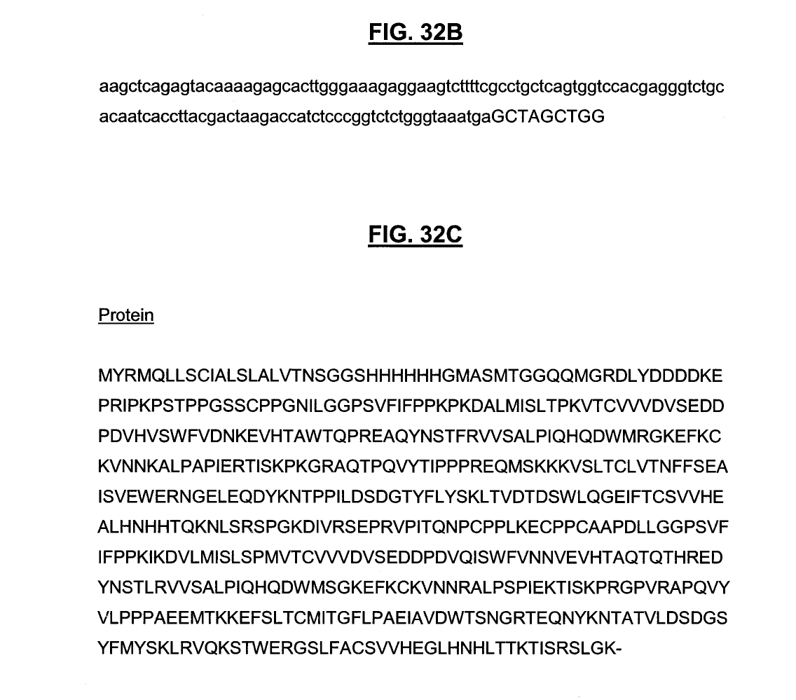

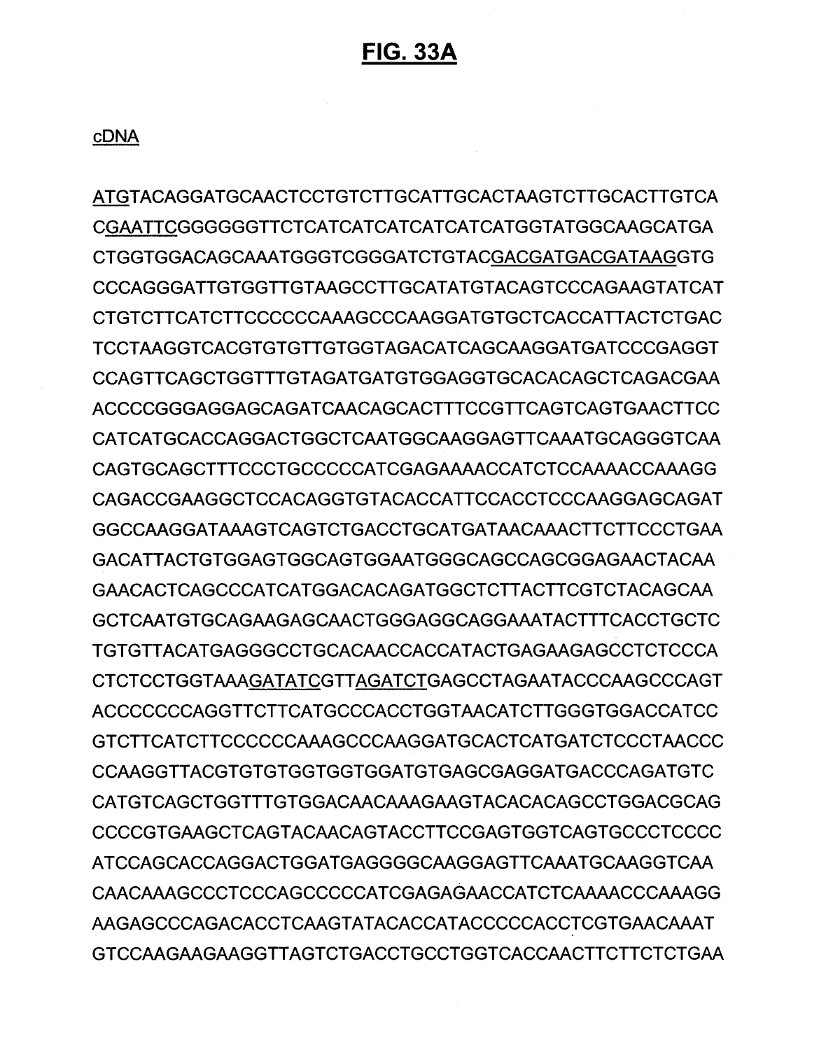

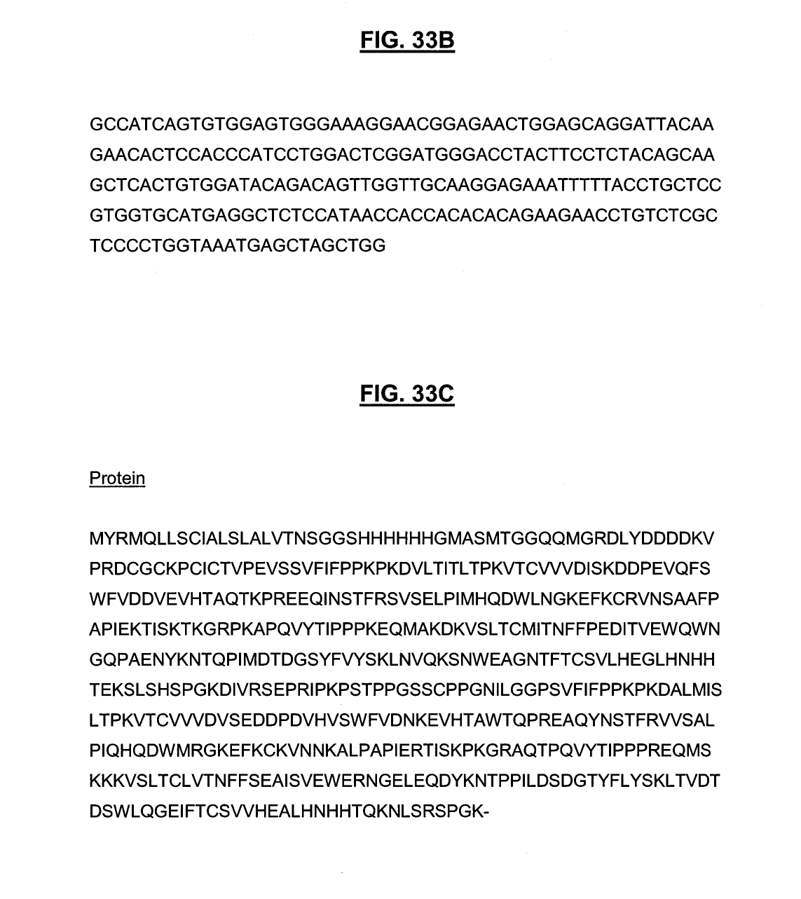

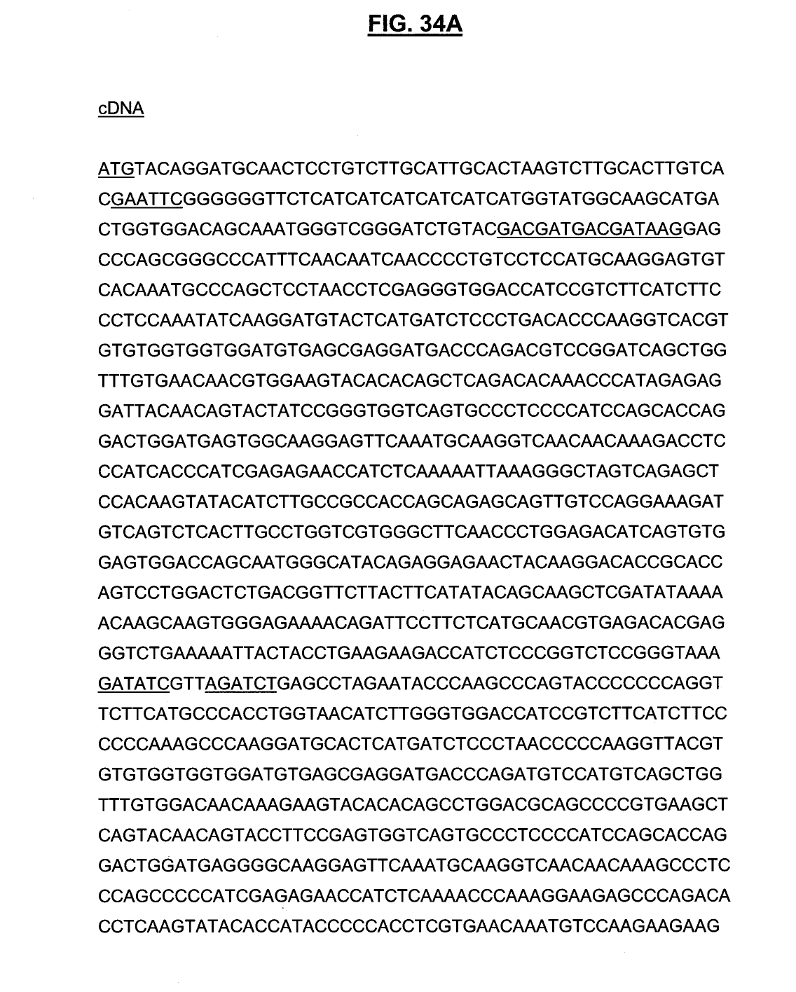

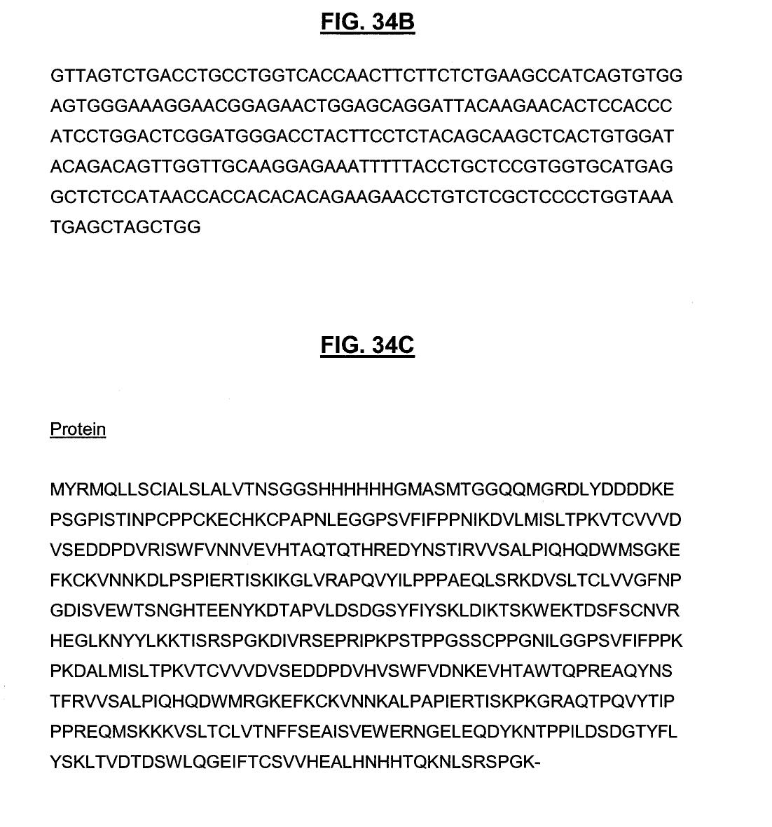

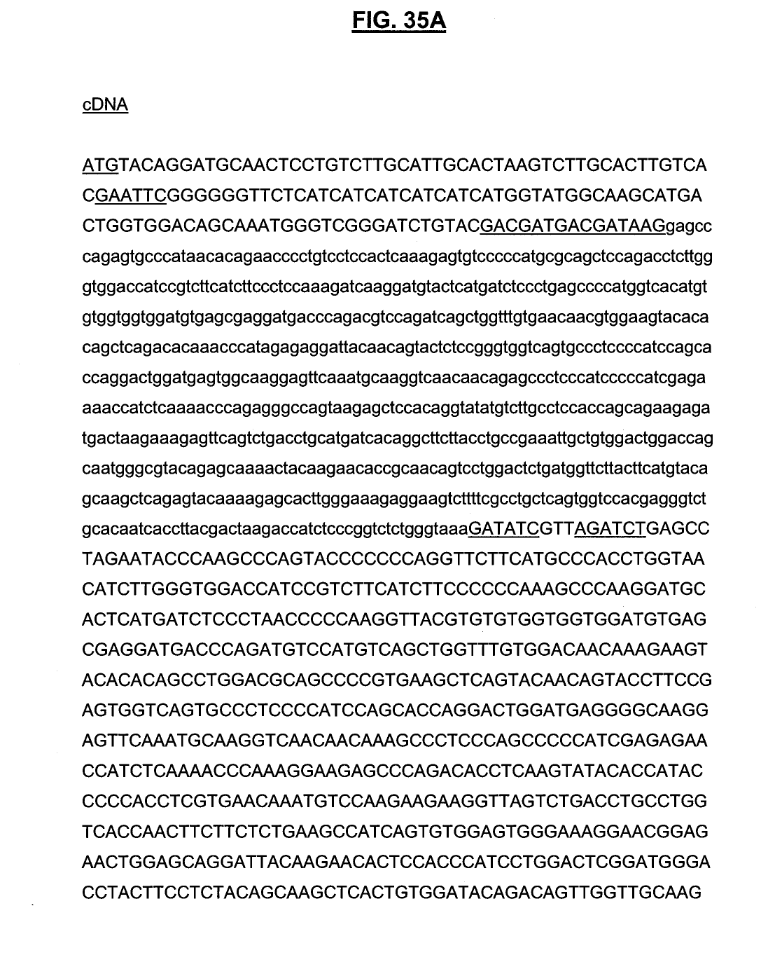

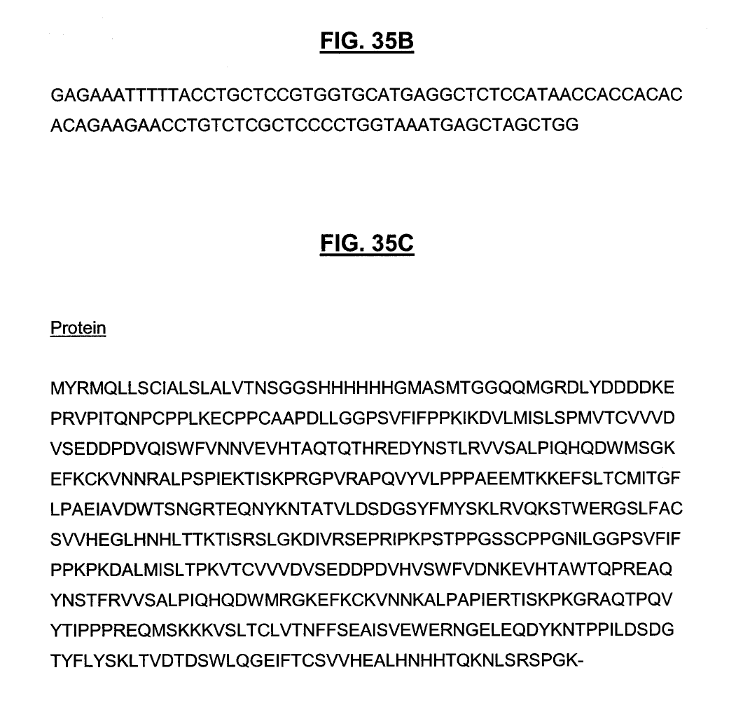

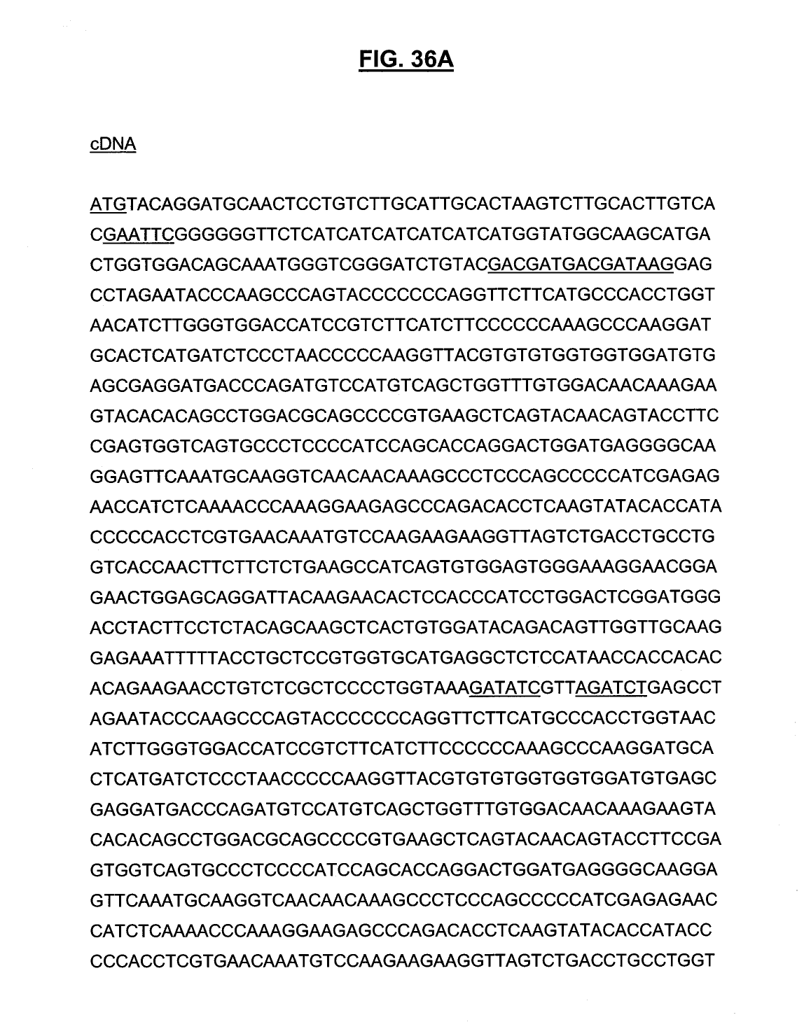

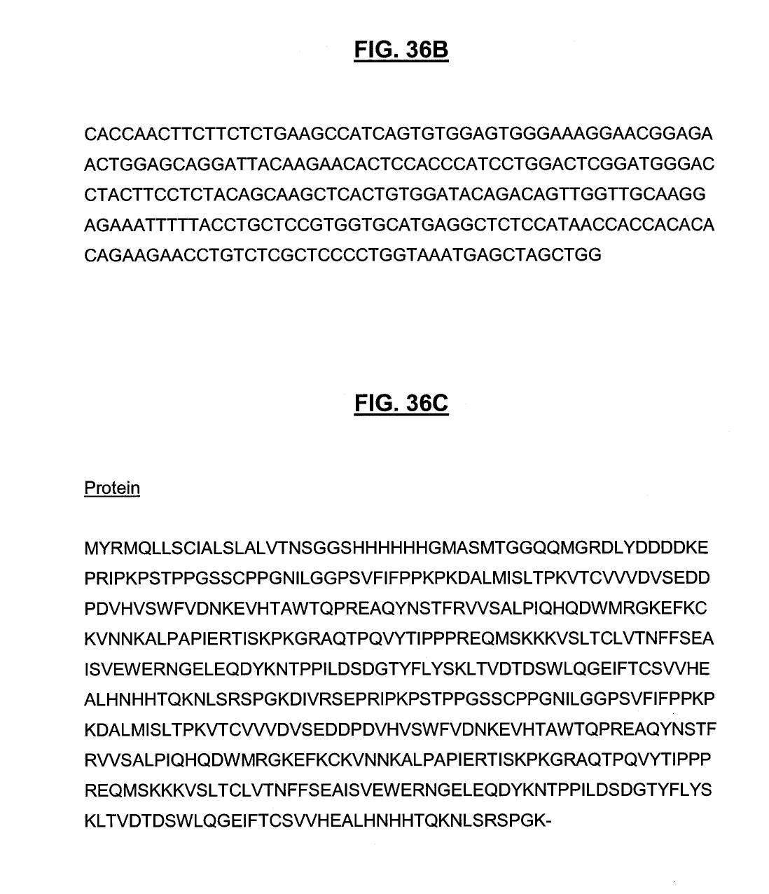

The murine C57BL/6 IgG1 cDNA, IgG2b cDNA, IgG2c cDNA, or IgG3 cDNA was then subcloned into the pFuse vector comprising the second Fc fragment of murine C57BL/6 IgG1 cDNA, IgG2b cDNA, IgG2c cDNA, or IgG3 cDNA described in Example 6. The first Fc fragment was subcloned upstream of the second Fc fragment to construct a pFuse vector comprising the first and second Fc fragment of murine C57BL/6 IgG1 cDNA, IgG2b cDNA, IgG2c cDNA, or IgG3 cDNA and any combinations of Fc fragments thereof (See FIGS. 20-36).

### Examples 8

### Cloning of pFuse Vector Comprising a Second Fc Fragment of Human IgG cDNA

Human spleen cDNA was purchased from Ambion Inc (#AM3328). Humans have different isotypes of IgG. For example, isotypes of IgG for humans include IgG1, IgG2b, IgG3 and IgG4.

The second Fc fragment of human IgG1 cDNA, IgG2 cDNA, IgG3 cDNA, or IgG4 cDNA was amplified by PCR using the following primers:

hIgG1 sense:
(SEQ ID NO: 133)
5′-TTAGATCTGAGCCCAAATCTTGTGACAAA-3′
hIgG1 antisense:
(SEQ ID NO: 134)
5′-CAGCTAGCTCATTTACCCGGAGACAGG-3′
hIgG2 sense:
(SEQ ID NO: 135)
5′-TTAGATCTGAGCGCAAATGTTGTGTCG-3′
hIgG2 antisense:
(SEQ ID NO: 136)
5′-CAGCTAGCTCATTTACCCGGAGACAGG-3′
hIgG3 sense:
(SEQ ID NO: 137)
5′-TTAGATCTGAGCTCAAAACCCCACTTG-3′
hIgG3 antisense:
(SEQ ID NO: 138)
5′-CAGCTAGCTCATTTACCCGGAGACAGG-3′
hIgG4 sense:
(SEQ ID NO: 139)
5′-TTAGATCTGAGTCCAAATATGGTCCCCCA-3′
hIgG4 antisense:
(SEQ ID NO: 140)
5′-CAGCTAGCTCATTTACCCAGAGACAGGGAG-3′

Using the same approach as set forth in Example 1, a pFuse vector comprising a second Fc fragment of human IgG1 cDNA, IgG2 cDNA, IgG3 cDNA, or IgG4 cDNA was constructed. The second Fc fragment of human IgG1 cDNA, IgG2 cDNA, IgG3 cDNA, or IgG4 cDNA comprised a hinge region, a C_{H}2 domain and C_{H}3 domain.

### Example 9

### Cloning of pFuse Vector Comprising a First and Second Fc Fragment of Human IgG cDNA

The first Fc fragment of human IgG1 cDNA, IgG2 cDNA, IgG3 cDNA, or IgG4 cDNA was amplified by PCR using the following primers:

hIgG1 fragment 1 sense:
(SEQ ID NO: 141)
5′-ACGAATTCGGGGGGTTCTC-3′
hIgG1 fragment 1 antisense:
(SEQ ID NO: 142)
5′-CTAGATCTAACGATATCTTTACCCGGAGACAGGGAG-3′
hIgG2 fragment 1 sense:
(SEQ ID NO: 143)
5′-ACGAATTCGGGGGGTTCTC-3′
hIgG2 fragment 1 antisense:
(SEQ ID NO: 144)
5′-CTAGATCTAACGATATCTTTACCCGGAGACAGGGAG-3′
hIgG3 fragment 1 sense:
(SEQ ID NO: 145)
5′-ACGAATTCGGGGGGTTCTC-3′
hIgG3 fragment 1 antisense:
(SEQ ID NO: 146)
5′-CTAGATCTAACGATATCTTTACCCGGAGACAGGGAG-3′
hIgG4 fragment 1 sense:
(SEQ ID NO: 147)
5′-ACGAATTCGGGGGGTTCTC-3′
hIgG4 fragment 1 antisense:
(SEQ ID NO: 148)
5′-CTAGATCTAACGATATCTTTACCCAGAGACAGGGAG-3′

Using the same approach as set forth in Example 2, a pFuse vector comprising a first Fc fragment of human IgG1 cDNA, IgG2 cDNA, IgG3 cDNA, or IgG4 cDNA was constructed.

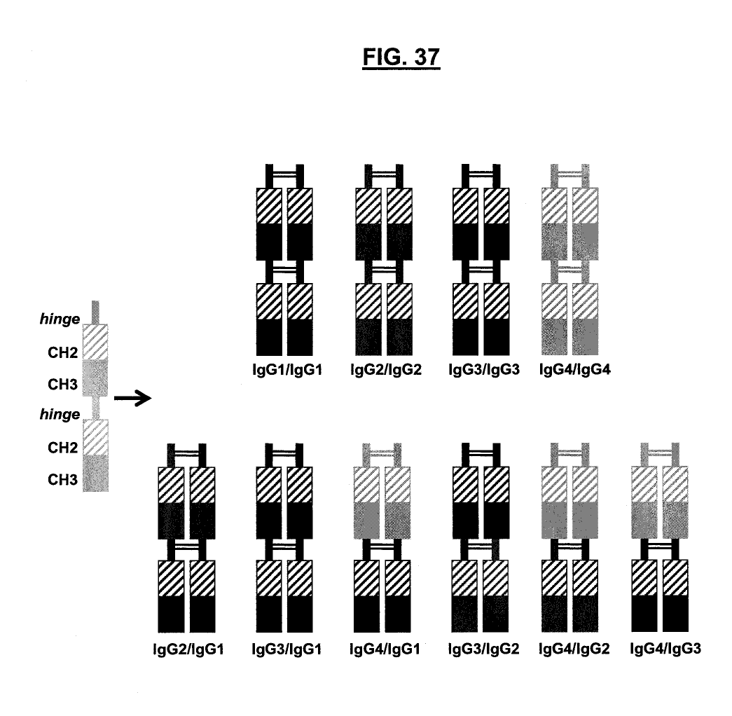

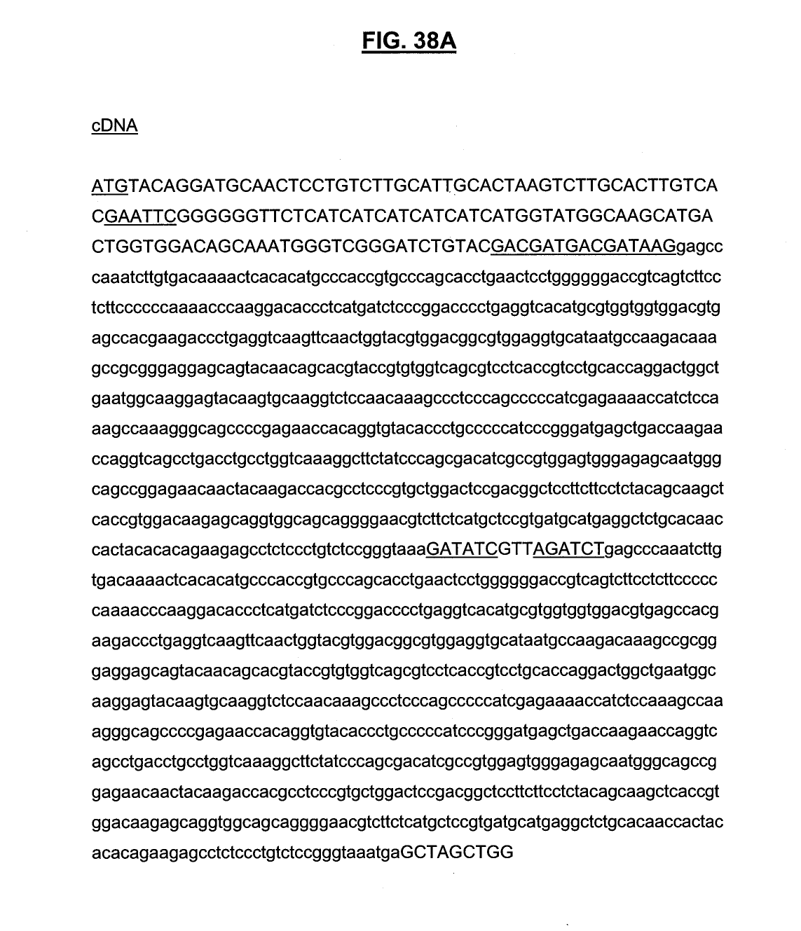

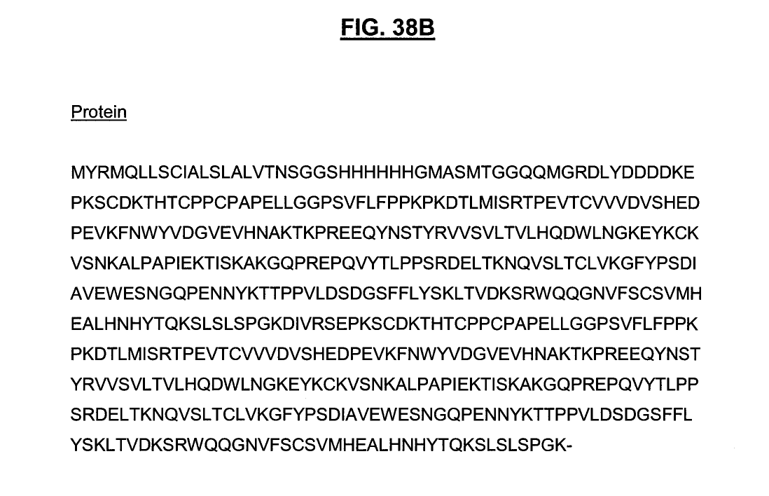

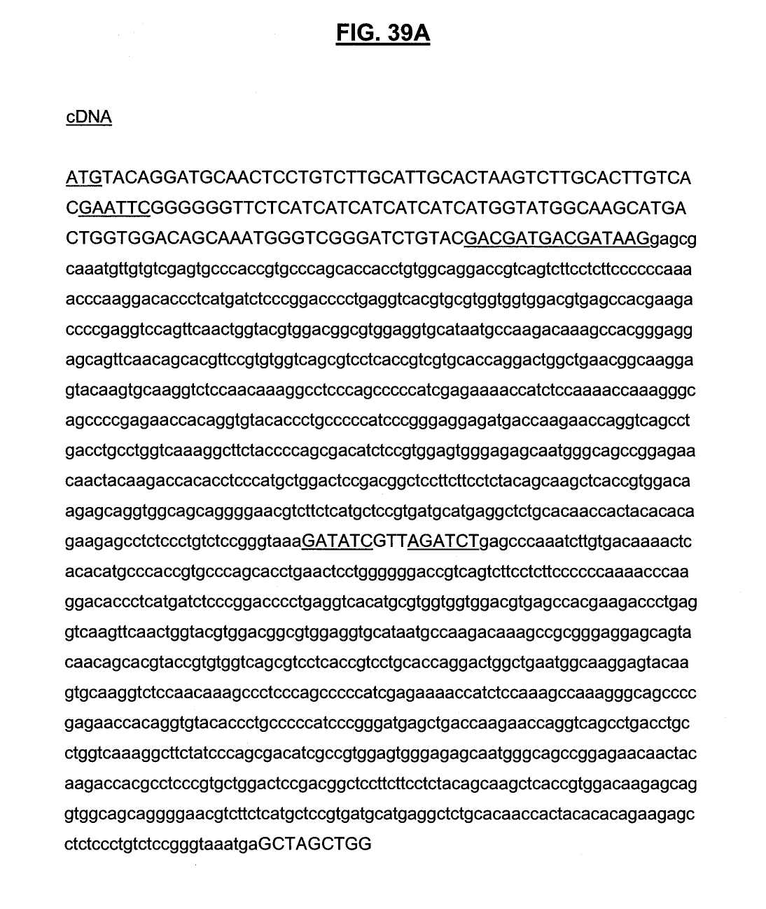

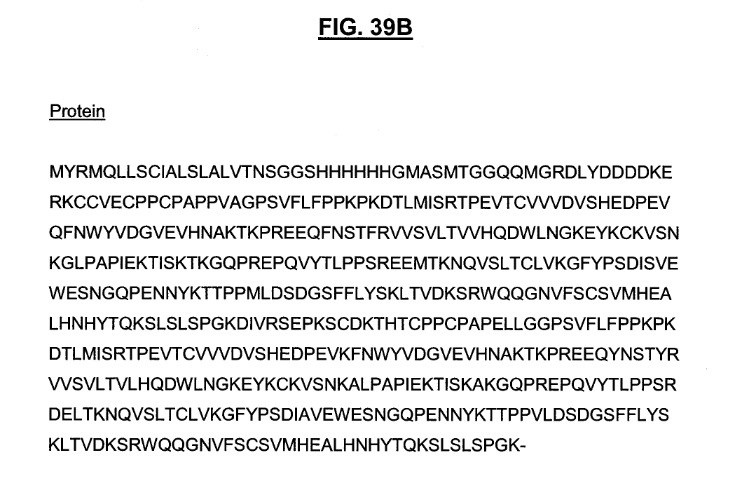

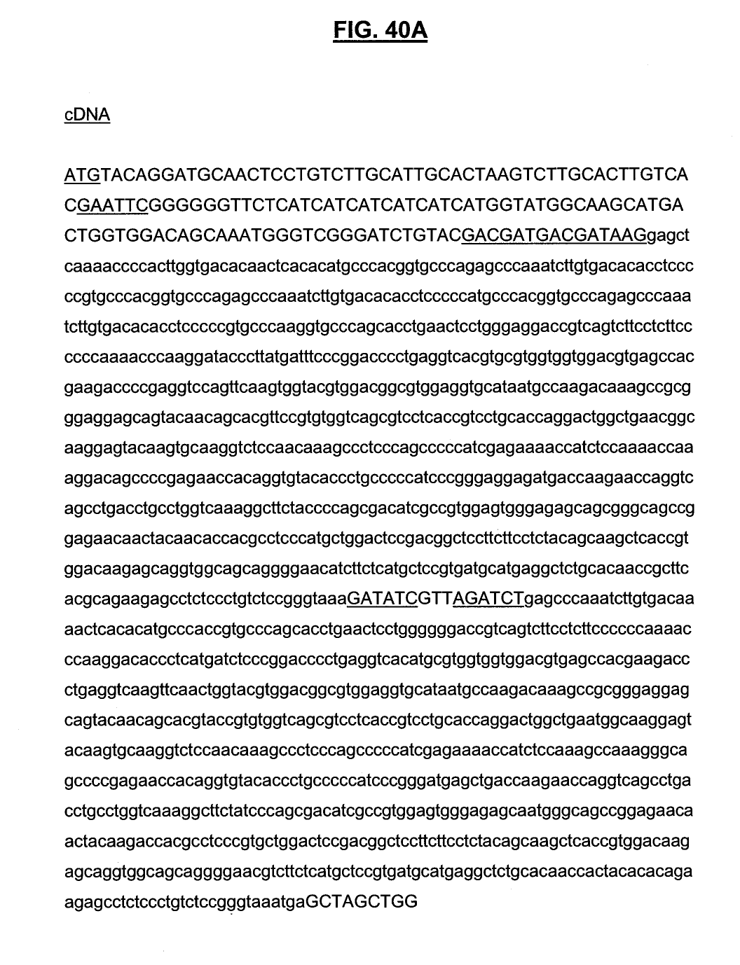

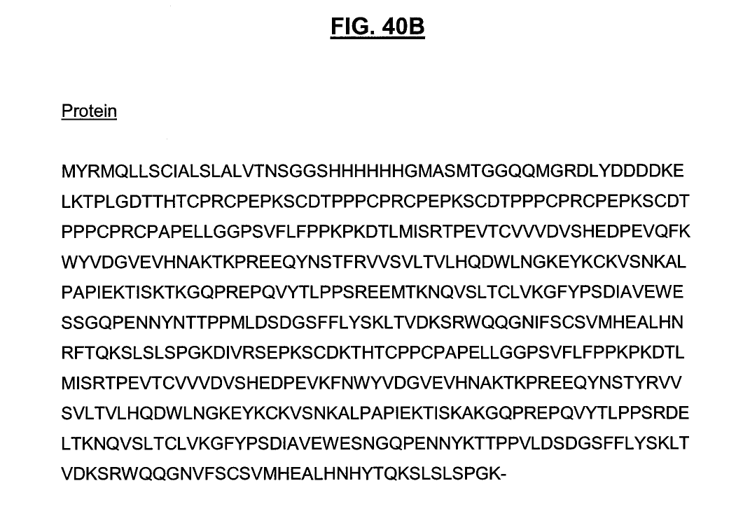

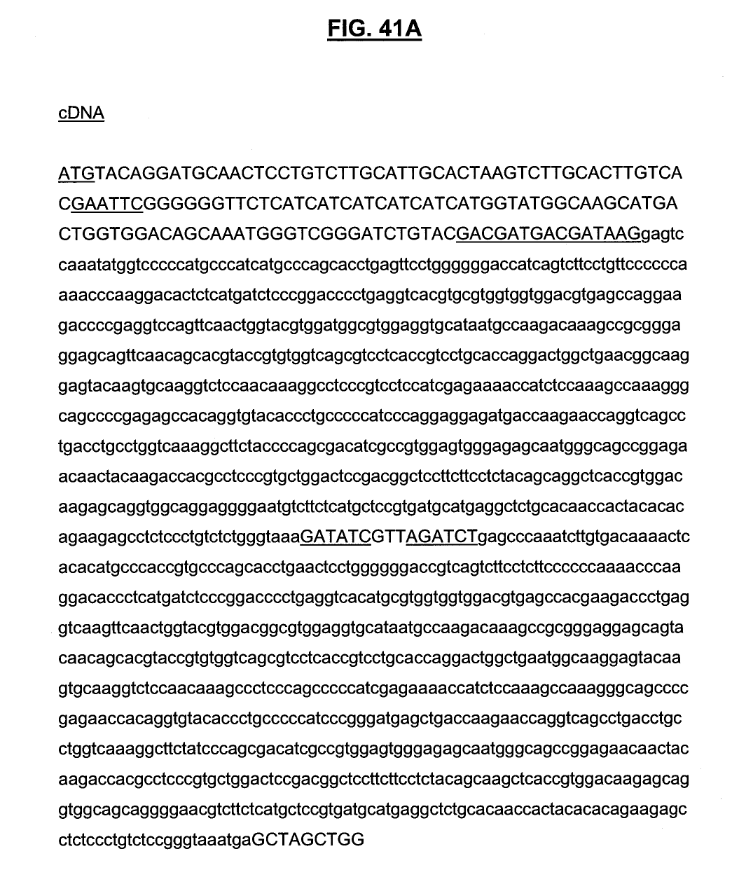

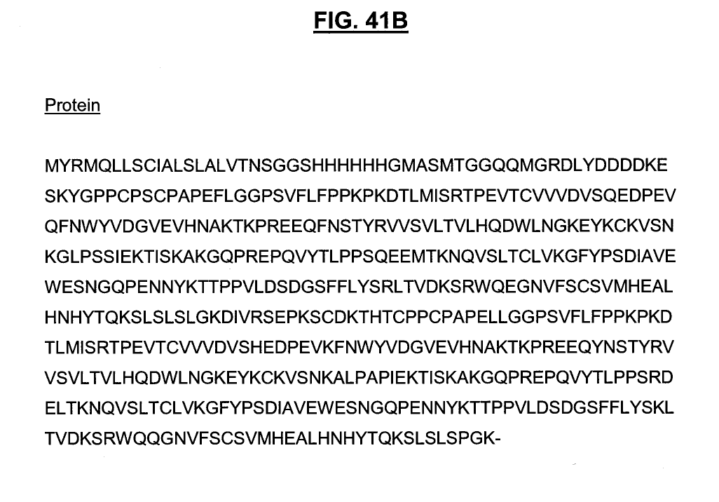

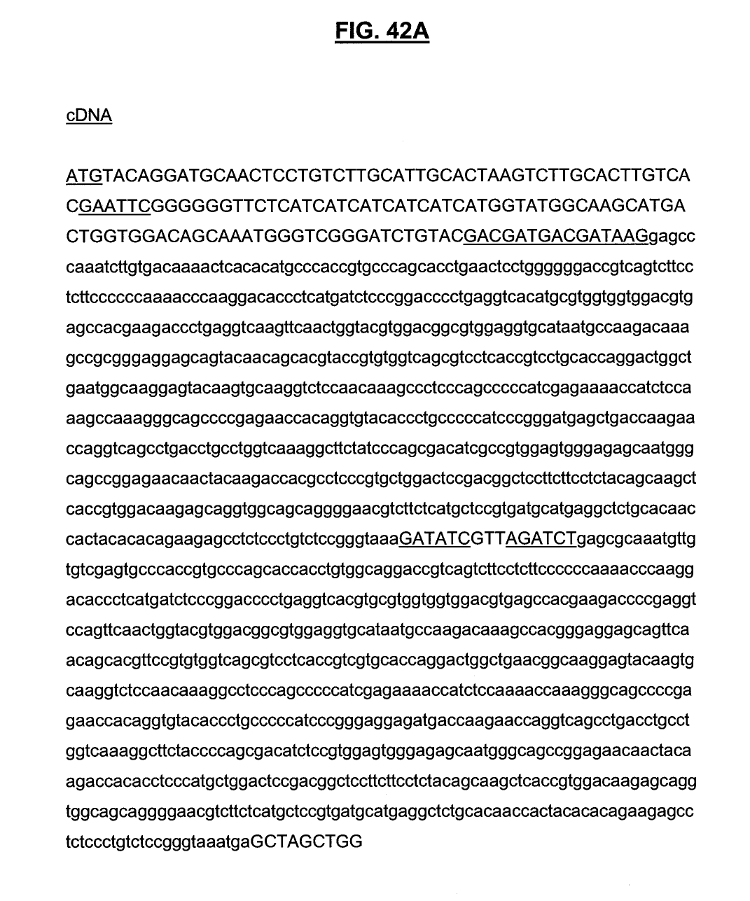

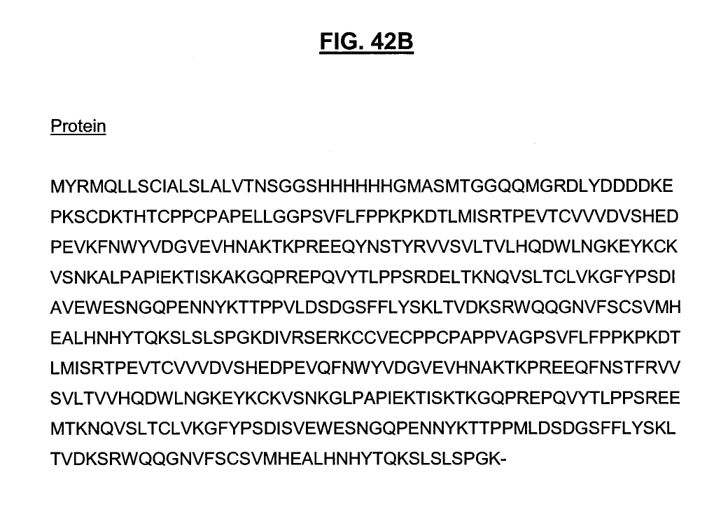

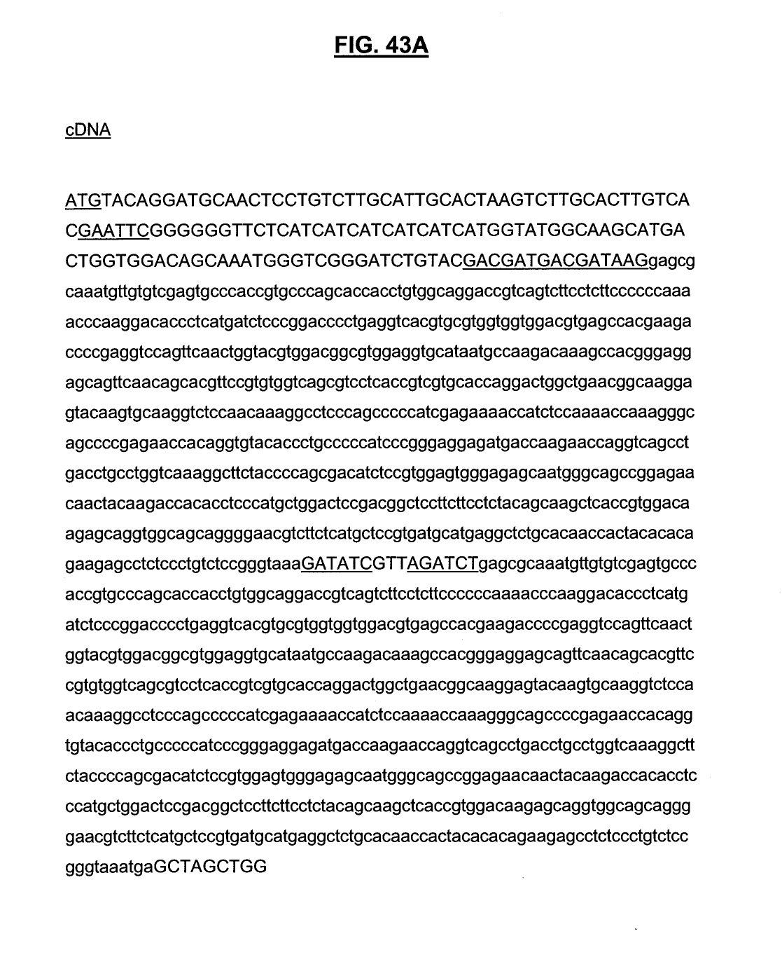

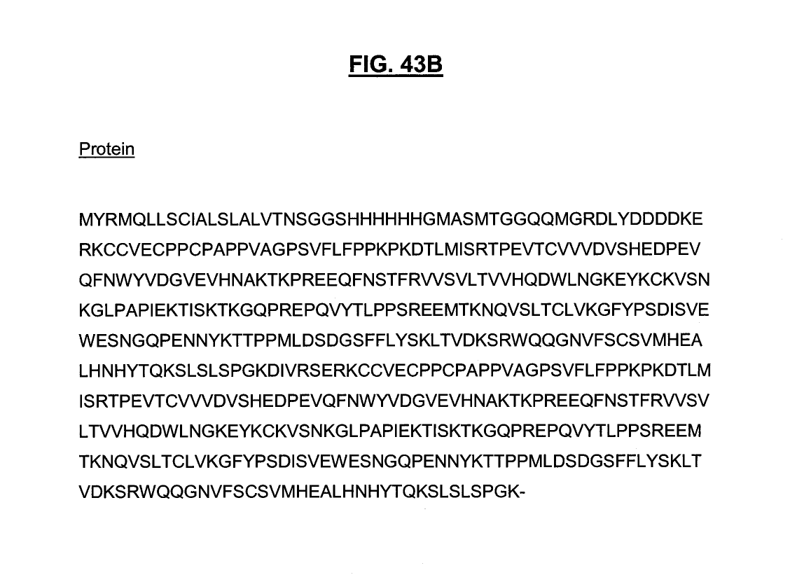

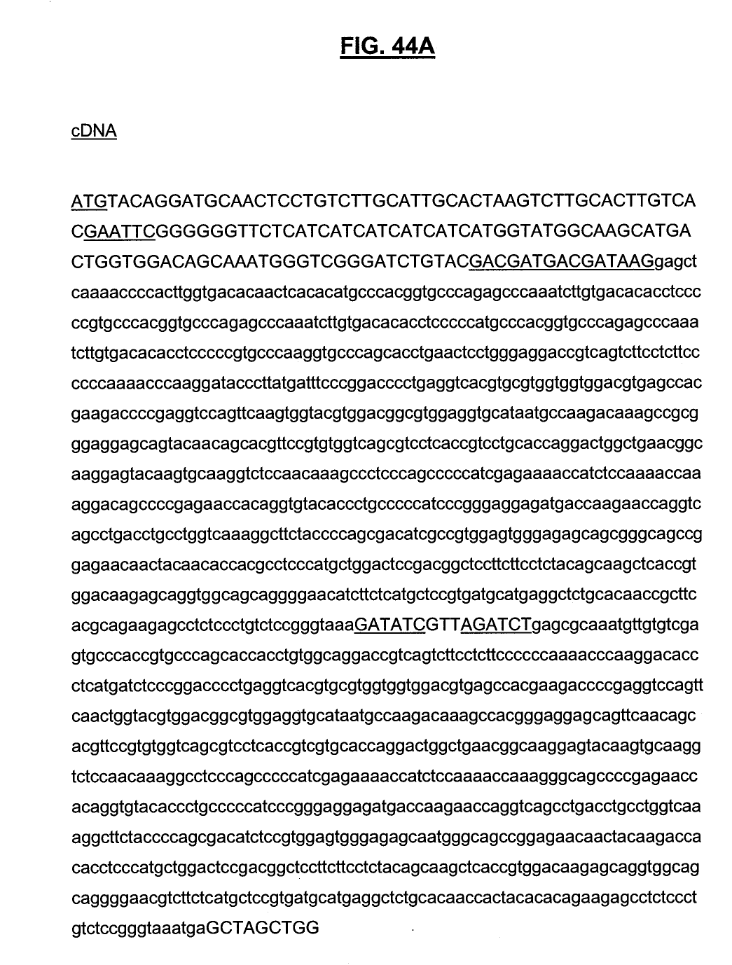

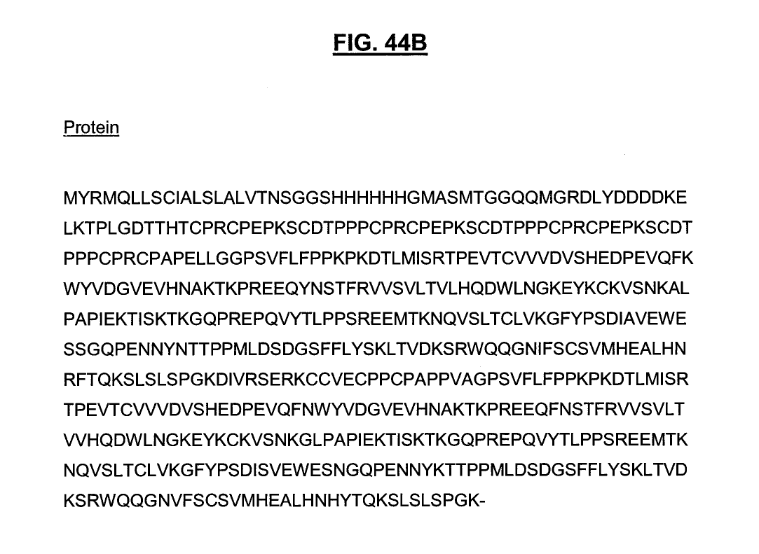

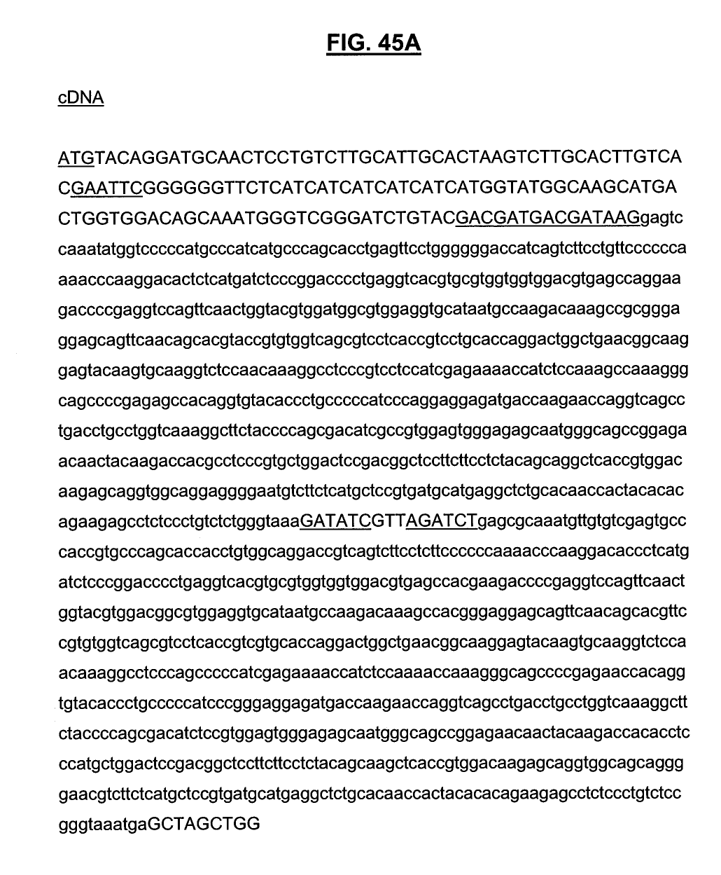

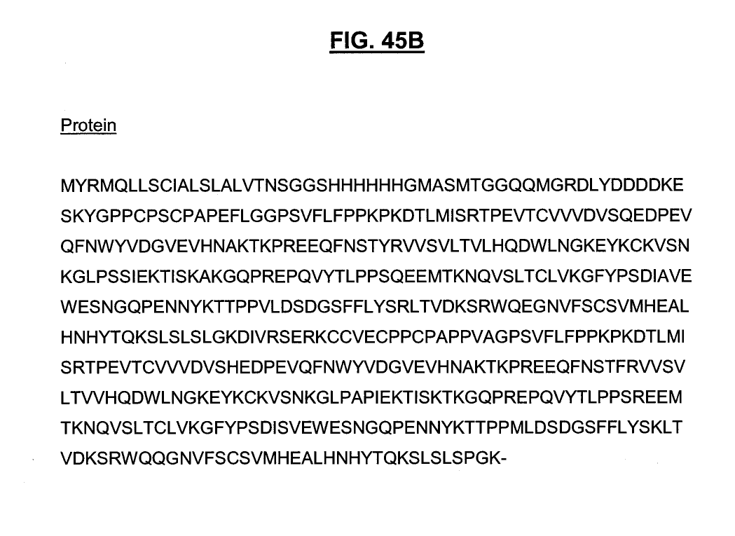

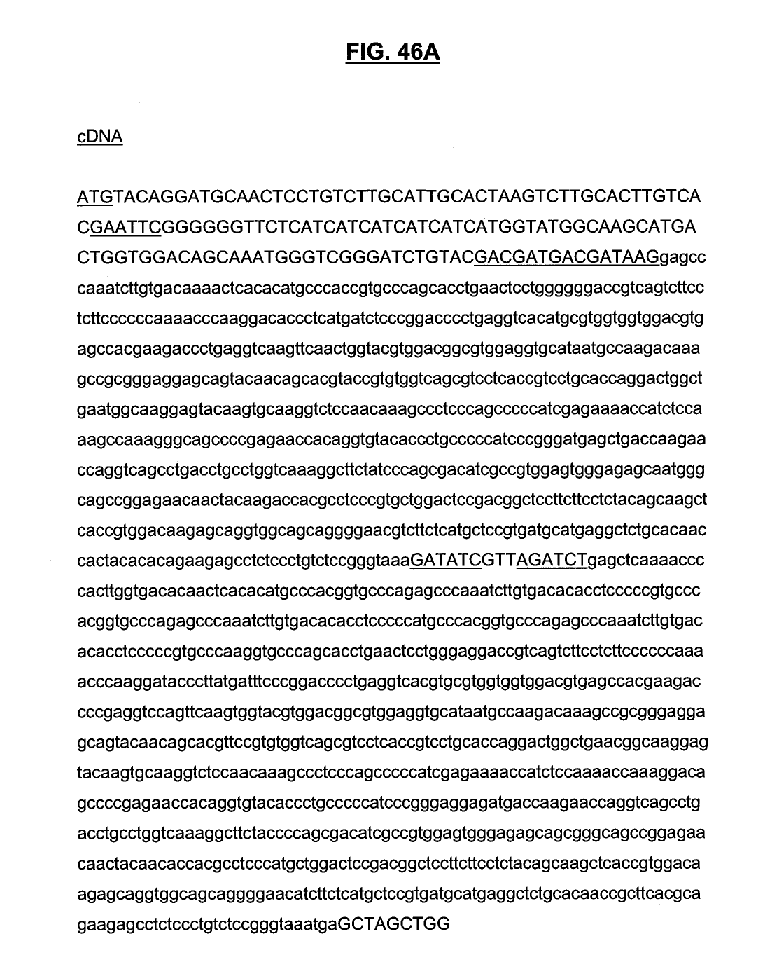

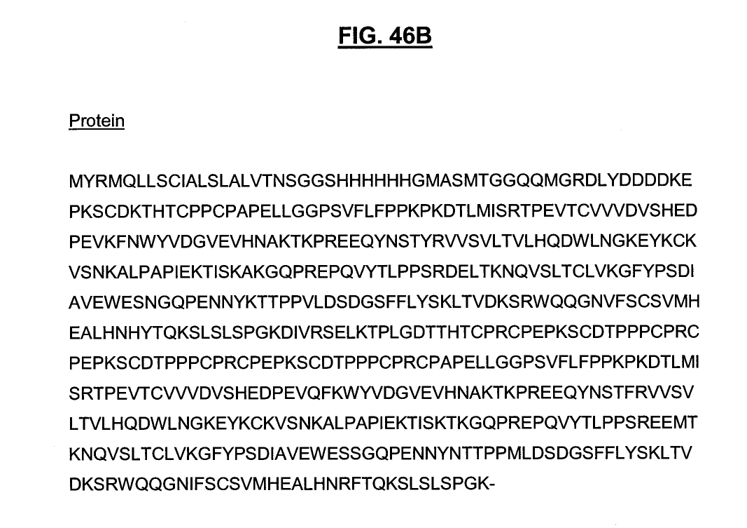

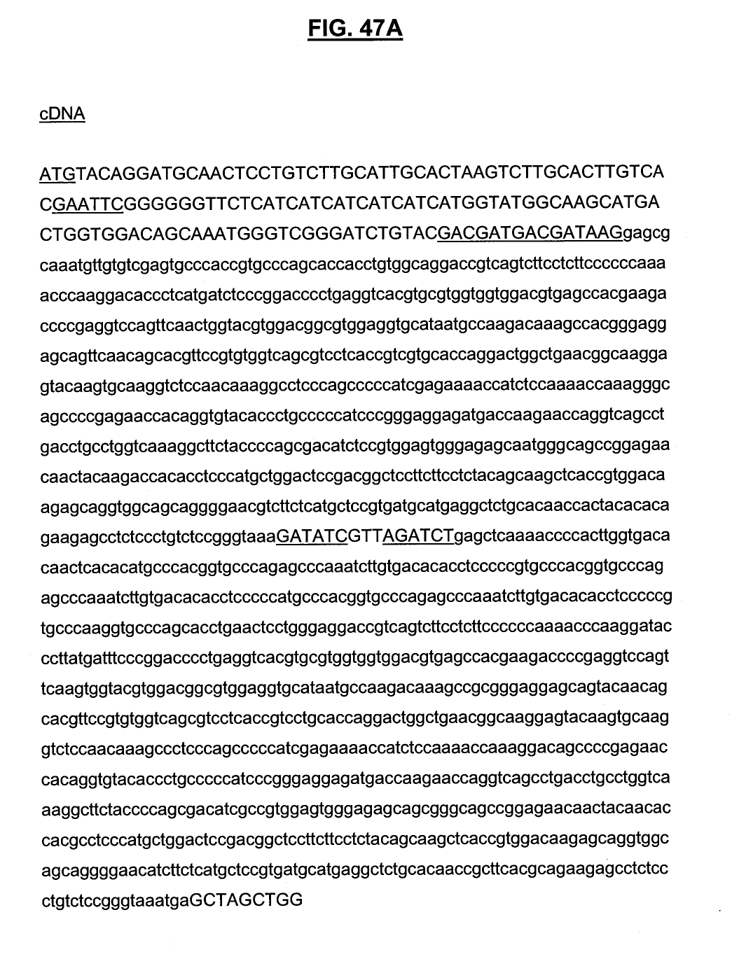

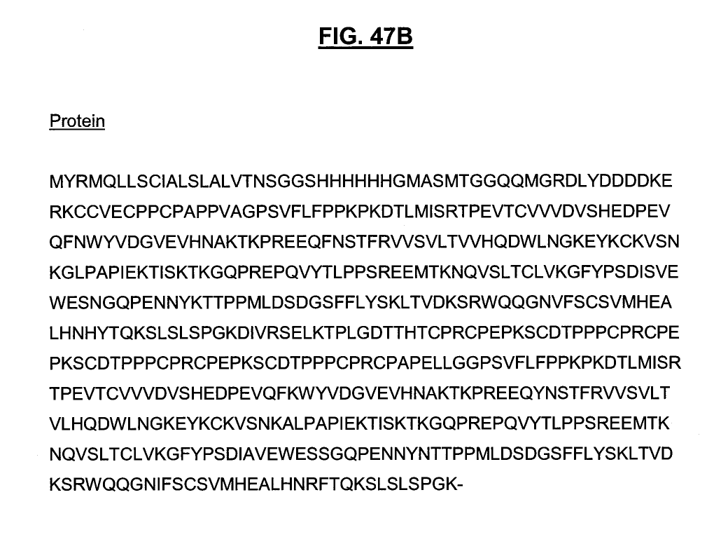

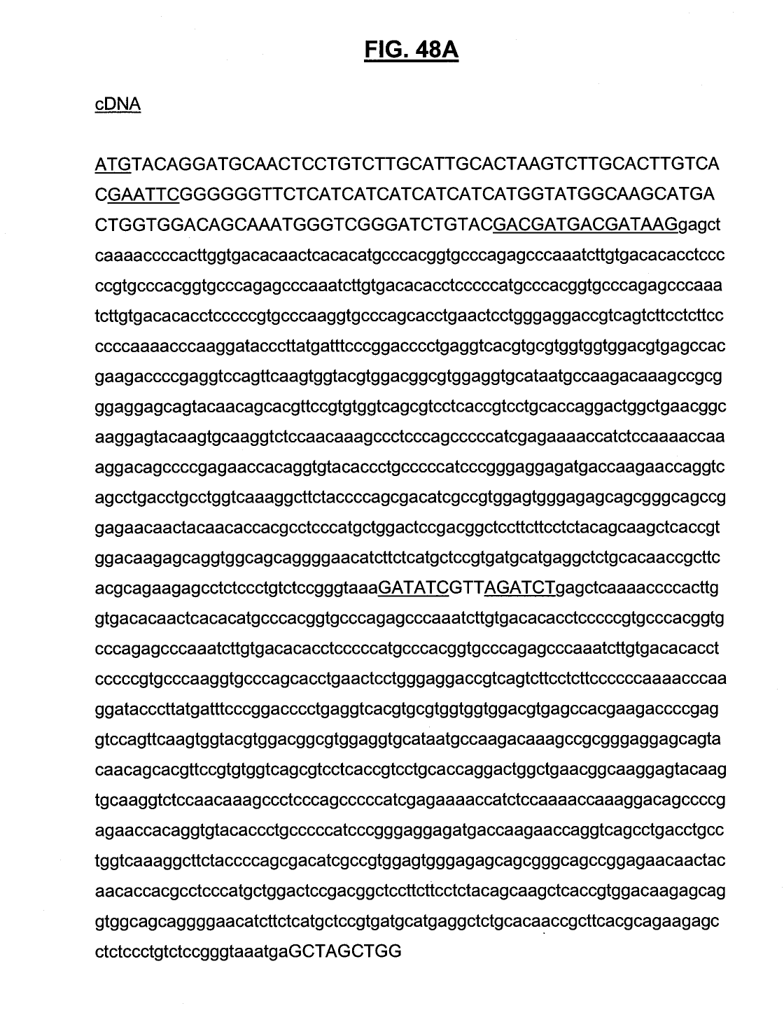

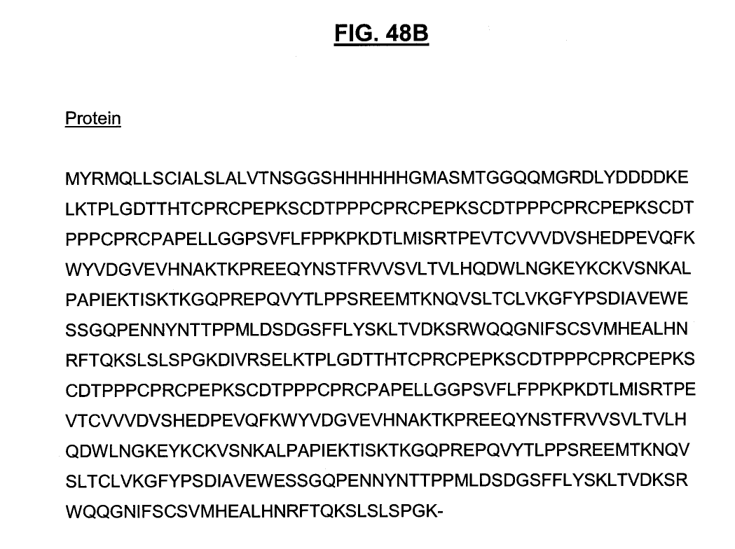

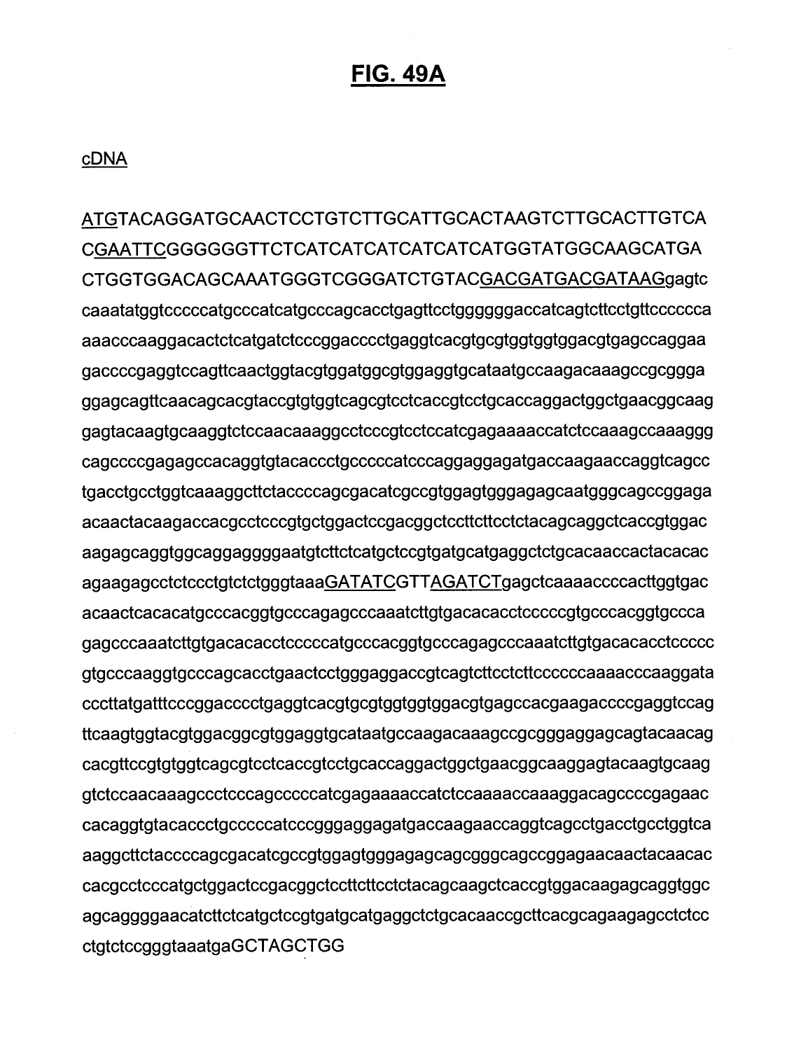

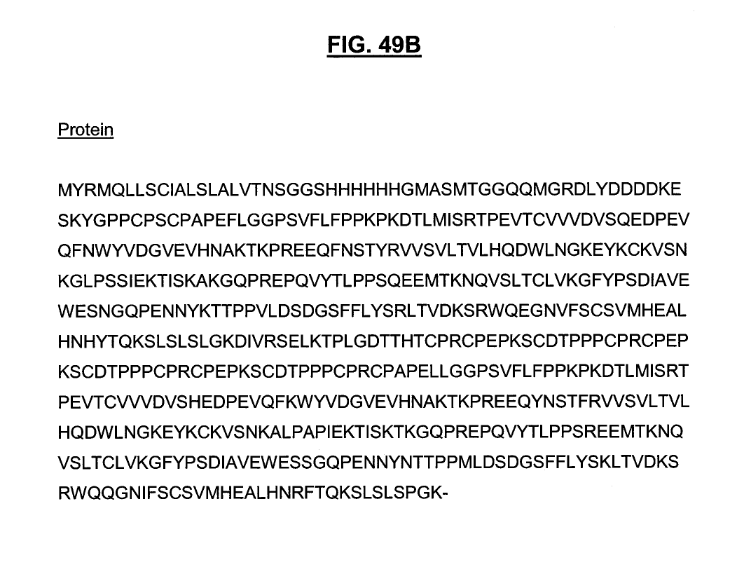

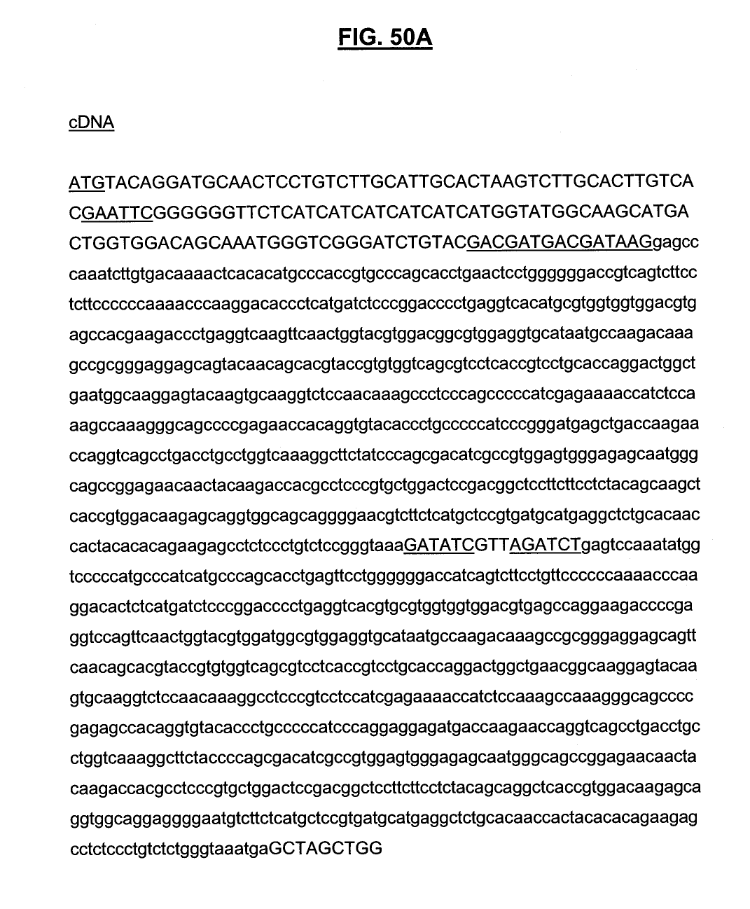

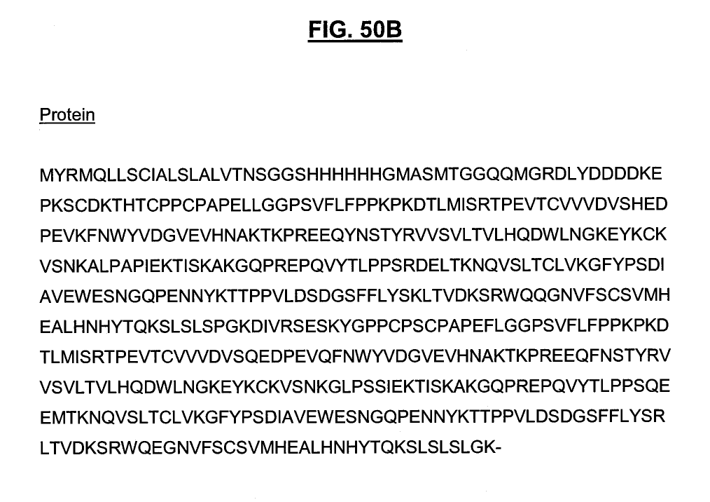

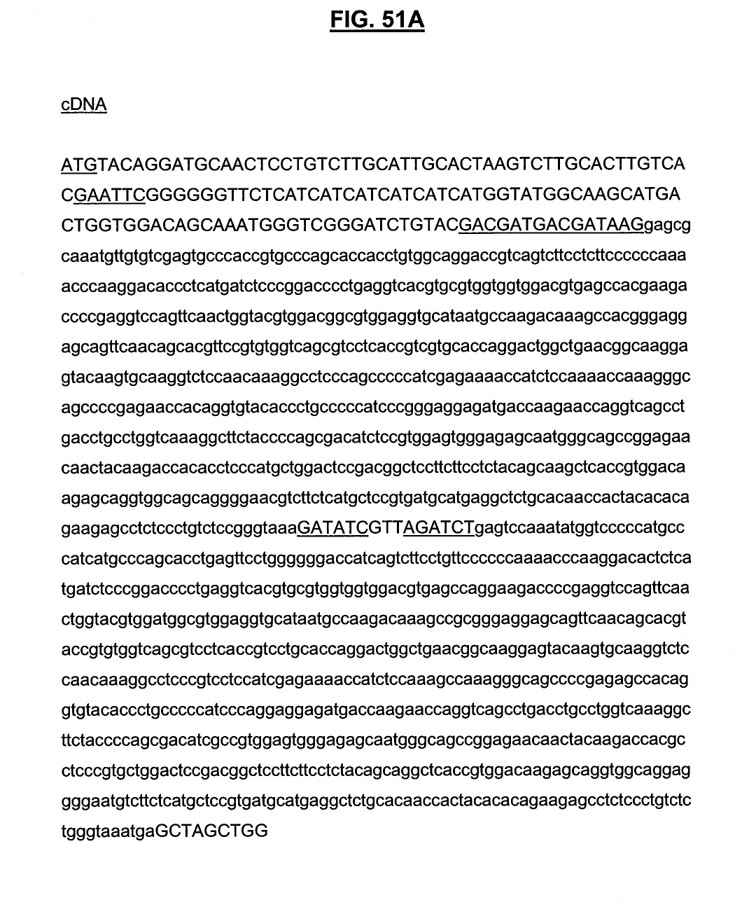

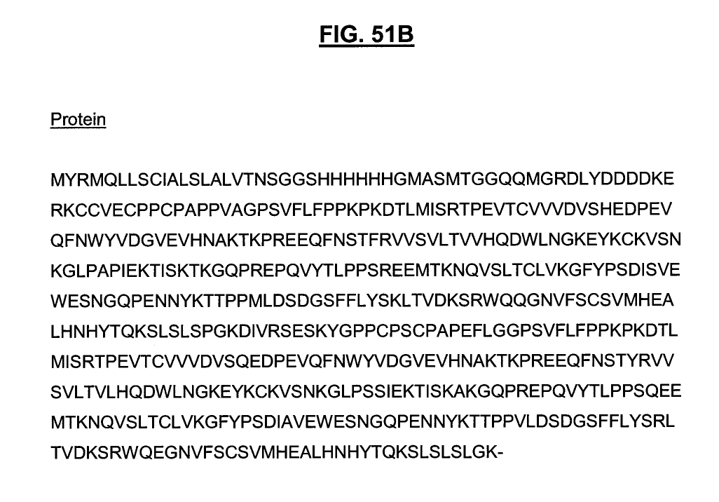

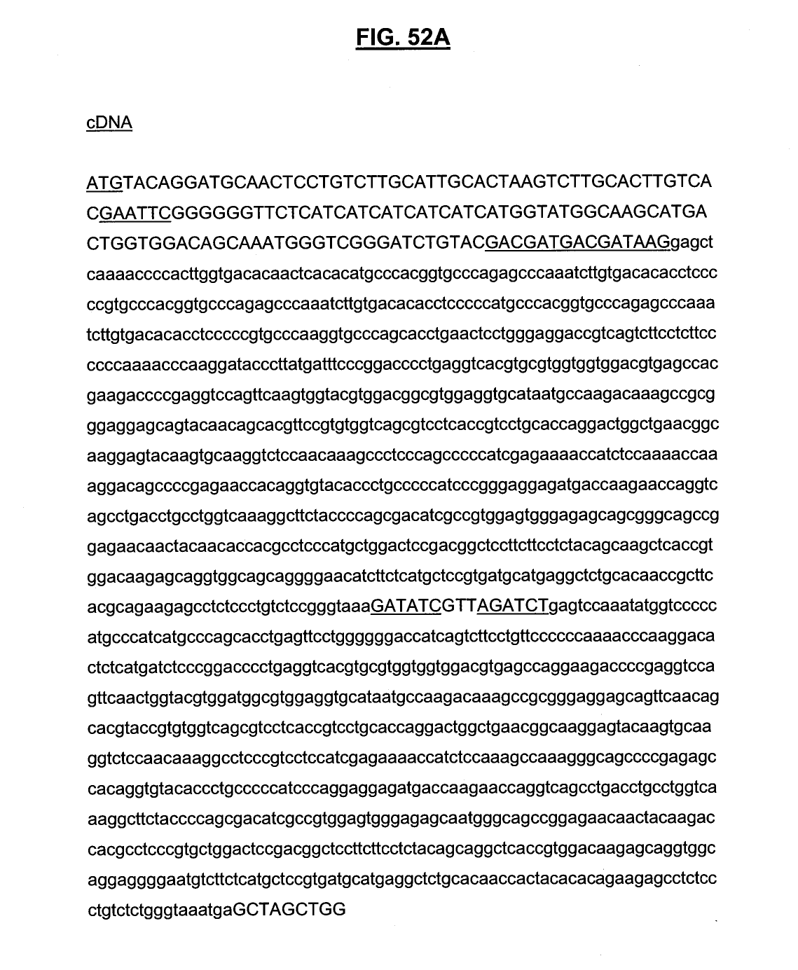

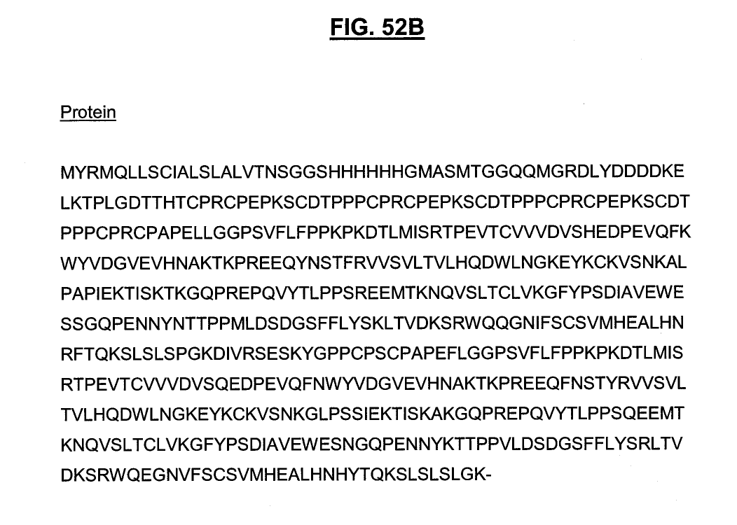

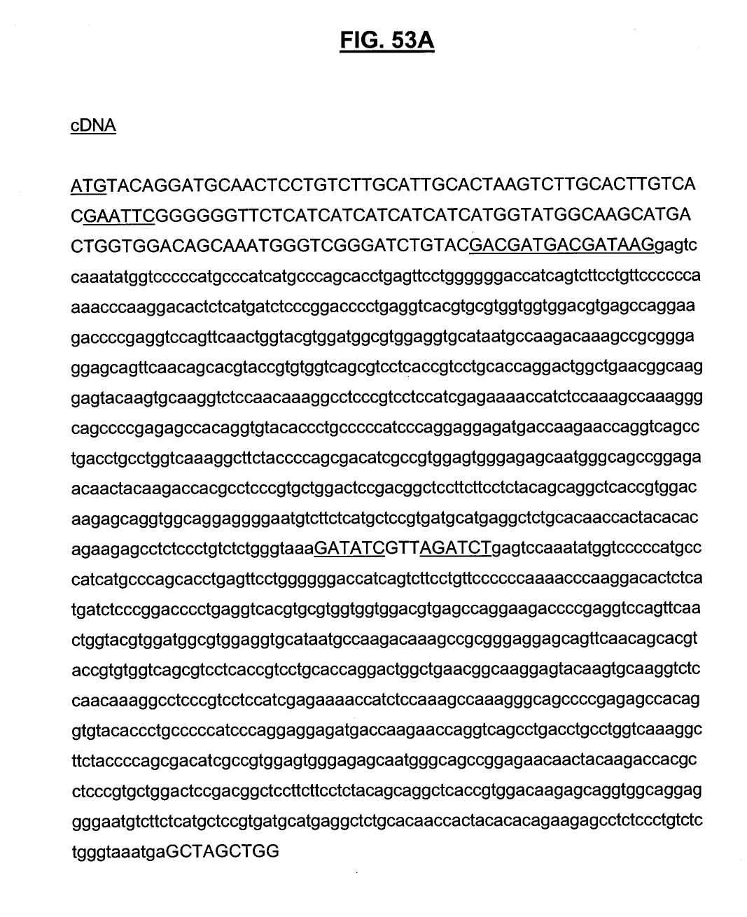

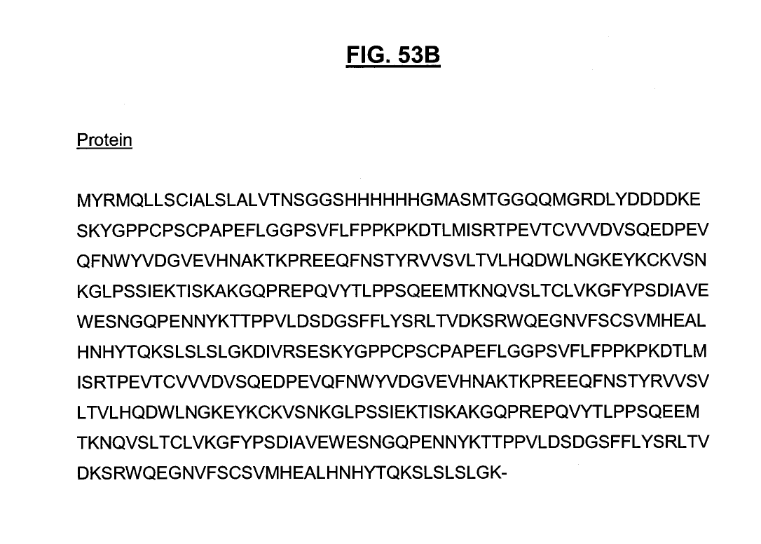

The human IgG1 cDNA, IgG2 cDNA, IgG3 cDNA, or IgG4 cDNA was then subcloned into the pFuse vector comprising the second Fc fragment of human IgG1 cDNA, IgG2 cDNA, IgG3 cDNA, or IgG4 cDNA described in Example 8. The first Fc fragment was subcloned upstream of the second Fc fragment to construct a pFuse vector comprising the first and second Fc fragments of human IgG1 cDNA, IgG2 cDNA, IgG3 cDNA, or IgG4 cDNA and any combinations of Fc fragments thereof (See FIGS. 37-53).

### Example 10

Transfection of HeLa Cells with a pFuse Vector Comprising a First and Second Fc Fragment of IgG cDNA

HeLa cells were added to a 6-well plate at a concentration of 1×10⁶ cells per well. A mixture was prepared of 1 μg of a pFuse vector construct from Examples 2, 3, 5, 7, or 9 and 3.5 μg Fugene®HD (Roche™) in 100 μl of RPMI and incubated for 15 minutes at room temperature. The 100 μl mixture set forth herein was added to the cells for 3-4 hours and the cells were transfected.

### Example 11

### Detection of HeLa Cellular Secretion of Polypeptides Comprising a First and Second Fc Fragment of Rabbit IgG in Monomeric and Dimeric Form

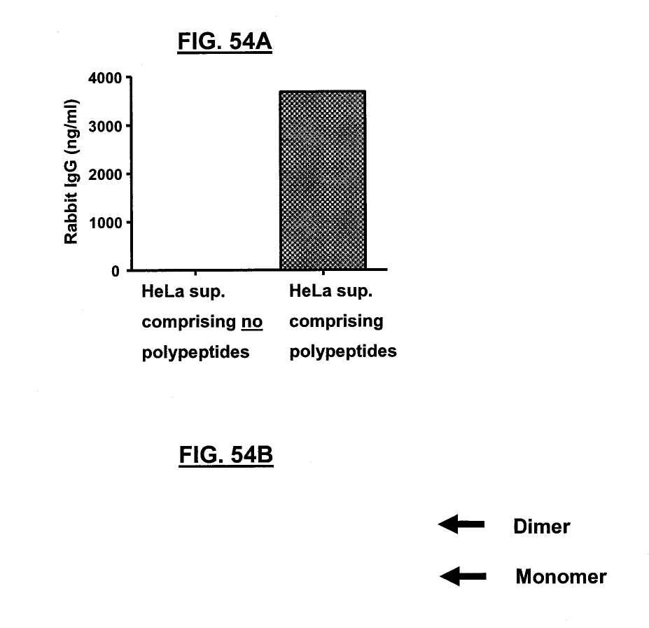

Supernatants were collected from transfected HeLa cells described in Example 10. An Enzyme-Linked Immunosorbent Assay (ELISA) was performed on the supernatants using anti-rabbit IgG antibodies to detect high levels of polypeptides comprising a first and second Fc fragment of rabbit IgG (See FIG. 54A, HeLa sup. comprising polypeptides) compared to supernatants from non-transfected HeLa cells, which contain no detectable levels of polypeptides comprising a first and second Fc fragment of rabbit IgG (See FIG. 54A, HeLa sup comprising no polypeptides).

Without wishing to be bound by theory, the transfected HeLa cells secrete polypeptides comprising the first and second Fc fragment of rabbit IgG in monomeric form, which spontaneously dimerize to form dimers (See FIG. 1D). As set forth herein, the polypeptides comprising the first and second Fc fragment of rabbit IgG in monomeric form are unable to bind FcγRs and are therefore, unable to induce IL-10. Therefore, although, subsequent experiments involve the use of supernatants containing both polypeptides comprising the first and second Fc fragment in monomeric form and polypeptides comprising the first and second Fc fragment in dimeric form, only the polypeptides comprising the first and second Fc fragment in dimeric form are able to bind and cross-link the FcγR and induce IL-10 production. The polypeptides comprising the first and second Fc fragment of rabbit IgG in monomeric form merely spontaneously dimerize to form the polypeptides comprising the first and second Fc fragment in dimeric form. Therefore, the supernatants will be subsequently referred to as “polypeptides comprising the first and second Fc fragment of IgG in dimeric form” or ‘polypeptides in dimeric form” despite the supernatants comprising both polypeptides comprising the first and second Fc fragment of IgG in monomeric form and polypeptides comprising the first and second Fc fragment of IgG in dimeric form.

One of ordinary skill in the art would recognize that “polypeptides in multimeric form” would work similar to “polypeptides in dimeric form”.

In addition, supernatants containing polypeptides comprising a first and second Fc fragment of rabbit IgG in dimeric form from transfected HeLa cells were passed through a protein A bead column. The column was washed several times to wash away any unbound protein. The polypeptides comprising a first and second Fc fragment of rabbit IgG in dimeric form that were bound to the column were then eluted from the column and collected. The collected samples were then tested for high polypeptide content by spectrophotometry measuring A₂₈₀.

In addition, supernatants containing polypeptides comprising a first and second Fc fragment of rabbit IgG in dimeric form from transfected HeLa cells may also be purified using a Nickel column instead of using a protein A bead column as set forth herein.

Furthermore, after purifying the polypeptide comprising a first Fc fragment of rabbit IgG in dimeric form, the enzyme enterokinase may be used to cleave all unnecessary sequences, such as 6×His and Xpress epitope, thus leaving only the essential biologically active domains of the polypeptide and reducing the overall immunogenicity of the polypeptide.

The samples that contained high levels of polypeptides comprising a first and second Fc fragment of rabbit IgG in monomeric and dimeric form were run in a Sodium Dodecyl Sulfate Polyacrylamide Gel Electrophoresis (SDS-PAGE) and detected by Western Blot (See FIG. 54B) using anti-histidine antibodies or anti-rabbit antibodies conjugated with horse-radish peroxidase. Samples contained polypeptides comprising a first and second Fc fragment of rabbit IgG in monomeric and dimeric form (See FIG. 54B, final two samples).

The experiment may also be conducted using polypeptides comprising a first and second Fc fragment of murine BALB/c IgG, polypeptides comprising a first and second Fc fragment of murine C57BL/6 IgG or polypeptides comprising a first and second Fc fragment of human IgG and the appropriate reagents including, but not limited, to antibodies and conjugated antibodies. One of ordinary skill in the art would recognize that polypeptides comprising a first and second Fc fragment of murine BALB/c IgG, polypeptides comprising a first and second Fc fragment of murine C57BL/6 IgG or polypeptides comprising a first and second Fc fragment of human IgG would work in an equivalent manner with similar results.

### Example 12

### Binding of Polypeptides Comprising at Least a First and Second Fc Fragment of Rabbit IgG in Dimeric Form to Macrophages

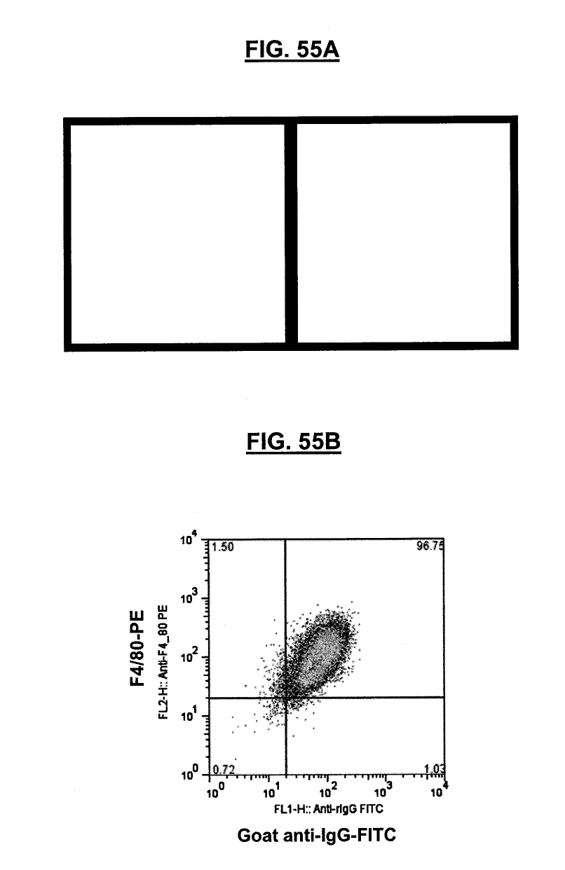

In order to demonstrate that polypeptides comprising a first and second Fc fragment of rabbit IgG in dimeric form bind to macrophages, 6-day bone-marrow derived macrophages (BMMφs) (2×10⁵) were inoculated onto a glass slide overnight and the cells were incubated with carboxyfluorescein succinimidyl ester (CFSE) to show the contours of the macrophages. The cells were then incubated with polypeptides comprising a first and second Fc fragment of rabbit IgG in dimeric form for 30 minutes. The slide containing the cells incubated with the polypeptides was then fixed with 4% paraformaldehyde. The slide was washed and then treated with goat anti-rabbit F(ab′)₂-Cy3. The slide was then mounted with fluorescent mounting media for confocal imaging purposes. The results indicated that the polypeptides comprising a first and second Fc fragment of rabbit IgG in dimeric form bind to macrophages (See FIG. 55A, red outline of cell in right and left panels).

In addition, BMMφs (2×10⁶) were incubated with polypeptides comprising a first and second Fc fragment of rabbit IgG in dimeric form for 30 minutes. After 30 minutes, the cells were washed and then incubated with anti-CD16/CD32 to block the FcγRs. The cells were then treated with Phycoerythrin (PE)-labeled anti-F4/80 and goat anti-rabbit IgG conjugated with fluorescein isothiocyanate (FITC). The stained cells were analyzed using flow cytometry. The flow cytometry results indicate that polypeptides comprising a first and second Fc fragment of rabbit IgG in dimeric form bind to macrophages (See FIG. 55B).

The experiments may also be conducted using polypeptides comprising a first and second Fc fragment of murine BALB/c IgG, polypeptides comprising a first and second Fc fragment of murine C57BL/6 IgG or polypeptides comprising a first and second Fc fragment of human IgG. One of ordinary skill in the art would recognize that polypeptides comprising a first and second Fc fragment of murine BALB/c IgG, polypeptides comprising a first and second Fc fragment of murine C57BL/6 IgG or polypeptides comprising a first and second Fc fragment of human IgG would work in an equivalent manner with similar results.

### Example 13

### Polypeptides Comprising the First and Second Fc Fragment of Rabbit IgG in Dimeric Form Bind to FcγRs

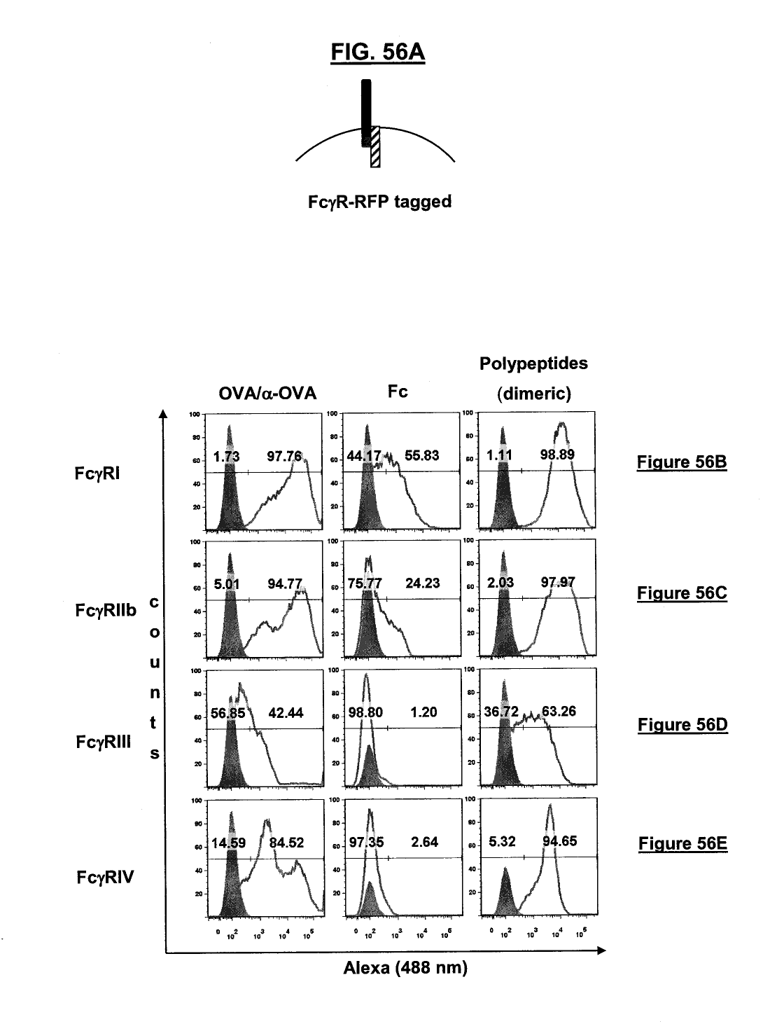

There are four Fc-gamma receptors (FcγRs) in mice including: Fcγ-receptor I, Fcγ-receptor III, Fcγ-receptor IV, and Fcγ-receptor IIb. The genes for each of the Fcγ-receptor I, Fcγ-receptor III, Fcγ-receptor IV, and Fcγ-receptor IIb were cloned into four separate plasmids. HeLa cells, cells which do not normally express FcγRs, were transfected with one of the four different FcγR plasmids generating HeLa cells that express FcγRI, HeLa cells that express FcγRIIb, HeLa cells that express FcγRIII, and HeLa cells that express FcγRIV. A red fluorescent protein tag (RFP) is attached to the intracellular portion of the FcγR (FIG. 56A). Thus, binding to the FcγR of a transfected HeLa cell will result in a signal transduction that causes the cells to fluoresce red, which may be measured by flow cytometry.

HeLa cells expressing FcγRI on their surface were treated with polypeptides containing a first Fc fragment of rabbit IgG in dimeric form (“Fc”) (FIG. 56B, center panel) and polypeptides comprising a first and second Fc fragment of rabbit IgG in dimeric form (FIG. 56B, right panel). The flow cytometry results indicate that 98.89% of the HeLa cells expressing FcγRI were bound by polypeptides comprising a first and second Fc fragment of rabbit IgG in dimeric form, while 55.83% of the HeLa cells expressing FcγRI were bound by “Fc”. HeLa cells expressing FcγRIIb on their surface were treated with “Fc” (FIG. 56C, center panel) and polypeptides comprising a first and second Fc fragment of rabbit IgG in dimeric form (FIG. 56C, right panel). The flow cytometry results indicate that 97.97% of the HeLa cells expressing FcγRIIb were bound by polypeptides comprising a first and second Fc fragment of rabbit IgG in dimeric form, while 24.23% of the HeLa cells expressing FcγRIIb were bound by “Fc”. HeLa cells expressing FORM on their surface were treated with “Fc” (FIG. 56D, center panel) and polypeptides comprising a first and second Fc fragment of rabbit IgG in dimeric form (FIG. 56D, right panel). The flow cytometry results indicate that 63.26% of the HeLa cells expressing FcγRIII were bound by polypeptides comprising a first and second Fc fragment of rabbit IgG in dimeric form, while 1.20% of the HeLa cells expressing FcγRIII were bound by “Fc”. HeLa cells expressing FcγRIV on their surface were treated with “Fc” (FIG. 56E, center panel) and polypeptides comprising a first and second Fc fragment of rabbit IgG in dimeric form (FIG. 56E, right panel). The flow cytometry results indicate that 94.65% of the HeLa cells expressing FcγRIV were bound by polypeptides comprising a first and second Fc fragment of rabbit IgG in dimeric form, while 2.64% of the HeLa cells expressing FcγRIV were bound by “Fc”. HeLa cells expressing FcγRI, FcγRIIb, FcγRIII, or FcγRIV were also treated with immune complexes as a positive control (FIGS. 56B-E, left panels). The immune complexes were prepared by adding polyclonal anti-Ovalbumin (OVA) to OVA.

The results in FIGS. 56B-E (right panels) indicate that polypeptides comprising a first and second Fc fragment of rabbit IgG in dimeric form are able to bind FcγRI, FcγRIIb, FcγRIII, and FcγRIV.

The experiment may also be conducted using polypeptides comprising a first and second Fc fragment of murine BALB/c IgG, polypeptides comprising a first and second Fc fragment of murine C57BL/6 IgG or polypeptides comprising a first and second Fc fragment of human IgG and the appropriate reagents including, but not limited, to antibodies and conjugated antibodies. One of ordinary skill in the art would recognize that polypeptides comprising a first and second Fc fragment of murine BALB/c IgG, polypeptides comprising a first and second Fc fragment of murine C57BL/6 IgG or polypeptides comprising a first and second Fc fragment of human IgG would work in an equivalent manner with similar results.

### Example 14

### IL-10 Production by Macrophages Stimulated with LPS in the Presence of Polypeptides Comprising a First and Second Fc Fragment of Rabbit IgG in Dimeric Form

BMMφs of wild-type BALB/c mice were plated in petri dishes in Dulbecco's Modified Eagle's Medium (DMEM/F12)(from GIBCO/BRL) supplemented with 10% Fetal Bovine Serum (FBS), glutamine, penicillin/streptomycin, and 20% L-929 cell conditioned medium. Cells were fed on days 2 and 5. On day 7, cells were removed from petri dishes and cultured on tissue culture dishes in complete medium without L-929 cell conditioned medium. On the next day, media was changed and cells were ready for future experiments.

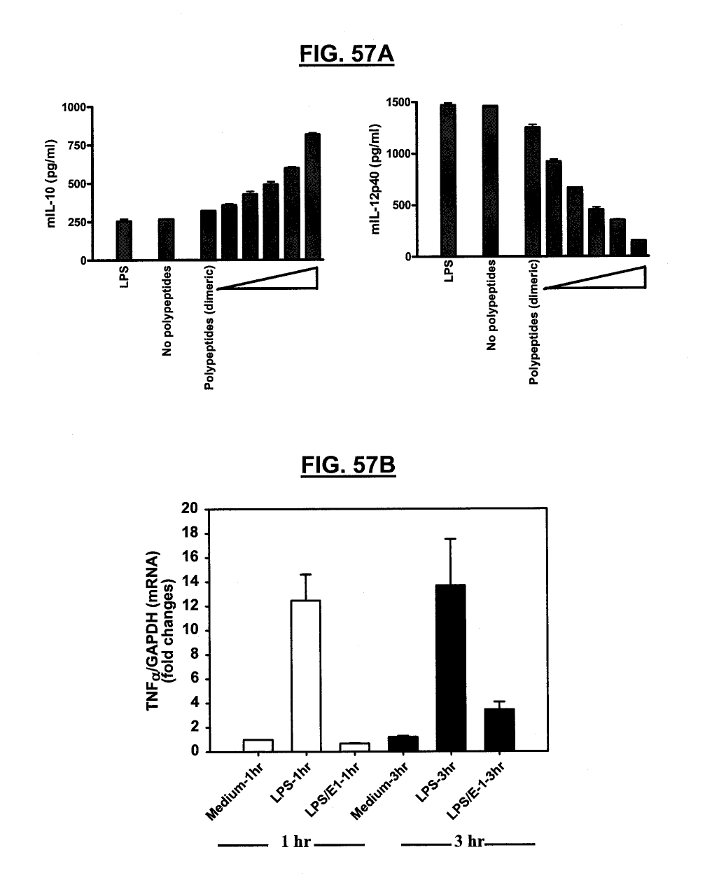

BMMφs were added at 0.3×10⁶/well with 0.5 ml medium in 48-well culture plates. LPS (10 ng/mL) was added alone or together with increasingly concentrated supernatants from HeLa cells that express polypeptides comprising a first and second Fc fragment of rabbit IgG in dimeric form (dimeric polypeptides). After an incubation of 16 hrs, the supernatants were collected from LPS-treated BMMφs (LPS, lane 1), BMMφs treated with supernatants from HeLa cells that do not express polypeptides comprising a first and second Fc fragment of rabbit IgG (no polypeptides, lane 2), and BMMφs treated with both LPS and supernatants from HeLa cells that express polypeptides comprising a first and second Fc fragment of rabbit IgG in dimeric form (dimeric polypeptides, lanes 3-8). The collected BMW supernatants were subjected to an ELISA to detect IL-10 and IL-12p40. Results indicated that IL-10 was increased in cells treated with LPS and polypeptides comprising a first and second Fc fragment of rabbit IgG in dimeric form. The level of IL-10 increased as the concentration of polypeptides comprising a first and second Fc fragment of rabbit IgG in dimeric form increased (See FIG. 57A, left panel). In addition, results indicated that IL-12p40 levels decreased in cells treated with LPS and polypeptides comprising a first and second Fc fragment of rabbit IgG in dimeric form. The level of IL-12p40 decreased as the concentration of polypeptides comprising a first and second Fc fragment of rabbit IgG in dimeric form increased (See FIG. 57A, right panel).

### Example 15

### Decrease in TNF-α Production by Macrophages Stimulated with LPS in the Presence of Polypeptides Comprising a First and Second Fc Fragment of Rabbit IgG in Dimeric Form

RAW 264.7 are murine macrophage cells from ATCC (Cat#. TIB-71). Cells were maintained in DMEM/F12 supplemented with 10% FBS, glutamine, and penicillin/streptomycin. RAW 264.7 cells were added at 2×10⁶/well with 1 ml medium in 6-well culture plates. LPS (10 ng/mL) was added alone or together with polypeptides comprising a first and second Fc fragment of rabbit IgG in dimeric form that were obtained from a chromatography fraction E1 obtained from the protein A bead column purification process described in Example 11. After incubation for 1 or 3 hrs, the supernatants were removed and 1 ml of TRIZOL® (Invitrogen™ Life Technologies) was added to each well. Total RNA was isolated following the procedures provided by Invitrogen™. The samples were treated with RNase-free DNase I (Roche™ Diagnostics) to remove contaminated genomic DNA. ThermoScript™ RT-PCR system (Invitrogen™ Life Technologies) was used to generate cDNA from approximately 3 μg of total RNA per sample using random hexamers or oligo(dT)₂₀. Real-time PCR was performed on a LightCycler®480 Real-time PCR System (Roche™ Applied Science, USA) with SYBR® Green PCR reagents (BIO-RAD™, USA). Melting curve analyses were carried out to ensure that a single product with the expected melting curve characteristics was obtained. The relative differences among samples were analyzed using the ΔΔCt method. The Ct value for GAPDH was used as an internal control to correct for variations in RNA quantity and cDNA synthesis. A ΔΔCt value was then obtained by subtracting the ΔCt value for the sample of medium alone from the corresponding experimental ΔCt. ΔCt equals to Ct of TNF-α minus Ct of GAPDH. The ΔΔCt values were converted to fold difference compared with the control by raising 2 to the ΔΔCt power.

The addition of LPS to RAW 264.7 cells induced the production of high levels of the inflammatory cytokine TNF-α (FIG. 57B, lanes 2 and 5). However, the addition of supernatants from macrophages that were stimulated with LPS and polypeptides comprising a first and second Fc fragment of rabbit IgG in dimeric form almost completely ablates TNF-α mRNA production (FIG. 57B, lanes 3 and 6).

### Example 16

### IL-10 Production by Macrophages Stimulated with LPS in the Presence of Polypeptides Comprising at Least a First and Second Fc Fragment of Murine BALB/c IgG in Dimeric Form

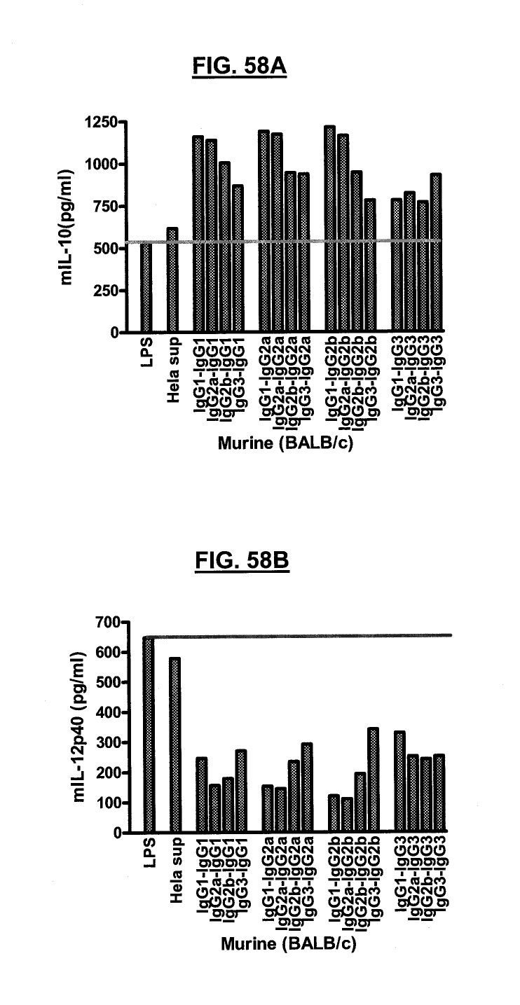

An experiment as set forth in Example 14 was conducted using supernatants from HeLa cells that express polypeptides comprising a first and second Fc fragment of murine BALB/c IgG in dimeric form (FIG. 58A-B). Supernatants from HeLa cells that express polypeptides comprising the first and second Fc fragment of murine C57BL/6 IgG in dimeric form may also be used. One of ordinary skill in the art would recognize that polypeptides comprising a first and second Fc fragment of murine C57BL/6 IgG would work in an equivalent manner with similar results.

FIG. 58A shows that polypeptides comprising a first Fc fragment of murine BALB/c IgG1 and second Fc fragment of murine BALB/c IgG1, polypeptides comprising a first Fc fragment of murine BALB/c IgG2a and second Fc fragment of murine BALB/c IgG2a, polypeptides comprising a first Fc fragment of murine BALB/c IgG2b and second Fc fragment of murine BALB/c IgG2b, and polypeptides comprising a first Fc fragment of murine BALB/c IgG3 and second Fc fragment of murine BALB/c IgG3 were all equally effective at inducing IL-10. Polypeptides comprising first and second Fc fragments of IgG comprising any combination of murine BALB/c IgG1, murine BALB/c IgG2a, and murine BALB/c IgG2b also effectively induced IL-10. Also, polypeptides comprising one Fc fragment of murine BALB/c IgG3 and another Fc fragment selected from a group consisting of murine BALB/c IgG1, murine BALB/c IgG2a, and murine BALB/c IgG2b were also less effective at inducing IL-10. All of the polypeptides comprising a first Fc fragment of murine BALB/c IgG and a second fragment of murine BALB/c IgG set forth herein were able to induce IL-10 production (See FIG. 58A) and downregulate IL-12 (See FIG. 58B) compared to the control samples (LPS and HeLa sup.).

### Example 17

### Decrease in TNF-α Production by Macrophages Stimulated with LPS in the Presence of Polypeptides Comprising a First and Second Fc Fragment of Murine IgG in Dimeric Form

An experiment as set forth in Example 15 may be conducted using supernatants from HeLa cells that express polypeptides comprising a first and second Fc fragment of murine BALB/c IgG in dimeric form or supernatants from HeLa cells that express polypeptides comprising the first and second Fc fragment of murine C57BL/6 IgG in dimeric form. One of ordinary skill in the art would recognize that polypeptides comprising a first and second Fc fragment of murine BALB/c IgG, polypeptides comprising a first and second Fc fragment of murine C57BL/6 IgG would work in an equivalent manner with similar results.

### Example 18

### IL-10 Production by Macrophages Stimulated with LPS in the Presence of Polypeptides Comprising at Least a First and Second Fc Fragment of Human IgG in Dimeric Form

An experiment as set forth in Example 14 may be conducted using supernatants from HeLa cells that express polypeptides comprising a first and second Fc fragment of human IgG in dimeric form. One of ordinary skill in the art would recognize that polypeptides comprising a first and second Fc fragment of human IgG would work in an equivalent manner with similar results.

### Example 19

### Decrease in TNF-α Production by Macrophages Stimulated with LPS in the Presence of Polypeptides Comprising a First and Second Fc Fragment of Human IgG in Dimeric Form

An experiment as set forth in Example 15 may be conducted using supernatants from HeLa cells that express polypeptides comprising a first and second Fc fragment of human IgG in dimeric form. One of ordinary skill in the art would recognize that polypeptides comprising a first and second Fc fragment of human IgG would work in an equivalent manner with similar results.

### Example 20

### Polypeptides Comprising the First and Second Fc Fragment of Rabbit IgG in Dimeric Form Signal Through the FcγR to Induce IL-10

As set forth herein, there are four FcγRs in mice including: FcγRI, FcγRIIb, FcγRIII, and FcγRIV. FcγRI, FcγRIII, and FcγRIV require the FcγR gamma chain for an intact signal transduction or signaling to occur. Thus, FcγR gamma chain knockout mouse are unable to properly signaling through FcγRI, FcγRIII, and FcγRIV. In contrast, FcγRIIb is a single chain receptor. Thus, Fc-receptor IIb knockout mice are unable to signal through FcγRIIb, but can signal through FcγRI, FcγRIII, and FcγRIV similar to wild-type mice.

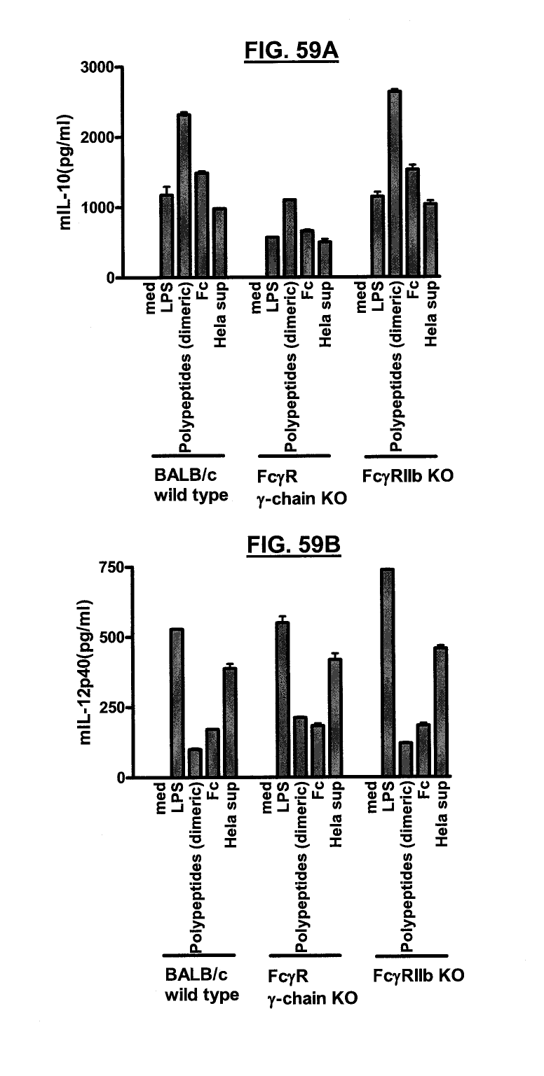

BMMφs of wild-type BALB/c mice, FcγR-gamma chain knockout mice, and FcγR IIb knockout mice were isolated from the femurs and tibias of mice 6-8 weeks of age on a BALB/c background and cultured. Day 6 BMMφs were subcultured at 2×10⁵ cells/well and stimulated with 10 ng/mL lipopolysaccharide (LPS) and polypeptides comprising a first and second Fc-fragment of rabbit IgG in dimeric form (lanes 3, 8, and 13), polypeptides containing only a first Fc fragment in dimeric form (“Fc”) (lanes 4, 9, and 14) or controls (lanes 1-2, 5; lanes 6-7, 10; and lanes 11-12, 15). Supernatants were collected from the treated cells after 6 hours and used to detect IL-10 (FIG. 59A) and IL-12p40 (FIG. 59B) via ELISA.

The experiment may also be conducted using polypeptides comprising a first and second Fc fragment of murine BALB/c IgG in dimeric form, polypeptides comprising a first and second Fc fragment of murine C57BL/6 IgG in dimeric form or polypeptides comprising a first and second Fc fragment of human IgG in dimeric form. One of ordinary skill in the art would recognize that polypeptides comprising a first and second Fc fragment of murine BALB/c IgG, polypeptides comprising a first and second Fc fragment of murine C57BL/6 IgG, and polypeptides comprising a first and second Fc fragment of human IgG would work in an equivalent manner with similar results.

### Example 21

### Polypeptides Comprising the First and Second Fc Fragment of Rabbit IgG in Dimeric Form Provide Anti-Inflammatory Protection in Mice

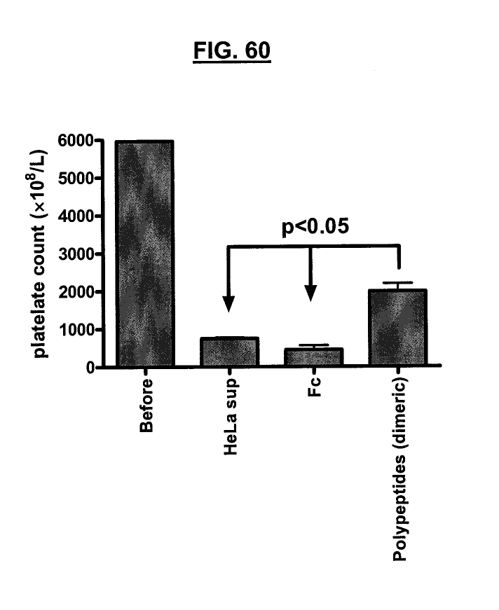

Mice were injected intraperitoneally with 1 mL (3 μl) of HeLa cell supernatant containing polypeptides comprising a first and second Fc fragment of rabbit IgG in dimeric form (“dimeric polypeptides”), polypeptides containing only a first Fc fragment in dimeric form (“Fc”) or a control supernatant from mock transfected HeLa cells. Mock transfected HeLa cells are cells transfected with a pFuse vector that does not comprise first and second Fc fragment genes of rabbit IgG (HeLa sup). Therefore, these mock transfected HeLa cells do not produce polypeptides comprising a first and second Fc fragment of rabbit IgG in dimeric form. After 24-hours, immune thrombocytopenic purpura (ITP) was induced in the mice that were intraperitoneally injected with either HeLa cell supernatant containing polypeptides comprising a first and second Fc fragment of rabbit IgG in dimeric form, polypeptides containing only a first Fc fragment in dimeric form (“Fc”) or a control supernatant. ITP was induced in these mice by intraperitoneally injecting 2 μg of anti-CD41 (integrin αIIb) antibody in 200 μl PBS. Twenty four hours later, the mice were bled by tail vein and the blood was diluted 10,000 times in PBS/citrate buffer. Platelets were counted using a flow rate-calibrated FACScan flow cytometer (Becton Dickinson) and compared to platelet numbers in control mice that were not intraperitoneally injected with supernatants and induced with ITP (FIG. 60).

### Example 22

### Polypeptides Comprising the First and Second Fc Fragment of Rabbit IgG in Dimeric Form have an Enhanced Binding for FcγRs on Cells

Polypeptides comprising a first and second Fc fragment of rabbit IgG in dimeric form and whole rabbit IgG were quantified by ELISA, using the whole rabbit IgG as the standards. HeLa cells that express FcγRI, HeLa cells that express FcγRIIb, HeLa cells that express FcγRIII, and HeLa cells that express FcγRIV as described in Example 13 were stained with 2-fold series diluted either with polypeptides comprising a first and second Fc fragment of rabbit IgG in dimeric form or whole rabbit IgG. The cells were then stained with Zenon Alexa Fluor 488 rabbit IgG labeling kits. Flow cytometry was performed. The HeLa cells that express FcγRI, HeLa cells that express FcγRIIb, HeLa cells that express FcγRIII, and HeLa cells that express FcγRIV were gated by red fluorescence (RFP); and the mean fluorescence of Alexa Fluor was measured for the Saturation Binding Curves (FIGS. 61A-D).

Having now fully described this invention, it will be understood to those of ordinary skill in the art that the same can be performed within a wide and equivalent range of conditions, formulations and other parameters without affecting the scope of the invention or any embodiment thereof. It will be appreciated by those skilled in the art that changes could be made to the embodiments described herein without departing from the broad concept of the invention. It is understood, therefore, that this invention is not limited to the particular embodiments disclosed, but is intended to cover modifications that are within the spirit and scope of the invention as defined by the claims.

1-26. (canceled)

27. A method of reducing macrophage-mediated inflammation, the method comprising:
administering a therapeutically effective amount of a polypeptide, the polypeptide comprising:
a first Fc fragment of IgG and a second Fc fragment of IgG, the first and the second Fc fragments of IgG being attached in a series;
the first Fc fragment of IgG consisting of a full-length hinge region and a full-length C_{H}3 domain;
the second Fc fragment of IgG consisting of a full-length C_{H}3 domain;
wherein the hinge region of the first Fc fragment of IgG is the N-terminus of the first Fc fragment of IgG and wherein the N-terminus of the first Fc fragment of IgG is the N-terminus of the polypeptide;
wherein the C_{H}3 domain of the second Fc fragment of IgG is the C-terminus of the second Fc fragment of IgG and wherein the C-terminus of the second Fc fragment of IgG is the C-terminus of the polypeptide.

28. The method of claim 27, wherein the first Fc fragment of IgG and the second Fc fragment of IgG form a chain and said polypeptide comprises two said chains in dimeric form.

29. The method of claim 28, wherein the polypeptide comprising two chains in dimeric form is configured to bind and cross-link at least two Fc-gamma receptors on a stimulated cell.

30. The method of claim 27, wherein the first Fc fragment of IgG and the second Fc fragment of IgG form a chain and said polypeptide comprises multiple said chains in multimeric form.

31. The method of claim 27, wherein the first Fc fragment of IgG and the second Fc fragment of IgG are each selected from one of a group consisting of an Fc fragment of murine IgG, an Fc fragment of rabbit IgG, and an Fc fragment of human IgG.

32. The method of claim 27, wherein the first Fc fragment of IgG and the second Fc fragment of IgG are Fc fragments of human IgG.

33. The method of claim 32, wherein the Fc fragments of human IgG are selected from a group consisting of Fc fragments of human IgG1, Fc fragments of human IgG2, Fc fragments of human IgG3 and Fc fragments of human IgG4.

34. A method of reducing macrophage-mediated inflammation, the method comprising:
administering a therapeutically effective amount of a polypeptide, the polypeptide comprising:
a first Fc fragment of IgG and a second Fc fragment of IgG, the first and the second Fc fragments of IgG being attached in a series;
the first Fc fragment of IgG consisting of a full-length hinge region, a full-length C_{H}3 domain, and a C_{H}2 domain positioned intermediate to the hinge region and C_{H}3 domain;
the second Fc fragment of IgG consisting of a C_{H}2 domain and a full-length C_{H}3 domain;
wherein the hinge region of the first Fc fragment of IgG is the N-terminus of the first Fc fragment of IgG and wherein the N-terminus of the first Fc fragment of IgG is the N-terminus of the polypeptide;
wherein the C_{H}3 domain of the second Fc fragment of IgG is the C-terminus of the second Fc fragment of IgG and wherein the C-terminus of the second Fc fragment of IgG is the C-terminus of the polypeptide.

35. The method of claim 34, wherein the first Fc fragment of IgG and the second Fc fragment of IgG form a chain and said polypeptide comprises two said chains in dimeric form.

36. The method of claim 35, wherein the polypeptide comprising two chains in dimeric form is configured to bind and cross-link at least two Fc-gamma receptors on a stimulated cell.

37. The method of claim 34, wherein the first Fc fragment of IgG and the second Fc fragment of IgG form a chain and said polypeptide comprises multiple said chains in multimeric form.

38. The method of claim 34, wherein the first Fc fragment of IgG and the second Fc fragment of IgG are each selected from one of a group consisting of an Fc fragment of murine IgG, an Fc fragment of rabbit IgG, and an Fc fragment of human IgG.

39. The method of claim 34, wherein the first Fc fragment of IgG and the second Fc fragment of IgG are Fc fragments of human IgG.

40. The method of claim 39, wherein the Fc fragments of human IgG are selected from a group consisting of Fc fragments of human IgG1, Fc fragments of human IgG2, Fc fragments of human IgG3 and Fc fragments of human IgG4.

41. A method of reducing macrophage-mediated inflammation, the method comprising:
administering a therapeutically effective amount of a polypeptide, the polypeptide comprising:
a first Fc fragment of IgG and a second Fc fragment of IgG, the first and the second Fc fragments of IgG being attached in a series;
the first Fc fragment of IgG consisting of a full-length hinge region, a full-length C_{H}3 domain, and a full-length C_{H}2 domain positioned intermediate to the hinge region and C_{H}3 domain;
the second Fc fragment of IgG consisting of a full-length C_{H}3 domain;
wherein the hinge region of the first Fc fragment of IgG is the N-terminus of the first Fc fragment of IgG and wherein the N-terminus of the first Fc fragment of IgG is the N-terminus of the polypeptide;
wherein the C_{H}3 domain of the second Fc fragment of IgG is the C-terminus of the second Fc fragment of IgG and wherein the C-terminus of the second Fc fragment of IgG is the C-terminus of the polypeptide.

42. The method of claim 41, wherein the first Fc fragment of IgG and the second Fc fragment of IgG form a chain and said polypeptide comprises two said chains in dimeric form.

43. The method of claim 42, wherein the polypeptide comprising two chains in dimeric form is configured to bind and cross-link at least two Fc-gamma receptors on a stimulated cell.

44. The method of claim 41, wherein the first Fc fragment of IgG and the second Fc fragment of IgG form a chain and said polypeptide comprises multiple said chains in multimeric form.

45. The method of claim 41, wherein the first Fc fragment of IgG and the second Fc fragment of IgG are each selected from one of a group consisting of an Fc fragment of murine IgG, an Fc fragment of rabbit IgG, and an Fc fragment of human IgG.

46. The method of claim 41, wherein the first Fc fragment of IgG and the second Fc fragment of IgG are Fc fragments of human IgG.

47. The method of claim 46, wherein the Fc fragments of human IgG are selected from a group consisting of Fc fragments of human IgG1, Fc fragments of human IgG2, Fc fragments of human IgG3 and Fc fragments of human IgG4.

48. A method of reducing macrophage-mediated inflammation, the method comprising:
administering a therapeutically effective amount of a polypeptide, the polypeptide comprising:
a first Fc fragment of IgG and a second Fc fragment of IgG, the first and the second Fc fragments of IgG being attached in a series;
the first Fc fragment of IgG consisting of a full-length hinge region, a full-length C_{H}3 domain, and a full-length C_{H}2 domain positioned intermediate to the hinge region and C_{H}3 domain;
the second Fc fragment of IgG consisting of a full-length hinge region, a full-length C_{H}3 domain, and a full-length C_{H}2 domain positioned intermediate to the hinge region and C_{H}3 domain;
wherein the hinge region of the first Fc fragment of IgG is the N-terminus of the first Fc fragment of IgG and wherein the N-terminus of the first Fc fragment of IgG is the N-terminus of the polypeptide;
wherein the C_{H}3 domain of the second Fc fragment of IgG is the C-terminus of the second Fc fragment of IgG and wherein the C-terminus of the second Fc fragment of IgG is the C-terminus of the polypeptide;
wherein the first Fc fragment of IgG and the second Fc fragment of IgG are bound through one hinge region, the one hinge region being the hinge region of the second Fc fragment of IgG;
wherein the first Fc fragment of IgG and the second Fc fragment of IgG form a chain and said polypeptide comprises two said chains in dimeric form configured to bind and cross-link at least two Fc-gamma receptors on a stimulated cell.

49. The method of claim 48, wherein the first Fc fragment of IgG and the second Fc fragment of IgG are each selected from one of a group consisting of an Fc fragment of murine IgG, an Fc fragment of rabbit IgG, and an Fc fragment of human IgG.

50. The method of claim 48, wherein the first Fc fragment of IgG and the second Fc fragment of IgG are Fc fragments of human IgG.

51. The method of claim 50, wherein the Fc fragments of human IgG are selected from a group consisting of Fc fragments of human IgG1, Fc fragments of human IgG2, Fc fragments of human IgG3 and Fc fragments of human IgG4.

52. A method of reducing macrophage-mediated inflammation, the method comprising:
administering a therapeutically effective amount of a polypeptide, the polypeptide comprising:
a first Fc fragment of IgG and a second Fc fragment of IgG, the first and the second Fc fragments of IgG being directly attached in a series;
the first Fc fragment of IgG consisting of a full-length hinge region, a full-length C_{H}3 domain, and a full-length C_{H}2 domain positioned intermediate to the hinge region and C_{H}3 domain;
the second Fc fragment of IgG consisting of a full-length hinge region, a full-length C_{H}3 domain, and a full-length C_{H}2 domain positioned intermediate to the hinge region and C_{H}3 domain;
wherein the hinge region of the first Fc fragment of IgG is the N-terminus of the first Fc fragment of IgG and wherein the N-terminus of the first Fc fragment of IgG is the N-terminus of the polypeptide;
wherein the C_{H}3 domain of the second Fc fragment of IgG is the C-terminus of the second Fc fragment of IgG and wherein the C-terminus of the second Fc fragment of IgG is the C-terminus of the polypeptide;
wherein the first Fc fragment of IgG and the second Fc fragment of IgG are bound through one hinge region, the one hinge region being the hinge region of the second Fc fragment of IgG;
wherein the first Fc fragment of IgG and the second Fc fragment of IgG form a chain and said polypeptide comprises multiple said chains in multimeric form configured to bind and cross-link at least two Fc-gamma receptors on a stimulated cell.


SEQUENCE LISTING (148 sequences)

SEQ ID NO: 1 - DNA, 1530 bp, rabbit
atgtacaggatgcaactcctgtcttgcattgcactaagtcttgcacttgtcacgaattcggggggttctcatcatcatcatcatcatggtatggcaagcatgactggtggacagcaaatgggtcgggatctgtacgacgatgacgataaggcgccctcgacatgcagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaagatatcgttagatctagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatgagctagctgg

SEQ ID NO: 2 - protein, 506 aa, rabbit
MYRMQLLSCIALSLALVTNSGGSHHHHHHGMASMTGGQQMGRDLYDDDDKAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGKDIVRSSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 3 - DNA, 1544 bp, rabbit
atgtacaggatgcaactcctgtcttgcattgcactaagtcttgcacttgtcacgaattcggggggttctcatcatcatcatcatcatggtatggcaagcatgactggtggacagcaaatgggtcgggatctgtacgacgatgacgataaggcgccctcgacatgcagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaagatatcgttagatctagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatattattactattattgagctagctg

SEQ ID NO: 4 - protein, 511 aa, rabbit
MYRMQLLSCIALSLALVTNSGGSHHHHHHGMASMTGGQQMGRDLYDDDDKAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGKDIVRSSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGKYYYYY

SEQ ID NO: 5 - DNA, 1539 bp, mouse
atgtacaggatgcaactcctgtcttgcattgcactaagtcttgcacttgtcacgaattcggggggttctcatcatcatcatcatcatggtatggcaagcatgactggtggacagcaaatgggtcgggatctgtacgacgatgacgataaggtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaagatatcgttagatctgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatgagctagctgg

SEQ ID NO: 6 - protein, 509 aa, mouse
MYRMQLLSCIALSLALVTNSGGSHHHHHHGMASMTGGQQMGRDLYDDDDKVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKDIVRSVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 7 - DNA, 1557 bp, mouse
atgtacaggatgcaactcctgtcttgcattgcactaagtcttgcacttgtcacgaattcggggggttctcatcatcatcatcatcatggtatggcaagcatgactggtggacagcaaatgggtcgggatctgtacgacgatgacgataaggagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaagatatcgttagatctgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatgagctagctgg

SEQ ID NO: 8 - protein, 515 aa, mouse
MYRMQLLSCIALSLALVTNSGGSHHHHHHGMASMTGGQQMGRDLYDDDDKEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKDIVRSVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 9 - DNA, 1575 bp, mouse
atgtacaggatgcaactcctgtcttgcattgcactaagtcttgcacttgtcacgaattcggggggttctcatcatcatcatcatcatggtatggcaagcatgactggtggacagcaaatgggtcgggatctgtacgacgatgacgataaggagcccagcgggcccatttcaacaatcaacccctgtcctccatgcaaggagtgtcacaaatgcccagctcctaacctcgagggtggaccatccgtcttcatcttccctccaaatatcaaggatgtactcatgatctccctgacacccaaggtcacgtgtgtggtggtggatgtgagcgaggatgacccagacgtccagatcagctggtttgtgaacaacgtggaagtacacacagctcagacacaaacccatagagaggattacaacagtactatccgggtggtcagcaccctccccatccagcaccaggactggatgagtggcaaggagttcaaatgcaaggtcaacaacaaagacctcccatcacccatcgagagaaccatctcaaaaattaaagggctagtcagagctccacaagtatacatcttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagtcctggactctgacggttcttacttcatatatagcaagctcaatatgaaaacaagcaagtgggagaaaacagattccttctcatgcaacgtgagacacgagggtctgaaaaattactacctgaagaagaccatctcccggtctccgggtaaagatatcgttagatctgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatgagctagctgg

SEQ ID NO: 10 - protein, 521 aa, mouse
MYRMQLLSCIALSLALVTNSGGSHHHHHHGMASMTGGQQMGRDLYDDDDKEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGKDIVRSVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 11 - DNA, 1557 bp, mouse
atgtacaggatgcaactcctgtcttgcattgcactaagtcttgcacttgtcacgaattcggggggttctcatcatcatcatcatcatggtatggcaagcatgactggtggacagcaaatgggtcgggatctgtacgacgatgacgataaggagcctagaatacccaagcccagtacccccccaggttcttcatgcccacctggtaacatcttgggtggaccatccgtcttcatcttccccccaaagcccaaggatgcactcatgatctccctaacccccaaggttacgtgtgtggtggtggatgtgagcgaggatgacccagatgtccatgtcagctggtttgtggacaacaaagaagtacacacagcctggacacagccccgtgaagctcagtacaacagtaccttccgagtggtcagtgccctccccatccagcaccaggactggatgaggggcaaggagttcaaatgcaaggtcaacaacaaagccctcccagcccccatcgagagaaccatctcaaaacccaaaggaagagcccagacacctcaagtatacaccatacccccacctcgtgaacaaatgtccaagaagaaggttagtctgacctgcctggtcaccaacttcttctctgaagccatcagtgtggagtgggaaaggaacggagaactggagcaggattacaagaacactccacccatcctggactcagatgggacctacttcctctacagcaagctcactgtggatacagacagttggttgcaaggagaaatttttacctgctccgtggtgcatgaggctctccataaccaccacacacagaagaacctgtctcgctcccctggtaaagatatcgttagatctgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatgagctagctgg

SEQ ID NO: 12 - protein, 515 aa, mouse
MYRMQLLSCIALSLALVTNSGGSHHHHHHGMASMTGGQQMGRDLYDDDDKEPRIPKPSTPPGSSCPPGNILGGPSVFIFPPKPKDALMISLTPKVTCVVVDVSEDDPDVHVSWFVDNKEVHTAWTQPREAQYNSTFRVVSALPIQHQDWMRGKEFKCKVNNKALPAPIERTISKPKGRAQTPQVYTIPPPREQMSKKKVSLTCLVTNFFSEAISVEWERNGELEQDYKNTPPILDSDGTYFLYSKLTVDTDSWLQGEIFTCSVVHEALHNHHTQKNLSRSPGKDIVRSVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 13 - DNA, 1557 bp, mouse
atgtacaggatgcaactcctgtcttgcattgcactaagtcttgcacttgtcacgaattcggggggttctcatcatcatcatcatcatggtatggcaagcatgactggtggacagcaaatgggtcgggatctgtacgacgatgacgataaggtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaagatatcgttagatctgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatgagctagctgg

SEQ ID NO: 14 - protein, 515 aa, mouse
MYRMQLLSCIALSLALVTNSGGSHHHHHHGMASMTGGQQMGRDLYDDDDKVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKDIVRSEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 15 - DNA, 1575 bp, mouse
atgtacaggatgcaactcctgtcttgcattgcactaagtcttgcacttgtcacgaattcggggggttctcatcatcatcatcatcatggtatggcaagcatgactggtggacagcaaatgggtcgggatctgtacgacgatgacgataaggagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaagatatcgttagatctgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatgagctagctgg

SEQ ID NO: 16 - protein, 521 aa, mouse
MYRMQLLSCIALSLALVTNSGGSHHHHHHGMASMTGGQQMGRDLYDDDDKEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKDIVRSEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 17 - DNA, 1593 bp, mouse
atgtacaggatgcaactcctgtcttgcattgcactaagtcttgcacttgtcacgaattcggggggttctcatcatcatcatcatcatggtatggcaagcatgactggtggacagcaaatgggtcgggatctgtacgacgatgacgataaggagcccagcgggcccatttcaacaatcaacccctgtcctccatgcaaggagtgtcacaaatgcccagctcctaacctcgagggtggaccatccgtcttcatcttccctccaaatatcaaggatgtactcatgatctccctgacacccaaggtcacgtgtgtggtggtggatgtgagcgaggatgacccagacgtccagatcagctggtttgtgaacaacgtggaagtacacacagctcagacacaaacccatagagaggattacaacagtactatccgggtggtcagcaccctccccatccagcaccaggactggatgagtggcaaggagttcaaatgcaaggtcaacaacaaagacctcccatcacccatcgagagaaccatctcaaaaattaaagggctagtcagagctccacaagtatacatcttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagtcctggactctgacggttcttacttcatatatagcaagctcaatatgaaaacaagcaagtgggagaaaacagattccttctcatgcaacgtgagacacgagggtctgaaaaattactacctgaagaagaccatctcccggtctccgggtaaagatatcgttagatctgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatgagctagctgg

SEQ ID NO: 18 - protein, 527 aa, mouse
MYRMQLLSCIALSLALVTNSGGSHHHHHHGMASMTGGQQMGRDLYDDDDKEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGKDIVRSEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 19 - DNA, 1575 bp, mouse
atgtacaggatgcaactcctgtcttgcattgcactaagtcttgcacttgtcacgaattcggggggttctcatcatcatcatcatcatggtatggcaagcatgactggtggacagcaaatgggtcgggatctgtacgacgatgacgataaggagcctagaatacccaagcccagtacccccccaggttcttcatgcccacctggtaacatcttgggtggaccatccgtcttcatcttccccccaaagcccaaggatgcactcatgatctccctaacccccaaggttacgtgtgtggtggtggatgtgagcgaggatgacccagatgtccatgtcagctggtttgtggacaacaaagaagtacacacagcctggacacagccccgtgaagctcagtacaacagtaccttccgagtggtcagtgccctccccatccagcaccaggactggatgaggggcaaggagttcaaatgcaaggtcaacaacaaagccctcccagcccccatcgagagaaccatctcaaaacccaaaggaagagcccagacacctcaagtatacaccatacccccacctcgtgaacaaatgtccaagaagaaggttagtctgacctgcctggtcaccaacttcttctctgaagccatcagtgtggagtgggaaaggaacggagaactggagcaggattacaagaacactccacccatcctggactcagatgggacctacttcctctacagcaagctcactgtggatacagacagttggttgcaaggagaaatttttacctgctccgtggtgcatgaggctctccataaccaccacacacagaagaacctgtctcgctcccctggtaaagatatcgttagatctgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatgagctagctgg

SEQ ID NO: 20 - protein, 521 aa, mouse
MYRMQLLSCIALSLALVTNSGGSHHHHHHGMASMTGGQQMGRDLYDDDDKEPRIPKPSTPPGSSCPPGNILGGPSVFIFPPKPKDALMISLTPKVTCVVVDVSEDDPDVHVSWFVDNKEVHTAWTQPREAQYNSTFRVVSALPIQHQDWMRGKEFKCKVNNKALPAPIERTISKPKGRAQTPQVYTIPPPREQMSKKKVSLTCLVTNFFSEAISVEWERNGELEQDYKNTPPILDSDGTYFLYSKLTVDTDSWLQGEIFTCSVVHEALHNHHTQKNLSRSPGKDIVRSEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 21 - DNA, 1575 bp, mouse
atgtacaggatgcaactcctgtcttgcattgcactaagtcttgcacttgtcacgaattcggggggttctcatcatcatcatcatcatggtatggcaagcatgactggtggacagcaaatgggtcgggatctgtacgacgatgacgataaggtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaagatatcgttagatctgagcccagcgggcccatttcaacaatcaacccctgtcctccatgcaaggagtgtcacaaatgcccagctcctaacctcgagggtggaccatccgtcttcatcttccctccaaatatcaaggatgtactcatgatctccctgacacccaaggtcacgtgtgtggtggtggatgtgagcgaggatgacccagacgtccagatcagctggtttgtgaacaacgtggaagtacacacagctcagacacaaacccatagagaggattacaacagtactatccgggtggtcagcaccctccccatccagcaccaggactggatgagtggcaaggagttcaaatgcaaggtcaacaacaaagacctcccatcacccatcgagagaaccatctcaaaaattaaagggctagtcagagctccacaagtatacatcttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagtcctggactctgacggttcttacttcatatatagcaagctcaatatgaaaacaagcaagtgggagaaaacagattccttctcatgcaacgtgagacacgagggtctgaaaaattactacctgaagaagaccatctcccggtctccgggtaaatgagctagctgg

SEQ ID NO: 22 - protein, 521 aa, mouse
MYRMQLLSCIALSLALVTNSGGSHHHHHHGMASMTGGQQMGRDLYDDDDKVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKDIVRSEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 23 - DNA, 1593 bp, mouse
atgtacaggatgcaactcctgtcttgcattgcactaagtcttgcacttgtcacgaattcggggggttctcatcatcatcatcatcatggtatggcaagcatgactggtggacagcaaatgggtcgggatctgtacgacgatgacgataaggagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaagatatcgttagatctgagcccagcgggcccatttcaacaatcaacccctgtcctccatgcaaggagtgtcacaaatgcccagctcctaacctcgagggtggaccatccgtcttcatcttccctccaaatatcaaggatgtactcatgatctccctgacacccaaggtcacgtgtgtggtggtggatgtgagcgaggatgacccagacgtccagatcagctggtttgtgaacaacgtggaagtacacacagctcagacacaaacccatagagaggattacaacagtactatccgggtggtcagcaccctccccatccagcaccaggactggatgagtggcaaggagttcaaatgcaaggtcaacaacaaagacctcccatcacccatcgagagaaccatctcaaaaattaaagggctagtcagagctccacaagtatacatcttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagtcctggactctgacggttcttacttcatatatagcaagctcaatatgaaaacaagcaagtgggagaaaacagattccttctcatgcaacgtgagacacgagggtctgaaaaattactacctgaagaagaccatctcccggtctccgggtaaatgagctagctgg

SEQ ID NO: 24 - protein, 527 aa, mouse
MYRMQLLSCIALSLALVTNSGGSHHHHHHGMASMTGGQQMGRDLYDDDDKEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKDIVRSEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 25 - DNA, 1611 bp, mouse
atgtacaggatgcaactcctgtcttgcattgcactaagtcttgcacttgtcacgaattcggggggttctcatcatcatcatcatcatggtatggcaagcatgactggtggacagcaaatgggtcgggatctgtacgacgatgacgataaggagcccagcgggcccatttcaacaatcaacccctgtcctccatgcaaggagtgtcacaaatgcccagctcctaacctcgagggtggaccatccgtcttcatcttccctccaaatatcaaggatgtactcatgatctccctgacacccaaggtcacgtgtgtggtggtggatgtgagcgaggatgacccagacgtccagatcagctggtttgtgaacaacgtggaagtacacacagctcagacacaaacccatagagaggattacaacagtactatccgggtggtcagcaccctccccatccagcaccaggactggatgagtggcaaggagttcaaatgcaaggtcaacaacaaagacctcccatcacccatcgagagaaccatctcaaaaattaaagggctagtcagagctccacaagtatacatcttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagtcctggactctgacggttcttacttcatatatagcaagctcaatatgaaaacaagcaagtgggagaaaacagattccttctcatgcaacgtgagacacgagggtctgaaaaattactacctgaagaagaccatctcccggtctccgggtaaagatatcgttagatctgagcccagcgggcccatttcaacaatcaacccctgtcctccatgcaaggagtgtcacaaatgcccagctcctaacctcgagggtggaccatccgtcttcatcttccctccaaatatcaaggatgtactcatgatctccctgacacccaaggtcacgtgtgtggtggtggatgtgagcgaggatgacccagacgtccagatcagctggtttgtgaacaacgtggaagtacacacagctcagacacaaacccatagagaggattacaacagtactatccgggtggtcagcaccctccccatccagcaccaggactggatgagtggcaaggagttcaaatgcaaggtcaacaacaaagacctcccatcacccatcgagagaaccatctcaaaaattaaagggctagtcagagctccacaagtatacatcttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagtcctggactctgacggttcttacttcatatatagcaagctcaatatgaaaacaagcaagtgggagaaaacagattccttctcatgcaacgtgagacacgagggtctgaaaaattactacctgaagaagaccatctcccggtctccgggtaaatgagctagctgg

SEQ ID NO: 26 - protein, 533 aa, mouse
MYRMQLLSCIALSLALVTNSGGSHHHHHHGMASMTGGQQMGRDLYDDDDKEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGKDIVRSEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 27 - DNA, 1593 bp, mouse
atgtacaggatgcaactcctgtcttgcattgcactaagtcttgcacttgtcacgaattcggggggttctcatcatcatcatcatcatggtatggcaagcatgactggtggacagcaaatgggtcgggatctgtacgacgatgacgataaggagcctagaatacccaagcccagtacccccccaggttcttcatgcccacctggtaacatcttgggtggaccatccgtcttcatcttccccccaaagcccaaggatgcactcatgatctccctaacccccaaggttacgtgtgtggtggtggatgtgagcgaggatgacccagatgtccatgtcagctggtttgtggacaacaaagaagtacacacagcctggacacagccccgtgaagctcagtacaacagtaccttccgagtggtcagtgccctccccatccagcaccaggactggatgaggggcaaggagttcaaatgcaaggtcaacaacaaagccctcccagcccccatcgagagaaccatctcaaaacccaaaggaagagcccagacacctcaagtatacaccatacccccacctcgtgaacaaatgtccaagaagaaggttagtctgacctgcctggtcaccaacttcttctctgaagccatcagtgtggagtgggaaaggaacggagaactggagcaggattacaagaacactccacccatcctggactcagatgggacctacttcctctacagcaagctcactgtggatacagacagttggttgcaaggagaaatttttacctgctccgtggtgcatgaggctctccataaccaccacacacagaagaacctgtctcgctcccctggtaaagatatcgttagatctgagcccagcgggcccatttcaacaatcaacccctgtcctccatgcaaggagtgtcacaaatgcccagctcctaacctcgagggtggaccatccgtcttcatcttccctccaaatatcaaggatgtactcatgatctccctgacacccaaggtcacgtgtgtggtggtggatgtgagcgaggatgacccagacgtccagatcagctggtttgtgaacaacgtggaagtacacacagctcagacacaaacccatagagaggattacaacagtactatccgggtggtcagcaccctccccatccagcaccaggactggatgagtggcaaggagttcaaatgcaaggtcaacaacaaagacctcccatcacccatcgagagaaccatctcaaaaattaaagggctagtcagagctccacaagtatacatcttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagtcctggactctgacggttcttacttcatatatagcaagctcaatatgaaaacaagcaagtgggagaaaacagattccttctcatgcaacgtgagacacgagggtctgaaaaattactacctgaagaagaccatctcccggtctccgggtaaatgagctagctgg

SEQ ID NO: 28 - protein, 527 aa, mouse
MYRMQLLSCIALSLALVTNSGGSHHHHHHGMASMTGGQQMGRDLYDDDDKEPRIPKPSTPPGSSCPPGNILGGPSVFIFPPKPKDALMISLTPKVTCVVVDVSEDDPDVHVSWFVDNKEVHTAWTQPREAQYNSTFRVVSALPIQHQDWMRGKEFKCKVNNKALPAPIERTISKPKGRAQTPQVYTIPPPREQMSKKKVSLTCLVTNFFSEAISVEWERNGELEQDYKNTPPILDSDGTYFLYSKLTVDTDSWLQGEIFTCSVVHEALHNHHTQKNLSRSPGKDIVRSEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 29 - DNA, 1557 bp, mouse
atgtacaggatgcaactcctgtcttgcattgcactaagtcttgcacttgtcacgaattcggggggttctcatcatcatcatcatcatggtatggcaagcatgactggtggacagcaaatgggtcgggatctgtacgacgatgacgataaggtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaagatatcgttagatctgagcctagaatacccaagcccagtacccccccaggttcttcatgcccacctggtaacatcttgggtggaccatccgtcttcatcttccccccaaagcccaaggatgcactcatgatctccctaacccccaaggttacgtgtgtggtggtggatgtgagcgaggatgacccagatgtccatgtcagctggtttgtggacaacaaagaagtacacacagcctggacacagccccgtgaagctcagtacaacagtaccttccgagtggtcagtgccctccccatccagcaccaggactggatgaggggcaaggagttcaaatgcaaggtcaacaacaaagccctcccagcccccatcgagagaaccatctcaaaacccaaaggaagagcccagacacctcaagtatacaccatacccccacctcgtgaacaaatgtccaagaagaaggttagtctgacctgcctggtcaccaacttcttctctgaagccatcagtgtggagtgggaaaggaacggagaactggagcaggattacaagaacactccacccatcctggactcagatgggacctacttcctctacagcaagctcactgtggatacagacagttggttgcaaggagaaatttttacctgctccgtggtgcatgaggctctccataaccaccacacacagaagaacctgtctcgctcccctggtaaatgagctagctgg

SEQ ID NO: 30 - protein, 515 aa, mouse
MYRMQLLSCIALSLALVTNSGGSHHHHHHGMASMTGGQQMGRDLYDDDDKVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKDIVRSEPRIPKPSTPPGSSCPPGNILGGPSVFIFPPKPKDALMISLTPKVTCVVVDVSEDDPDVHVSWFVDNKEVHTAWTQPREAQYNSTFRVVSALPIQHQDWMRGKEFKCKVNNKALPAPIERTISKPKGRAQTPQVYTIPPPREQMSKKKVSLTCLVTNFFSEAISVEWERNGELEQDYKNTPPILDSDGTYFLYSKLTVDTDSWLQGEIFTCSVVHEALHNHHTQKNLSRSPGK

SEQ ID NO: 31 - DNA, 1575 bp, mouse
atgtacaggatgcaactcctgtcttgcattgcactaagtcttgcacttgtcacgaattcggggggttctcatcatcatcatcatcatggtatggcaagcatgactggtggacagcaaatgggtcgggatctgtacgacgatgacgataaggagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaagatatcgttagatctgagcctagaatacccaagcccagtacccccccaggttcttcatgcccacctggtaacatcttgggtggaccatccgtcttcatcttccccccaaagcccaaggatgcactcatgatctccctaacccccaaggttacgtgtgtggtggtggatgtgagcgaggatgacccagatgtccatgtcagctggtttgtggacaacaaagaagtacacacagcctggacacagccccgtgaagctcagtacaacagtaccttccgagtggtcagtgccctccccatccagcaccaggactggatgaggggcaaggagttcaaatgcaaggtcaacaacaaagccctcccagcccccatcgagagaaccatctcaaaacccaaaggaagagcccagacacctcaagtatacaccatacccccacctcgtgaacaaatgtccaagaagaaggttagtctgacctgcctggtcaccaacttcttctctgaagccatcagtgtggagtgggaaaggaacggagaactggagcaggattacaagaacactccacccatcctggactcagatgggacctacttcctctacagcaagctcactgtggatacagacagttggttgcaaggagaaatttttacctgctccgtggtgcatgaggctctccataaccaccacacacagaagaacctgtctcgctcccctggtaaatgagctagctgg

SEQ ID NO: 32 - protein, 521 aa, mouse
MYRMQLLSCIALSLALVTNSGGSHHHHHHGMASMTGGQQMGRDLYDDDDKEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKDIVRSEPRIPKPSTPPGSSCPPGNILGGPSVFIFPPKPKDALMISLTPKVTCVVVDVSEDDPDVHVSWFVDNKEVHTAWTQPREAQYNSTFRVVSALPIQHQDWMRGKEFKCKVNNKALPAPIERTISKPKGRAQTPQVYTIPPPREQMSKKKVSLTCLVTNFFSEAISVEWERNGELEQDYKNTPPILDSDGTYFLYSKLTVDTDSWLQGEIFTCSVVHEALHNHHTQKNLSRSPGK

SEQ ID NO: 33 - DNA, 1593 bp, mouse
atgtacaggatgcaactcctgtcttgcattgcactaagtcttgcacttgtcacgaattcggggggttctcatcatcatcatcatcatggtatggcaagcatgactggtggacagcaaatgggtcgggatctgtacgacgatgacgataaggagcccagcgggcccatttcaacaatcaacccctgtcctccatgcaaggagtgtcacaaatgcccagctcctaacctcgagggtggaccatccgtcttcatcttccctccaaatatcaaggatgtactcatgatctccctgacacccaaggtcacgtgtgtggtggtggatgtgagcgaggatgacccagacgtccagatcagctggtttgtgaacaacgtggaagtacacacagctcagacacaaacccatagagaggattacaacagtactatccgggtggtcagcaccctccccatccagcaccaggactggatgagtggcaaggagttcaaatgcaaggtcaacaacaaagacctcccatcacccatcgagagaaccatctcaaaaattaaagggctagtcagagctccacaagtatacatcttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagtcctggactctgacggttcttacttcatatatagcaagctcaatatgaaaacaagcaagtgggagaaaacagattccttctcatgcaacgtgagacacgagggtctgaaaaattactacctgaagaagaccatctcccggtctccgggtaaagatatcgttagatctgagcctagaatacccaagcccagtacccccccaggttcttcatgcccacctggtaacatcttgggtggaccatccgtcttcatcttccccccaaagcccaaggatgcactcatgatctccctaacccccaaggttacgtgtgtggtggtggatgtgagcgaggatgacccagatgtccatgtcagctggtttgtggacaacaaagaagtacacacagcctggacacagccccgtgaagctcagtacaacagtaccttccgagtggtcagtgccctccccatccagcaccaggactggatgaggggcaaggagttcaaatgcaaggtcaacaacaaagccctcccagcccccatcgagagaaccatctcaaaacccaaaggaagagcccagacacctcaagtatacaccatacccccacctcgtgaacaaatgtccaagaagaaggttagtctgacctgcctggtcaccaacttcttctctgaagccatcagtgtggagtgggaaaggaacggagaactggagcaggattacaagaacactccacccatcctggactcagatgggacctacttcctctacagcaagctcactgtggatacagacagttggttgcaaggagaaatttttacctgctccgtggtgcatgaggctctccataaccaccacacacagaagaacctgtctcgctcccctggtaaatgagctagctgg

SEQ ID NO: 34 - protein, 527 aa, mouse
MYRMQLLSCIALSLALVTNSGGSHHHHHHGMASMTGGQQMGRDLYDDDDKEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGKDIVRSEPRIPKPSTPPGSSCPPGNILGGPSVFIFPPKPKDALMISLTPKVTCVVVDVSEDDPDVHVSWFVDNKEVHTAWTQPREAQYNSTFRVVSALPIQHQDWMRGKEFKCKVNNKALPAPIERTISKPKGRAQTPQVYTIPPPREQMSKKKVSLTCLVTNFFSEAISVEWERNGELEQDYKNTPPILDSDGTYFLYSKLTVDTDSWLQGEIFTCSVVHEALHNHHTQKNLSRSPGK

SEQ ID NO: 35 - DNA, 1575 bp, mouse
atgtacaggatgcaactcctgtcttgcattgcactaagtcttgcacttgtcacgaattcggggggttctcatcatcatcatcatcatggtatggcaagcatgactggtggacagcaaatgggtcgggatctgtacgacgatgacgataaggagcctagaatacccaagcccagtacccccccaggttcttcatgcccacctggtaacatcttgggtggaccatccgtcttcatcttccccccaaagcccaaggatgcactcatgatctccctaacccccaaggttacgtgtgtggtggtggatgtgagcgaggatgacccagatgtccatgtcagctggtttgtggacaacaaagaagtacacacagcctggacacagccccgtgaagctcagtacaacagtaccttccgagtggtcagtgccctccccatccagcaccaggactggatgaggggcaaggagttcaaatgcaaggtcaacaacaaagccctcccagcccccatcgagagaaccatctcaaaacccaaaggaagagcccagacacctcaagtatacaccatacccccacctcgtgaacaaatgtccaagaagaaggttagtctgacctgcctggtcaccaacttcttctctgaagccatcagtgtggagtgggaaaggaacggagaactggagcaggattacaagaacactccacccatcctggactcagatgggacctacttcctctacagcaagctcactgtggatacagacagttggttgcaaggagaaatttttacctgctccgtggtgcatgaggctctccataaccaccacacacagaagaacctgtctcgctcccctggtaaagatatcgttagatctgagcctagaatacccaagcccagtacccccccaggttcttcatgcccacctggtaacatcttgggtggaccatccgtcttcatcttccccccaaagcccaaggatgcactcatgatctccctaacccccaaggttacgtgtgtggtggtggatgtgagcgaggatgacccagatgtccatgtcagctggtttgtggacaacaaagaagtacacacagcctggacacagccccgtgaagctcagtacaacagtaccttccgagtggtcagtgccctccccatccagcaccaggactggatgaggggcaaggagttcaaatgcaaggtcaacaacaaagccctcccagcccccatcgagagaaccatctcaaaacccaaaggaagagcccagacacctcaagtatacaccatacccccacctcgtgaacaaatgtccaagaagaaggttagtctgacctgcctggtcaccaacttcttctctgaagccatcagtgtggagtgggaaaggaacggagaactggagcaggattacaagaacactccacccatcctggactcagatgggacctacttcctctacagcaagctcactgtggatacagacagttggttgcaaggagaaatttttacctgctccgtggtgcatgaggctctccataaccaccacacacagaagaacctgtctcgctcccctggtaaatgagctagctgg

SEQ ID NO: 36 - protein, 521 aa, mouse
MYRMQLLSCIALSLALVTNSGGSHHHHHHGMASMTGGQQMGRDLYDDDDKEPRIPKPSTPPGSSCPPGNILGGPSVFIFPPKPKDALMISLTPKVTCVVVDVSEDDPDVHVSWFVDNKEVHTAWTQPREAQYNSTFRVVSALPIQHQDWMRGKEFKCKVNNKALPAPIERTISKPKGRAQTPQVYTIPPPREQMSKKKVSLTCLVTNFFSEAISVEWERNGELEQDYKNTPPILDSDGTYFLYSKLTVDTDSWLQGEIFTCSVVHEALHNHHTQKNLSRSPGKDIVRSEPRIPKPSTPPGSSCPPGNILGGPSVFIFPPKPKDALMISLTPKVTCVVVDVSEDDPDVHVSWFVDNKEVHTAWTQPREAQYNSTFRVVSALPIQHQDWMRGKEFKCKVNNKALPAPIERTISKPKGRAQTPQVYTIPPPREQMSKKKVSLTCLVTNFFSEAISVEWERNGELEQDYKNTPPILDSDGTYFLYSKLTVDTDSWLQGEIFTCSVVHEALHNHHTQKNLSRSPGK

SEQ ID NO: 37 - DNA, 1539 bp, mouse
atgtacaggatgcaactcctgtcttgcattgcactaagtcttgcacttgtcacgaattcggggggttctcatcatcatcatcatcatggtatggcaagcatgactggtggacagcaaatgggtcgggatctgtacgacgatgacgataaggtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgaaaccccgggaggagcagatcaacagcactttccgttcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacaaacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaagatatcgttagatctgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgaaaccccgggaggagcagatcaacagcactttccgttcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacaaacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatgagctagctgg

SEQ ID NO: 38 - protein, 509 aa, mouse
MYRMQLLSCIALSLALVTNSGGSHHHHHHGMASMTGGQQMGRDLYDDDDKVPRDCGCKPCICTVPEVSSVFIFPPKPKDVLTITLTPKVTCVVVDISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGKDIVRSVPRDCGCKPCICTVPEVSSVFIFPPKPKDVLTITLTPKVTCVVVDISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGK

SEQ ID NO: 39 - DNA, 1518 bp, mouse
atgtacaggatgcaactcctgtcttgcattgcactaagtcttgcacttgtcacgaattcggggggttctcatcatcatcatcatcatggtatggagcccagcgggcccatttcaacaatcaacccctgtcctccatgcaaggagtgtcacaaatgcccagctcctaacctcgagggtggaccatccgtcttcatcttccctccaaatatcaaggatgtactcatgatctccctgacacccaaggtcacgtgtgtggtggtggatgtgagcgaggatgacccagacgtccggatcagctggtttgtgaacaacgtggaagtacacacagctcagacacaaacccatagagaggattacaacagtactatccgggtggtcagtgccctccccatccagcaccaggactggatgagtggcaaggagttcaaatgcaaggtcaacaacaaagacctcccatcacccatcgagagaaccatctcaaaaattaaagggctagtcagagctccacaagtatacatcttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagtcctggactctgacggttcttacttcatatacagcaagctcgatataaaaacaagcaagtgggagaaaacagattccttctcatgcaacgtgagacacgagggtctgaaaaattactacctgaagaagaccatctcccggtctccgggtaaagatatcgttagatctgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgaaaccccgggaggagcagatcaacagcactttccgttcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacaaacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatgagctagctgg

SEQ ID NO: 40 - protein, 502 aa, mouse
MYRMQLLSCIALSLALVTNSGGSHHHHHHGMEPSGPISTINPCPPCKECHKCPAPNLEGGPSVFIFPPNIKDVLMISLTPKVTCVVVDVSEDDPDVRISWFVNNVEVHTAQTQTHREDYNSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRSPGKDIVRSVPRDCGCKPCICTVPEVSSVFIFPPKPKDVLTITLTPKVTCVVVDISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGK

SEQ ID NO: 41 - DNA, 1572 bp, mouse
atgtacaggatgcaactcctgtcttgcattgcactaagtcttgcacttgtcacgaattcggggggttctcatcatcatcatcatcatggtatggcaagcatgactggtggacagcaaatgggtcgggatctgtacgacgatgacgataaggagcccagagtgcccataacacagaacccctgtcctccactcaaagagtgtcccccatgcgcagctccagacctcttgggtggaccatccgtcttcatcttccctccaaagatcaaggatgtactcatgatctccctgagccccatggtcacatgtgtggtggtggatgtgagcgaggatgacccagacgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacagagccctcccatcccccatcgagaaaaccatctcaaaacccagagggccagtaagagctccacaggtatatgtcttgcctccaccagcagaagagatgactaagaaagagttcagtctgacctgcatgatcacaggcttcttacctgccgaaattgctgtggactggaccagcaatgggcgtacagagcaaaactacaagaacaccgcaacagtcctggactctgatggttcttacttcatgtacagcaagctcagagtacaaaagagcacttgggaaagaggaagtcttttcgcctgctcagtggtccacgagggtctgcacaatcaccttacgactaagaccatctcccggtctctgggtaaagatatcgttagatctgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgaaaccccgggaggagcagatcaacagcactttccgttcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacaaacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatgagctagctgg

SEQ ID NO: 42 - protein, 520 aa, mouse
MYRMQLLSCIALSLALVTNSGGSHHHHHHGMASMTGGQQMGRDLYDDDDKEPRVPITQNPCPPLKECPPCAAPDLLGGPSVFIFPPKIKDVLMISLSPMVTCVVVDVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKTISRSLGKDIVRSVPRDCGCKPCICTVPEVSSVFIFPPKPKDVLTITLTPKVTCVVVDISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGK

SEQ ID NO: 43 - DNA, 1557 bp, mouse
atgtacaggatgcaactcctgtcttgcattgcactaagtcttgcacttgtcacgaattcggggggttctcatcatcatcatcatcatggtatggcaagcatgactggtggacagcaaatgggtcgggatctgtacgacgatgacgataaggagcctagaatacccaagcccagtacccccccaggttcttcatgcccacctggtaacatcttgggtggaccatccgtcttcatcttccccccaaagcccaaggatgcactcatgatctccctaacccccaaggttacgtgtgtggtggtggatgtgagcgaggatgacccagatgtccatgtcagctggtttgtggacaacaaagaagtacacacagcctggacgcagccccgtgaagctcagtacaacagtaccttccgagtggtcagtgccctccccatccagcaccaggactggatgaggggcaaggagttcaaatgcaaggtcaacaacaaagccctcccagcccccatcgagagaaccatctcaaaacccaaaggaagagcccagacacctcaagtatacaccatacccccacctcgtgaacaaatgtccaagaagaaggttagtctgacctgcctggtcaccaacttcttctctgaagccatcagtgtggagtgggaaaggaacggagaactggagcaggattacaagaacactccacccatcctggactcggatgggacctacttcctctacagcaagctcactgtggatacagacagttggttgcaaggagaaatttttacctgctccgtggtgcatgaggctctccataaccaccacacacagaagaacctgtctcgctcccctggtaaagatatcgttagatctgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgaaaccccgggaggagcagatcaacagcactttccgttcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacaaacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatgagctagctgg

SEQ ID NO: 44 - protein, 515 aa, mouse
MYRMQLLSCIALSLALVTNSGGSHHHHHHGMASMTGGQQMGRDLYDDDDKEPRIPKPSTPPGSSCPPGNILGGPSVFIFPPKPKDALMISLTPKVTCVVVDVSEDDPDVHVSWFVDNKEVHTAWTQPREAQYNSTFRVVSALPIQHQDWMRGKEFKCKVNNKALPAPIERTISKPKGRAQTPQVYTIPPPREQMSKKKVSLTCLVTNFFSEAISVEWERNGELEQDYKNTPPILDSDGTYFLYSKLTVDTDSWLQGEIFTCSVVHEALHNHHTQKNLSRSPGKDIVRSVPRDCGCKPCICTVPEVSSVFIFPPKPKDVLTITLTPKVTCVVVDISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGK

SEQ ID NO: 45 - DNA, 1575 bp, mouse
atgtacaggatgcaactcctgtcttgcattgcactaagtcttgcacttgtcacgaattcggggggttctcatcatcatcatcatcatggtatggcaagcatgactggtggacagcaaatgggtcgggatctgtacgacgatgacgataaggtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgaaaccccgggaggagcagatcaacagcactttccgttcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacaaacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaagatatcgttagatctgagcccagcgggcccatttcaacaatcaacccctgtcctccatgcaaggagtgtcacaaatgcccagctcctaacctcgagggtggaccatccgtcttcatcttccctccaaatatcaaggatgtactcatgatctccctgacacccaaggtcacgtgtgtggtggtggatgtgagcgaggatgacccagacgtccggatcagctggtttgtgaacaacgtggaagtacacacagctcagacacaaacccatagagaggattacaacagtactatccgggtggtcagtgccctccccatccagcaccaggactggatgagtggcaaggagttcaaatgcaaggtcaacaacaaagacctcccatcacccatcgagagaaccatctcaaaaattaaagggctagtcagagctccacaagtatacatcttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagtcctggactctgacggttcttacttcatatacagcaagctcgatataaaaacaagcaagtgggagaaaacagattccttctcatgcaacgtgagacacgagggtctgaaaaattactacctgaagaagaccatctcccggtctccgggtaaatgagctagctgg

SEQ ID NO: 46 - protein, 521 aa, mouse
MYRMQLLSCIALSLALVTNSGGSHHHHHHGMASMTGGQQMGRDLYDDDDKVPRDCGCKPCICTVPEVSSVFIFPPKPKDVLTITLTPKVTCVVVDISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGKDIVRSEPSGPISTINPCPPCKECHKCPAPNLEGGPSVFIFPPNIKDVLMISLTPKVTCVVVDVSEDDPDVRISWFVNNVEVHTAQTQTHREDYNSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRSPGK

SEQ ID NO: 47 - DNA, 1611 bp, mouse
atgtacaggatgcaactcctgtcttgcattgcactaagtcttgcacttgtcacgaattcggggggttctcatcatcatcatcatcatggtatggcaagcatgactggtggacagcaaatgggtcgggatctgtacgacgatgacgataaggagcccagcgggcccatttcaacaatcaacccctgtcctccatgcaaggagtgtcacaaatgcccagctcctaacctcgagggtggaccatccgtcttcatcttccctccaaatatcaaggatgtactcatgatctccctgacacccaaggtcacgtgtgtggtggtggatgtgagcgaggatgacccagacgtccggatcagctggtttgtgaacaacgtggaagtacacacagctcagacacaaacccatagagaggattacaacagtactatccgggtggtcagtgccctccccatccagcaccaggactggatgagtggcaaggagttcaaatgcaaggtcaacaacaaagacctcccatcacccatcgagagaaccatctcaaaaattaaagggctagtcagagctccacaagtatacatcttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagtcctggactctgacggttcttacttcatatacagcaagctcgatataaaaacaagcaagtgggagaaaacagattccttctcatgcaacgtgagacacgagggtctgaaaaattactacctgaagaagaccatctcccggtctccgggtaaagatatcgttagatctgagcccagcgggcccatttcaacaatcaacccctgtcctccatgcaaggagtgtcacaaatgcccagctcctaacctcgagggtggaccatccgtcttcatcttccctccaaatatcaaggatgtactcatgatctccctgacacccaaggtcacgtgtgtggtggtggatgtgagcgaggatgacccagacgtccggatcagctggtttgtgaacaacgtggaagtacacacagctcagacacaaacccatagagaggattacaacagtactatccgggtggtcagtgccctccccatccagcaccaggactggatgagtggcaaggagttcaaatgcaaggtcaacaacaaagacctcccatcacccatcgagagaaccatctcaaaaattaaagggctagtcagagctccacaagtatacatcttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagtcctggactctgacggttcttacttcatatacagcaagctcgatataaaaacaagcaagtgggagaaaacagattccttctcatgcaacgtgagacacgagggtctgaaaaattactacctgaagaagaccatctcccggtctccgggtaaatgagctagctgg

SEQ ID NO: 48 - protein, 533 aa, mouse
MYRMQLLSCIALSLALVTNSGGSHHHHHHGMASMTGGQQMGRDLYDDDDKEPSGPISTINPCPPCKECHKCPAPNLEGGPSVFIFPPNIKDVLMISLTPKVTCVVVDVSEDDPDVRISWFVNNVEVHTAQTQTHREDYNSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRSPGKDIVRSEPSGPISTINPCPPCKECHKCPAPNLEGGPSVFIFPPNIKDVLMISLTPKVTCVVVDVSEDDPDVRISWFVNNVEVHTAQTQTHREDYNSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRSPGK

SEQ ID NO: 49 - DNA, 1608 bp, mouse
atgtacaggatgcaactcctgtcttgcattgcactaagtcttgcacttgtcacgaattcggggggttctcatcatcatcatcatcatggtatggcaagcatgactggtggacagcaaatgggtcgggatctgtacgacgatgacgataaggagcccagagtgcccataacacagaacccctgtcctccactcaaagagtgtcccccatgcgcagctccagacctcttgggtggaccatccgtcttcatcttccctccaaagatcaaggatgtactcatgatctccctgagccccatggtcacatgtgtggtggtggatgtgagcgaggatgacccagacgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacagagccctcccatcccccatcgagaaaaccatctcaaaacccagagggccagtaagagctccacaggtatatgtcttgcctccaccagcagaagagatgactaagaaagagttcagtctgacctgcatgatcacaggcttcttacctgccgaaattgctgtggactggaccagcaatgggcgtacagagcaaaactacaagaacaccgcaacagtcctggactctgatggttcttacttcatgtacagcaagctcagagtacaaaagagcacttgggaaagaggaagtcttttcgcctgctcagtggtccacgagggtctgcacaatcaccttacgactaagaccatctcccggtctctgggtaaagatatcgttagatctgagcccagcgggcccatttcaacaatcaacccctgtcctccatgcaaggagtgtcacaaatgcccagctcctaacctcgagggtggaccatccgtcttcatcttccctccaaatatcaaggatgtactcatgatctccctgacacccaaggtcacgtgtgtggtggtggatgtgagcgaggatgacccagacgtccggatcagctggtttgtgaacaacgtggaagtacacacagctcagacacaaacccatagagaggattacaacagtactatccgggtggtcagtgccctccccatccagcaccaggactggatgagtggcaaggagttcaaatgcaaggtcaacaacaaagacctcccatcacccatcgagagaaccatctcaaaaattaaagggctagtcagagctccacaagtatacatcttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagtcctggactctgacggttcttacttcatatacagcaagctcgatataaaaacaagcaagtgggagaaaacagattccttctcatgcaacgtgagacacgagggtctgaaaaattactacctgaagaagaccatctcccggtctccgggtaaatgagctagctgg

SEQ ID NO: 50 - protein, 532 aa, mouse
MYRMQLLSCIALSLALVTNSGGSHHHHHHGMASMTGGQQMGRDLYDDDDKEPRVPITQNPCPPLKECPPCAAPDLLGGPSVFIFPPKIKDVLMISLSPMVTCVVVDVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKTISRSLGKDIVRSEPSGPISTINPCPPCKECHKCPAPNLEGGPSVFIFPPNIKDVLMISLTPKVTCVVVDVSEDDPDVRISWFVNNVEVHTAQTQTHREDYNSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRSPGK

SEQ ID NO: 51 - DNA, 1593 bp, mouse
atgtacaggatgcaactcctgtcttgcattgcactaagtcttgcacttgtcacgaattcggggggttctcatcatcatcatcatcatggtatggcaagcatgactggtggacagcaaatgggtcgggatctgtacgacgatgacgataaggagcctagaatacccaagcccagtacccccccaggttcttcatgcccacctggtaacatcttgggtggaccatccgtcttcatcttccccccaaagcccaaggatgcactcatgatctccctaacccccaaggttacgtgtgtggtggtggatgtgagcgaggatgacccagatgtccatgtcagctggtttgtggacaacaaagaagtacacacagcctggacgcagccccgtgaagctcagtacaacagtaccttccgagtggtcagtgccctccccatccagcaccaggactggatgaggggcaaggagttcaaatgcaaggtcaacaacaaagccctcccagcccccatcgagagaaccatctcaaaacccaaaggaagagcccagacacctcaagtatacaccatacccccacctcgtgaacaaatgtccaagaagaaggttagtctgacctgcctggtcaccaacttcttctctgaagccatcagtgtggagtgggaaaggaacggagaactggagcaggattacaagaacactccacccatcctggactcggatgggacctacttcctctacagcaagctcactgtggatacagacagttggttgcaaggagaaatttttacctgctccgtggtgcatgaggctctccataaccaccacacacagaagaacctgtctcgctcccctggtaaagatatcgttagatctgagcccagcgggcccatttcaacaatcaacccctgtcctccatgcaaggagtgtcacaaatgcccagctcctaacctcgagggtggaccatccgtcttcatcttccctccaaatatcaaggatgtactcatgatctccctgacacccaaggtcacgtgtgtggtggtggatgtgagcgaggatgacccagacgtccggatcagctggtttgtgaacaacgtggaagtacacacagctcagacacaaacccatagagaggattacaacagtactatccgggtggtcagtgccctccccatccagcaccaggactggatgagtggcaaggagttcaaatgcaaggtcaacaacaaagacctcccatcacccatcgagagaaccatctcaaaaattaaagggctagtcagagctccacaagtatacatcttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagtcctggactctgacggttcttacttcatatacagcaagctcgatataaaaacaagcaagtgggagaaaacagattccttctcatgcaacgtgagacacgagggtctgaaaaattactacctgaagaagaccatctcccggtctccgggtaaatgagctagctgg

SEQ ID NO: 52 - protein, 527 aa, mouse
MYRMQLLSCIALSLALVTNSGGSHHHHHHGMASMTGGQQMGRDLYDDDDKEPRIPKPSTPPGSSCPPGNILGGPSVFIFPPKPKDALMISLTPKVTCVVVDVSEDDPDVHVSWFVDNKEVHTAWTQPREAQYNSTFRVVSALPIQHQDWMRGKEFKCKVNNKALPAPIERTISKPKGRAQTPQVYTIPPPREQMSKKKVSLTCLVTNFFSEAISVEWERNGELEQDYKNTPPILDSDGTYFLYSKLTVDTDSWLQGEIFTCSVVHEALHNHHTQKNLSRSPGKDIVRSEPSGPISTINPCPPCKECHKCPAPNLEGGPSVFIFPPNIKDVLMISLTPKVTCVVVDVSEDDPDVRISWFVNNVEVHTAQTQTHREDYNSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRSPGK

SEQ ID NO: 53 - DNA, 1572 bp, mouse
atgtacaggatgcaactcctgtcttgcattgcactaagtcttgcacttgtcacgaattcggggggttctcatcatcatcatcatcatggtatggcaagcatgactggtggacagcaaatgggtcgggatctgtacgacgatgacgataaggtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgaaaccccgggaggagcagatcaacagcactttccgttcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacaaacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaagatatcgttagatctgagcccagagtgcccataacacagaacccctgtcctccactcaaagagtgtcccccatgcgcagctccagacctcttgggtggaccatccgtcttcatcttccctccaaagatcaaggatgtactcatgatctccctgagccccatggtcacatgtgtggtggtggatgtgagcgaggatgacccagacgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacagagccctcccatcccccatcgagaaaaccatctcaaaacccagagggccagtaagagctccacaggtatatgtcttgcctccaccagcagaagagatgactaagaaagagttcagtctgacctgcatgatcacaggcttcttacctgccgaaattgctgtggactggaccagcaatgggcgtacagagcaaaactacaagaacaccgcaacagtcctggactctgatggttcttacttcatgtacagcaagctcagagtacaaaagagcacttgggaaagaggaagtcttttcgcctgctcagtggtccacgagggtctgcacaatcaccttacgactaagaccatctcccggtctctgggtaaatgagctagctgg

SEQ ID NO: 54 - protein, 520 aa, mouse
MYRMQLLSCIALSLALVTNSGGSHHHHHHGMASMTGGQQMGRDLYDDDDKVPRDCGCKPCICTVPEVSSVFIFPPKPKDVLTITLTPKVTCVVVDISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGKDIVRSEPRVPITQNPCPPLKECPPCAAPDLLGGPSVFIFPPKIKDVLMISLSPMVTCVVVDVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKTISRSLGK

SEQ ID NO: 55 - DNA, 1608 bp, mouse
atgtacaggatgcaactcctgtcttgcattgcactaagtcttgcacttgtcacgaattcggggggttctcatcatcatcatcatcatggtatggcaagcatgactggtggacagcaaatgggtcgggatctgtacgacgatgacgataaggagcccagcgggcccatttcaacaatcaacccctgtcctccatgcaaggagtgtcacaaatgcccagctcctaacctcgagggtggaccatccgtcttcatcttccctccaaatatcaaggatgtactcatgatctccctgacacccaaggtcacgtgtgtggtggtggatgtgagcgaggatgacccagacgtccggatcagctggtttgtgaacaacgtggaagtacacacagctcagacacaaacccatagagaggattacaacagtactatccgggtggtcagtgccctccccatccagcaccaggactggatgagtggcaaggagttcaaatgcaaggtcaacaacaaagacctcccatcacccatcgagagaaccatctcaaaaattaaagggctagtcagagctccacaagtatacatcttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagtcctggactctgacggttcttacttcatatacagcaagctcgatataaaaacaagcaagtgggagaaaacagattccttctcatgcaacgtgagacacgagggtctgaaaaattactacctgaagaagaccatctcccggtctccgggtaaagatatcgttagatctgagcccagagtgcccataacacagaacccctgtcctccactcaaagagtgtcccccatgcgcagctccagacctcttgggtggaccatccgtcttcatcttccctccaaagatcaaggatgtactcatgatctccctgagccccatggtcacatgtgtggtggtggatgtgagcgaggatgacccagacgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacagagccctcccatcccccatcgagaaaaccatctcaaaacccagagggccagtaagagctccacaggtatatgtcttgcctccaccagcagaagagatgactaagaaagagttcagtctgacctgcatgatcacaggcttcttacctgccgaaattgctgtggactggaccagcaatgggcgtacagagcaaaactacaagaacaccgcaacagtcctggactctgatggttcttacttcatgtacagcaagctcagagtacaaaagagcacttgggaaagaggaagtcttttcgcctgctcagtggtccacgagggtctgcacaatcaccttacgactaagaccatctcccggtctctgggtaaatgagctagctgg

SEQ ID NO: 56 - protein, 532 aa, mouse
MYRMQLLSCIALSLALVTNSGGSHHHHHHGMASMTGGQQMGRDLYDDDDKEPSGPISTINPCPPCKECHKCPAPNLEGGPSVFIFPPNIKDVLMISLTPKVTCVVVDVSEDDPDVRISWFVNNVEVHTAQTQTHREDYNSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRSPGKDIVRSEPRVPITQNPCPPLKECPPCAAPDLLGGPSVFIFPPKIKDVLMISLSPMVTCVVVDVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKTISRSLGK

SEQ ID NO: 57 - DNA, 1605 bp, mouse
atgtacaggatgcaactcctgtcttgcattgcactaagtcttgcacttgtcacgaattcggggggttctcatcatcatcatcatcatggtatggcaagcatgactggtggacagcaaatgggtcgggatctgtacgacgatgacgataaggagcccagagtgcccataacacagaacccctgtcctccactcaaagagtgtcccccatgcgcagctccagacctcttgggtggaccatccgtcttcatcttccctccaaagatcaaggatgtactcatgatctccctgagccccatggtcacatgtgtggtggtggatgtgagcgaggatgacccagacgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacagagccctcccatcccccatcgagaaaaccatctcaaaacccagagggccagtaagagctccacaggtatatgtcttgcctccaccagcagaagagatgactaagaaagagttcagtctgacctgcatgatcacaggcttcttacctgccgaaattgctgtggactggaccagcaatgggcgtacagagcaaaactacaagaacaccgcaacagtcctggactctgatggttcttacttcatgtacagcaagctcagagtacaaaagagcacttgggaaagaggaagtcttttcgcctgctcagtggtccacgagggtctgcacaatcaccttacgactaagaccatctcccggtctctgggtaaagatatcgttagatctgagcccagagtgcccataacacagaacccctgtcctccactcaaagagtgtcccccatgcgcagctccagacctcttgggtggaccatccgtcttcatcttccctccaaagatcaaggatgtactcatgatctccctgagccccatggtcacatgtgtggtggtggatgtgagcgaggatgacccagacgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacagagccctcccatcccccatcgagaaaaccatctcaaaacccagagggccagtaagagctccacaggtatatgtcttgcctccaccagcagaagagatgactaagaaagagttcagtctgacctgcatgatcacaggcttcttacctgccgaaattgctgtggactggaccagcaatgggcgtacagagcaaaactacaagaacaccgcaacagtcctggactctgatggttcttacttcatgtacagcaagctcagagtacaaaagagcacttgggaaagaggaagtcttttcgcctgctcagtggtccacgagggtctgcacaatcaccttacgactaagaccatctcccggtctctgggtaaatgagctagctgg

SEQ ID NO: 58 - protein, 531 aa, mouse
MYRMQLLSCIALSLALVTNSGGSHHHHHHGMASMTGGQQMGRDLYDDDDKEPRVPITQNPCPPLKECPPCAAPDLLGGPSVFIFPPKIKDVLMISLSPMVTCVVVDVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKTISRSLGKDIVRSEPRVPITQNPCPPLKECPPCAAPDLLGGPSVFIFPPKIKDVLMISLSPMVTCVVVDVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKTISRSLGK

SEQ ID NO: 59 - DNA, 1590 bp, mouse
atgtacaggatgcaactcctgtcttgcattgcactaagtcttgcacttgtcacgaattcggggggttctcatcatcatcatcatcatggtatggcaagcatgactggtggacagcaaatgggtcgggatctgtacgacgatgacgataaggagcctagaatacccaagcccagtacccccccaggttcttcatgcccacctggtaacatcttgggtggaccatccgtcttcatcttccccccaaagcccaaggatgcactcatgatctccctaacccccaaggttacgtgtgtggtggtggatgtgagcgaggatgacccagatgtccatgtcagctggtttgtggacaacaaagaagtacacacagcctggacgcagccccgtgaagctcagtacaacagtaccttccgagtggtcagtgccctccccatccagcaccaggactggatgaggggcaaggagttcaaatgcaaggtcaacaacaaagccctcccagcccccatcgagagaaccatctcaaaacccaaaggaagagcccagacacctcaagtatacaccatacccccacctcgtgaacaaatgtccaagaagaaggttagtctgacctgcctggtcaccaacttcttctctgaagccatcagtgtggagtgggaaaggaacggagaactggagcaggattacaagaacactccacccatcctggactcggatgggacctacttcctctacagcaagctcactgtggatacagacagttggttgcaaggagaaatttttacctgctccgtggtgcatgaggctctccataaccaccacacacagaagaacctgtctcgctcccctggtaaagatatcgttagatctgagcccagagtgcccataacacagaacccctgtcctccactcaaagagtgtcccccatgcgcagctccagacctcttgggtggaccatccgtcttcatcttccctccaaagatcaaggatgtactcatgatctccctgagccccatggtcacatgtgtggtggtggatgtgagcgaggatgacccagacgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacagagccctcccatcccccatcgagaaaaccatctcaaaacccagagggccagtaagagctccacaggtatatgtcttgcctccaccagcagaagagatgactaagaaagagttcagtctgacctgcatgatcacaggcttcttacctgccgaaattgctgtggactggaccagcaatgggcgtacagagcaaaactacaagaacaccgcaacagtcctggactctgatggttcttacttcatgtacagcaagctcagagtacaaaagagcacttgggaaagaggaagtcttttcgcctgctcagtggtccacgagggtctgcacaatcaccttacgactaagaccatctcccggtctctgggtaaatgagctagctgg

SEQ ID NO: 60 - protein, 526 aa, mouse
MYRMQLLSCIALSLALVTNSGGSHHHHHHGMASMTGGQQMGRDLYDDDDKEPRIPKPSTPPGSSCPPGNILGGPSVFIFPPKPKDALMISLTPKVTCVVVDVSEDDPDVHVSWFVDNKEVHTAWTQPREAQYNSTFRVVSALPIQHQDWMRGKEFKCKVNNKALPAPIERTISKPKGRAQTPQVYTIPPPREQMSKKKVSLTCLVTNFFSEAISVEWERNGELEQDYKNTPPILDSDGTYFLYSKLTVDTDSWLQGEIFTCSVVHEALHNHHTQKNLSRSPGKDIVRSEPRVPITQNPCPPLKECPPCAAPDLLGGPSVFIFPPKIKDVLMISLSPMVTCVVVDVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKTISRSLGK

SEQ ID NO: 61 - DNA, 1557 bp, mouse
atgtacaggatgcaactcctgtcttgcattgcactaagtcttgcacttgtcacgaattcggggggttctcatcatcatcatcatcatggtatggcaagcatgactggtggacagcaaatgggtcgggatctgtacgacgatgacgataaggtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgaaaccccgggaggagcagatcaacagcactttccgttcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacaaacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaagatatcgttagatctgagcctagaatacccaagcccagtacccccccaggttcttcatgcccacctggtaacatcttgggtggaccatccgtcttcatcttccccccaaagcccaaggatgcactcatgatctccctaacccccaaggttacgtgtgtggtggtggatgtgagcgaggatgacccagatgtccatgtcagctggtttgtggacaacaaagaagtacacacagcctggacgcagccccgtgaagctcagtacaacagtaccttccgagtggtcagtgccctccccatccagcaccaggactggatgaggggcaaggagttcaaatgcaaggtcaacaacaaagccctcccagcccccatcgagagaaccatctcaaaacccaaaggaagagcccagacacctcaagtatacaccatacccccacctcgtgaacaaatgtccaagaagaaggttagtctgacctgcctggtcaccaacttcttctctgaagccatcagtgtggagtgggaaaggaacggagaactggagcaggattacaagaacactccacccatcctggactcggatgggacctacttcctctacagcaagctcactgtggatacagacagttggttgcaaggagaaatttttacctgctccgtggtgcatgaggctctccataaccaccacacacagaagaacctgtctcgctcccctggtaaatgagctagctgg

SEQ ID NO: 62 - protein, 515 aa, mouse
MYRMQLLSCIALSLALVTNSGGSHHHHHHGMASMTGGQQMGRDLYDDDDKVPRDCGCKPCICTVPEVSSVFIFPPKPKDVLTITLTPKVTCVVVDISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGKDIVRSEPRIPKPSTPPGSSCPPGNILGGPSVFIFPPKPKDALMISLTPKVTCVVVDVSEDDPDVHVSWFVDNKEVHTAWTQPREAQYNSTFRVVSALPIQHQDWMRGKEFKCKVNNKALPAPIERTISKPKGRAQTPQVYTIPPPREQMSKKKVSLTCLVTNFFSEAISVEWERNGELEQDYKNTPPILDSDGTYFLYSKLTVDTDSWLQGEIFTCSVVHEALHNHHTQKNLSRSPGK

SEQ ID NO: 63 - DNA, 1593 bp, mouse
atgtacaggatgcaactcctgtcttgcattgcactaagtcttgcacttgtcacgaattcggggggttctcatcatcatcatcatcatggtatggcaagcatgactggtggacagcaaatgggtcgggatctgtacgacgatgacgataaggagcccagcgggcccatttcaacaatcaacccctgtcctccatgcaaggagtgtcacaaatgcccagctcctaacctcgagggtggaccatccgtcttcatcttccctccaaatatcaaggatgtactcatgatctccctgacacccaaggtcacgtgtgtggtggtggatgtgagcgaggatgacccagacgtccggatcagctggtttgtgaacaacgtggaagtacacacagctcagacacaaacccatagagaggattacaacagtactatccgggtggtcagtgccctccccatccagcaccaggactggatgagtggcaaggagttcaaatgcaaggtcaacaacaaagacctcccatcacccatcgagagaaccatctcaaaaattaaagggctagtcagagctccacaagtatacatcttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagtcctggactctgacggttcttacttcatatacagcaagctcgatataaaaacaagcaagtgggagaaaacagattccttctcatgcaacgtgagacacgagggtctgaaaaattactacctgaagaagaccatctcccggtctccgggtaaagatatcgttagatctgagcctagaatacccaagcccagtacccccccaggttcttcatgcccacctggtaacatcttgggtggaccatccgtcttcatcttccccccaaagcccaaggatgcactcatgatctccctaacccccaaggttacgtgtgtggtggtggatgtgagcgaggatgacccagatgtccatgtcagctggtttgtggacaacaaagaagtacacacagcctggacgcagccccgtgaagctcagtacaacagtaccttccgagtggtcagtgccctccccatccagcaccaggactggatgaggggcaaggagttcaaatgcaaggtcaacaacaaagccctcccagcccccatcgagagaaccatctcaaaacccaaaggaagagcccagacacctcaagtatacaccatacccccacctcgtgaacaaatgtccaagaagaaggttagtctgacctgcctggtcaccaacttcttctctgaagccatcagtgtggagtgggaaaggaacggagaactggagcaggattacaagaacactccacccatcctggactcggatgggacctacttcctctacagcaagctcactgtggatacagacagttggttgcaaggagaaatttttacctgctccgtggtgcatgaggctctccataaccaccacacacagaagaacctgtctcgctcccctggtaaatgagctagctgg

SEQ ID NO: 64 - protein, 527 aa, mouse
MYRMQLLSCIALSLALVTNSGGSHHHHHHGMASMTGGQQMGRDLYDDDDKEPSGPISTINPCPPCKECHKCPAPNLEGGPSVFIFPPNIKDVLMISLTPKVTCVVVDVSEDDPDVRISWFVNNVEVHTAQTQTHREDYNSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRSPGKDIVRSEPRIPKPSTPPGSSCPPGNILGGPSVFIFPPKPKDALMISLTPKVTCVVVDVSEDDPDVHVSWFVDNKEVHTAWTQPREAQYNSTFRVVSALPIQHQDWMRGKEFKCKVNNKALPAPIERTISKPKGRAQTPQVYTIPPPREQMSKKKVSLTCLVTNFFSEAISVEWERNGELEQDYKNTPPILDSDGTYFLYSKLTVDTDSWLQGEIFTCSVVHEALHNHHTQKNLSRSPGK

SEQ ID NO: 65 - DNA, 1590 bp, mouse
atgtacaggatgcaactcctgtcttgcattgcactaagtcttgcacttgtcacgaattcggggggttctcatcatcatcatcatcatggtatggcaagcatgactggtggacagcaaatgggtcgggatctgtacgacgatgacgataaggagcccagagtgcccataacacagaacccctgtcctccactcaaagagtgtcccccatgcgcagctccagacctcttgggtggaccatccgtcttcatcttccctccaaagatcaaggatgtactcatgatctccctgagccccatggtcacatgtgtggtggtggatgtgagcgaggatgacccagacgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacagagccctcccatcccccatcgagaaaaccatctcaaaacccagagggccagtaagagctccacaggtatatgtcttgcctccaccagcagaagagatgactaagaaagagttcagtctgacctgcatgatcacaggcttcttacctgccgaaattgctgtggactggaccagcaatgggcgtacagagcaaaactacaagaacaccgcaacagtcctggactctgatggttcttacttcatgtacagcaagctcagagtacaaaagagcacttgggaaagaggaagtcttttcgcctgctcagtggtccacgagggtctgcacaatcaccttacgactaagaccatctcccggtctctgggtaaagatatcgttagatctgagcctagaatacccaagcccagtacccccccaggttcttcatgcccacctggtaacatcttgggtggaccatccgtcttcatcttccccccaaagcccaaggatgcactcatgatctccctaacccccaaggttacgtgtgtggtggtggatgtgagcgaggatgacccagatgtccatgtcagctggtttgtggacaacaaagaagtacacacagcctggacgcagccccgtgaagctcagtacaacagtaccttccgagtggtcagtgccctccccatccagcaccaggactggatgaggggcaaggagttcaaatgcaaggtcaacaacaaagccctcccagcccccatcgagagaaccatctcaaaacccaaaggaagagcccagacacctcaagtatacaccatacccccacctcgtgaacaaatgtccaagaagaaggttagtctgacctgcctggtcaccaacttcttctctgaagccatcagtgtggagtgggaaaggaacggagaactggagcaggattacaagaacactccacccatcctggactcggatgggacctacttcctctacagcaagctcactgtggatacagacagttggttgcaaggagaaatttttacctgctccgtggtgcatgaggctctccataaccaccacacacagaagaacctgtctcgctcccctggtaaatgagctagctgg

SEQ ID NO: 66 - protein, 526 aa, mouse
MYRMQLLSCIALSLALVTNSGGSHHHHHHGMASMTGGQQMGRDLYDDDDKEPRVPITQNPCPPLKECPPCAAPDLLGGPSVFIFPPKIKDVLMISLSPMVTCVVVDVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKTISRSLGKDIVRSEPRIPKPSTPPGSSCPPGNILGGPSVFIFPPKPKDALMISLTPKVTCVVVDVSEDDPDVHVSWFVDNKEVHTAWTQPREAQYNSTFRVVSALPIQHQDWMRGKEFKCKVNNKALPAPIERTISKPKGRAQTPQVYTIPPPREQMSKKKVSLTCLVTNFFSEAISVEWERNGELEQDYKNTPPILDSDGTYFLYSKLTVDTDSWLQGEIFTCSVVHEALHNHHTQKNLSRSPGK

SEQ ID NO: 67 - DNA, 1575 bp, mouse
atgtacaggatgcaactcctgtcttgcattgcactaagtcttgcacttgtcacgaattcggggggttctcatcatcatcatcatcatggtatggcaagcatgactggtggacagcaaatgggtcgggatctgtacgacgatgacgataaggagcctagaatacccaagcccagtacccccccaggttcttcatgcccacctggtaacatcttgggtggaccatccgtcttcatcttccccccaaagcccaaggatgcactcatgatctccctaacccccaaggttacgtgtgtggtggtggatgtgagcgaggatgacccagatgtccatgtcagctggtttgtggacaacaaagaagtacacacagcctggacgcagccccgtgaagctcagtacaacagtaccttccgagtggtcagtgccctccccatccagcaccaggactggatgaggggcaaggagttcaaatgcaaggtcaacaacaaagccctcccagcccccatcgagagaaccatctcaaaacccaaaggaagagcccagacacctcaagtatacaccatacccccacctcgtgaacaaatgtccaagaagaaggttagtctgacctgcctggtcaccaacttcttctctgaagccatcagtgtggagtgggaaaggaacggagaactggagcaggattacaagaacactccacccatcctggactcggatgggacctacttcctctacagcaagctcactgtggatacagacagttggttgcaaggagaaatttttacctgctccgtggtgcatgaggctctccataaccaccacacacagaagaacctgtctcgctcccctggtaaagatatcgttagatctgagcctagaatacccaagcccagtacccccccaggttcttcatgcccacctggtaacatcttgggtggaccatccgtcttcatcttccccccaaagcccaaggatgcactcatgatctccctaacccccaaggttacgtgtgtggtggtggatgtgagcgaggatgacccagatgtccatgtcagctggtttgtggacaacaaagaagtacacacagcctggacgcagccccgtgaagctcagtacaacagtaccttccgagtggtcagtgccctccccatccagcaccaggactggatgaggggcaaggagttcaaatgcaaggtcaacaacaaagccctcccagcccccatcgagagaaccatctcaaaacccaaaggaagagcccagacacctcaagtatacaccatacccccacctcgtgaacaaatgtccaagaagaaggttagtctgacctgcctggtcaccaacttcttctctgaagccatcagtgtggagtgggaaaggaacggagaactggagcaggattacaagaacactccacccatcctggactcggatgggacctacttcctctacagcaagctcactgtggatacagacagttggttgcaaggagaaatttttacctgctccgtggtgcatgaggctctccataaccaccacacacagaagaacctgtctcgctcccctggtaaatgagctagctgg

SEQ ID NO: 68 - protein, 521 aa, mouse; identical to SEQ ID NO: 36

SEQ ID NO: 69 - DNA, 1569 bp, human
atgtacaggatgcaactcctgtcttgcattgcactaagtcttgcacttgtcacgaattcggggggttctcatcatcatcatcatcatggtatggcaagcatgactggtggacagcaaatgggtcgggatctgtacgacgatgacgataaggagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaagatatcgttagatctgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaatgagctagctgg

SEQ ID NO: 70 - protein, 519 aa, human
MYRMQLLSCIALSLALVTNSGGSHHHHHHGMASMTGGQQMGRDLYDDDDKEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKDIVRSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - DNA, 1557 bp, human
atgtacaggatgcaactcctgtcttgcattgcactaagtcttgcacttgtcacgaattcggggggttctcatcatcatcatcatcatggtatggcaagcatgactggtggacagcaaatgggtcgggatctgtacgacgatgacgataaggagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgtcgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatctccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaagatatcgttagatctgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaatgagctagctgg

SEQ ID NO: 72 - protein, 515 aa, human
MYRMQLLSCIALSLALVTNSGGSHHHHHHGMASMTGGQQMGRDLYDDDDKERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGKDIVRSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - DNA, 1710 bp, human
atgtacaggatgcaactcctgtcttgcattgcactaagtcttgcacttgtcacgaattcggggggttctcatcatcatcatcatcatggtatggcaagcatgactggtggacagcaaatgggtcgggatctgtacgacgatgacgataaggagctcaaaaccccacttggtgacacaactcacacatgcccacggtgcccagagcccaaatcttgtgacacacctcccccgtgcccacggtgcccagagcccaaatcttgtgacacacctcccccatgcccacggtgcccagagcccaaatcttgtgacacacctcccccgtgcccaaggtgcccagcacctgaactcctgggaggaccgtcagtcttcctcttccccccaaaacccaaggatacccttatgatttcccggacccctgaggtcacgtgcgtggtggtggacgtgagccacgaagaccccgaggtccagttcaagtggtacgtggacggcgtggaggtgcataatgccaagacaaagccgcgggaggagcagtacaacagcacgttccgtgtggtcagcgtcctcaccgtcctgcaccaggactggctgaacggcaaggagtacaagtgcaaggtctccaacaaagccctcccagcccccatcgagaaaaccatctccaaaaccaaaggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaagatatcgttagatctgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaatgagctagctgg

SEQ ID NO: 74 - protein, 566 aa, human
MYRMQLLSCIALSLALVTNSGGSHHHHHHGMASMTGGQQMGRDLYDDDDKELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKDIVRSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - DNA, 1560 bp, human
atgtacaggatgcaactcctgtcttgcattgcactaagtcttgcacttgtcacgaattcggggggttctcatcatcatcatcatcatggtatggcaagcatgactggtggacagcaaatgggtcgggatctgtacgacgatgacgataaggagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagagcctctccctgtctctgggtaaagatatcgttagatctgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaatgagctagctgg

SEQ ID NO: 76 - protein, 516 aa, human
MYRMQLLSCIALSLALVTNSGGSHHHHHHGMASMTGGQQMGRDLYDDDDKESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKDIVRSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - DNA, 1557 bp, human
atgtacaggatgcaactcctgtcttgcattgcactaagtcttgcacttgtcacgaattcggggggttctcatcatcatcatcatcatggtatggcaagcatgactggtggacagcaaatgggtcgggatctgtacgacgatgacgataaggagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaagatatcgttagatctgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgtcgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatctccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaatgagctagctgg

SEQ ID NO: 78 - protein, 515 aa, human
MYRMQLLSCIALSLALVTNSGGSHHHHHHGMASMTGGQQMGRDLYDDDDKEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKDIVRSERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 79 - DNA, 1545 bp, human
atgtacaggatgcaactcctgtcttgcattgcactaagtcttgcacttgtcacgaattcggggggttctcatcatcatcatcatcatggtatggcaagcatgactggtggacagcaaatgggtcgggatctgtacgacgatgacgataaggagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgtcgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatctccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaagatatcgttagatctgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgtcgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatctccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaatgagctagctgg

SEQ ID NO: 80 - protein, 511 aa, human
MYRMQLLSCIALSLALVTNSGGSHHHHHHGMASMTGGQQMGRDLYDDDDKERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGKDIVRSERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 81 - DNA, 1698 bp, human
atgtacaggatgcaactcctgtcttgcattgcactaagtcttgcacttgtcacgaattcggggggttctcatcatcatcatcatcatggtatggcaagcatgactggtggacagcaaatgggtcgggatctgtacgacgatgacgataaggagctcaaaaccccacttggtgacacaactcacacatgcccacggtgcccagagcccaaatcttgtgacacacctcccccgtgcccacggtgcccagagcccaaatcttgtgacacacctcccccatgcccacggtgcccagagcccaaatcttgtgacacacctcccccgtgcccaaggtgcccagcacctgaactcctgggaggaccgtcagtcttcctcttccccccaaaacccaaggatacccttatgatttcccggacccctgaggtcacgtgcgtggtggtggacgtgagccacgaagaccccgaggtccagttcaagtggtacgtggacggcgtggaggtgcataatgccaagacaaagccgcgggaggagcagtacaacagcacgttccgtgtggtcagcgtcctcaccgtcctgcaccaggactggctgaacggcaaggagtacaagtgcaaggtctccaacaaagccctcccagcccccatcgagaaaaccatctccaaaaccaaaggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaagatatcgttagatctgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgtcgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatctccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaatgagctagctgg

SEQ ID NO: 82 - protein, 562 aa, human
MYRMQLLSCIALSLALVTNSGGSHHHHHHGMASMTGGQQMGRDLYDDDDKELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKDIVRSERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 83 - DNA, 1548 bp, human
atgtacaggatgcaactcctgtcttgcattgcactaagtcttgcacttgtcacgaattcggggggttctcatcatcatcatcatcatggtatggcaagcatgactggtggacagcaaatgggtcgggatctgtacgacgatgacgataaggagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagagcctctccctgtctctgggtaaagatatcgttagatctgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgtcgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatctccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaatgagctagctgg

SEQ ID NO: 84 - protein, 512 aa, human
MYRMQLLSCIALSLALVTNSGGSHHHHHHGMASMTGGQQMGRDLYDDDDKESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKDIVRSERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 85 - DNA, 1710 bp, human
atgtacaggatgcaactcctgtcttgcattgcactaagtcttgcacttgtcacgaattcggggggttctcatcatcatcatcatcatggtatggcaagcatgactggtggacagcaaatgggtcgggatctgtacgacgatgacgataaggagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaagatatcgttagatctgagctcaaaaccccacttggtgacacaactcacacatgcccacggtgcccagagcccaaatcttgtgacacacctcccccgtgcccacggtgcccagagcccaaatcttgtgacacacctcccccatgcccacggtgcccagagcccaaatcttgtgacacacctcccccgtgcccaaggtgcccagcacctgaactcctgggaggaccgtcagtcttcctcttccccccaaaacccaaggatacccttatgatttcccggacccctgaggtcacgtgcgtggtggtggacgtgagccacgaagaccccgaggtccagttcaagtggtacgtggacggcgtggaggtgcataatgccaagacaaagccgcgggaggagcagtacaacagcacgttccgtgtggtcagcgtcctcaccgtcctgcaccaggactggctgaacggcaaggagtacaagtgcaaggtctccaacaaagccctcccagcccccatcgagaaaaccatctccaaaaccaaaggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaatgagctagctgg

SEQ ID NO: 86 - protein, 566 aa, human
MYRMQLLSCIALSLALVTNSGGSHHHHHHGMASMTGGQQMGRDLYDDDDKEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKDIVRSELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 87 - DNA, 1698 bp, human
atgtacaggatgcaactcctgtcttgcattgcactaagtcttgcacttgtcacgaattcggggggttctcatcatcatcatcatcatggtatggcaagcatgactggtggacagcaaatgggtcgggatctgtacgacgatgacgataaggagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgtcgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatctccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaagatatcgttagatctgagctcaaaaccccacttggtgacacaactcacacatgcccacggtgcccagagcccaaatcttgtgacacacctcccccgtgcccacggtgcccagagcccaaatcttgtgacacacctcccccatgcccacggtgcccagagcccaaatcttgtgacacacctcccccgtgcccaaggtgcccagcacctgaactcctgggaggaccgtcagtcttcctcttccccccaaaacccaaggatacccttatgatttcccggacccctgaggtcacgtgcgtggtggtggacgtgagccacgaagaccccgaggtccagttcaagtggtacgtggacggcgtggaggtgcataatgccaagacaaagccgcgggaggagcagtacaacagcacgttccgtgtggtcagcgtcctcaccgtcctgcaccaggactggctgaacggcaaggagtacaagtgcaaggtctccaacaaagccctcccagcccccatcgagaaaaccatctccaaaaccaaaggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaatgagctagctgg

SEQ ID NO: 88 - protein, 562 aa, human
MYRMQLLSCIALSLALVTNSGGSHHHHHHGMASMTGGQQMGRDLYDDDDKERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGKDIVRSELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 89 - DNA, 1851 bp, human
atgtacaggatgcaactcctgtcttgcattgcactaagtcttgcacttgtcacgaattcggggggttctcatcatcatcatcatcatggtatggcaagcatgactggtggacagcaaatgggtcgggatctgtacgacgatgacgataaggagctcaaaaccccacttggtgacacaactcacacatgcccacggtgcccagagcccaaatcttgtgacacacctcccccgtgcccacggtgcccagagcccaaatcttgtgacacacctcccccatgcccacggtgcccagagcccaaatcttgtgacacacctcccccgtgcccaaggtgcccagcacctgaactcctgggaggaccgtcagtcttcctcttccccccaaaacccaaggatacccttatgatttcccggacccctgaggtcacgtgcgtggtggtggacgtgagccacgaagaccccgaggtccagttcaagtggtacgtggacggcgtggaggtgcataatgccaagacaaagccgcgggaggagcagtacaacagcacgttccgtgtggtcagcgtcctcaccgtcctgcaccaggactggctgaacggcaaggagtacaagtgcaaggtctccaacaaagccctcccagcccccatcgagaaaaccatctccaaaaccaaaggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaagatatcgttagatctgagctcaaaaccccacttggtgacacaactcacacatgcccacggtgcccagagcccaaatcttgtgacacacctcccccgtgcccacggtgcccagagcccaaatcttgtgacacacctcccccatgcccacggtgcccagagcccaaatcttgtgacacacctcccccgtgcccaaggtgcccagcacctgaactcctgggaggaccgtcagtcttcctcttccccccaaaacccaaggatacccttatgatttcccggacccctgaggtcacgtgcgtggtggtggacgtgagccacgaagaccccgaggtccagttcaagtggtacgtggacggcgtggaggtgcataatgccaagacaaagccgcgggaggagcagtacaacagcacgttccgtgtggtcagcgtcctcaccgtcctgcaccaggactggctgaacggcaaggagtacaagtgcaaggtctccaacaaagccctcccagcccccatcgagaaaaccatctccaaaaccaaaggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaatgagctagctgg

SEQ ID NO: 90 - protein, 613 aa, human [sequence omitted]

SEQ ID NO: 91 - DNA, 1701 bp, human
atgtacaggatgcaactcctgtcttgcattgcactaagtcttgcacttgtcacgaattcggggggttctcatcatcatcatcatcatggtatggcaagcatgactggtggacagcaaatgggtcgggatctgtacgacgatgacgataaggagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagagcctctccctgtctctgggtaaagatatcgttagatctgagctcaaaaccccacttggtgacacaactcacacatgcccacggtgcccagagcccaaatcttgtgacacacctcccccgtgcccacggtgcccagagcccaaatcttgtgacacacctcccccatgcccacggtgcccagagcccaaatcttgtgacacacctcccccgtgcccaaggtgcccagcacctgaactcctgggaggaccgtcagtcttcctcttccccccaaaacccaaggatacccttatgatttcccggacccctgaggtcacgtgcgtggtggtggacgtgagccacgaagaccccgaggtccagttcaagtggtacgtggacggcgtggaggtgcataatgccaagacaaagccgcgggaggagcagtacaacagcacgttccgtgtggtcagcgtcctcaccgtcctgcaccaggactggctgaacggcaaggagtacaagtgcaaggtctccaacaaagccctcccagcccccatcgagaaaaccatctccaaaaccaaaggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaatgagctagctgg

SEQ ID NO: 92 - protein, 563 aa, human
MYRMQLLSCIALSLALVTNSGGSHHHHHHGMASMTGGQQMGRDLYDDDDKESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKDIVRSELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 93 - DNA, 1560 bp, human
atgtacaggatgcaactcctgtcttgcattgcactaagtcttgcacttgtcacgaattcggggggttctcatcatcatcatcatcatggtatggcaagcatgactggtggacagcaaatgggtcgggatctgtacgacgatgacgataaggagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaagatatcgttagatctgagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagagcctctccctgtctctgggtaaatgagctagctgg

SEQ ID NO: 94 - protein, 516 aa, human
MYRMQLLSCIALSLALVTNSGGSHHHHHHGMASMTGGQQMGRDLYDDDDKEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKDIVRSESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 95 - DNA, 1548 bp, human
atgtacaggatgcaactcctgtcttgcattgcactaagtcttgcacttgtcacgaattcggggggttctcatcatcatcatcatcatggtatggcaagcatgactggtggacagcaaatgggtcgggatctgtacgacgatgacgataaggagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgtcgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatctccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaagatatcgttagatctgagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagagcctctccctgtctctgggtaaatgagctagctgg

SEQ ID NO: 96 - protein, 512 aa, human
MYRMQLLSCIALSLALVTNSGGSHHHHHHGMASMTGGQQMGRDLYDDDDKERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGKDIVRSESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 97 - DNA, 1701 bp, human
atgtacaggatgcaactcctgtcttgcattgcactaagtcttgcacttgtcacgaattcggggggttctcatcatcatcatcatcatggtatggcaagcatgactggtggacagcaaatgggtcgggatctgtacgacgatgacgataaggagctcaaaaccccacttggtgacacaactcacacatgcccacggtgcccagagcccaaatcttgtgacacacctcccccgtgcccacggtgcccagagcccaaatcttgtgacacacctcccccatgcccacggtgcccagagcccaaatcttgtgacacacctcccccgtgcccaaggtgcccagcacctgaactcctgggaggaccgtcagtcttcctcttccccccaaaacccaaggatacccttatgatttcccggacccctgaggtcacgtgcgtggtggtggacgtgagccacgaagaccccgaggtccagttcaagtggtacgtggacggcgtggaggtgcataatgccaagacaaagccgcgggaggagcagtacaacagcacgttccgtgtggtcagcgtcctcaccgtcctgcaccaggactggctgaacggcaaggagtacaagtgcaaggtctccaacaaagccctcccagcccccatcgagaaaaccatctccaaaaccaaaggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaagatatcgttagatctgagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagagcctctccctgtctctgggtaaatgagctagctgg

SEQ ID NO: 98 - protein, 563 aa, human
MYRMQLLSCIALSLALVTNSGGSHHHHHHGMASMTGGQQMGRDLYDDDDKELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKDIVRSESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 99 - DNA, 1551 bp, human
atgtacaggatgcaactcctgtcttgcattgcactaagtcttgcacttgtcacgaattcggggggttctcatcatcatcatcatcatggtatggcaagcatgactggtggacagcaaatgggtcgggatctgtacgacgatgacgataaggagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagagcctctccctgtctctgggtaaagatatcgttagatctgagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagagcctctccctgtctctgggtaaatgagctagctgg

SEQ ID NO: 100 - protein, 513 aa, human
MYRMQLLSCIALSLALVTNSGGSHHHHHHGMASMTGGQQMGRDLYDDDDKESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKDIVRSESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 101 - DNA, 23 bp, Artificial Sequence
  Primer
tagatctagcaagcccacgtgcc

SEQ ID NO: 102 - DNA, 27 bp, Artificial Sequence
  Primer
ccagctagctcatttacccggagagcg

SEQ ID NO: 103 - DNA, 19 bp, Artificial Sequence
  Primer
acgaattcggggggttctc

SEQ ID NO: 104 - DNA, 37 bp, Artificial Sequence
  Primer
ctagatctaacgatatctttacccggagagcgggaga

SEQ ID NO: 105 - DNA, 26 bp, Artificial Sequence
  Primer
ttagatctagcaagcccacgtgccca

SEQ ID NO: 106 - DNA, 44 bp, Artificial Sequence
  Primer
cagctagctcaataatagtaataatatttacccggagagcggga

SEQ ID NO: 107 - DNA, 26 bp, Artificial Sequence
  Primer
ttagatctgtgcccagggattgtggt

SEQ ID NO: 108 - DNA, 30 bp, Artificial Sequence
  Primer
cagctagctcatttaccaggagagtgggag

SEQ ID NO: 109 - DNA, 26 bp, Artificial Sequence
  Primer
ttagatctgagcccagagggcccaca

SEQ ID NO: 110 - DNA, 26 bp, Artificial Sequence
  Primer
cagctagctcatttacccggagtccg

SEQ ID NO: 111 - DNA, 26 bp, Artificial Sequence
  Primer
ttagatctgagcccagcgggcccatt

SEQ ID NO: 112 - DNA, 26 bp, Artificial Sequence
  Primer
cagctagctcatttacccggagaccg

SEQ ID NO: 113 - DNA, 30 bp, Artificial Sequence
  Primer
ttagatctgagcctagaatacccaagccca

SEQ ID NO: 114 - DNA, 27 bp, Artificial Sequence
  Primer
cagctagctcatttaccaggggagcga

SEQ ID NO: 115 - DNA, 19 bp, Artificial Sequence
  Primer
acgaattcggggggttctc

SEQ ID NO: 116 - DNA, 39 bp, Artificial Sequence
  Primer
ctagatctaacgatatctttaccaggagagtgggagagg

SEQ ID NO: 117 - DNA, 34 bp, Artificial Sequence
  Primer
ctagatctaacgatatctttacccggagtccggg

SEQ ID NO: 118 - DNA, 33 bp, Artificial Sequence
  Primer
ctagatctaacgatatctttacccggagaccgg

SEQ ID NO: 119 - DNA, 36 bp, Artificial Sequence
  Primer
ctagatctaacgatatctttaccaggggagcgagac

SEQ ID NO: 120 - DNA, 26 bp, Artificial Sequence
  Primer
ttagatctgtgcccagggattgtggt

SEQ ID NO: 121 - DNA, 30 bp, Artificial Sequence; identical to SEQ ID NO: 108
  Primer

SEQ ID NO: 122 - DNA, 26 bp, Artificial Sequence
  Primer
ttagatctgagcccagcgggcccatt

SEQ ID NO: 123 - DNA, 26 bp, Artificial Sequence
  Primer
cagctagctcatttacccggagaccg

SEQ ID NO: 124 - DNA, 26 bp, Artificial Sequence
  Primer
ttagatctgagcccagagtgcccata

SEQ ID NO: 125 - DNA, 27 bp, Artificial Sequence
  Primer
cagctagctcatttacccagagaccgg

SEQ ID NO: 126 - DNA, 30 bp, Artificial Sequence; identical to SEQ ID NO: 113
  Primer

SEQ ID NO: 127 - DNA, 27 bp, Artificial Sequence
  Primer
cagctagctcatttaccaggggagcga

SEQ ID NO: 128 - DNA, 19 bp, Artificial Sequence
  Primer
acgaattcggggggttctc

SEQ ID NO: 129 - DNA, 39 bp, Artificial Sequence; identical to SEQ ID NO: 116
  Primer

SEQ ID NO: 130 - DNA, 33 bp, Artificial Sequence; identical to SEQ ID NO: 118
  Primer

SEQ ID NO: 131 - DNA, 36 bp, Artificial Sequence
  Primer
ctagatctaacgatatctttacccagagaccgggag

SEQ ID NO: 132 - DNA, 36 bp, Artificial Sequence; identical to SEQ ID NO: 119
  Primer

SEQ ID NO: 133 - DNA, 29 bp, Artificial Sequence
  Primer
ttagatctgagcccaaatcttgtgacaaa

SEQ ID NO: 134 - DNA, 27 bp, Artificial Sequence
  Primer
cagctagctcatttacccggagacagg

SEQ ID NO: 135 - DNA, 27 bp, Artificial Sequence
  Primer
ttagatctgagcgcaaatgttgtgtcg

SEQ ID NO: 136 - DNA, 27 bp, Artificial Sequence
  Primer
cagctagctcatttacccggagacagg

SEQ ID NO: 137 - DNA, 27 bp, Artificial Sequence
  Primer
ttagatctgagctcaaaaccccacttg

SEQ ID NO: 138 - DNA, 27 bp, Artificial Sequence
  Primer
cagctagctcatttacccggagacagg

SEQ ID NO: 139 - DNA, 29 bp, Artificial Sequence
  Primer
ttagatctgagtccaaatatggtccccca

SEQ ID NO: 140 - DNA, 30 bp, Artificial Sequence
  Primer
cagctagctcatttacccagagacagggag

SEQ ID NO: 141 - DNA, 19 bp, Artificial Sequence
  Primer
acgaattcggggggttctc

SEQ ID NO: 142 - DNA, 36 bp, Artificial Sequence
  Primer
ctagatctaacgatatctttacccggagacagggag

SEQ ID NO: 143 - DNA, 19 bp, Artificial Sequence
  Primer
acgaattcggggggttctc

SEQ ID NO: 144 - DNA, 36 bp, Artificial Sequence; identical to SEQ ID NO: 142
  Primer

SEQ ID NO: 145 - DNA, 19 bp, Artificial Sequence
  Primer
acgaattcggggggttctc

SEQ ID NO: 146 - DNA, 36 bp, Artificial Sequence; identical to SEQ ID NO: 142
  Primer

SEQ ID NO: 147 - DNA, 19 bp, Artificial Sequence
  Primer
acgaattcggggggttctc

SEQ ID NO: 148 - DNA, 36 bp, Artificial Sequence
  Primer
ctagatctaacgatatctttacccagagacagggag